(19) United States
(12) Patent Application Publication
(10) Pub. No.: US 2012/0207744 A1
(43) Pub. Date: Aug. 16, 2012

(54) REPROGRAMMING COMPOSITIONS AND METHODS OF USING THE SAME

(76) Inventors: John D. Mendlein, Leucadia, CA (US); Francine S. Farouz, La Jolla, CA (US); R. Scott Thies, San Diego, CA (US); Daniel Shoemaker, San Diego, CA (US)

(21) Appl. No.: 13/257,291

(22) PCT Filed: Mar. 19, 2010
(86) PCT No.: PCT/US10/28022

§ 371 Date: Apr. 30, 2012

Related U.S. Application Data
(60) Provisional application No. 61/161,705, filed on Mar. 19, 2009.
(60) Provisional application No. 61/171,807, filed on Apr. 22, 2009.
(60) Provisional application No. 61/241,647, filed on Sep. 11, 2009.

(51) Int. Cl. A61K 35/12; C12N 5/074; C12N 5/0735; A61K 38/43; A61K 31/713; A61K 38/02; A61K 39/395; C12N 5/071; A61K 31/7088

(52) U.S. Cl. 424/130.1; 435/377; 424/93.7; 514/44 R; 514/44 A; 514/1.1; 424/94.1

(57) ABSTRACT

The present invention provides compositions and methods of using the compositions to alter the developmental potency of a cell. The present invention provides in vivo and ex vivo cell reprogramming and programming methods suitable for autologous cell therapy and regenerative medicine.

### CROSS REFERENCE TO RELATED APPLICATIONS

This application claims benefit under 35 U.S.C. §119(e) of U.S. Provisional Application No. 61/161,705, filed Mar. 19, 2009; U.S. Provisional Application No. 61/171,807, filed Apr. 22, 2009; and U.S. Provisional Application No. 61/241,647, filed Sep. 11, 2009, each of which is incorporated by reference in its entirety.

### SEQUENCE LISTING

The Sequence Listing associated with this application is provided in text format in lieu of a paper copy, and is hereby incorporated by reference into the specification. The name of the text file containing the Sequence Listing is 320051_{—}451 PC SEQUENCE LISTING.txt. The text file is 582 KB, was created on Mar. 19, 2010, and is being submitted electronically via EFS-Web.

### BACKGROUND

1. Technical Field

The present invention relates generally to compositions and methods of using the same to alter the developmental potency of a cell. The present invention provides cells suitable for autologous cell therapy and in vivo and ex vivo reprogramming and programming of cells.

2. Description of the Related Art

Stem cells are partially of fully undifferentiated cells found in most, if not all, multi-cellular organisms. Stem cells have the ability to self-renew through mitotic cell division and to differentiate into a diverse range of specialized cell types, including but not limited to brain, muscle, liver, pancreatic cells, skin, neural, and blood cells. Stem cells are generally classified as either embryonic stem cells (ESCs), or adult tissue derived-stem cells, depending on the source of the tissue from which they are derived. ESCs are pluripotent and can give rise during development to all derivatives of the three primary germ layers: ectoderm, endoderm and mesoderm. In other words, they can develop into each of the more than 200 cell types of the adult body when given sufficient and necessary stimulation for a specific cell type. Adult stem cells are multipotent and retain the ability to give rise to cells within a given embyronic lineage.

Because stem cells have the potential of developing into specific types of cells and can proliferate indefinitely or undergo renewal for extended periods of time, they hold particular, but so far unrealized, potential in the context of therapeutic applications. Stem cells, whether they are adult or progenitor cells or other cell types, may be used for organ repair and replacement, cell therapies for a variety of diseases including degenerative diseases, gene therapy, and testing of new drugs for toxicities or desired activities.

However, available sources of stem cells, as well as more differentiated cells, useful for experimental and therapeutic applications have been limited, often of poor quality, unsuitable for therapy, and controversial. Further, although ESCs represent promising donor sources for cell transplantation therapies, they face immune rejection after transplantation. In addition, there are a number of controversial ethical issues relating to the use human embryos as a stem cell source.

To date, attempts to generate human cells with a desired cell fate, including pluripotent cells or multipotent cells as well as cells differentiated to a desired fate, from non embryonic sources have focused on genetic and chemical manipulations of somatic cells. These attempts may create cells with pluripotent or multipotent potential, however, such attempts typically require genetic engineering of the cells, and in some cases require the use of chemicals that are potentially toxic or epigenetically altering. Further the “reprogramming” community has largely focused on directly increasing the expression of certain control genes that facilitate pluripotent or multipotent potential by transfecting those genes into cells to derive increased levels of transfected gene product. Thus, the creation of clinical grade human cells of a desired cell fate is thwarted by many factors, including poor cellular or genetic characterization of the cells, long protocols for generating desired cells, impractical generation methods for reproducible therapies, lack of powerful non-genetic modulation agents (or therapies), particularly in vivo but also ex vivo), low frequency or yield of desired cell fate for therapeutic or discovery purposes, and potential mismatches in cell therapy versus patient that lead to undesired conditions.

Thus, there is a significant and unmet need for identifying approaches by which stem cells, particularly clinical or pharmaceutical grade cells, can be directly derived from a patient's somatic cells, a non-embryonic human source or adult human source, and safely used in a cell-based therapy. The inventions described herein overcome these and other limitations of these fields.

### BRIEF SUMMARY

In various embodiments, the present invention contemplates, in part, a method of altering the potency of a cell, comprising contacting the cell with one or more repressors, wherein said one or more repressors modulates at least one component of a cellular pathway associated with the potency of the cell, thereby altering the potency of the cell. In a particular embodiment, the one or more repressors is a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a DNAzyme, a ssDNA, polypeptide or active fragment thereof, an antibody, an intrabody, a transbody, a protein, an enzyme, a peptidomimetic, a peptoid, a transcriptional factor, or a small organic molecule, and the like.

In one embodiment, the present invention provides a method of altering the potency of a cell, comprising contacting the cell with one or more activators, wherein said one or more activators modulates at least one component of a cellular pathway associated with the potency of the cell, thereby altering the potency of the cell. In a particular embodiment, the one or more activators can be any number and/or combination of the following molecules: an antibody or an antibody fragment, an mRNA, a bifunctional antisense oligonucleotide, a dsDNA, a polypeptide or an active fragment thereof, a transcription factor, a peptidomimetic, a peptoid, or a small organic molecule, and the like.

In a particular embodiment, a polypeptide or active fragment thereof is a pluripotency factor or a component of a cellular pathway associate with the potency of a cell. In a certain embodiment, the polypeptide is a transcription factor selected from the group consisting of: transcriptional activators, transcriptional repressors, artificial transcription factors, and hormone binding domain transcription factor fusion polypeptides.

In another particular embodiment, the modulation of at least one component of a cellular pathway associated with the potency of the cell comprises a change in epigenetic state, chromatin structure, transcription, mRNA splicing, post-transcriptional modification, mRNA stability and/or half-life, translation, post-translational modification, protein stability and/or half-life and/or protein activity of the at least one component. In a related embodiment, the component being modulated is selected from the group consisting of: a members of the Hedgehog pathway, components of the Wnt pathway, receptor tyrosine kinases, non-receptor tyrosine kinases, TGF family members, BMP family members, Jak/Stat family members, Hox family members, Sox family members, Klf family members, Myc family members, Oct family members, components of a chromatin modulation pathway, components of a histone modulation pathway, miRNAs regulated by pluripotency factors, miRNAs that regulate pluripotency factors and/or components of cellular pathway associated with the developmental potency of a cell, members of the NuRD complex, Polycomb group proteins, SWI/SNF chromatin remodeling enzymes, Ac133, Alp, Atbf1, Axin2, BAF155, bFgf, Bmi1, Boc, C/EBPβ, CD9, Cdon, Cdx-2, c-Kit, c-Myc, Coup-Tf1, Coup-Tf2, Csl, Ctbp, Dax1, Dnmt3A, Dnmt3B, Dnmt3L, Dppa2, Dppa4, Dppa5, Ecat1, Ecat8, Eomes, Eras, Esg1, Esrrb, Fbx15, Fgf2, Fgf4, Flt3, Foxc1, Foxd3, Fzd9, Gbx2, Gcnf, Gdf10, Gdf3, Gdf5, Grb2, Groucho, Gsh1, Hand1, Hdac1, Hdac2, HesX1, His-5, HoxA10, HoxA11, HoxB1, HP1α, HP1β, HPV16 E6, HPV16 E7, Irx2, Isl1, Jarid2, Jmjd1a, Jmjd2c, Klf-3, Klf-4, Klf-5, Left Lefty-1, Lefty-2, Lif, Lin-28, Mad 1, Mad3, Mad4, Mafa, Mbd3, Meis1, MeI-18, Meox2, Mta1, Mxi1, Myf5, Myst3, Nac1, Nanog, Neurog2, Ngn3, Nkx2.2, Nodal, Oct-4, Olig2, Onecut, Otx1, Oxt2, Pax5, Pax6, Pdx1, Pias1, Pias2, Pias3, Piasy, REST, Rex-1, Rfx4, Rif1, Rnf2, Rybp, Sal1l4, Sal1l1, Scf, Scgf, Set, Sip1, Skil, Smarcad1, Sox-15, Sox-2, Sox-6, Ssea-1, Ssea-2, Ssea-4, Stat3, Stella, SV40 large T antigen, Tbx3, Tcf1, Tcf2, Tcf3, Tcf4, Tcf-7, Tcf7l1, Tcl1, Tdgf-1, Tert, hTert, Tif1, Tra-1-60, Tra-1-81, Utf-1, Wnt3a, Wnt8a, YY1, Zeb2, Zfhx1b, Zfp281, Zfp57, Zic3, β-catenin, histone acetylases, histone de-acetylases, histone methyltransferases, histone demethylases or substrates, cofactors, co-activators, co-repressors and/or a downstream effectors thereof.

In a certain embodiments, the component being modulated is selected from the group consisting of Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof. In a particular embodiment, the one or more repressors modulates the at least one component by repressing the at least one component, de-repressing a repressor of the at least one component, or repressing an activator of the at least one component. In another particular embodiment, the one or more repressors modulates the at least one component by de-repressing the at least one component, repressing a repressor of the at least one component, or de-repressing an activator of the at least one component. In yet another particular embodiment, the one or more activators modulates the at least one component by activating the at least one component, activating a repressor of a repressor of the at least one component, or activating an activator of the at least one component.

In certain embodiments, the potency of the cell is altered to decrease potency (e.g., wherein the altered cell is in a more differentiated state after the at least one component is modulated).

In other certain embodiments, the potency of the cell is altered to increase potency (e.g., the altered cell is in a less differentiated state after the at least one component is modulated).

In a particular embodiment, one or more repressors modulates the at least one component by repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity or de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In another particular embodiment, one or more activators modulates the at least one component by activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity or activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In certain embodiments, a component from the cellular pathway selected from a Wnt pathway, a Hedgehog pathway, a TGF-b pathway, a receptor tyrosine kinase pathway, a Jak/STAT pathway, and a Notch pathway is being modulated. In a particular embodiment, one or more repressors modulates the at least one component by repressing the at least one component, de-repressing a repressor of the at least one component, or repressing an activator of the at least one component. In another particular embodiment, one or more repressors modulates the at least one component by de-repressing the at least one component, repressing a repressor of the at least one component, or de-repressing an activator of the at least one component. In yet another particular embodiment, one or more activators modulates the at least one component by activating the at least one component, activating a repressor of a repressor of the at least one component, or activating an activator of the at least one component.

In certain embodiments, the potency of the cell is altered to decrease potency (e.g., wherein the altered cell is in a more differentiated state after the at least one component is modulated).

In other certain embodiments, the potency of the cell is altered to increase potency (e.g., the altered cell is in a less differentiated state after the at least one component is modulated).

In various embodiments, the potency of a cell is modulated. In a particular embodiment, the cell is a stem cell or a progenitor cell. In certain embodiments, the cell is an embryonic stem or progenitor cell. In other certain embodiments, the cell is an adult stem cell or progenitor cell.

In another particular embodiment, the cell is an adult somatic cell. In certain embodiments, the somatic cell is selected from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an epithelial cell, an immune cell or an endothelial cell.

In one embodiment, the cell is a mammalian cell. In another embodiment, the cell is a human cell.

In a particular embodiment, the cell is associated with an in vivo tissue in a subject. In a related particular embodiment, the tissue is selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In one embodiment, the cell is contacted with the one or more repressors ex vivo, and is administered to a subject.

In another embodiment, the cell is associated with an in vivo tissue in a subject. In a particular embodiment, the tissue is selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue. In a certain embodiment, the cell is contacted with the one or more activators ex vivo, and wherein the method further comprises the step of administering the cell to a subject.

In a particular embodiment, the subject is suffering from cancer and/or a disease, disorder, or condition associated with pancreatic tissue, neural tissue, cardiac tissue, bone marrow, muscle tissue, bone tissue, skin tissue, liver tissue, hair follicles, vascular tissue, adipose tissue, lung tissue, or kidney tissue. In another particular embodiment, the subject is about to undergo, is undergoing, or has undergone a surgical procedure. In yet another particular embodiment, the subject is about to undergo, is undergoing, or has undergone a tissue or organ transplant procedure. In certain embodiments, the tissue or organ transplant procedure is selected from a liver transplant, heart transplant, neural tissue transplant, kidney transplant, bone marrow transplant, stem cell transplant, skin transplant, lung transplant.

In various other embodiments, the present invention contemplates, in part, a method of increasing the totipotency a cell, comprising contacting the cell with a composition comprising one or more repressors, wherein the one or more repressors modulates at least one component of a cellular pathway associated with the totipotency of the cell, thereby increasing the totipotency of the cell. In yet various other embodiments, the present invention contemplates, in part, a method of increasing the pluripotency a cell, comprising contacting the cell with one or more repressors, wherein the one or more repressors modulates at least one component of a cellular pathway associated with the pluripotency of the cell, thereby increasing the pluripotency of the cell. In still yet various other embodiments, the present invention contemplates, in part, a method of increasing the multipotency a cell, comprising contacting the cell with one or more repressors, wherein the one or more repressors modulates at least one component of a cellular pathway associated with the multipotency of the cell, thereby increasing the multipotency of the cell. In a particular embodiment, the one or more repressors modulates the at least one component by de-repressing the at least one component, repressing a repressor of the at least one component, or derepressing an activator of the at least one component. In another particular embodiment, a method of increasing the potency of a cell further comprises a step of contacting the totipotent cell, the pluripotent cell or the multipotent cell with a second wherein the second composition modulates the at least one component by repressing the at least one component, de-repressing a repressor of the at least one component, or repressing an activator of the at least one component, wherein the totipotency, pluripotency or multipotency of the cell is decreased, and wherein the cell is differentiated into a mature somatic cell.

In a particular embodiment, the mature somatic cell is selected from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an epithelial cell, an immune cell, and an endothelial cell.

In various other embodiments, the present invention contemplates, in part, a method of increasing the totipotency a cell, comprising contacting the cell with a composition comprising one or more activators, wherein the one or more activators modulates at least one component of a cellular pathway associated with the totipotency of the cell, thereby increasing the totipotency of the cell. In yet various other embodiments, the present invention contemplates, in part, a method of increasing the pluripotency a cell, comprising contacting the cell with a composition comprising one or moreactivators, wherein the one or more activators modulates at least one component of a cellular pathway associated with the pluripotency of the cell, thereby increasing the pluripotency of the cell. In still yet various other embodiments, the present invention contemplates, in part, a method of increasing the multipotency a cell, comprising contacting the cell with a composition comprising one or moreactivators, wherein the one or more activators modulates at least one component of a cellular pathway associated with the multipotency of the cell, thereby increasing the multipotency of the cell. In a particular embodiment, the one or more activators modulates the at least one component by activating the at least one component, activating a repressor of a repressor of the at least one component, or activating an activator of the at least one component. In a certain embodiment a method of increasing the potency of a cell comprises a further step of contacting the totipotent cell, the pluripotent cell or the multipotent cell with a second composition wherein the second composition modulates the at least one component by activating a repressor of the at least one component or activating an activator of a repressor of the at least one component, wherein the totipotency, pluripotency or multipotency of the cell is decreased, and wherein the cell is differentiated into a mature somatic cell.

In a particular embodiment, the second composition comprises one or more repressors of at least one component of a cellular pathway associated with the potency of the cell. In another particular embodiment, the second composition comprises one or more activators of at least one component of a cellular pathway associated with the potency of the cell.

In a certain embodiment, the mature somatic cell is selected from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an eptithelial cell, an immune cell, and an endothelial cell.

In various other embodiments, the present invention contemplates, in part, a method of reprogramming a cell, comprising contacting the cell with one or more repressors, wherein the one or more repressors modulates at least one component of a cellular pathway associated with the reprogramming of a cell, thereby reprogramming the cell.

In various other embodiments, the present invention contemplates, in part, a method of in vivo cell therapy, comprising administering to a subject a composition comprising one or more repressors, wherein the one or more repressors modulates at least one component of a cellular pathway associated with the pluripotency of a cell.

In various other embodiments, the present invention contemplates, in part, a method of ex vivo cell therapy, comprising the steps of isolating a cell; contacting the cell with a composition comprising one or more repressors, wherein the one or more repressors modulates at least one component of a cellular pathway associated with the pluripotency of the cell; and administering the cell to a subject.

In a particular embodiment, the one or more repressors modulates the at least one component by de-repressing the at least one component, repressing a repressor of the at least one component, or derepressing an activator of the at least one component. In a related embodiment, the modulation of the at least one component comprises a change in epigenetic state, chromatin structure, transcription, mRNA splicing, post-transcriptional modification, mRNA stability and/or half-life, translation, post-translational modification, protein stability and/or half-life and/or protein activity of the at least one component, wherein the at least one component is selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In another particular embodiment, the one or more repressors modulates the at least one component by repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity or de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In various other embodiments, the present invention contemplates, in part, a method of reprogramming a cell, comprising contacting the cell with a composition comprising one or more activators, wherein the one or more activators modulates at least one component of a cellular pathway associated with the reprogramming of a cell, thereby re-programming the cell.

In various other embodiments, the present invention contemplates, in part, a method of in vivo cell therapy, comprising administering to a subject a composition comprising one or more activators, wherein the one or more activators modulates at least one component of a cellular pathway associated with the pluripotency of a cell.

In various other embodiments, the present invention contemplates, in part, a method of ex vivo cell therapy, comprising the steps of isolating a cell; contacting the cell with a composition comprising one or more activators, wherein the one or more activator modulates at least one component of a cellular pathway associated with the pluripotency of the cell; and administering the cell to a subject.

In a particular embodiment, the one or more activators modulates the at least one component by activating the at least one component, activating a repressor of a repressor of the at least one component, or activating an activator of the at least one component. In a related embodiment, the modulation of the at least one component comprises a change in epigenetic state, chromatin structure, transcription, mRNA splicing, post-transcriptional modification, mRNA stability and/or half-life, translation, post-translational modification, protein stability and/or half-life and/or protein activity of the at least one component, wherein the at least one component is selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In a certain embodiment, the one or more activators modulates the at least one component by activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity or activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In various other embodiments, the present invention contemplates, in part, a culture comprising a cell, a composition comprising one or more repressors in contact with the cell, and a pharmaceutically acceptable culture medium wherein the one or more repressors modulates at least one component of a cellular pathway associated with the pluripotency of the cell. In a particular embodiment, the one or more repressors modulates the at least one component by de-repressing the at least one component, repressing a repressor of the at least one component, or derepressing an activator of the at least one component.

In another particular embodiment, the composition comprises conditioned medium from another culture, wherein said medium comprises a component of a Wnt pathway, a Hedgehog pathway, a TGF-b pathway, a receptor tyrosine kinase pathway, a Jak/STAT pathway, or a Notch pathway.

In a certain embodiment, the at least one component is secreted.

In one embodiment, the modulation of the at least one component comprises a change in epigenetic state, chromatin structure, transcription, mRNA splicing, post-transcriptional modification, mRNA stability and/or half-life, translation, post-translational modification, protein stability and/or half-life and/or protein activity of the at least one component, wherein the at least one component is selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In a certain embodiment, the one or more repressors modulates the at least one component by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity. In a related embodiment, the composition further comprises a secondary agent, wherein the secondary agent increases the efficacy of the one or more repressors. In a certain related embodiment, the secondary agent is PD0325901.

In various other embodiments, the present invention contemplates, in part, a culture comprising a cell, a composition comprising one or more activators in contact with the cell, and a pharmaceutically acceptable culture medium wherein the one or more activators modulates at least component of a cellular pathway associated with the pluripotency of the cell. In a particular embodiment, the one or more activators modulates the at least one component by a) activating the at least one component; b) activating a repressor of a repressor of the at least one component; or c) activating an activator of the at least one component. In another particular embodiment, the composition comprises conditioned medium from another culture, wherein said medium comprises a component of a Wnt pathway, a Hedgehog pathway, a TGF-b pathway, a receptor tyrosine kinase pathway, a Jak/STAT pathway, or a Notch pathway.

In a certain embodiment, the at least one component is secreted.

In a particular embodiment, the modulation of the at least one component comprises a change in epigenetic state, chromatin structure, transcription, mRNA splicing, post-transcriptional modification, mRNA stability and/or half-life, translation, post-translational modification, protein stability and/or half-life and/or protein activity of the at least one component, wherein the at least one component is selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In another particular embodiment, the one or more activators modulates the at least one component by a) activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity; or b) activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In one embodiment, a culture comprises a cell that is initially an adult somatic cell. In particular embodiment, the cell is a mammalian cell. In another particular embodiment, the cell is a human cell.

In another embodiment, the somatic cell is selected from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an epithelial cell, an immune cell or an endothelial cell. In a particular embodiment, the somatic cell is isolated from an in vivo tissue in a subject.

In another particular embodiment, the tissue is selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In a certain embodiment, the cell is obtained from a cell line.

In various other embodiments, the present invention contemplates, in part, an implant device, comprising a biocompatible material and a cell, and a composition comprising one or more repressors, wherein the one or more repressors modulates at least one component of a cellular pathway associated with the pluripotency of the cell. In various other embodiments, the present invention contemplates, in part, an implant device, comprising a biocompatible material and a cell, and a composition comprising one or more activators, wherein the one or more activators modulates at least one component of a cellular pathway associated with the pluripotency of the cell.

In a particular embodiment, an implant comprises a cell obtained from an in vivo tissue of a subject.

In another particular embodiment, the device is implanted in a patent.

In one embodiment, the in vivo tissue of a subject is allogenic to a patient. In another embodiment, the in vivo tissue of a subject is syngenic to a patient. In another embodiment, the in vivo tissue of a subject is autogenic to a patient. In another embodiment, the in vivo tissue of a subject is xenogenic to a patient.

In a particular embodiment, the implant comprises a biocompatible matrix or an artificial tissue matrix.

In various other embodiments, the present invention contemplates, in part, a pharmaceutical composition comprising one or more of the foregoing culture systems.

In various other embodiments, the present invention contemplates, in part, a method of ex vivo cell therapy, comprising administering the composition of claim 101 to a subject.

In various other embodiments, the present invention contemplates, in part, a composition comprising one or more repressors and a cell, wherein the one or more repressors modulates at least one component of a cellular pathway associated with the pluripotency of a cell. In a particular embodiment, the one or more repressors is a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a DNAzyme, a ssDNA, polypeptide or active fragment thereof, an antibody, an intrabody, a transbody, a protein, an enzyme, a peptidomimetic, a peptoid, a transcriptional factor, or a small organic molecule, and the like.

In various other embodiments, the present invention contemplates, in part, a composition comprising one or more activators and a cell, wherein the one or more activators modulates at least one component of a cellular pathway associated with the pluripotency of a cell. In a particular embodiment, the one or more activators is Illustrative activators of the present invention can be any number and/or combination of the following molecules: an antibody or an antibody fragment, an mRNA, a bifunctional antisense oligonucleotide, a dsDNA, a polypeptide or an active fragment thereof, a transcription factor, a peptidomimetic, a peptoid, or a small organic molecule, and the like.

In one embodiment, a polypeptide or active fragment thereof is a pluripotency factor or a component of a cellular pathway associate with the potency of a cell. In a related embodiment, the polypeptide is a transcription factor selected from the group consisting of: transcriptional activators, transcriptional repressors, artificial transcription factors, and hormone binding domain transcription factor fusion polypeptides.

In a particular embodiment, the modulation of the at least one component comprises a change in epigenetic state, chromatin structure, transcription, mRNA splicing, post-transcriptional modification, mRNA stability and/or half-life, translation, post-translational modification, protein stability and/or half-life and/or protein activity of the at least one component.

In a certain embodiment, the at least one component is selected from the group consisting of: members of the Hedgehog pathway, components of the Wnt pathway, receptor tyrosine kinases, non-receptor tyrosine kinases, TGF family members, BMP family members, Jak/Stat family members, Hox family members, Sox family members, Klf family members, Myc family members, Oct family members, components of a chromatin modulation pathway, components of a histone modulation pathway, miRNAs regulated by pluripotency factors, miRNAs that regulate pluripotency factors and/or components of cellular pathway associated with the developmental potency of a cell, members of the NuRD complex, Polycomb group proteins, SWI/SNF chromatin remodeling enzymes, Ac133, Alp, Atbf1, Axin2, BAF155, bFgf, Bmi1, Boc, C/EBPβ, CD9, Cdon, Cdx-2, c-Kit, c-Myc, Coup-Tf1, Coup-Tf2, Csl, Ctbp, Dax1, Dnmt3A, Dnmt3B, Dnmt3L, Dppa2, Dppa4, Dppa5, Ecat1, Ecat8, Eomes, Eras, Esg1, Esrrb, Fbx15, Fgf2, Fgf4, Flt3, Foxc1, Foxd3, Fzd9, Gbx2, Gcnf, Gdf10, Gdf3, GdfS, Grb2, Groucho, Gsh1, Hand 1, Hdac1, Hdac2, HesX1, Hic-5, HoxA10, HoxA11, HoxB1, HP1a, HP1β, HPV16 E6, HPV16 E7, Irx2, Isl1, Jarid2, Jmjd1a, Jmjd2c, Klf-3, Klf-4, Klf-5, Left Lefty-1, Lefty-2, Lif, Lin-28, Mad1, Mad3, Mad4, Mafa, Mbd3, Meis1, MeI-18, Meox2, Mta1, Mxi1, Myf5, Myst3, Nac1, Nanog, Neurog2, Ngn3, Nkx2.2, Nodal, Oct-4, Olig2, Onecut, Otx1, Oxt2, Pax5, Pax6, Pdx1, Pias1, Pias2, Pias3, Piasy, REST, Rex-1, Rfx4, Rif1, Rnf2, Rybp, Sal1l4, Sal1l1, Scf, Scgf, Set, Sip1, Skil, Smarcad1, Sox-15, Sox-2, Sox-6, Ssea-1, Ssea-2, Ssea-4, Stat3, Stella, SV40 large T antigen, Tbx3, Tcf1, Tcf2, Tcf3, Tcf4, Tcf-7, Tcf711, Tcl1, Tdgf-1, Teri, hTert, Tif1, Tra-1-60, Tra-1-81, Utf-1, Wnt3a, Wnt8a, YY1, Zeb2, Zfhx1b, Zfp281, Zfp57, Zic3, β-catenin, histone acetylases, histone de-acetylases, histone methyltransferases, histone demethylases or substrates, cofactors, co-activators, co-repressors and/or a downstream effectors thereof.

In another embodiment, the at least one component selected from the group consisting of: Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In a particular embodiment, the one or more repressors modulates the at least one component by repressing the at least one component, de-repressing a repressor of the at least one component, or repressing an activator of the at least one component.

In another particular embodiment, the one or more repressors modulates the at least one component by de-repressing the at least one component, repressing a repressor of the at least one component, or de-repressing an activator of the at least one component.

In another particular embodiment, the one or more activators modulates the at least one component by activating the at least one component, activating a repressor of a repressor of the at least one component, or activating an activator of the at least one component.

In one embodiment, the pluripotency of the cell is altered to decrease pluripotency (e.g., the altered cell is in a more differentiated state after the at least one component is modulated.). In another embodiment, the pluripotency of the cell is altered to increase pluripotency (e.g., the altered cell is in a less differentiated state after the at least one component is modulated).

In a particular embodiment, the one or more repressors modulates the at least one component by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In another particular embodiment, the one or more activators modulates the at least one component by a) activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity; or b) activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In a certain embodiment, a modulated component belongs to a cellular pathway selected from a Wnt pathway, a Hedgehog pathway, a TGF-b pathway, a receptor tyrosine kinase pathway, a Jak/STAT pathway, and a Notch pathway.

In a particular embodiment, the one or more repressors modulate the at least one component by repressing the at least one component, de-repressing a repressor of the at least one component, or repressing an activator of the at least one component. In another particular embodiment, the repressor modulates the at least one component by de-repressing the at least one component, repressing a repressor of the at least one component, or derepressing an activator of the at least one component. In yet another particular embodiment, the activator modulates the at least one component by activating the at least one component, activating a repressor of a repressor of the at least one component, or activating an activator of the at least one component.

In one embodiment, the pluripotency of the cell is altered to decrease pluripotency (e.g., the altered cell is in a more differentiated state after the at least one component is modulated.). In another embodiment, the pluripotency of the cell is altered to increase pluripotency (e.g., the altered cell is in a less differentiated state after the at least one component is modulated).

In various other embodiments, the present invention contemplates, in part, a composition comprising a repressor and a cell, wherein the repressor modulates the epigenetic state, chromatin structure, transcription, mRNA splicing, post-transcriptional modification, mRNA stability and/or half-life, translation, post-translational modification, protein stability and/or half-life and/or protein activity of a pluripotency factor, wherein the pluripotency factor is the selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

In one embodiment, the pluripotency factor is Oct3/4 and/or Nanog and a target of the repressor is one or more of a member of the NuRD complex, Sin3A, a member of the Pmt complex, Hdac1/2, Mta1/2, or Mbd3.

In another embodiment, the pluripotency factor is Nanog and wherein a target of the repressor is one or more of Tcf1, Tcf3, Tcf4, or Tcf7.

In yet another embodiment, the pluripotency factor is Nanog and wherein a target of the repressor is one or more of Groucho, Ctbp, or Hic-5.

In yet another embodiment, the pluripotency factor is Nanog and wherein the repressor de-represses a member of the Wnt signaling pathway.

In yet another embodiment, the pluripotency factor is Oct3/4 and wherein a target of the repressor is one or more of Cdx-2, Coup-Tf1, or Gcnf.

In yet another embodiment, the pluripotency factor is Oct3/4 and wherein a target of the repressor is one or more of Piasy, Pias1, Pias2, or Pias3.

In yet another embodiment, the pluripotency factor is Sox2 and wherein a target of the repressor is one or more of HP1α, HP1γ, Cdx, Sip1, Zfhx1b, Zeb2, CtBP, p300/CBP or Pcaf.

In yet another embodiment, the pluripotency factor is Sox2 and wherein a target of the repressor is one or more of HP1α, Cdx, or Sip1.

In yet another embodiment, the pluripotency factor is c-Myc and wherein a target of the repressor is one or more of Apc, Mel-18, or HIV-1 tat protein.

In yet another embodiment, the pluripotency factor is c-Myc and wherein a target of the repressor is one or more of Mad1, Mxi1, Mad3, or Mad4.

In a particular embodiment, the one or more repressors is an antibody or antibody fragment thereof, an ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a ssDNA; a polypeptide or active fragment thereof, a peptidomimetic, a peptoid, a small organic molecule, or any combination thereof.

In another particular embodiment, a polypeptide or active fragment thereof is a pluripotency factor or a component of a cellular pathway associate with the potency of a cell. In a related embodiment, the polypeptide is a transcription factor selected from the group consisting of: transcriptional activators, transcriptional repressors, artificial transcription factors, and hormone binding domain transcription factor fusion polypeptides.

In various other embodiments, the present invention contemplates, in part, a method of dedifferentiating a cell to a more pluripotent state, comprising contacting the cell with the composition of claim 103, wherein the one or more repressors modulates a component of a cellular pathway associated with the dedifferentiation of the cell to the pluripotent state, thereby dedifferentiating the cell to the pluripotent state.

In various other embodiments, the present invention contemplates, in part, a method of dedifferentiating a cell to a more pluripotent state, comprising contacting the cell with the composition of claim 105, wherein the one or more activators modulates a component of a cellular pathway associated with the dedifferentiation of the cell to the pluripotent state, thereby dedifferentiating the cell to the pluripotent state.

In various other embodiments, the present invention contemplates, in part, a method of dedifferentiating a cell to a pluripotent state, comprising contacting the cell with one or more repressors selected from a ssRNA, a dsRNA an mRNA, an antisense RNA, a pri-miRNA, an shRNA, an antagomir, an aptamer, an siRNA, a dsDNA, a ssDNA; a polypeptide, a peptidomimetic, or a small organic molecule or any combination thereof, wherein the one or more repressors or activators modulates a component of a cellular pathway associated with the dedifferentiation of the cell to the pluripotent state, thereby dedifferentiating the cell to the pluripotent state.

In various other embodiments, the present invention contemplates, in part, a method of dedifferentiating a cell to a pluripotent state, comprising contacting the cell with one or more activators selected from a ssRNA, a dsRNA an mRNA, an antisense RNA, a pri-miRNA, an shRNA, an antagomir, an aptamer, an siRNA, a dsDNA, a ssDNA; a polypeptide, a peptidomimetic, or a small organic molecule or any combination thereof, wherein the one or more repressors or activators modulates a component of a cellular pathway associated with the dedifferentiation of the cell to the pluripotent state, thereby dedifferentiating the cell to the pluripotent state. In a particular embodiment, the polypeptide or active fragment thereof is a pluripotency factor or a component of a cellular pathway associate with the potency of a cell. In a related embodiment, the polypeptide is a transcription factor selected from the group consisting of: transcriptional activators, transcriptional repressors, artificial transcription factors, and hormone binding domain transcription factor fusion polypeptides.

In another particular embodiment, the one or more repressors or activators are small molecules.

In another particular embodiment, the one or more repressors or activators induce the cell to express at least one pluripotency factor, wherein the at least one pluripotency factor is the selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof, thereby dedifferentiating the cell.

In a certain embodiment, the at least one pluripotency factor is selected from Sox-2, c-Myc, Oct3/4, Klf4, Nanog, and Lin28, thereby dedifferentiating the cell.

### TABLE OF CONTENTS FOR THE DETAILED DESCRIPTION

I. Overview of Somatic Cell Reprogramming
II. Stem Cells of Different Origins
   A. Embryonic Carcinoma Cells (EC)
   B. Mouse Embryonic Stem Cells
   C. Pluripotent Cell Lines Derived from Germ Cells
   D. Human Embryonic Stem Cells
   E. Induced Pluripotent Stem Cells (iPS)
   F. Adult Stem Cells
III. Cells of the Present Invention
   A. Cells Suitable for Reprogramming
   B. Reprogrammed Cells
   C. Programmed Cells
      1. Differentiation of Stem Cells
IV. Epigenetic Modulation: Chromatin Remodeling
   A. Epigenetic Modifications of Stem Cells
   B. Chromatin and Histone Modifications
   C. Histone-Modifying Enzymes
   D. Acetylation
   E. Deacetylation
   F. Lysine Methylation
   G. Lysine Demethylation
   H. Arginine Methylation
   I. Phosphorylation
   J. Ubiquitylation
   K. Deubiquitylation
   L. Proline Isomerization
   M. Deimination
   N. Sumoylation
   O. ADP Ribosylation
   P. Epigenetics and Pluripotency Factors
V. Pluripotency Factors
   A. Oct Family
   B. Sox Family
   C. Klf Family
   D. Myc Family
   E. Nanog
   F. Lin-28
   G. Components
VI. Pluripotency Pathways
   A. Wnt Pathway
   B. Hedgehog Pathway
   C. Notch Pathway
   D. LIF
   E. TGF-beta
   F. FGF Signaling Pathway
   G. PI3K/AKT Signaling Pathway
   H. Grb2/MEK Pathway
   I. PI3K/AKT;MAPK/ERK
VII. Transcriptional Networks Affecting Pluripotency
VIII. Methods to Assess Pluripotency
IX. Repressors and Activators
   A. DNAzymes
   B. RNAi Interference
   C. MicroRNAs
   D. Short Hairpin RNAs
   E. Ribozymes
   F. Antagomirs
   G. Aptamers
   H. Antisense Oligonucleotides
   I. Bifunctional Antisense Oligonucleotides
   J. Locked Nucleic Acids
   K. Peptide Nucleic Acids
   L. Artificial Transcription Factors
   M. Hormone Binding Domain-Transcription Factor Fusion Proteins
   N. Peptidomimetics
   O. Peptoids
   P. Intrabodies
   Q. Transbodies
   R. Small Molecules
   S. Other Repressors and Activators
      1. Repressors and Activators of Sox2
      2. Repressors and Activators of Nanog
      3. Repressors and Activators of Oct-4
      4. Repressors and Activators of Klf4
      5. Repressors and Activators of Myc
      6. Exemplary Indirect Repressors and Activators
X. Polynucleotides
XI. Polypeptides
XII. Antibodies
   A. Antibody Fragments
   B. Humanized Antibodies
   C. Human Antibodies
   D. Antibody Variants
   E. Antibody Derivatives
   F. Selection and Transformation of Host Cells
XIII. Formulations and Pharmaceutical Compositions
XIV. Methods of Delivery
   A. Adenovirus Vectors
   B. Retrovirus Vectors
   C. Adeno-Associated Virus Vectors
   D. Other Viral Vectors as Expression Constructs
   E. Non-Viral Methods
   F. Electroporation
XV. Cell Targeting
XVI. Implants
XVII. Cell Culture and Cell Culture Compositions
   A. Mouse Embryonic Stem Cell Culture
   B. Human Embryonic Stem Cell Culture
   C. Increasing Efficiency of Stem Cell Cloning
   D. Medium Formulations
XVIII. Methods of Use

### DETAILED DESCRIPTION

The present invention generally relates to compositions and methods for altering the potency of a cell and related therapeutic applications involving the same. More particularly, the present invention relates to compositions and methods for altering the potency of a cell by reprogramming or programming the cell by non-genetic means. In various embodiments, altering the developmental potency of a cell is achieved by modulating a component of a cellular pathway associated with determining, establishing, or maintaining the potency of the cell.

A component may be regulated by any of a variety of mechanisms, including modulation (i.e., activation or repression) of a pathway associated with the fate of a cell, such as a transcriptional pathway that regulates the expression of a gene that affects cell potency, a cellular reprogramming pathway, a dedifferentiation pathway, a programming pathway, a differentiation pathway, a maintenance pathway, a WNT pathway, a Hedgehog pathway, or a Notch signaling pathway. Accordingly, illustrative examples of components of cellular pathways associated with the potency of a cell, include, but are not limited to members of Wnt pathways, Hedgehog pathways, Notch signaling pathways, receptor tyrosine kinase pathways, non-receptor tyrosine kinase pathways, PI3K/AKT pathways, Grb2/MEK pathways, MAPK/ERK pathways, TGF-β pathways, BMP pathways, GDF pathways, LIF pathways, Jak/Stat pathways, Hox pathways, the Sox gene family, the Klf gene family, the Myc gene family, the Oct gene family, the Lin 28 gene family, the Polycomb group proteins, miRNAs, epigenetic pathways, and chromatin remodeling pathways, which includes histone modification pathways.

The present invention contemplates, in part, to reprogram and program cells in vitro, in vivo or ex vivo, by modulation of specific cellular pathways, either directly or indirectly, using polynucleotide-, polypeptide- and/or small molecule-based approaches. As used herein, the terms “reprogramming” or “dedifferentiation” refers to a method of increasing the potency of a cell or dedifferentiating the cell to a less differentiated state. For example, a reprogrammed cell refers to a cell that has an increased cell potency compared to the same cell in the non-reprogrammed state. In other words, a reprogrammed cell is one that is in a less differentiated state than the same cell in a non-reprogrammed state. In certain embodiments, somatic cells are reprogrammed to a pluripotent state. Cells of this type are known as induced pluripotent cells (iPS).

As used herein, the term “programming” or “differentiation” refers to a method of decreasing the potency of a cell or differentiating the cell to a more differentiated state. For example, a programmed cell refers to a cell that has a decreased cell potency compared to the same cell in the reprogrammed state. In other words, a programmed cell is one that is in a more differentiated state than the same cell in a reprogrammed state.

As used herein, the terms “transdifferentiation” or “differentiation plasticity” refers to the notion that somatic stem cells, e.g., adult stem cells, have broadened potency and are able to generate cells of other lineages. For example, a hematopoietic stem cell cultured in such a way as to differentiate into a cell of the neural lineage is said to transdifferentiate or have differentiation plasticity.

In various embodiments, methods of the present invention may be utilized to alter the potency of a cell by modulating one or more components of a cellular pathway that affects cell potency.

As used herein, the term “potency” refers to the sum of all developmental options accessible to the cell (i.e., the developmental potency). One having ordinary skill in the art would recognize that cell potency is a continuum, ranging from the totipotent stem cell to the terminally differentiated cell.

The continuum of cell potency includes, but is not limited to, totipotent cells, pluripotent cells, multipotent cells, oligopotent cells, unipotent cells, and terminally differentiated cells. In the strictest sense, stem cells are either totipotent or pluripotent; thus, being able to give rise to any mature cell type. However, multipotent, oligopotent or unipotent progenitor cells are sometimes referred to as lineage restricted stem cells (e.g., mesenchymal stem cells, adipose tissue derived stem cells, etc.) and/or progenitor cells.

It would also be clear to one having skill in the art that potency can be partially or completely altered to any point along the developmental lineage of a cell (i.e., from totipotent to terminally differentiated cell), regardless of cell lineage. One having ordinary skill in the art would further recognize that terminally differentiated somatic cells may be reprogrammed or dedifferentiated into totipotent, pluripotent, and multipotent cells; thus, providing another source of cells suitable for use as a cell-based therapeutic in various embodiments of the present invention.

As used herein, the term “totipotent” means the ability of a cell to form all cell lineages of an organism. For example, in mammals, only the zygote and the first cleavage stage blastomeres are totipotent.

As used herein, the term “pluripotent” means the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm.

As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

In various embodiments, the present invention provides methods to alter the potency of a cell by contacting the cell with a composition that modulates one or more components of a cellular pathway or developmental signaling pathway associated with the potency of the cell.

In various related embodiments, the present invention provides a method of altering the potency of a cell, comprising contacting the cell with one or more repressors that modulate at least one component of a cellular pathway associated with the potency of the cell. As used herein, the term “repressor” means a molecule that suppresses, decreases, inhibits, reduces, represses, lowers, abates, or lessens a component's epigenetic state, chromatin structure, transcription, mRNA splicing, post-transcriptional modification, mRNA stability and/or half-life, translation, post-translational modification, protein stability and/or half-life and/or protein activity. Inhibitors described herein are also considered repressors.

Repressors of the present invention modulate a component of a potency pathway either directly or indirectly, for example, by repressing the component, de-repressing a repressor of the component, repressing an activator of the component, de-repressing the component, repressing a repressor of the component, and/or de-repressing an activator of the component. Repressors can modulate one or more components of a cellular pathway associated with the developmental potency of a cell from a ground potency state to either a more or less potent state, depending on the one or more components being modulated.

For instance, by way of non-limiting example, contacting a differentiated cell with a repressor that modulates a component of a cellular pathway associated with the potency of a cell, wherein the component normally acts to decrease or restrict potency, would act to increase the potency of the cell.

In another non-limiting example, contacting a non-differentiated cell with a repressor that modulates a component of a cellular pathway associated with the potency of a cell, wherein the component normally acts to increase potency, would act to decrease or further restrict the potency of the cell.

In various other related embodiments, the present invention provides a method of altering the potency of a cell, comprising contacting the cell with one or more activators that modulate at least one component of a cellular pathway associated with the potency of a cell. As used herein, the term “activator” means a molecule that facilitates, increases, promotes, enhances, heightens or activates a component's epigenetic state, chromatin structure, transcription, mRNA splicing, post-transcriptional modification, mRNA stability and/or half-life, translation, post-translational modification, protein stability and/or half-life and/or protein activity.

Activators of the present invention modulate a component of a potency pathway either directly or indirectly, for example, by activating the component, activating a repressor of a repressor of the component or activating an activator of the component. Activators can modulate one or more components of a cellular pathway associated with the developmental potency of a cell from a ground potency state to either a more or less potent state, depending on the one or more components being modulated.

For instance, by way of non-limiting example, contacting a non-differentiated cell with an activator that modulates a component of a cellular pathway associated with the potency of a cell, wherein the component normally acts to decrease or restrict potency, would act to further decrease or restrict the potency of the cell.

In another non-limiting example, contacting a differentiated cell with an activator that modulates a component of a cellular pathway associated with the potency of a cell, wherein the component normally acts to increase potency, would act to increase the potency of the cell.

In another related embodiment, the present invention provides a method of altering the potency of a cell, comprising contacting the cell with one or more repressors and activators in any number and combination, or a composition comprising the same, that modulate at least one component of a cellular pathway associated with the potency of a cell.

In other various embodiments, the present invention provides methods to alter the potency of a cell by contacting the cell with at least one repressor and/or activator that modulates one or more components of a cellular pathway or developmental signaling pathway associated with the potency of the cell. In a related embodiment, the present invention provides a method of altering the potency of a cell, comprising contacting the cell with a combination of 1, 2, 3, 4, 5, 6, 7, 8, 9, 10 or more repressors and/or activators, in any combination, or a composition comprising the same, that modulate at least one component of a cellular pathway associated with the potency of the cell.

Illustrative repressors of the present invention can be any number and/or combination of the following molecules: a polynucleotide (e.g., a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a DNAzyme, a ssDNA, and the like), polypeptide or active fragment thereof (e.g., an antibody, an intrabody, a transbody, a protein, an enzyme, a peptidomimetic, a peptoid, a transcriptional factor, and the like), or a small organic molecule, and the like.

Illustrative activators of the present invention can be any number and/or combination of the following molecules: an antibody or an antibody fragment, an mRNA, a bifunctional antisense oligonucleotide, a dsDNA, a polypeptide or an active fragment thereof, a peptidomimetic, a peptoid, or a small organic molecule, and the like.

Repressors and activators of the present invention can be formulated together, for example, in a single composition or in multiple compositions that can be administered simultaneously to a patient or subject. In some embodiments, a composition comprising both activators and repressors is preferred. Without wishing to be bound by a particular theory, a composition comprising both activators and repressors can produce a synergistic effect on one or more components of a cellular pathway or pathways associated with the potency of a cell. For instance, in a non-limiting example, administration of repressor A or activator B reprograms a cell from a terminally differentiated state to a multipotent state, or pluripotent state. However, upon administration of repressor A and activator B, the cell is reprogrammed from the terminally differentiated state to a pluripotent state or totipotent state, respectively. One having skill in the art would also recognize that the above example is equally illustrative of differentiating or programming cells.

Repressors and activators of the present invention can also be used separately, for example, administered in separate compositions, wherein one composition is administered prior to the other, wherein the time between administrations is minutes, hours, days, weeks or months. In other embodiments, repressors and activators can be administered in different compositions, but at the same time, and optionally, administration of the two or more compositions can be at a single administration site or multiple administration sites. When administered at multiple sites the method of administration can be the same or different for each composition administered. One having ordinary skill in the art would understand that multiple administrations are desirable in particular embodiments and often preferred in embodiments in which the cells are reprogrammed to a more potent state and then subsequently programmed to a less potent state.

In one embodiment, a composition of the present invention comprises one or more repressors or a single repressor. In particular embodiments, the repressor is a transcriptional repressor (i.e., a transcription factor that negatively influences transcription) that alters the potency of a cell by repressing one or more components of a cellular pathway associated with the potency of a cell either directly or indirectly; for example, by repressing the component, de-repressing a repressor of the component, repressing an activator of the component, de-repressing the component, repressing a repressor of the component, and/or de-repressing an activator of the component. Repression by a transcriptional repression can lead to an increase in the potency of a cell compared to the ground potency state. Repression by a transcriptional repressor can also lead to a decrease in the potency of a cell compared to the ground potency state. One having ordinary skill in the art would recognize that the transcriptional repressor would either contribute to the decrease or increase in cell potency relative to a ground potency state based, in part, on the identity and function of the gene being transcriptionally repressed.

In another embodiment, a composition of the present invention comprises one or more activators or a single activator. In particular embodiments, the activator is a transcriptional activator (i.e., a transcription factor that positively influences transcription) that alters the potency of a cell by activating one or more components of a cellular pathway associated with the potency of a cell either directly or indirectly; for example, by activating the component, activating a repressor of a repressor of the component or activating an activator of the component. Activation by a transcriptional activator can lead to either an increase in the potency of a cell compared to the ground potency state. Activation by a transcriptional activator can also lead to a decrease in the potency of a cell compared to the ground potency state. One having ordinary skill in the art would recognize that the transcriptional activator would either contribute to the decrease or increase in cell potency relative to a ground potency state based, in part, on the identity and function of the gene being transcriptionally activated.

In yet another embodiment, a composition of the present invention comprises both activators and repressors in any number and/or combination.

Any of the compositions described herein, supra or infra, can modulate a single component or multiple components of a cellular pathway or pathways associated with the potency of a cell. Compositions of the present invention can be used in any number and/or combination in order to increase the efficacy of a method of reprogramming, dedifferentiating, programming, or differentiating cells of the present invention. Additionally, the administration of more than one composition can be used to reprogram or dedifferentiate a cell, and, subsequently, to program or differentiate the cell.

A starting population of cells may be derived from essentially any suitable source, and may be heterogeneous or homogeneous. In certain embodiments, the cells to be treated according to the invention are adult cells, including essentially any accessible adult cell types. In other embodiments, the cells used according to the invention are adult stem cells, progenitor cells, or somatic cells. In still other embodiments, the cells treated according to the invention include any type of cell from a newborn, including, but not limited to newborn stem cells, progenitor cells, and tissue-derived cells (e.g., somatic cells). Accordingly, a starting population of cells that is reprogrammed or dedifferentiated by the methods of the present invention as described elsewhere herein, can be programmed or differentiated into any of the somatic cell types discussed herein, supra and infra.

Thus, in various embodiments, the present invention provides methods for increasing the potency of a cell, which further comprise a step of contacting a totipotent cell, a pluripotent cell, or a multipotent cell with a second composition that modulates one or more components associated with a cellular potency pathway(s) in order to differentiate the previously reprogrammed cell into a mature somatic cell of a particular lineage.

In other various embodiments, the present invention provides a culture, culture composition or culture system comprising i) a cell; ii) a composition comprising one or more repressors and/or activators; and iii) a pharmaceutically acceptable cell culture medium.

It would be clear to one having ordinary skill in the art that the foregoing methods and compositions are useful in methods of ex vivo and in vivo therapy, including, but not limited to, cell, tissue, and/or organ regenerative therapy. The compositions may be administered directly or in combination with cells of the invention, in either a reprogrammed or programmed state, or a combination of states. The present invention also contemplates, in part, that in certain embodiments, treatment regimens comprise multiple administrations of compositions described elsewhere herein, in order to achieve therapeutic treatment. Additionally, in one embodiment, cells and compositions of the invention can be administered to a subject or patient in an implant device.

The treatment methods encompassed by the present invention are suitable to prevent, ameliorate, and/or treat cancer, degenerative disease, autoimmune disease, age related disorders, genetic disorders, cell, tissue, or organ related injury or degeneration as described elsewhere herein. Treatment methods of the present invention also provide cells and/or compositions suitable for cell, tissue, and organ transplantation.

### I. Overview of Somatic Cell Reprogramming

Mammalian cloning from differentiated donor cells has demonstrated that an oocyte is capable of reprogramming adult somatic cell nuclei to an embryonic state that can direct development of a new organism. However, alternatives to deriving patient-specific embryonic stem cells by nuclear transfer are needed due to the extreme inefficiency of reprogramming by this method and the ethical issues of obtaining human oocytes. Alternative strategies for somatic cell reprogramming have emerged, but to date, are not suitable for widespread experimental studies or safe for in vivo or ex vivo cell-based therapies. Strategies to induce the conversion of a differentiated cell into a more potent state (e.g., from an adult somatic cell to a multipotent cell or pluripotent cell), include nuclear transfer, cellular fusion, the use of cell extracts, and culture-induced reprogramming.

Takahashi and Yamanaka 2006 conducted somatic cell reprogramming experiments using mouse somatic cells and found that a combination of the transcription factors Oct-3/4, Sox-2, c-Myc, and Klf-4 were sufficient to reprogram mouse fibroblasts to cells closely resembling mouse ESCs, although not completely pluripotent. These results were rapidly confirmed and extended in mouse material (Maherali et al., 2007; Okita et al., 2007; Wernig et al., 2007) and eventually successfully applied to human material (Takahashi et al., 2007; Lowry et al., 2008; Park et al., 2008).

Yu et al., 2007 conducted similar somatic cell reprogramming screens using human material, and found that a combination of Oct-3/4, Sox-2, Nanog, and Lin28 were sufficient to reprogram human cells, with Oct-3/4 and Sox-2 appearing essential and the other two factors either strongly (Nanog) or modestly (Lin28) influencing the efficiency of reprogramming.

Oct-3/4, Sox-2, and Nanog are clearly sufficient to reprogram fetal, neonatal, and adult human cells in the absence of Lin28; moreover, c-Myc and Klf-do not appear to be required for human somatic cell reprogramming, but these factors do increase the rate and efficacy of somatic cell reprogramming.

Lowry et al., 2008 ectopically expressed the combination of the defined transcription factors KLF4, OCT4, SOX2, and C-MYC to generate human induced pluripotent stem (iPS) cells from dermal fibroblasts. Additionally, Lowry et al., shoed that reprogrammed somatic cells can further be induced to differentiate along lineages representative of the three embryonic germ layers indicating the pluripotency of these cells.

Dimos et al., 2008 successfully reprogrammed somatic cells from an 82-year-old woman diagnosed with a familial form of amyotrophic lateral sclerosis (ALS). These patient-specific reprogrammed pluripotent cells possess properties of embryonic stem cells and were successfully directed to differentiate into motor neurons, the cell type destroyed in ALS. These cells could in turn be used for disease modeling, drug discovery, and eventually autologous cell replacement therapies.

Aasen et al., 2008 showed that reprogrammed somatic cells derived reprogrammed juvenile human primary keratinocytes by retroviral transduction with OCT4, SOX2, KLF4 and c-MYC are reprogrammed at least 100-fold more efficient and two-fold faster compared with reprogramming using human fibroblasts. Keratinocyte-derived iPS cells appear indistinguishable from human embryonic stem cells in colony morphology, growth properties, expression of pluripotency-associated transcription factors and surface markers, global gene expression profiles and differentiation potential in vitro and in vivo. Aasen et al. also generated KiPS cells from single adult human hairs.

Qin et al., 2008 demonstrated that cells from the mouse meningeal membranes express elevated levels of the embryonic master regulator Sox-2, and were successfully reprogrammed to a pluripotent state using viral transduction of Oct-3/4, Sox-2, c-Myc, and Klf-4. Meningeal cell derived iPS clones were generated without selection, and were found to be pluripotent on the basis of DNA methylation analysis, and ability to transmit through the germline.

Di Stefano et al., 2008 found that retroviral transduction of the transcription factors Oct-3/4, Sox-2, Klf-4 and c-Myc successfully reverted mouse NSCs to a pluripotent embryonic stem (ES) cell-like state with a two-fold efficiency increase, faster kinetic and with a lower number of viral integrations compared to the reprogramming of MEFs. Di Stefano et al. further showed that the high levels of endogenous Sox-2 and c-Myc in mouse NSCs enables somatic cell reprogramming to pluripotency through the ectopic viral expression of Oct-3/4 and Klf-4. Thus, endogenous expression of reprogramming genes facilitates somatic cell reprogramming.

Eminli et al., 2008 reprogrammed mouse neural progenitor cells by infection with viral vectors expressing Oct-3/4, Sox-2, Klf-4, and c-Myc. Infected NPCs gave rise to iPS cells that expressed markers of embryonic stem cells, showed demethylation of pluripotency genes, formed teratomas, and contributed to viable chimeras. Like mouse neural stem cells, the neural progenitor cells endogenously express a relatively high level of Sox-2, and thus, only require viral transduction with Oct-3/4, Klf-4, and c-Myc to attain a pluripotent state.

Mali et al., 2008 improved efficiency and pace of generating induced pluripotent stem cells from human adult and fetal fibroblasts. Efficiency of somatic cell reprogramming to a pluripotent state was increased by 23-70-fold from both human adult and fetal fibroblasts. This was achieved by viral transduction of SV40 large T antigen (T) in combination with Oct-3/4, Sox-2, Klf-4, and c-Myc or Oct-3/4, Sox-2, Nanog, and Lin-28.

In addition, Zhou et al., 2008 showed that in methods relating to reprogramming and subsequent programming, it is not necessary to revert to a completely pluripotent state prior to the programming event. Zhou et al. expressed key developmental regulators of pancreatic β-cells, namely, Neurog3 (Ngn3), Pdx1, and Mafa. By virally transducing adult mouse pancreatic exocrine cells with these factors, the cells were reprogrammed into cells that are indistinguishable from endogenous islet β-cells in size, shape and ultrastructure. Furthermore, the reprogrammed cells express genes essential for β-cell function and can ameliorate hyperglycaemia by remodelling local vasculature and secreting insulin. Thus, this study provides an example of cellular reprogramming using defined factors in an adult organ and suggests a general paradigm for directing cell reprogramming without reversion to a completely pluripotent state.

A number of various cell types from all three germ layers have been shown to be suitable for somatic cell reprogramming, including, but not limited to liver and stomach (Aoi et al., 2008); pancreatic β cells (Stadtfeld et al., 2008); mature B lymphocytes (Hanna et al., 2008); human dermal fibroblasts (Takahashi et al., 2007; Yu et al., 2007; Lowry et al., 2008; Aasen et al., 2008); meningiocytes (Qin et al., 2008); neural stem cells (DiSteffano et al., 2008); and neural progenitor cells (Eminli et al., 2008). Thus, the present invention contemplates, in part, methods to reprogram and/or program cells from any cell lineage.

Inclusion of additional factors, such as TERT, T genes, and down-regulation of somatic cell-specific transcription factors (e.g., down-regulation of Pax5 in mature B cells), can improve the reprogramming efficiency (Hanna et al., 2008; Mali et al., 2008). Although reprogrammed clones can be consistently recovered and expanded with the existing gene combinations, for practical applications, the current reprogramming efficiency is low and culturing likely selects for abnormal genetic or epigenetic events that are stably propagated in the resulting iPS cell lines. It appears that retroviral integration into specific sites in the somatic cell genome is not required (Aoi et al., 2008; Stadtfeld et al., 2008), but expression of the oncogenes c-Myc and Klf-4 is required. Thus, issues regarding the integrity of reprogrammed somatic cells and patient safety have still failed to be adequately addressed.

In contrast, the present invention provides, in part, methods and compositions for reprogramming or dedifferentiating and/or programming or differentiating a cell that are flexible, efficient, and safe. The present invention contemplates, in part, to alter the potency of a cell by contacting the cell with one or more repressors and/or activators to modulate the epigenetic state, chromatin structure, transcription, mRNA splicing, post-transcriptional modification, mRNA stability and/or half-life, translation, post-translational modification, protein stability and/or half-life and/or protein activity of a component of a cellular pathway associated with determining or influencing cell potency.

Thus, in various embodiments, the present invention uses predictable and highly controlled methods for gene expression, as discussed elsewhere herein, that enable the reprogramming or de-differentiation and programming or differentiation of somatic cells ex vivo or in vivo. As, noted above, the intentional genetic engineering of cells, however, is not preferred, since it alters the cellular genome and would likely result in genetic or epigenetic abnormalities. In contrast, the compositions and methods of the present invention provide repressors and/or activators that non-genetically alter the potency of a cell by mimicking the cell's endogenous developmental potency pathways to achieve reprogramming and/or programming of the cell.

Small Molecules in Reprogramming

Reprogramming of somatic cells into induced pluripotent stem cells has also been achieved by retroviral infection of defined genes (e.g., Oct-3/4, Sox-2, Klf-4, c-Myc, and Lin28, and the like) in combination with small molecules.

Shi et al., 2008 identified a small-molecule combination, BIX-01294 and BayK8644, which enables reprogramming of Oct-3/4/Klf-4 virally transduced mouse embryonic fibroblasts, which do not endogenously express the factors essential for reprogramming. This study demonstrates that small molecules identified through a phenotypic screen can compensate for viral transduction of critical factors, such as Sox-2, and improve reprogramming efficiency.

Lluis et al., 2008 demonstrated that cyclic activation of Wnt/β-catenin signaling in ESCs with Wnt3a or the glycogen synthase kinase-3 (GSK-3) inhibitor 6-bromoindirubin-3′-oxime (B10) strikingly enhances the ability of ESCs to reprogram somatic cells after fusion.

Silva et al., 2008 successfully produce induced pluripotent cells from mouse neural stem cells by retroviral transduction with Oct-3/4 and Klf-4 in combination with a dual inhibition of mitogen-activated protein kinase signalling and glycogen synthase kinase-3 (GSK3) with the self-renewal cytokine leukaemia inhibitory factor (LIF).

Huangfu et al., 2008b found that valproic acid (VPA), a histone deacetylase inhibitor, enables reprogramming of primary human fibroblasts with viral transduction of only two factors, Oct-3/4 and Sox-2, without the need for the oncogenes c-Myc or Klf-4.

Hockemeyer et al., 2008 derived a small molecule-based system to efficiently reprogram genetically homogeneous “secondary” somatic cells, which carry the reprogramming factors Oct-3/4, Sox-2, c-Myc, and Klf-4 as defined doxycycline (DOX)-inducible transgenes. Marson et al., 2008 reported the successfully reprogramming of somatic cells by viral transduction of Oct-3/4, Sox-2, and Klf-4, in combination with Wnt3a.

Huangfu et al., 2008a reported that DNA methyltransferase and histone deacetylase (HDAC) inhibitors improve reprogramming efficiency. In particular, valproic acid (VPA), an HDAC inhibitor, improves reprogramming efficiency by more than 100-fold, using Oct-3/4-GFP as a reporter. VPA also enables efficient induction of pluripotent stem cells without introduction of the oncogene c-Myc.

However, despite all these advances, to date, no reprogramming solution exists to address the complete non-genetic reprogramming of a somatic cell. As noted above, the intentional genetic engineering of cells is not preferred, since it alters the cellular genome and would likely result in genetic or epigenetic abnormalities. Thus, issues regarding the integrity of reprogrammed somatic cells and patient safety have still failed to be adequately addressed.

The compositions and methods of the present invention provide solutions to these and related issues surrounding the safety and efficacy of cell reprogramming and/or programming.

Thus, in one embodiment, the present invention provides a method of altering the potency of a cell that comprises contacting the cell with one or more repressors and/or activators or a composition comprising the same, wherein said one or more repressors and/or activators modulates at least one component of a cellular pathway associated with the potency of the cell, thereby altering the potency of the cell. In particular embodiments, the one or more repressors and/or activators modulate one or more components of a cellular pathway associated with the potency of the cell and thereby alter the potency of the cell. In certain embodiments, the one or more repressors and/or activators modulate one or more components of one or more cellular pathways associated with the potency of the cell and thereby alter the potency of the cell. In certain related embodiments, the modulation of the component(s) is synergistic and increases the overall efficacy of altering the potency of a cell. The potency of the cell can be altered, compared to the ground potency state, to a more potent state (e.g., from a differentiated cell to a multipotent, pluripotent, or totipotent cell) or a less potent state (e.g., from a totipotent, pluripotent, or multipotent cell to a differentiated somatic cell). In still yet other embodiments, the potency of a cell may be altered more than once. For example, a cell may first be reprogrammed to a more potent state, then programmed to a particular somatic cell.

In another embodiment, the methods of the present invention provide for increasing the potency a cell, wherein the cell is reprogrammed or dedifferentiated to a totipotent state, comprising contacting the cell with a composition comprising one or more repressors and/or activators, wherein the one or more repressors and/or activators modulates at least one component of a cellular pathway associated with the totipotency of the cell, thereby increasing the potency of the cell to a totipotent state.

In a particular embodiment, a method of increasing the potency a cell to a pluripotent state comprises contacting the cell with one or more repressors and/or activators, wherein the one or more repressors and/or activators modulates at least one component of a cellular pathway associated with the potency of the cell, thereby increasing the potency of the cell to a pluripotent state.

In another particular embodiment, a method of increasing the potency a cell to a multipotent state comprises contacting the cell with one or more repressors and/or activators, wherein the one or more repressors and/or activators modulates at least one component of a cellular pathway associated with the potency of the cell, thereby increasing the potency of the cell to a multipotent state.

In certain embodiments, a method of increasing the potency of a cell further comprises a step of contacting the totipotent cell, the pluripotent cell or the multipotent cell with a second composition, wherein the second composition modulates the at least one component of a cellular potency pathway to decrease the totipotency, pluripotency or multipotency of the cell and differentiate the cell to a mature somatic cell.

In another related embodiment, the present invention provides a method of reprogramming a cell that comprises contacting the cell with a composition comprising one or more repressors and/or activators, wherein the one or more repressors and/or activators modulates at least one component of a cellular pathway or pathways associated with the reprogramming of a cell, thereby reprogramming the cell.

In other embodiments, the present invention provides a method of dedifferentiating a cell to a more potent state, comprising contacting the cell with the composition comprising one/or more activators, wherein the one or more repressors and/or activators modulates at least one component of a cellular pathway or pathways associated with the dedifferentiation of the cell to the more potent state, thereby dedifferentiating the cell to a impotent state.

According to various embodiments of the present invention a repressor can be an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule. Polypeptide-based repressors include, but are note limited to fusion polypeptides. Polypeptide-based repressors also include transcriptional repressors, which can further be fusion polypeptides and/or artificially designed transcriptional repressors as described elsewhere herein.

According to other various embodiments, an activator can be an antibody or an antibody fragment, an mRNA, a bifunctional antisense oligonucleotide, a dsDNA, a polypeptide or an active fragment thereof, a peptidomimetic, a peptoid, or a small organic molecule.

In some embodiments, repressors modulate at least one component of a cellular potency pathway by a) repressing the at least one component; b) de-repressing a repressor of the at least one component; or c) repressing an activator of the at least one component. In related embodiments, one or more repressors can modulate at least one component of a pathway associated with the potency of a cell by a) de-repressing the at least one component; b) repressing a repressor of the at least one component; or c) de-repressing an activator of the at least one component.

In certain embodiments, one or more repressors modulates at least one component of a cellular pathway associated with the potency of a cell by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In related embodiments, activators modulate at least one component of a cellular pathway associated with the potency of a cell by a) activating the at least one component; b) activating a repressor of a repressor of the at least one component; or c) activating an activator of the at least one component.

In certain embodiments, one or more activators modulates at least one component by a) activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity; or b) activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

### II. Stem Cells of Different Origins

A. Embryonic Carcinoma Cells (EC)

Teratocarcinomas are malignant germ cell tumors that comprise an undifferentiated EC component and a differentiated component that can include all three germ layers. A single EC cell is capable of both unlimited self-renewal and multilineage differentiation, thus establishing that EC are a type of pluripotent stem cell. This was also the first experimental demonstration of a cancer stem cell. EC cell lines have limited developmental potential and contribute poorly to chimeric mice, likely due to the accumulation of genetic changes during teratocarcinoma formation and growth (Atkin et al., 1974).

Human EC cells are different from mouse EC cells. For example, SSEA-1, a cell-surface marker specifically expressed on mouse EC cells, is absent on human EC cells, while SSEA-3, SSEA-4, TRA-1-60, and TRA-1-81 are absent on mouse EC cells but are present on human EC cells (Andrews et al., 1982, 1984; Kannagi et al., 1983). Also, in contrast to mouse EC cells, human EC cells are highly aneuploid, which likely accounts for their inability to differentiate into a wide range of somatic cell types, and which drastically limits their utility as an in vivo or ex vivo therapeutic treatment for mammals. Thus, hEC cells are neither a safe nor suitable source of pluripotent cells for use in the methods of the present invention.

B. Mouse Embryonic Stem Cells

The first mouse ESC lines were derived from the ICM of mouse blastocysts using culture conditions (fibroblast feeder layers and serum) previously used for mouse EC cells (Evans and Kaufman 1981; Martin 1981). ESC cultures clonally derived from a single cell differentiate into a wide variety of cell types in vitro and form teratocarcinomas when injected into mice (Martin 1981). More importantly, unlike EC cells, these karyotypically normal cells can contribute at a high frequency to a variety of tissues in chimeras, including germ cells, thus providing a practical way to introduce modifications to the mouse germline (Bradley et al., 1984).

Pluripotent stem cell lines (epiblast stem cells or EpiSCs) have been established from epiblasts isolated from E5.5 to E6.5 post-implantation mouse embryos that differ significantly from mouse ESCs but share key features with human ESCs (Brons et al., 2007; Tesar et al., 2007). For example, EpiSCs derivation failed in the presence of LIF and/or BMP4, the two factors required for the derivation and self-renewal of mouse ESCs. However, similar to human ES and iPS cells, FGF and Activin/Nodal signaling appear to play a role in EpiSC derivation and self-renewal. Gene expression by EpiSCs closely reflects their post-implantation epiblast origin and is distinct from mouse ESCs. Nevertheless, EpiSCs do share the two key features characteristic of ESCs: prolonged proliferation in vitro and multilineage differentiation.

C. Pluripotent Cell Lines Derived from Germ Cells

Despite the evidence that teratocarcinomas were derived from primordial germ cells (PGCs) (Stevens 1962), it was not until 1992 that pluripotent stem cells (embryonic germ cells or EG cells) were successfully derived from PGCs directly in vitro (Matsui et al., 1992; Resnick et al., 1992). In contrast to mouse ESCs, the initial derivation of mouse EG cells required a combination of stem cell factor (SCF), LIF, and FGF in the presence of a feeder layer. In culture, EG cells are morphologically indistinguishable from mouse ESCs and express typical ESC markers such as SSEA-1 and Oct-3/4. And similar to ESCs, upon blastocyst injection, they can contribute extensively to chimeric mice including germ cells (Labosky et al., 1994; Stewart et al., 1994). Unlike ESCs, however, EG cells retain some features of the original PGCs, including genome-wide demethylation, erasure of genomic imprints, and reactivation of X-chromosomes (Labosky et al., 1994; Tada et al., 1997), the degree of which likely reflects the developmental stages of the PGCs from which they are derived (Shovlin et al., 2008).

Multipotent germline stem cells (mGSCs) share a similar morphology with mouse ESCs and express typical mouse ESC-specific markers, differentiate into multiple lineages in vitro, form teratomas, and contribute extensively to chimeras including germline cells upon injection into blastocysts. However, mGSCs have an epigenetic status distinct from both ESCs and germline stem cells (Kanatsu-Shinohara et al., 2004). The mouse testis contains different subpopulations of germline stem cells (Izadyar et al., 2008). The origin of mGSCs is still somewhat unclear, though it might be possible that in vitro culture of germline stem cells reprograms a minority of these cells to resume an ESC-like state. For example, culture of GPR125+ (c-Kit−) spermatogonial progenitor cells (GSPCs) were able to convert these cells into pluripotent stem cells (multipotent adult spermatogonia-derived stem cells, or MASCs), which could differentiate into derivatives of all three primary germ layers both in vitro and in vivo (Seandel et al., 2007). The MASCs, however, have a gene expression pattern distinct from either GSPCs or ESCs.

The derivation of human EG cells was reported in 1998 (Shamblott et al., 1998), but in spite of efforts by several groups, their long-term proliferative potential appears to be limited (Turnpenny et al., 2003). Early passage human EG cells have been reported to differentiate into multiple lineages in vitro, but this has yet to be demonstrated from a clonally derived, stable cell line, nor to date have any human EG cell lines been reported to form teratomas. Besides having different growth factor requirements from human ESCs, human EG cells have a very distinct morphology and express SSEA-1, a cell-surface marker absent on human ESCs but present on early human germ cells. Thus, hEG cells would not be a suitable source of pluripotent stem cells for use in the in vivo and ex vivo therapies of the present invention.

D. Human Embryonic Stem Cells

There was a considerable delay between the derivation of mouse ESCs in 1981 and the derivation of human ESCs in 1998 (Thomson et al., 1998), in spite of several earlier attempts at human ESC derivation. Human ESCs are karyotypically normal and, even after prolonged undifferentiated proliferation, maintain the developmental potential to contribute to advanced derivatives of all three germ layers, even after clonal derivation (Amit et al., 2000). Similar to mouse ESCs, human ESCs have been derived from morula, later blastocyst stage embryos (Stojkovic et al., 2004; Strelchenko et al., 2004), single blastomeres (Klimanskaya et al., 2006), and parthenogenetic embryos (Lin et al., 2007; Mai et al., 2007; Revazova et al., 2007). It is not yet known whether pluripotent cell lines derived from these various sources have any consistent developmental differences or whether they have an equivalent potential. In contrast to mouse ESCs, FGF and TGFβ/Activin/Nodal signaling are of central importance to the self-renewal of human ESCs, making human ESCs similar to the recently described mouse epiblast-derived stem cells (Brons et al., 2007; Tesar et al., 2007). However, to date, the isolation and use of human embryonic stem cells is surrounded by ethical controversies. Thus, hESCs, while showing great therapeutic promise are not a suitable source of pluripotent cells for use in the methods of the present invention.

E. Induced Pluripotent Stem Cells (iPS)

Mouse iPS cells are remarkably similar to mouse ESCs. Although the initial mouse iPS cells did not contribute to the germline in chimeras (Takahashi and Yamanaka 2006), subsequent modification of the procedure to select iPS cells based on the reactivation of Oct-3/4 or Nanog promoter resulted in iPS cells that more closely resembled mouse ESCs (Maherali et al., 2007; Okita et al., 2007; Wernig et al., 2007), including the ability to contribute to germlines. Genetic selection applied during reprogramming, however, was later shown to be unnecessary for obtaining iPS cells closely resembling ESCs, as such cells could be selected based on colony morphology alone (Blelloch et al., 2007; Meissner et al., 2007). Despite the high similarity between mouse iPS and ESCs, tumor formation in iPS cell chimeric mice was high, presumably due to the expression of c-Myc in iPS cell-derived somatic cells (Maherali et al., 2007; Okita et al., 2007; Wernig et al., 2007). More recently, it has been shown that Oct-3/4, Sox-2, and Klf-4 are sufficient to allow reprogramming of both mouse and human somatic cells, albeit at a much lower efficiency than when c-Myc is included (Nakagawa et al., 2008).

Human iPS cells, produced either by expression of Oct-3/4, Sox-2, c-Myc, and Klf-4 or by Oct-3/4, Sox-2, Nanog, and Lin28 are also remarkably similar to human ESCs. These cells are morphologically similar to human ESCs, express typical human ESC-specific cell surface antigens and genes, differentiate into multiple lineages in vitro, and form teratomas containing differentiated derivatives of all three primary germ layers when injected into immunocompromised mice. Indeed, these new pluripotent cell lines satisfy all the original criteria proposed for human ESCs (Thomson et al., 1998), except that they are not derived from embryos. However, the efficiency of reprogramming adult fibroblasts remains low (<0.1%), and inefficient. In addition, such reprogrammed cells are not safe use in ex vivo or in vivo therapies because the cells require genetic alterations (i.e., viral integration of pluripotency factors) to achieve successful reprogramming.

F. Adult Stem Cells

Population of adult stem cells and supporting cells reside within specific areas of the human body designated as niches, including most of adult mammalian tissues/organs, such as bone marrow, heart, kidneys, brain, skin, eyes, gastrointestinal tract, liver, pancreas, lungs, breast, ovaries, prostate, and testis. In fact, adult stem cells appear to originate during ontogeny and persist in specialized niches within organs where they may remain quiescent for short or long periods of time. Adult stem cells can notably undergo proliferation and differentiation into more mature and specialized tissue-specific cell types following changes in their microenvironment within the niche. More specifically, stem cells and their supporting cells appear to interact reciprocally by forming diverse intercellular connections, such as gap and adherens junctions, for maintaining the niche integrity. Hence, latent adult stem cells appear to be activated during cell replenishment to repopulate the tissue compartments under physiological and pathological conditions. The compositions of the present invention can further facilitate this replenishment using the reprogrammed cells of the present invention beit cells in a totipotent, pluripotent, or multipotent state.

Adult stem cells of endodermal origin include, without limitation, pulmonary epithelial stem cells, gastrointestinal tract stem cells, pancreatic stem cells, hepatic oval cells, mammary and prostatic gland stem cells, and ovarian and testicular stem cells. Adult stem cells of mesodermal origin include, without limitation, bone marrow stem cells, hematopoietic stem cells, stromal stem cells, and cardiac stem cells. Adult stem cells of ectodermal origin include, without limitation, neural stem cells, skin stem cells, and ocular stem cells.

Hematopoietic stem cells (HSGs) and their progenitors differentiate in vitro and ex vivo into different hematopoietic cell lineages. Administration of particular compounds such as prostaglandins or agonists of prostaglandin pathways results in vivo and ex vivo differentiation of HSCs into different hematopoietic cell lineages. The ex vivo expansion and maturation of BM and MPB progenitors into the specific hematopoietic cell lineages have also been performed by using growth factors such as SCF, G-CSF, GM-CSF, ILs, Flk2/Flt3 ligand, and TPO. More specifically, it has been reported that the downregulation of the expression of the endogenous myelomonocytic cytokine receptors for GM-CSF and M-CSF on the HSC progenitors may be related with their maturation into a common lymphoid precursor. In contrast, the upregulated expression of these cytokine receptors, which are induced by using IL-2, appears to lead to myeloid cell development.

In vivo Proliferation and Differentiation of NSCs. Among the numerous growth factors and adhesion molecules that are be involved in the regulation of proliferation, maturation, and/or migration of adult NSCs, there are EGF, bFGF, SHH, Wnt/β-catenin, Notch 1 ligand jagged 1, platelet-derived growth factors (PDGFs), ciliary neutrophic factor, VEGF, thyroid hormone T3, dopamine, NGF, neuregulins, BMPs, TGF-β, Ephrins/Ephs, leukemia inhibitory factor (LIF), and integrins. More specifically, the EGF-EGFR system and β1-integrins appear to act in cooperation to promote the proliferation, survival, and migration of NSCs. In contrast, ephrin-A2 and Eph-A7 can reduce the proliferation and/or migration of neural progenitor cells. Furthermore, SHH is also expressed locally in both adult cortex and cerebellum, the regions that are associated with an elevated rate of cell proliferation and gliogenesis. In vivo analyses of SHH expression and activity have indicated that the quiescent NSCs and their TA cell progenitors in the SVZ and dentate gyrus region in the adult mouse forebrain respond to SHH by undergoing a marked expansion. Sustained activation of EGF-EGFR and SHH-patched receptor (PTCH) pathways contributes to brain tumor formation. A brain tumor stem cell population expressing the NSC marker CD133 and able to self-renew was isolated from tumors of patients with medulloblastoma; thus, the malignant transformation of NSCs can lead to brain tumor development. In addition, it has been observed that the adult mammalian NSCs also express Flk-1/VEGFR-2 and that the infusion of VEGF in the lateral ventricle can stimulate their proliferation. This suggests that the endogenous VEGF from endothelial cells might also contribute of paracrine fashion to the NSC activation in vivo. Based on the knowledge of the factors involved in the regulation of embryonic and adult NSC growth, survival and differentiation in vivo, several new methods for in vitro expansion and differentiation of embryonic and adult NSCs have been conceived.

In vitro Expansion and Differentiation of NSCs. Human and rodent NSC progenitors derived from ESCs, UCB, fetal brain, MSCs, or skin-derived stem cells or isolated from adult brain tissues can be expanded in vitro or ex vivo in floating clusters called neurospheres in the presence of exogenous EGF, bFGF, SHH, and/or LIF. Moreover, the withdrawal of these mitogens and the addition of serum, RA, BNP, TGF-β type III, and/or ascorbic acid may promote their differentiation in the three major neuronal cell types, including neurons, astrocytes, and oligodendrocytes. In addition, the coculture of NSCs from mouse cerebral cortex at embryonic day E10-11 with endothelial cells leads to an extensive production of neuron-like cells in vitro, supporting the fact that the endothelium within the niche can also contribute to the stimulation of NSC self-renewal.

One having ordinary skill in the art would recognize that the types of stem cells discussed herein are merely illustrative examples, and do not limit the invention in any way. Thus, the present invention contemplates, in part, to provide compositions and methods of using the same that can supplement the endogenous role of stem cells, including the various types of adult stems cells mentioned herein and known in the art. In particular embodiments, a subject of therapy of the instant invention will have one or more defects, disorders, diseases, or conditions affecting a natural adult stem cell process. In other embodiments, the treatment will be preventative, and thus, the subject may have no indications of a defects, disorders, diseases, or conditions affecting a natural adult stem cell process.

In particular embodiments, cells of the invention, may be reprogrammed into any one of the adult stem cell types discussed herein or known in the art. In addition, the adult stem cells themselves may serve as the cellular starting material for reprogramming. In another embodiment, totipotent or pluripotent cells of the invention can be programmed into an adult stem cell, as described herein or that is known in the art.

### III. Cells of the Present Invention

A. Cells Suitable for Reprogramming

A starting population of cells that is suitable for reprogramming or dedifferentiating according to the methods of the present invention, may be from a reptilian species, an avian species, a species of fish, or any mammalian species. In particular embodiments, the starting population of cells is isolated from a mammal selected from the group consisting of: a rodent, a sheep, a horse, a goat, a pig, a cat, a dog, or a primate. In certain embodiments, the primate is a human.

A starting population of cells that is suitable for reprogramming or dedifferentiating according to the methods of the present invention, may be may be of any type of cell or a mixture of cell types. Illustrative types of human cells are: keratinizing epithelial cells, including, but not limited to epidermal keratinocytes (differentiating epidermal cells), epidermal basal cells (stem cells), keratinocytes of fingernails and toenails, nail bed basal cells (stem cells), medullary hair shaft cells, eortical hair shaft cells, euticular hair shaft cells, cuticular hair root sheath cells, hair root sheath cells of Huxley's layer, hair root sheath cells of Henle's layer, external hair root sheath cells, hair matrix cells (stem cells), and the like; wet stratified barrier epithelial cells, including, but not limited to surface epithelial cells of stratified squamous epithelium of cornea, tongue, oral cavity, esophagus, anal canal, distal urethra and vagina, basal cells (stem cells) of epithelia of cornea, tongue, oral cavity, esophagus, anal canal, distal urethra and vagina, urinary epithelium cells (lining urinary bladder and urinary ducts), and the like; exocrine secretory epithelial cells, including, but not limited to a salivary gland mucous cells (polysaccharide-rich secretion), salivary gland serous cells (glycoprotein enzyme-rich secretion), Von Ebner's gland cells in tongue (washes taste buds), mammary gland cells (milk secretion), lacrimal gland cells (tear secretion), ceruminous gland cells in ear (wax secretion), eccrine sweat gland dark cells (glycoprotein secretion), eccrine sweat gland clear cells (small molecule secretion), apocrine sweat gland cells (odoriferous secretion, sex-hormone sensitive), gland of Moll cells in eyelid (specialized sweat gland), sebaceous gland cells (lipid-rich sebum secretion), Bowman's gland cells in nose (washes olfactory epithelium), Brunner's gland cells in duodenum (enzymes and alkaline mucus), seminal vesicle cells (secretes seminal fluid components, including fructose for swimming sperm), prostate gland cells (secretes seminal fluid components), bulbourethral gland cells (mucus secretion), Bartholin's gland cells (vaginal lubricant secretion), gland of Littre cells (mucus secretion), uterus endometrium cells (carbohydrate secretion), isolated goblet cells of respiratory and digestive tracts (mucus secretion), stomach lining mucous cells (mucus secretion), gastric gland zymogenic cells (pepsinogen secretion), gastric gland oxyntic cells (hydrochloric acid secretion), pancreatic acinar cells (bicarbonate and digestive enzyme secretion), paneth cells of small intestine (lysozyme secretion), type II pneumocyte of lung (surfactant secretion), Clara cells of lung, and the like; hormone secreting cells, including, but not limited to anterior pituitary cells, somatotropes, lactotropes, thyrotropes, gonadotropes, corticotropes, intermediate pituitary cells, secreting melanocyte-stimulating hormone, magnocellular neurosecretory cells that secrete oxytocin or vasopressin, gut and respiratory tract cells that secrete serotonin, endorphin, somatostatin, gastrin, secretin, cholecystokinin, insulin, glucagon, or bombesin; thyroid gland cells, thyroid epithelial cells, parafollicular cells, parathyroid gland cells, parathyroid chief cells, oxyphil cells, adrenal gland cells, chromaffin cells, secreting steroid hormones (mineralcorticoids and gluco corticoids), Leydig cells of testes secreting testosterone, theca interna cells of ovarian follicle, secreting estrogen corpus luteum cells of ruptured ovarian follicle, secreting progesterone granulosa lutein cells, theca lutein cells, juxtaglomerular cells (renin secretion), macula densa cells of kidney, peripolar cells of kidney, mesangial cells of kidney, and the like; metabolism and storage cells, including, but not limited tohepatocytes (liver cells) white fat cells brown fat cells liver lipocytesbarrier function cells (Lung, Gut, Exocrine Glands and Urogenital Tract), and the like; kidney cells, including, but not limited to kidney glomerulus parietal cells, kidney glomerulus podocytes, kidney proximal tubule brush border cells, loop of Henle thin segment cells, kidney distal tubule cells, kidney collecting duct cells, and the like; epithelial cells lining closed internal body cavities, including, but not limited to blood vessel and lymphatic vascular endothelial fenestrated cells blood vessel and lymphatic vascular endothelial continuous cells, blood vessel and lymphatic vascular endothelial splenic cells, synovial cells (lining joint cavities, hyaluronic acid secretion), serosal cells (lining peritoneal, pleural, and pericardial cavities), squamous cells (lining perilymphatic space of ear), squamous cells (lining endolymphatic space of ear), columnar cells of endolymphatic sac with microvilli (lining endolymphatic space of ear), columnar cells of endolymphatic sac without microvilli (lining endolymphatic space of ear), dark cells (lining endolymphatic space of ear), vestibular membrane cells (lining endolymphatic space of ear), stria vascularis basal cells (lining endolymphatic space of ear), stria vascularis marginal cells (lining endolymphatic space of ear), cells of Claudius (lining endolymphatic space of ear), cells of Boettcher (lining endolymphatic space of ear), choroid plexus cells (cerebrospinal fluid secretion), pia-arachnoid squamous cells, pigmented ciliary epithelium cells of eye, nonpigmented ciliary epithelium cells of eye, corneal endothelial cells, and the like; ciliated cells with propulsive function, including, but not limited to respiratory tract ciliated cells, oviduct ciliated cells (in female), uterine endometrial ciliated cells (in female), rete testis ciliated cells (in male), ductulus efferens ciliated cells (in male), ciliated ependymal cells of central nervous system (lining brain cavities), and the like; extracellular matrix secretion cells, including, but not limited to ameloblast epithelial cells (tooth enamel secretion), planum semilunatum epithelial cells of vestibular apparatus of ear (proteoglycan secretion), organ of Corti interdental epithelial cells (secreting tectorial membrane covering hair cells), loose connective tissue fibroblasts, corneal fibroblasts, tendon fibroblasts, bone marrow reticular tissue fibroblasts, other nonepithelial fibroblasts, pericytes, nucleus pulposus cells of intervertebral disc, cementoblast/cementocyte (tooth root bonelike cementum secretion), odontoblast/odontocyte (tooth dentin secretion), hyaline cartilage chondrocyte, fibrocartilage chondrocyte, elastic cartilage chondrocyte, osteoblast/osteocyte, osteoprogenitor cells (stem cells of osteoblasts), hyalocyte of vitreous body of eye, stellate cells of perilymphatic space of ear, and the like; contractile cells, including, but not limited to skeletal muscle cells, red skeletal muscle cells (slow), white skeletal muscle cells (fast), intermediate skeletal muscle cells, nuclear bag cells of muscle spindle, nuclear chain cells of muscle spindle, satellite cells (stem cells), cardiac muscle cells, ordinary cardiac muscle cells, nodal cardiac muscle cells, purkinje fiber cells, smooth muscle cells (various types), myoepithelial cells of iris, myoepithelial cells of exocrine glands, and the like; blood and immune system cells, including, but not limited to erythrocytes (red blood cells), megakaryocytes (platelet precursor), monocytes, connective tissue macrophages (various types), epidermal Langerhans cells, osteoclasts (in bone), dendritic cells (in lymphoid tissues), microglial cells (in central nervous system), neutrophil granulocytes, eosinophil granulocytes, basophilgranulocytes, mast cells, helper T cells, suppressor T cells, cytotoxic T cells, natural Killer T cells, B cells natural killer cells, reticulocytes, stem cells and committed progenitors for the blood and immune system (various types), and the like; sensory transducer cells, including, but not limited to auditory inner hair cells of organ of Corti, auditory outer hair cells of organ of Corti, basal cells of olfactory epithelium (stem cells for olfactory neurons), cold-sensitive primary sensory neurons, heat-sensitive primary sensory neurons, Merkel cells of epidermis (touch sensor), olfactory receptor neurons, pain-sensitive primary sensory neurons (various types), photoreceptor cells of retina in eye, photoreceptor rod cells, photoreceptor blue-sensitive cone cells of eye, photoreceptor green-sensitive cone cells of eye, photoreceptor red-sensitive cone cells of eye, proprioceptive primary sensory neurons (various types), touch-sensitive primary sensory neurons (various types), type I carotid body cells (blood pH sensor), type II carotid body cells (blood pH sensor), type I hair cells of vestibular apparatus of ear (acceleration and gravity), type II hair cells of vestibular apparatus of ear (acceleration and gravity), type I taste bud cells, and the like; autonomic neuron cells, including, but not limited to cholinergic neural cells (various types), adrenergic neural cells (various types), peptidergic neural cells (various types), and the like; sense organ and peripheral neuron supporting cells, including, but not limited to inner pillar cells of organ of Corti, outer pillar cells of organ of Corti, inner phalangeal cells of organ of Corti, outer phalangeal cells of organ of Corti, border cells of organ of Corti, Hensen cells of organ of Corti vestibular apparatus supporting cells, type I taste bud supporting cells, olfactory epithelium supporting cells, Schwann cells, satellite cells (encapsulating peripheral nerve cells bodies), enteric glial cells, and the like; central nervous system neurons and glial cells, including, but not limited to astrocyte (various types), neuron cells (large variety of types, still poorly classified), oligodendrocytes, spindle neuron, and the like; lens cells, including, but not limited to anterior lens epithelial cells, crystallin-containing lens fiber cells, and the like; pigment cells, including, but not limited to melanocytes, retinal pigmented epithelial cells, and the like; germ cells, including, but not limited to oogonia/oocytes, spermatids, spermatocytes, spermatogonium cells (stem cells for spermatocyte), spermatozoa, and the like; nurse cells, including, but not limited to ovarian follicle cells, Sertoli cells (in testis), thymus epithelial cells, and the like; interstitial cells, including, but not limited to interstitial kidney cells and the like; and other cell type, including, but not limited to type I pneumocytes (lining air space of lung), pancreatic duct cells (centroacinar cells), nonstriated duct cells (of sweat gland, salivary gland, mammary gland, etc.), principal cells, Intercalated cells, duct cells (of seminal vesicle, prostate gland, etc.), intestinal brush border cells (with microvilli), exocrine gland striated duct cells, gall bladder epithelial cells, ductulus efferens nonciliated cells, epididymal principal cells, epididymal basal cells, and the like.

In one embodiment, the starting population of cells is selected from adult or neonatal stem/progenitor cells.

In particular embodiments, the starting population of stem/progenitor cells is selected from the group consisting of: mesodermal stem/progenitor cells, endodermal stem/progenitor cells, and ectodermal stem/progenitor cells.

In related embodiments, the starting population of stem/progenitor cells is a mesodermal stem/progenitor cell. Illustrative examples of mesodermal stem/progenitor cells include, but are not limited to mesodermal stem/progenitor cells, endothelial stem/progenitor cells, bone marrow stem/progenitor cells, umbilical cord stem/progenitor cells, adipose tissue derived stem/progenitor cells, hematopoietic stem/progenitor cells (HSGs), mesenchymal stem/progenitor cells, muscle stem/progenitor cells, kidney stem/progenitor cells, osteoblast stem/progenitor cells, chondrocyte stem/progenitor cells, and the like.

In other related embodiments, the starting population of stem/progenitor cells is an ectodermal stem/progenitor cell. Illustrative examples of ectodermal stem/progenitor cells include, but are not limited to neural stem/progenitor cells, retinal stem/progentior cells, skin stem/progenitor cells, and the like.

In other related embodiments, the starting population of stem/progenitor cells is an endodermal stem/progenitor cell. Illustrative examples of endodermal stem/progenitor cells include, but are not limited to liver stem/progenitor cells, pancreatic stem/progenitor cells, epithelial stem/progenitor cells, and the like.

In certain embodiments, the starting population of cells may be a heterogeneous or homogeneous population of cells selected from the group consisting of: pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like.

B. Reprogrammed Cells

The reprogrammed or dedifferentiated cells of the present invention are produced by the methods described herein throughout. In one embodiment, a starting population of cells is reprogrammed partially, e.g., from a differentiated cell to a state of multipotency or pluripotency; or from an initial state of potency to a higher level of potency. In other embodiments, a starting population of cells is reprogrammed completely, e.g., from a differentiated cell to a totipotent cell. One having ordinary skill in the art would understand that a cell that is partially reprogrammed to a pluripotent state, can be completely pluripotent or partially pluripotent, and that the state of pluripotency can be assessed by methods well-known to the skilled artisan, including, but not limited to morphological characteristics, epigenetic markers, inactive-X chromosome reactivation (in female stem cells), expression of pluripotency cell markers, in vitro differentiation, teratoma formation (e.g., implant reprogrammed cells into a nude mouse), chimeric formation (mouse), germline contribution (mouse), and tetraploid embyro complementation (mouse).

For example, without wishing to be bound by a particular theory, completely reprogrammed cells of the present invention possess epigenetic modifications characteristic of transcriptionally active chromatin (e.g., acetylation, H3K4 methylation, and the like) in regions where genes that contribute to the establishment or maintenance of cell potency are located, while the locus of genes involved in differentiation or programming pathways are heterochromatic or “transcriptionally silent”. Completely reprogrammed cells also display inactive-X chromosome reactivation, expression of pluripotency cell markers (described elsewhere herein), are capable of differentiating into the three embyronic lineages in vitro and in mouse models of teratoma formation, chimerism, germline transmission and tetraploid embryo complementation.

The degree of multipotency and totipotency of a reprogrammed cell can also be tested by methods well-known to the skilled artisan.

The reprogrammed cells of the present invention provide numerous advantages over the presently existing reprogrammed cells in the art. Namely, the reprogrammed cells of the present invention are produced without genetic modification, and thus, are safer than reprogrammed cells in the art. The methods described herein throughout can also be used to reprogram cells in vitro, ex vivo or in vivo; thus presenting a higher degree of flexibility over previously described methods. Art-known reprogramming methods also suffer from a lack of efficiency in the number of cells reprogrammed from a starting population of cells. This is problematic because methods of selecting such “reprogrammed” cells are based on more rapidly growing pluripotent colonies, which likely exhibit growth advantages due to undesired genetic modifications, e.g., genomic mutations dispositive of cancer.

Thus, in particular embodiments, the methods of the present invention reprogram cells with an efficiency of at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or any intervening percentage of reprogramming.

In related embodiments, the methods of the present invention reprogram cells with an efficiency of more than 0.1%, more than 0.5%, more than 1%, more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, or more than 95%.

In other embodiments, the methods of the present invention reprogram cells with an efficiency of about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% or any intervening percentage of reprogramming.

In still other embodiments, the methods of the present invention reprogram cells with an efficiency in a range of about 0.1% to about 100%, about 0.5% to about 95%, about 1% to about 90%, about 10% to about 85%, about 25% to about 75% or about 40% to about 60%, or any intervening range of reprogramming.

For example, a reprogramming efficiency of 50% means that if one started with a population of 100 differentiated cells in a heterogeneous or homogenous population, then 50 cells were reprogrammed to a more potent state, either partially or completely. In preferred embodiments, reprogramming efficiency is measured as the percentage of completely reprogrammed cells from a starting population of differentiated cells or less potent cells.

C. Programmed Cells

The present invention also contemplates, in part, programming cells in an initial state of potency (i.e., a ground potency state) to a less potent (e.g., more differentiated) state. For example, any of the cells described herein that are reprogrammed to a pluripotent of totipotent state may be differentiated to any type of cell described as a starting population of cells above. In particular embodiments, reprogrammed cells are programmed into neural cells, glial cells, cardiac cells, pancreatic islet cells, motor neuron cells, hepatocyte cells, renal cells, cells of the digestive tract, cells of the eye, lung cells, skin cells, vascular cells, bone cells, chrondrocytes, skeletal muscle cells, hematopoietic cells, immature progenitor cells, hair follicle cells, or stem/progenitor cells, including, but not limited to mesodermal stem/progenitor cells, endodermal stem/progenitor cells, or ectodermal stem/progenitor cells, among other cell types known to a person skilled in the art.

The present invention also contemplates, in part, highly efficient methods of differentiation or programming compared to the presently available methods in the art. In particular embodiments, the methods of the present invention program cells with an efficiency of at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or any intervening percentage of reprogramming.

In related embodiments, the methods of the present invention program cells with an efficiency of more than 0.1%, more than 0.5%, more than 1%, more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, or more than 95%.

In other embodiments, the methods of the present invention program cells with an efficiency of about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% or any intervening percentage of reprogramming.

In still other embodiments, the methods of the present invention program cells with an efficiency in a range of about 0.1% to about 100%, about 0.5% to about 95%, about 1% to about 90%, about 10% to about 85%, about 25% to about 75% or about 40% to about 60%, or any intervening range of reprogramming.

For example, a programming efficiency of 50% means that if one started with a population of 100 pluripotent cells, then 50 cells were programmed to a less potent state, either partially or completely. In preferred embodiments, programming efficiency is measured as the percentage of completely programmed cells from a starting population of differentiated cells or less potent cells.

The degree of cell programming can be determined by routine methods known to the skilled artisan. For example, one having ordinary skill in the art can assay for any one of the genes and/or proteins known to identify cells of a given lineage in order to determine the degree of programming or differentiation of a cell. Any one of the markers described herein is suitable for use in the methods of the present invention.

Illustrative gene expression markers for ectodermal cells include, but are not limited to astrocyte markers such as GFAP and S100B; early ectoderm markers such as Nestin and Notch1; neural crest cell markers such as CD271 (p75, NGFR/NTR), CD49d (Integrin α4), CD57 (HNK-1), MASH1, Neurogenin 3, and Notch1; neural stem cell markers such as CD146 (MCAM, MUC18), CD15 (SSEA-1, Lewis X), CD15s (Sialyl Lewis x), CD184 (CXCR4), CD24, CD271 (p75, NGFR/NTR), CD29 (Integrin β1), CD49f (Integrin α6), CD54 (ICAM-1), CD81 (TAPA-1), CD95 (Fas/APO-1), CDw338 (ABCG2), Nestin, Noggin, Notch1, Sox2, and Vimentin; neuronal markers such as α-Synuclein, α-Synuclein (pY125), ApoE, CD112, CD24, CD271 (p75, NGFR/NTR), CD56 (NCAM), CD81 (TAPA-1), CD90 (Thy-1), CD90.1 (Thy-1.1), CD90.2 (Thy-1.2), ChAT, Contactin, Doublecortin, GABA A Receptor, GABA B Receptor, GAP-43 (Neuromodulin), Gad65, GluR delta 2, GluR2, GluR5/6/7, Glutamine Synthetase, Jagged1, MAP2 (a+b), MAP2B, MASH1, N-Cadherin, Nestin, Neurofilament NF-H, Neurofilament NF-M, and Neuroglycan C; neuron-restricted progenitor cells such as Neuropilin-2, Nicastrin, P-glycoprotein, PSD-95, Pax-5, SMN, Serotonin Receptor 5-HT 2AR, Serotonin Receptor 5-HT 2BR, Synapsin I, Synaptophysin, Synaptotagmin, Syntaxin, Tau, TrkB, Tubby, Tyrosine Hydroxylase, Vimentin, mGluR1, and mGluR1 alpha; oligodendrocyte markers such as CD140a (PDGFR α), CD44, and CD44H (Pgp-1, H-CAM); and skin precursor cell markers such as CRABP2, Fibronectin, Nestin, Sca-1 (Ly6A/E), and Vimentin.

Illustrative gene expression markers for mesodermal cells include, but are not limited to early mesoderm markers such as CD31 (PECAM1), CD325 (M-Cadherin), CD34 (Mucosialin, gp 105-120), NF-YA, and Sca-1 (Ly6A/E); endothelial cell markers such as CD102, CD105 (Endoglin), CD106 (VCAM-1), CD109, CD112, CD116 (GM-CSF Receptor), CD117 (SCF R, c-kit), CD120a (TNF Receptor Type I), CD120b (TNF Receptor Type II), CD121a (IL-1 Receptor, Type I/p80), CD124 (IL-4 Receptor a), CD14, CD141 (Thrombomodulin) CD144 (VE-cadherin), CD146 (MCAM, MUC18), CD147 (Neurothelin), CD15 (SSEA-1, Lewis X), CD151, CD152 (CTLA-4), CD157, CD166 (ALCAM), CD18 (Integrin β2 chain, CR3/CR4), CD184 (CXCR4), CD192 (CCR2), CD201 (EPCR), CD202b (TIE2) CD202b (TIE2) (pY1102), CD202b (TIE2) (pY992), CD209, CD209a (CIRE, DC-SIGN), CD252 (OX-40 Ligand), CD253 (TRAIL), CD262 (TRAIL-R2, DR5), CD29 (Integrin β-1), CD31 (PECAM1), CD325 (M-Cadherin), CD34 (Mucosialin, gp 105-120), CD36, CD45 (Leukocyte Common Antigen, Ly-5), CD45R (B220), CD49d (Integrin α4), CD49e (Integrin α5), CD49f (Integrin α6), CD54 (ICAM-1), CD56 (NCAM), CD62E (E-Selectin), CD62L (L-Selectin), CD62P(P-Selectin), CDw93 (C1qRp), Flk-1 (KDR, VEGF-R2, Ly-73), HIF-1a, IP-10, Ly-6A/E (Sca-1), STAT3, STAT3 (pS727), STAT3 (pY705), and STAT3-interacting protein 1; heart or cardiogenesis markers such as α-Actinin, Annexin VI, CD106 (VCAM-1), CD117 (SCF R, c-kit), CD144 (VE-cadherin), CD166 (ALCAM), CD202b (TIE2), CD202b (TIE2) (pY1102), CD202b (TIE2) (pY992), CD31 (PECAM1), CD34 (Mucosialin, gp 105-120), CD66, CD66c, Caveolin-2, Caveolin-3, Connexin-43, Desmin, Flk-1 (KDR, VEGF-R2, Ly-73), GATA4, M-Cadherin, Myogenin, N-Cadherin, and NF-YA; hemangioblast markers such as CD144 (VE-cadherin), CD202b (TIE2), CD202b (TIE2) (pY1102), CD202b (TIE2) (pY992), CD31 (PECAM1), CD324 (E-Cadherin), CD34 (Mucosialin, gp 105-120), and Flk-1 (KDR, VEGF-R2, Ly-73); hematopoietic lineage markers including those of committed lymphoid progenitors such as CD10, CD117 (SCF R, c-kit), CD124 (IL-4 Receptor α), CD127 (IL-7 Receptor a), CD34 (Mucosialin, gp 105-120), CD38, CD90 (Thy-1), HLA-DR, and Terminal Transferase (TdT), megakaryoblasts such as CD34 (Mucosialin, gp 105-120), CD36, CD41, CD61 (Integrin β3), and HLA-DR; and monoblasts such as CD115 (FMS), CD116 (GM-CSF Receptor), CD11c, CD13, CD15 (SSEA-1, Lewis X), and CD33; myeloblasts such as CD114 (G-CSF Receptor), CD116 (GM-CSF Receptor), CD13, CD15 (SSEA-1, Lewis X), CD33, and CD91; proerythroblast cells such as CD105 (Endoglin), CD71 (Transferrin Receptor), PU.1, and TER-119/Erythroid cells (Ly-76); hematopoietic stem cells, including negative markers such as CD10, CD114 (G-CSF Receptor), CD13, CD138 (Syndecan-1), CD14, CD15 (SSEA-1, Lewis X), CD15s (Sialyl Lewis x), CD16, CD19, CD2, CD20, CD24, CD3, CD33, CD36, CD38, CD4, CD45 (Leukocyte Common Antigen, Ly-5), CD45R (B220), CD48, CD56 (NCAM), CD97, and GATA3, and positive markers such as CD105 (Endoglin), CD106 (VCAM-1), CD117 (SCF R, c-kit), CD164, CD184 (CXCR4), CD201 (EPCR), CD202b (TIE2), CD202b (TIE2) (pY1102), CD202b (TIE2) (pY992), CD31 (PECAM1), CD34 (Mucosialin, gp 105-120), CD44, CD59, CD84, CD90 (Thy-1), CD90.1 (Thy-1.1), CDw338 (ABCG2), CDw93 (C1qRp), CaM Kinase IV, Flk-1 (KDR, VEGF-R2, Ly-73), G-CSF, Ly-6A/E (Sca-1), MRP1, N-Cadherin, NF-YA, Notch1, P-glycoprotein, and WASP (Wiskott-Aldrich Syndrome Protein); and mesenchymal stem cell differentiation markers including adipocyte (fat) markers such as Acrp30 (Adiponectin); chondrocyte (cartilage) markers such as CD151 and CD44.

Illustrative gene expression markers for cells of the endodermal lineage, include, but are not limited to definitive endoderm markers such as β-Catenin, CD184 (CXCR4), GATA4, HNF-1β (TCF-2), and N-Cadherin; hepatic endoderm markers such as CD29 (Integrin β1), CD44H (Pgp-1, H-CAM), CD49f (Integrin α6), CD90 (Thy-1), HNF-1α, HNF-1β (TCF-2), and Tat-SF1; pancreatic endoderm markers such as CD49f (Integrin α6), Gad65, Gad67, Neurogenin 3, Neuropilin-2, and Synaptophysin; and primitive gut tube markers such as CDX2 and HNF-1β (TCF-2).

Cell type specific gene expression markers are also known to those having ordinary skill in the art and are suitable for use in the methods of the present invention for assessing the degree of cell programming or differentiation.

For example illustrative specific markers of adipogenic cells include, but are not limited to APOA2, APOD, APOE, APOC1, and PPARγ2.

Illustrative osteogenic specific markers include, but are not limited to BMP1, BMP2, OGN, and CTSK.

Illustrative neurogenic specific markers include, but are not limited to NTS, NRG1, MBP, MOBP, NCAM, and CD56.

Illustrative chondrogenic specific markers include, but are not limited to COL4, COL5, COL8, CSPG2, and AGC1.

Illustrative myogenic specific markers include, but are not limited to MYF5, TMP1, and MYH11.

Illustrative endothelial specific markers include, but are not limited to VWF and NOS.

1. Differentiation of Stem Cells

Human pluripotent stem cells are self renewing pluripotent cells which have the capacity to differentiate into a wide variety of cell types. This potentiality represents a promising source to overcome many human diseases by providing an unlimited supply of all cell types, including cells with particular mesodermal, endoderaml, and ectodermal characteristics.

As noted above, in various embodiments of the present invention, a method of reprogramming a cell to a more potent state is subsequently followed by a step of contacting the reprogrammed cell with one or more repressors and/or activators, or a composition comprising the same, that modulates a component of a cellular potency pathway in order to program the cell to a particular somatic cell type, that in some embodiments is the desired cell type for effecting a cell-based therapy as described elsewhere herein.

At least four basic methods have been developed to promote differentiation of pluripotent stem cells: (1) the formation of three-dimensional aggregates known as embryoid bodies (EBs), (2) the culture of pluripotent stem cells as monolayers on extracellular matrix proteins, (3) the culture of pluripotent stem cells directly on supportive stromal layers and (4) administration of pluripotent stem cells directly into an in vivo stem cell niche. Each method demonstrates that pluripotent stem cells can differentiate into a broad spectrum of cell types in culture and in vivo. The use of serum-free media with specific inducers to direct differentiation (Kubo et al., 2004, Ng et al., 2005a, Wiles and Johansson, 1999, Yasunaga et al., 2005) and the development of reporter pluripotent stem cells to monitor and access early differentiation steps (Fehling et al., 2003, Gadue et al., 2006, Ng et al., 2005a, Tada et al., 2005, Ying et al., 2003) have enhanced the efficacy or such cell programming strategies.

Human pluripotent stem cells can be differentiated to a wide range of somatic cell types, including, but not limited to hematopoietic, cardiac, neural, hepatic, and pancreatic lineages that can provide new therapies for some of society's most devastating diseases.

In programming cells, it is useful to understand the developmental signals that are responsible for patterning the three germs layers. Such information in combination with the cellular attributes of the desired programmed cells can lead to the successfully programming of any cell type. Developmental signaling related to endoderm induction is described in, for example, Kubo et al. 2004; Yasunaga et al., 2005; Gouon-Evans et al., 2006; Tada et al., 2005; Gadue et al., 2006; Schier, 2003; Wells and Melton, 1999; Gouon-Evans et al., 2006; and D'Amour et al., 2006. Developmental signaling related to mesoderm induction is described in, for example, Ema et al., 2006; Kataoka et al., 1997; Park et al., 2004; Nostro et al., 2008; Naito et al., 2006; Ueno et al., 2007; and Era et al., 2007. Developmental signaling related to ectoderm induction is described in, for example, Aubert et al., 2002; Kubo et al., 2004; Ying et al., 2003; and Kawasaki et al., 2000.

a) Hematopoietic Development of Human Pluripotent Stem Cells

Hematopoietic development of human pluripotent stem cells has been demonstrated by multiple groups using different induction schemes (Kaufman et al., 2001, Vodyanik et al., 2005, Chadwick et al., 2003, Ng et al., 2005b, Zambidis et al., 2005, Kennedy et al., 2007, Pick et al., 2007). Kinetic analysis revealed that the differentiating populations progressed through a PS stage defined by either BRACHYURY or MIXL1 expression, then to KDR+ (Flk-1+) or PDGFR+ mesoderm and subsequently to a yolk-sac hematopoietic program (Davis et al., 2008, Kennedy et al., 2007, Ng et al., 2005b, Zambidis et al., 2005). Hematopoietic progenitors were detected within the first week of differentiation (Davis et al., 2008, Kennedy et al., 2007, Vodyanik et al., 2006). The predominant population generated during the first 7-10 days of human pluripotent stem cell differentiation is primitive erythroid progenitors, indicating that the equivalent of yolk-sac hematopoiesis develops first in these cultures (Kennedy et al., 2007, Zambidis et al., 2005). The onset of hematopoiesis in human pluripotent stem cell cultures is marked by development of the hemangioblast between days 2 and 4 of differentiation, prior to establishment of the primitive erythroid lineage (Davis et al., 2008, Kennedy et al., 2007, Lu et al., 2007).

Although the early stages of development in human pluripotent stem cell cultures appear to represent the yolk-sac phase of hematopoiesis, more mature hematopoietic populations develop after extended periods of time. Analysis of cell surface phenotypes revealed progression from populations that expressed KDR, CD31, and CD34 to those that also expressed CD45, a marker found on fetal and adult hematopoietic cells (Kennedy et al., 2007, Vodyanik et al., 2005, Woll et al., 2007). T lymphoid progenitors have been generated from human pluripotent stem cells following differentiation directly on OP9 stromal cells in serum-containing media (Galic et al., 2006).

Several groups have described the development of human pluripotent stem cell-derived populations with in vivo hematopoietic repopulating potential (see, e.g., Wang et al., 2005; Tian et al., 2006; Narayan et al., 2006).

b) Differentiation of Pluripotent Stem Cells into Cardiomyocytes

The heart originates from lateral plate mesoderm and develops in at least two distinct waves of myogenesis from regions called the primary and secondary heart fields. Lineage-tracing studies indicate that both heart fields are marked by expression of Flk-1 and the transcription factor Nkx2.5, whereas the transcription factor Isl1 selectively marks the secondary heart field, giving rise to much of the right ventricle and outflow tracts (Ema et al., 2006, Moretti et al., 2006, Wu et al., 2006). These markers have proven useful in the identification of cardiac progenitors from pluripotent stem cells. Embryoid body-based differentiation of pluripotent stem cells stimulated with serum generates cardiomyocytes, which are readily detected by their spontaneous beating activity (Doetschman et al., 1985). The efficiency of this process is typically 1%-3% from mouse pluripotent stem cells and <1% from human pluripotent stem cells. An early approach for directing human pluripotent stem cells along a cardiac differentiation pathway involved using medium conditioned with the endodermal cell line, End-2 (which produces activin A and BMPs, among other factors). This technique was recently improved using a small molecule inhibitor of p38 MAP kinase, which almost doubled the yield of cardiomyocytes from human pluripotent stem cells (from 12% to 25%) by enhancing induction of mesoderm (Graichen et al., 2007).

A clearer picture is emerging of the signals that control cardiomyocyte differentiation (Zeineddine et al., 2005), and progenitors for cardiovascular cells have been defined. Signals mediated through Wnt/-catenin and TGF-family members including activin and BMPs promote differentiation of mouse pluripotent stem cells into mesoderm (Gadue et al., 2006, Lindsley et al., 2006, Naito et al., 2006, Ueno et al., 2007). Once mesoderm is induced, however, Wnt/-catenin signaling inhibits cardiac differentiation and can redirect the cells to alternate mesodermal fates (Naito et al., 2006, Ueno et al., 2007). Two groups have recently shown that human pluripotent stem cells can be induced to form cardiomyocytes efficiently (Laflamme et al., 2007, Yao et al., 2006). Both used defined media and induced differentiation with activin and BMP4 in serum-free cultures. Laflamme et al., 2007 reported that their populations contained >30% cardiomyocytes and could be enriched to 80%90% cardiomyocytes using density-gradient centrifugation.

Three recent studies used a developmental approach to identify multipotent cardiovascular progenitor cells in mouse pluripotent stem cell differentiation cultures. Wu et al., 2006 identified progenitors based on activity of the promoter for nkx2.5, a homeobox gene expressed in the earliest cardiomyocytes. These progenitors could be isolated both from developing transgenic mouse embryos and differentiating mouse pluripotent stem cell cultures, and they exhibited the capacity for both cardiac and smooth muscle differentiation (bipotential). Moretti et al., 2006 used the promoter for the secondary heart field marker, Isl-1, to identify progenitors from mouse embryos and differentiating mouse pluripotent stem cell. They showed that these progenitors could be expanded on feeder layers and that 12% of the resulting colonies gave rise to cardiomyocytes, endothelial cells, and smooth muscle cells (that is, they were tripotential). Kattman et al., 2006 used the VEGF receptor Flk-1, known to mark progenitors for multiple mesodermal lineages, to isolate hematopoietic and cardiovascular progenitors from mouse pluripotent stem cells. By analyzing embryoid bodies derived from mouse pluripotent stem cells over time, they found that the earliest Flk-1+ population to emerge contained hemangioblasts, progenitors for blood cells and endothelium. A later Flk-1+ population contained cardiovascular progenitors (cardiovascular colony-forming cells) that were able to generate cardiac, endothelial, and vascular smooth muscle cells (tripotential). Thus, commitment to the blood lineage occurs in mesoderm cells prior to cardiovascular commitment. Moreover, three of the major cell types in the heart can be derived from a common progenitor. These progenitors provide a new population for transplantation with the capability of contributing both to remuscularization and revascularization of the heart.

c) Differentiation of Pluripotent Stem Cells into Neural Phenotypes

Early methods to direct the differentiation of pluripotent stem cells to neural fates used treatment with retinoic acid (Bain et al., 1995), sequential culture in serum and serum-free media (Okabe et al., 1996), or coculture with specific stromal cell lines such as PA6 (Kawasaki et al., 2000). It is well established that trilineage neural progenitors capable of giving rise to neurons, astrocytes, and oligodendrocytes can be generated from pluripotent stem cells (reviewed in Joannides et al., 2007). Neural progenitors are commonly derived from differentiating pluripotent stem cell cultures by growing them under conditions optimized for adult neural progenitors, including growth as three-dimensional spheroids (neurospheres) in the presence of EGF and FGF2.

Although pluripotent stem cell-derived neural progenitors resemble adult and fetal neural progenitors in their trilineage capacity, microarray and DNA methylation assays indicate that there are many differences between these two progenitor populations (Shin et al., 2007).

Many signaling pathways known to regulate neural cell fate in the embryo have been exploited to control neural differentiation from pluripotent stem cell, including Notch (reviewed in Androutsellis-Theotokis et al., 2006, Hitoshi et al., 2002, Lowell et al., 2006), Sonic Hedgehog (Maye et al., 2004), Wnts (Davidson et al., 2007, Lamba et al., 2006), the FGF family (Rao and Zandstra, 2005), and members of the TGF-superfamily (Smith et al., 2008). The Notch pathway has emerged as a particularly important axis for controlling neural differentiation. Hitoshi et al., 2002 showed that neural progenitors could form in the absence of Notch signaling, but that these cells did not self-renew and hence were quickly lost to differentiation. Other investigators demonstrated that activation of Notch in mouse pluripotent stem cell derivatives after withdrawal of leukemia inhibitory factor (LIF) promoted exclusively neural differentiation, whereas inhibition of Notch blocked formation of neural progenitors. The ability of Notch ligands to promote neural progenitor formation required FGF receptor-mediated signaling (Lowell et al., 2006). Thus, Notch signaling is a key player in establishing neural progenitor cells, principally through effects on cell survival and promoting expansion of the progenitors by blocking their differentiation.

Joannides et al., 2007 have developed a protocol for neural induction of human pluripotent stem cells that uses chemically defined media at each step. Supplements include common amino acids and taurine; trace metals; vitamins; and the growth factors insulin, EGF, and FGF2. After optimizing techniques for passaging to generate small clumps of human ESCs, cells were induced to form neural progenitors and were expanded in defined media. Some cultures approached 90% nestin-negative/Pax6-positive cells that were trilineage-competent, and these cells could undergo 5-log expansion with a stable karyotype.

Wichterle et al., 2002 were the first to derive a protocol for the directed differentiation of pluripotent stem cells to a specific neural type, using induction with retinoic acid and a Sonic Hedgehog analog to induce transplantable murine spinal motor neurons (Wichterle et al., 2002). Following this pioneering work, multiple investigators developed techniques to induce differentiation of pluripotent stem cells into specific neuronal populations, including progenitors for retinal photoreceptors, cerebellar granule neurons, and cerebral-type neurons that use glutamate, GABA, and dopamine as their major neurotransmitters. Different lines of human ESCs appear to preferentially make one neuron type over another.

d) Differentiation of Pluripotent Stem Cells to Dopamine Neurons

Dopamine neurons are of particular interest because of their central role in Parkinson's disease. Many studies now show that mouse and human pluripotent stem cells can form dopamine neurons, and they appear to arise through the neural progenitor stage described above. These neurons express tyrosine hydroxylase (required for dopamine synthesis), release dopamine upon depolarization, and form at least rudimentary synapses in vitro with transmitter reuptake abilities (reviewed in Kim et al., 2007). The combined use of FGF8 and SHH effectively induces dopamine neurons from pluripotent stem cell-derived neural progenitors generated from either mouse pluripotent stem cells (Lee et al., 2000) or human pluripotent stem cells (Yan et al., 2005). Although recombinant factors are now routinely used, most protcols do include undefined reagents at one or more stages of dopamine neuron production, due to coculture with stromal cell lines or the use of conditioned media. One of the best-defined protocols for human pluripotent stem cell differentiation into dopamine neurons was validated in three human pluripotent stem cell lines and two monkey pluripotent stem cell lines (Perrier et al., 2004). Neural progenitors were induced in this study using stromal cell coculture, followed by SHH and FGF8 to specify a neuronal fate. Addition of ascorbate, BDNF, glial-derived neurotrophic factor, dibutyryl cyclic-AMP, and TGF-3 yielded cultures that were 30%-50% neurons expressing β-III tubulin. Of these neurons, 65%-80% expressed tyrosine hydroxylase, and the majority fired action potentials that could be blocked by tetrodotoxin, a Na+ channel blocker.

e) Differentiation of Pluripotent Stem Cells to Oligodendrocytes

Astrocytes and oligodendrocytes are the two neuroglial types in the central nervous system. Diseases of the central nervous system typically involve proliferation of astrocytes and loss of oligodendrocytes and the protective myelin sheath they produce. Thus, derivation of oligodendrocytes from pluripotent stem cells is an important goal for cell replacement therapy. The most common protocols involve an initial differentiation step to neural progenitors, followed by expansion, further differentiation, and selection. Oligodendrocytes were first efficiently derived from mouse pluripotent stem cells (Brustle et al., 1999), where medium containing FGF2 and EGF was used to expand progenitors, followed by a switch to FGF2 and PDGF to yield bipotential glial progenitors. These glial progenitors were transplanted into the spinal cords of rats with a genetic deficiency in myelin production, yielding myelinated fibers in the majority of animals. Transplantation of these glial progenitors into the brains of developing rats (at embryonic day 17) resulted in widespread myelin-producing cells of mouse origin. Oligodendrocytes were first generated from human pluripotent stem cells by Zhang et al., 2001b, who used a similar strategy involving FGF treatment followed by growth as neurospheres.

The first detailed protocol for directed differentiation of oligodendrocytes from human pluripotent stem cells involved generation of neurospheres, followed by several rounds of expansion and selection in various media containing, among other things, the multicomponent additive B27, thyroid hormone, retinoic acid, FGF2, EGF, and insulin (Nistor et al., 2005). After 42 days of culture, the desired cells were found in yellow spheroids, which upon differentiation as low-density monolayers formed 85%-95% oligodendrocytes (based on expression of the markers GalC, RIP, and O4). The remaining cells were astrocytes or neurons. Kang et al., 2007 recently reported a simplified protocol for isolation of oligodendrocyte progenitors from human pluripotent stem cell, using a multistep procedure that yielded 80% oligodendrocytes that were capable of myelinating fetal neural explants in vitro. These experiments show that human oligodendrocytes can be generated in large numbers and used to restore myelination under some circumstances.

f) Differentiation of Pluripotent Stem Cells to Pancreatic Cells

The potential to generate functional pancreatic cells from pluripotent stem cells differentiated in culture has raised the exciting possibility of a new source of insulin-producing cells for transplantation to treat type I diabetes. Given the therapeutic potential of pluripotent stem cell-derived cells, significant efforts have focused on isolating such cells in both mouse and human pluripotent stem cell cultures. Initial attempts to generate the pancreatic lineage used mouse pluripotent stem cells (reviewed in Spence and Wells, 2007), but the most successful differentiation along this pathway has been recently achieved with human pluripotent stem cells (D'Amour et al., 2006). The key to generating pancreatic lineage cells from human pluripotent stem cells relies on recapitulating the critical signals that regulate endocrine pancreas development in the embryo.

The pancreas develops from foregut endoderm, and the earliest stages of induction are controlled in part by retinoic acid (RA) and the inhibition of SHH signaling (reviewed in Collombat et al., 2006, Murtaugh, 2007, Spence and Wells, 2007). The first indication of pancreas morphogenesis is the upregulation of Pdx1, a gene encoding a transcription factor that is essential for development of this tissue. Although indicative of pancreas specification, expression of Pdx1 is not restricted to pancreatic tissues as it is also found in the region of the foregut that will give rise to the pyloric region of the stomach and the proximal duodenum. Coexpression of the transcription factor encoded by the Ptf1a/P48 gene together with Pdx1 marks the population that will give rise to the pancreas. Recent evidence suggests that expansion of the pancreatic progenitor population is supported by the surrounding mesenchyme through FGF10 secretion. FGF10 enhances Notch signaling, which represses expression of the transcription factor Ngn3 and promotes expansion of pancreatic progenitors. Expression of Ngn3 within the pancreatic epithelium defines the development of a progenitor population for all endocrine lineages, including the cells. With further maturation, cohorts of factors function to establish the different endocrine lineages. Pancreatic cell development is dependent, in part, on the combined activity of Nkx2.2, Nkx6.1, Pax4, Pax6, and MafA.

Through the sequential activation of different signaling pathways, D'Amour et al., 2006 demonstrated that it is possible to recapitulate many of these developmental stages in human pluripotent stem cell cultures. In this study, endoderm induced by activin signaling in monolayer cultures was specified to a pancreatic fate through a combination of FGF and retinoic acid signaling as well as inhibition of SHH signaling. Following specification, the cultures were treated with a γ-secretase inhibitor to inhibit Notch signaling and a combination of exendin-4, IGF1, and hepatocyte growth factor (HGF), which are known to promote cell maturation. With this protocol, the population progressed through normal stages associated with pancreas development, including the induction of FOXA2+SOX17+CXCR4+ endoderm, the formation of HNF1+HNF4+ gut tube-like cells, specification of PDX1+ progenitors, development of NGN3+NKX2.2+ endocrine progenitors, and finally maturation to insulin-producing cells. Differentiation with this protocol was fast and reasonably efficient: 7% of the population was insulin-positive within 16 days of differentiation. The cells generated in these cultures expressed high levels of insulin and released C-peptide following depolarization with potassium chloride. The presence of C-peptide, released when proinsulin is converted to insulin, is a clear demonstration that the insulin is produced by the human pluripotent stem cell-derived cells and not absorbed from the culture media.

Several other groups have analyzed the potential of activin-induced human pluripotent stem cell-derived populations to generate functional cells using different differentiation schemes. Jiang et al., 2007a induced endoderm with a combination of activin and sodium butyrate and promoted further maturation to PDX1+ populations and subsequently insulin+ cells by culturing the cells as aggregates, initially in the presence of bFGF, EGF, and the BMP inhibitor Noggin and finally in the presence of nicotinamide and IGF2. Development with this protocol was somewhat slower with cultures maintained for up to 36 days. At this stage, C-peptide-positive cells were detected in small clusters that also contained glucagon- and somatostatin-positive cells, reminiscent of pancreatic islets. The cells in these clusters release C-peptide in response to glucose, a key characteristic of mature cells.

### IV. Epigenetic Modulation: Chromatin Remodeling

A. Epigenetic Modifications of Stem Cells

In order to establish or maintain pluripotency in a cell, genes whose up-regulation leads to differentiation should be inactive. Polycomb group proteins (PcG) play important roles in silencing these developmental regulators of differentiation. The PcG proteins function in two distinct Polycomb Repressive Complexes, PRC1 and PRC2. Genome-wide binding site analyses have been carried out for PRC1 and PRC2 in mouse ESCs and for PRC2 in human ESCs (Lee T. I., et al., Control of developmental regulators by Polycomb in human embryonic stem cells. Cell (2006) 125:301-313 and Boyer L. A. et al., Polycomb complexes repress developmental regulators in murine embryonic stem cells. Nature (2006) 441:349-353). The genes regulated by the PcG proteins are co-occupied by nucleosomes with trimethylated H3K27. These genes are transcriptionally repressed in ESCs and are preferentially activated when differentiation is induced. Many of these genes encode transcription factors with important roles in development. For example, the pluripotency factors Oct-3/4, Sox-2 and Nanog co-occupy a significant fraction of the PcG protein regulated genes (Lee et al., 2006 and Boyer et al., 2006). These data suggest that the PcG proteins may facilitate pluripotency maintenance by suppressing developmental pathways.

Developmental regulators inactive in ESCs require activation upon differentiation. ESCs possess specific mechanisms to ensure that these genes are potent for activation. The recently discovered ‘bivalent’ histone code keeps its target gene in a state “poised” for transcription (Bernstein B. E., et al., A bivalent chromatin structure marks key developmental genes in embryonic stem cells. Cell (2006) 125:315-326 and Azuara V., et al., Chromatin signatures of pluripotent cell lines. Nat. Cell Biol. (2006) 8:532-538). The bivalent domain has both repressive and active histone markers: a large region of H3K27 trimethylation harboring a smaller region of H3K4 trimethylation. In ESCs, bivalent domains are frequently associated with developmentally regulated transcription factors that are expressed at low levels. Upon differentiation, most of the bivalent domains become either H3K4 methylated or H3H27 methylated, consistent with associated changes in gene expression (Bernstein et al., 2006). Although the bivalent histone code primarily regulates key developmental transcription factors, some tissue-specific genes, such as Ptcra, II12b and Alb1, are controlled by windows of unmethylated CpG dinucleotides and putative ‘pioneer’ factors in ESCs. These tissue-specific genes are silenced in ESCs, and most of the CpG dinucleotides in their promoter and enhancer regions are methylated. The unmethylated windows are located in the silent enhancers where the binding of transcription factors is required for maintaining the unmethylated state. These unmethylated windows are necessary for the activation of tissue-specific genes in differentiated cells (Xu J., et al., Pioneer factor interactions and unmethylated CpG dinucleotides mark silent tissue-specific enhancers in embryonic stem cells. Proc. Natl. Acad. Sci. USA (2007) 104:12377-12382).

Beyond the specific regulations of development-related genes, ESCs maintain chromatin in a highly dynamic and transcriptionally permissive state. First, fewer heterochromatin foci are detected in ESC nuclei, where they appear to be more diffuse than those in differentiated cells. Second, fluorescence recovery after photobleaching and biochemical analyses reveal that compared with differentiated cells, ESCs have an increased fraction of loosely bound or soluble architectural chromatin proteins, including core and linker histones, as well as the heterochromatin protein HP1. A hyperdynamic chromatin structure is functionally important for pluripotency maintenance, as restriction of the dynamic exchange of the linker histone H1 prevents ESC differentiation (Meshorer E., et al., Hyperdynamic plasticity of chromatin proteins in pluripotent embryonic stem cells. Dev. Cell (2006) 10:105-116). Third, the status of histone modifications also indicates that the chromatin in ESCs is more transcriptionally permissive than in differentiated cells. Consistent with the global dynamics of chromatin, ESC differentiation is associated with a decrease in global levels of active histone marks, such as acetylated histone H3 and H4, and an increase in repressive histone marks, specifically histone H3 lysine 9 methylation (Meshorer et al., 2006 and Lee J. H., et al., Histone deacetylase activity is required for embryonic stem cell differentiation. Genesis (2004) 38:32-38). Such a highly dynamic and transcriptionally permissive chromatin environment may facilitate rapid transcriptional profile alternations upon differentiation and allow various transcriptional profiles to be established.

The present invention contemplates, in part, to provide methods and compositions that reprogram or dedifferentiate and program or differentiate cells by altering the epigenetic state of the cell. Generally, when reprogramming or dedifferentiating a cell of the present invention, epigenetic marks on chromatin will be required to make the chromatin more accessible to transcriptional activation, i.e., to place the chromatin in a more naïve state in a reprogrammed cell than in the same non-reprogrammed cell. Without wishing to be bound by a particular theory, during reprogramming, genes that favor an increase in potency (e.g., Oct-3/4, Sox-2, Nanog, c-Myc, Klf-4, Lin 28, hTERT, and the targets of these genes, and the like) generally acquire epigenetic marks of transcriptionally active chromatin (e.g., DNA demethylation, histone acetylation, histone methylation at Lysine 4, Lysine 36, or Lysine 79 of Histone H3 (H3K4, H3K36, and H3K79, resp.), and histone demethylation at Lysine 9, or Lysine 27 of Histone H3 (H3K9 and H3K27, respectively) or Lysine 20 of Histone H4 (H4K20), and the like).

In contrast, genes that favor programming or differentiation, i.e., a reduction in potency, acquire epigenetic marks on DNA and histones that “silence” these genes and make them less accessible to the transcriptional machinery of the cell (e.g., DNA methylation, histone deacetylation, histone methylation at H3K9, H3K27, and H4K20, and histone demethylation at H3K4, H3K36, and H3K79, and the like). Thus, reprogramming or dedifferentiating cells of the present invention requires epigenetic modification and chromatin remodeling, which involves DNA and histone modifications.

Thus, in one embodiment, a method of altering the potency of a cell, comprising contacting the cell with one or more repressors, modulates at least one component of an epigenetic or chromatin remodeling pathway, and thereby alters the potency of the cell.

In a related embodiment, a method of reprogramming a cell, comprises contacting the cell with one or more repressors, and modulating at least one component of an epigenetic or chromatin remodeling pathway, thereby reprogramming or dedifferentiating the cell. Repression may occur by any one or more of the mechanisms provided herein, including but not limited to, directly or indirectly repressing a histone methyltransferase (HMT) that methylates, for example, Lysine 9, or Lysine 27 of Histone H3 (H3K9 and H3K27, respectively) or Lysine 20 of Histone H4 (H4K20), of one or more genes or factors that is associated with the establishment or maintenance of a multipotent, pluripotent or totipotent state. In a related embodiment, one or more repressors may repress a repressor of an HMT that methylates, for example, Lysine 4, Lysine 36, or Lysine 79 of Histone H3 (H3K4, H3K36, and H3K79, resp.), of one or more genes or factors that is associated with the establishment or maintenance of a multipotent, pluripotent or totipotent state.

In a particular embodiment, a method of reprogramming a cell, or of altering the potency of a cell to a more potent state compared to the ground potency state is achieved by repression including, but not limited to, direct or indirect repression of a histone demethylase (HDM) that removes methylation at sites on “activated” histones (e.g., H3K4, H3K36 or H3K79) of one or more genes or factors that is associated with the establishment or maintenance of a multipotent, pluripotent or totipotent state. In a related embodiment, one or more repressors can repress a repressor of an HDM that removes methylation at sites on transcriptionally inactive histones (e.g., H3K9, H3K27 or H4K20).

In certain embodiments, one or more repressors, represses, either directly or indirectly a histone deacetylase (HDAC) associated with the deacetylation (marker of heterchromatin) of at least one gene or factor that is associated with the establishment or maintenance of a multipotent, pluripotent or totipotent state. In a certain related embodiment, a composition comprising one or more repressors, represses a repressor of a histone acetyltransferase (HAT) that acetylates (marker of transcriptionally active chromatin) one or more genes or factors that is associated with the establishment or maintenance of a multipotent, pluripotent or totipotent state.

In another embodiment, a method of altering the potency of a cell, comprises contacting the cell with one or more activators in order to modulate at least one component of an epigenetic or chromatin remodeling pathway, and thereby alters the potency of the cell. In a related embodiment, a method of reprogramming a cell, comprises contacting the cell with one or more activators, and modulating at least one component of an epigenetic or chromatin remodeling pathway, thereby reprogramming or dedifferentiating the cell. Activation may occur by any one or more of the mechanisms provided herein, including but not limited to, directly or indirectly activating an HMT that methylates, for example, H3K4, H3K36 or H3K79 of one or more genes or factors that is associated with the establishment or maintenance of a multipotent, pluripotent or totipotent state. In a related embodiment, one or more activators may activate a repressor of an HMT that methylates, for example, H3K9, H3K27 or H4K20 of one or more genes or factors that is associated with the establishment or maintenance of a multipotent, pluripotent or totipotent state.

In a particular embodiment, a method of reprogramming a cell, or of altering the potency of a cell to a more potent state is achieved by activation including, but not limited to, direct or indirect activation of an HDM that removes methylation at sites on transcriptionally inactive histones (e.g., H3K9, H3K27 or H4K20) of one or more genes or factors important to the establishment or maintenance of a multipotent, pluripotent or totipotent state. In a related embodiment, one or more activators can activate a repressor of an HDM that removes methylation at sites on “activated” histones (e.g., H3K4, H3K36 or H3K79).

In certain embodiments, one or more activators, activates, either directly or indirectly a HAT that acetylates (marker of transcriptionally active chromatin) one or more genes or factors important to the establishment or maintenance of a multipotent, pluripotent or totipotent state. In a certain related embodiment, a composition comprising one or more activators, activates a repressor of an HDAC associated with the deacetylation (marker of heterchromatin) of at least one gene or factor important to the establishment or maintenance of a multipotent, pluripotent or totipotent state.

In other embodiments, one or more repressors and activators, or a composition comprising the same, acts synergistically to promote the same epigenetic or chromatin modifications or compatible modifications (i.e., either positively regulating transcription or negatively regulating transcription). Thus, in particular embodiments, a method of altering the potency of a cell, comprises contacting the cell with a composition comprising one or more repressors and/or activators, that synergistically modulate one or more components of cellular pathway associated with the pluripotency of the cell (e.g., an epigenetic or chromatin remodeling pathway), and thereby alter the potency of the cell. In a related embodiment, a method of reprogramming a cell, comprises contacting the cell with one or more repressors and/or activators, and modulating at least one component of an epigenetic or chromatin remodeling pathway in a synergistic fashion, thereby reprogramming or dedifferentiating the cell.

Illustrative repressors of components of epigenetic and chromatin modification pathways can be a polynucleotide (e.g., a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, or a ssDNA), polypeptide or active fragment thereof (e.g., an antibody, a protein, an enzyme, a peptidomimetic, a peptoid, or a transcriptional factor), or a small molecule, and the like.

Illustrative activators of components of epigenetic and chromatin modification pathways can be an antibody or an antibody fragment, an mRNA, a bifunctional antisense oligonucleotide, a dsDNA, a polypeptide or an active fragment thereof, a peptidomimetic, a peptoid, or a small organic molecule, and the like.

In particular embodiments, the activator or repressor is a transcription factor that activates or represses, either directly or indirectly, the transcription of a chromatin remodeling enzyme as described herein throughout. In other embodiments, the activator or repressor of an epigenetic or chromatin remodeling pathway is the chromatin remodeling enzyme itself, including but not limited to a histone methyltransferase, histone demethylase, histone acteylase, and the like.

It would be understood by those having ordinary skill in the art that the above embodiments are illustrative, and that the compositions and methods of the present invention are suitable for use in a method to alter the potency of a cell to a more potent state, or reprogram or dedifferentiate a cell by modulating components of all epigenetic and chromatin remodeling pathways, including, but not limited to DNA methylation, histone acetylation, methylation, phosphorylation, ubiquitination, sumoylation, ADP-ribosylation, deimination, and proline isomerization. The skilled artisan would also recognize that multiple components of epigenetic and chromatin remodeling pathways can be modulated in parallel or sequentially in order to enhance the transcriptionally active chromatin of one or more genes or factors associated with establishing or maintaining the pluripotency of a cell. Exemplary epigenetic and chromatin remodeling pathways are discussed in further detail below, along with exemplary activators and repressors for each pathway.

B. Chromatin and Histone Modifications

Chromatin is the state in which DNA is packaged within the cell. The nucleosome is the fundamental unit of chromatin and it is composed of an octamer of four core histones (H3, H4, H2A, H2B) around which 147 base pairs of DNA are wrapped. Core histone proteins are evolutionary conserved and consist mainly of flexible N-terminal tails protruding outward from the nucleosome, and globular C-terminal domains making up the nucleosome scaffold. Histones function as acceptors for a variety of post-translational modifications. At least eight different classes of nucleosome modifications have been characterized to date and many different sites have been identified for each class.

C. Histone-Modifying Enzymes

The identification of the enzymes that direct modification has been the focus of intense activity over the last 10 years. Enzymes have been identified for acetylation (Sterner et al., 2000), methylation (Zhang et al., 2006), phosphorylation (Nowak et al., 2004), ubiquitination (Shilatifard, 2006), sumoylation (Nathan et al., 2006), ADP-ribosylation (Hassa et al., 2006), deimination (Cuthbert et al., 2004, Wang et al., 2004), and proline isomerization (Nelson et al., 2006).

D. Acetylation

Histone acetylation is almost invariably associated with activation of transcription. Acetyltransferases are divided into three main families, GNAT, MYST, and CBP/p300 (Sterner et al., 2000). In general, these enzymes modify more than one lysine but some limited specificity can be detected for some enzymes. Most of the acetylation sites characterized to date fall within the N-terminal tail of the histones, which are more accessible for modification. However, a lysine within the core domain of H3 (K56) has recently been found to be acetylated. A yeast protein, SPT10, may be mediating acetylation of H3K56 at the promoters of histone genes to regulate gene expression (Xu et al., 2005), whereas the Rtt109 acetyltransferase mediates this modification more globally (Han et al., 2007, Driscoll et al., 2007, Schneider et al., 2006). The K56 residue is facing toward the major groove of the DNA within the nucleosome, so it is in a particularly good position to affect histone/DNA interactions when acetylated.

Histones and transcription factors such as p53, E2F1, and GATA1 are known to be substrates for HATs. (The Cancer Journal, 13,1, 2007, 23). Other non-histone HAT substrates include, for example, Sin 1p, HMG-17, EKLF, TFIIEbeta, and TFIIF.

Histone acetyltransfersases and their substrates, include, but are not limited to: HAT1 (H4K5 and H4K12); CBP/p300 (H3K14, H3K18, H4K5, H4K8, H2AK5, H₂BK12, and H2AK15); PCAF/GCN5 (H3K9, H3K14, and H3K18); TIP60 (H4K5, H4K8, H4K12, H4K16 and H3K14); HBO1 (H4K5, H4K8, and H4K12); ScSAS3 (H3K14, and H3K23); ScSAS2 (H4K16); and ScRTT109 (H3K56).

Illustrative examples of HAT inhibitors are anacardic acid, garcinol, curcumin, isothiazolones, butyrolactone, and MC1626 (2-methyl-3-carbethoxyquinoline), among others.

E. Deacetylation

The reversal of histone acetylation correlates with transcriptional repression. There are three distinct families of histone deacetylases: the class I and class II histone deacetylases and the class III NAD-dependant enzymes of the Sir family. They are involved in multiple signaling pathways and they are present in numerous repressive chromatin complexes. In general these enzymes do not appear to show much specificity for a particular acetyl group although some of the yeast enzymes have specificity for a particular histone: Hda1 for H3 and H₂B; Hos2 for H3 and H4. The fission yeast class III deacetylase Sir2 has some selectivity for H4K16ac, and recently the human Sir family member SirT2 has been demonstrated to have a similar preference (Vaquero et al., 2006).

For example, HDAC inhibitors can induce an open chromatin conformation through the accumulation of acetylated histones, facilitating the transcription of numerous regulatory genes. There are 4 classes of HDAC enzymes. Class I, II, and IV share sequence and structural homology within their catalytic domains and share a related catalytic mechanism that does not require a co-factor, but does require a zinc (Zn) metal ion. In contrast, class III (sirtuins) do not share sequence or structural homology with the other HDAC families and use a distinct catalytic mechanism that is dependant on the oxidized form of nicotinamide adenine dinucleotide (NAD+) as a co-factor. Sirtuins have been linked to counteracting age associated diseases such as type II diabetes, obesity and neurodegenerative diseases (Oncogene, 2007, 26, 5528).

Illustrative proteins that are non-histone substrates of HDAC's and that may be targeted in order to effect chromatin remodeling include, for example, DNA binding transcription factors (e.g., p53, c-myc, AML-1, BCL-6, E2F1, E2F2, E2F3, GATA-1, GATA-2, GATA-3, GATA-4, YY1, NF-kb, MEF-2, CREB, HIF-1α, BETA-2, POP-1, IRF-2, IRF-7, SRY, EKLF), steroid receptors (e.g., androgen receptor, estrogen receptor alpha, glucocorticoid receptor), transcription co-regulators (e.g., Rb, DEK, MSL-3, HMGI(Y)/HMGA1, CtBP2, PGC-1alpha), signaling mediators (e.g., STAT-3, Smad-7, β-catenin, IRS-1), DNA repair enzymes (e.g., KU70, WRN, TDG, NEIL2, FEN1), nuclear import proteins (Rch1, importin-alpha7),chaperone proteins (e.g., HSP90), structural proteins (e.g., alpha-tubulin), inflammation mediators (e.g., HMGB1) and/or viral proteins (e.g., E1A, L-HDAg, SHDAg, T-antigen, HIV tat).

Particular illustrative examples of HDAC inhibitors include, for example, butyrate; suberoylanilide hydroxamic acid (SAHA, a.k.a. Vorinostat); Belinostat/PXD101; MS275; LAQ824/LBH589; CI994; MGCD0103; nicotinamide, as well derivatives of NAD, dihydrocoumarin, naphthopyranone, and 2-hydroxynaphaldehydes; Trichostatin A; Chlamydocin; cyclic tetrapeptide trapoxin A and trapoxin B; electrophilic ketones; aliphatic acid compounds such as phenylbutyrate and valproic acid; and the natural product Apicidin, among others.

F. Lysine Methylation

Lysine methyltransferases have enormous specificity compared to acetyltransferases. They usually modify one single lysine on a single histone and their output can be either activation or repression of transcription (Bannister et al., 2005).

Three methylation sites on histones are implicated in activation of transcription: H3K4, H3K36, and H3K79. Two of these, H3K4me and H3K36me, have been implicated in transcriptional elongation. In budding yeast H3K4me3 localizes to the 5′ end of active genes and is found associated with the initiated form of RNA Pol II (phosphorylated at serine 5 of its C-terminal domain). H3K36me3 is found to accumulate at the 3′ end of active genes and is found associated with the serine 2 phosphorylated elongating form of RNA pol II. One role for H3K36me is the suppression of inappropriate initiation from cryptic start sites within the coding region (Carrozza et al., 2005, Cuthbert et al., 2004, Joshi et al., 2005, Keogh et al., 2005). To achieve this, methylation at H3K36 recruits the EAF3 protein, which in turn brings the Rpd35 deacetylase complex to the coding region. Deacetylation then removes any acetylation that was placed in the coding region during the process of transcription, thus resetting chromatin into its stable state. This “closing up” of chromatin, following the passage of RNA pol II, prevents access of internal initiation sites that may be inappropriately used. On aspect of the function of methylation at H3K79 is in the activation of HOXA9 and it has a role in maintaining heterochromatin, probably indirectly, by limiting the spreading of the Sir2 and Sir3 proteins into euchromatin.

Three lysine methylation sites are connected to transcriptional repression: H3K9, H3K27, and H4K20. Methylation at H3K9 is implicated in the silencing of enchromatic genes as well as forming silent heterochromatin mentioned above. Repression involves the recruitment of methylating enzymes and HP1 to the promoter of repressed genes. Delivery of these components of methylation-based silencing is mediated by corepressors such as RB and KAP1.

H3K27 methylation has been implicated in the silencing of HOX gene expression. A similar mechanism is likely to be operational for the involvement of H3K27me in silencing of the inactive X chromosome and during genomic imprinting. It has a role in the formation of heterochromatin and has a role in DNA repair. Recently a protein has been identified that may mediate its functions. The JMJD2A lysine demethylase has been demonstrated to bind H3K20me (Huang et al., 2006, Kim et al., 2006) via a tudor domain. JMJD2A can also bind the positively acting methylation site at H3K4.

Links between histone methylation and DNA methylation have been demonstrated in Neurospora crassa and in plants, and experimental evidence has shown that histone methylation may be a prerequisite for DNA methylation and transcriptional silencing in Neurospora and Arabidopsis. There are also reports that DNA methylation may trigger H3-K9 methylation in Arabidopsis, suggesting interplay between histone and DNA methylation in maintaining the silent status of the chromatin.

Histone methyltransfersases and their substrates, include, but are not limited to: SUV39H1 (H3K9); SUV39H2 (H3K9); G9a (H3K9); ESET/SETDB1 (H3K9); EuHMTase/GLP (H3K9); CLL8 (H3K9); SpClr4 (H3K9); MLL1 (H3K4); MLL2 (H3K4); MLL3 (H3K4); MLL4 (H3K4); MLL5 (H3K4); SET1A (H3K4); SET1B (H2K4); ASH1 (H3K4); Sc/Sp SET1 (H3K4); SET2 (H3K36); NSD1 (H3K36); SYMD2 (H3K36); DOT1 (H3K79); Sc/SpDOT1 (H3K79); Pr-SET 7/8 (H4K20); SUV4 20H1 (H4K20); SUV4 20H2 (H4K20); SpSET 9 (H4K20); EZH2 (H3K27); and RIZ1 (H3K9).

G. Lysine Demethylation

For a number of years following the discovery of histone methyltransferases, the existence of demethylases was contentious. The discovery of the first histone demethylase LSD1 (Shi et al., 2004) has opened the way for the discovery of many other such enzymes. So far there are two types of demethylase domains, with distinct catalytic reactions: the LSD1 domain and the JmjC domain. LSD1 acts to demethylate H3K4 and repress transcription (Shi et al., 2004). However, when LSD1 is present in a complex with the androgen receptor, it demethylates H3K9 and activates transcription (Metzger et al., 2005). H3K9 can also be demethylated by JHDM2A (Yamane et al., 2006), JMJD2A/JHDM3A (Tsukada et al., 2006, Whetstine et al., 2006), JMJD2B (Fodor et al., 2006), JMJD2C/GASC1 (Cloos et al., 2006), and JMJD2D (Shin et al., 2006). Methylation at H3K36 can be reversed by JHDM1 (Tsukada et al., 2006, Whetstine et al., 2006), JMJD2A/JHDM3A (Klose et al., 2006), and JMJD2C/GASC1 (Cloos et al., 2006). Structural analysis of JMJD2A has shown that three distinct domains, in addition to the JmjC domain, are necessary for catalytic activity (Chen et al., 2006).

It is clear that these HDMs will antagonize methylation by being delivered to the right place at the right time (Yamane et al., 2006). Also, the activity of the enzymes are under the influence of the proteins they bind, as in the case of LSD1/BHC110, which acts on nucleosomal substrates in the presence of CoREST (Lee et al., 2005). A very important part of the specificity of these new demethylases also comes down to the state of methylation they act on. Their selectivity for mono-, di-, or trimethylated lysines allows for a larger functional control of lysine methylation (Shi et al., 2007).

Inhibitors of LSD1 may be useful biological tools and have therapeutic properties in the treatment of diseases involving abnormal epigenetic regulation, such as cancer (Biochemistry, 2007, 46, 23, 6897 and Biochemistry, 2007, 46, 14, 4410).

Illustrative examples of inhibitors of histone demethylase include trans-2-phenyl cyclopropylamine, which is an irreversible inhibitor of LSD1. Peptides-based inhibitors may also be used.

Histone lysine demethylases and their substrates, include, but are not limited to: LSD1/BHC110 (H2K4); JHDM1a (H3K36); JHDM1b (H3K36); JHDM2a (H3K9); JHDM2b (H3K9); JMJD2A/JHDM3A (H3K9 and H3K36); JMJD2B (H3K9); JMJD2C/GASC1 (H3K9 and H3K36); and JMJD2D (H3K9).

H. Arginine Methylation

Like lysine methylation, arginine methylation can be either activating or repressive for transcription, and the enzymes (protein arginine methyltransferases, PRMT's) are recruited to promoters by transcription factors (Lee et al., 2005). The most studied promoter regarding arginine methylation is the estrogen-regulated pS2 promoter. One observation regarding this promoter is that modifications are cycling (appear and disappear) during the activation process (Metivier et al., 2003).

Histone lysine demethylases and their substrates, include, but are not limited to: CARM1 (H3R2, H3R17, and H3R26); PRMT4 (H4R3); and PRMT5 (H3R8 and H4R3).

I. Phosphorylation

Little is known about histone phosphorylation and gene expression. MSK1/2 and RSK2 in mammals, and SNF1 in budding yeast, have been shown to target H3S10. A role for H3S10 phosphorylation has been demonstrated for the activation of NFKB-regulated genes and also “immediate early” genes such as c-fos and c-jun. Concomitant with this phosphorylation is the appearance on chromatin of a phosphor-binding protein 14-3-3 (Macdonald et al., 2005). Recently, a global ChIP on CHIP analysis of many kinases in budding yeast has shown that they are present on the chromatin of specific genes (Pokholok et al., 2006). This has important implications regarding signal transduction. It suggests that the mainly cytoplasmic protein phosphorylation cascades that have dominated signal transduction processes for many years may have a more direct effect on gene expression through the phosphorylation of chromatin. Condensation and decondensation of chromatin are important processes during the replicative cell cycle. Two phosphorylation events in mammalian cells may play an important role in these processes during mitosis. The first is phosphorylation of H3S10 during mitosis by the Aurora B kinase. Recent data suggest that one of the mechanisms by which H3S10 phosphorylation may function is via the displacement of HP1 from H3K9me, which normally compacts chromatin (Fischle et al., 2005). The second phosphorylation event is at H3T3 (Dai et al., 2005). This modification is mediated by the Haspin kinase and is required for normal metaphase chromosome alignment. A number of other phosphorylation sites have been implicated in this process in budding yeast. Phosphorylation of H4S1 regulates sporulation (Krishnamoorthy et al., 2006), and phosphorylation of H₂BS10 regulates peroxide-induced apoptosis (Ahn et al., 2005). The latter modification is on a residue that is not conserved in mammals. However, phosphorylation of mammalian H₂BS14 by Mst1 is thought to play an analogous function.

Histone kinases and their substrates, include, but are not limited to: Haspin (H3T3); MSK1 (H3S28); MSK2 (H3S28); CKII (H4S1); and Mst1 (H₂BS14).

J. Ubiquitylation

Ubiquitylation is a relatively large modification that has been found on H2A (K119) and H₂B (K20 in human and K123 in yeast). Ubiquitylation of H2AK119 is mediated by the Bmi/Ring1A protein found in the human polycomb complex and is associated with transcriptional repression (Wang et al., 2006). This modification is not conserved in yeast. In contrast, H₂BK120 ubiquitylation is mediated by human RNF20/RNF40 and UbCH6 and in budding yeast by Rad6/Bre1 and is activatory for transcription (Zhu et al., 2005). A role for this modification has been demonstrated in transcriptional elongation by the histone chaperone FACT (Pavri et al., 2006). Ubiquitylation functions by recruiting additional factors to chromatin but may also function to physically keep chromatin open by a “wedging” process, given its large size.

Ubiquitilases and their substrates, include, but are not limited to: Bmi/Ring1A (H2AK119) and RNF20/RNF40 (H₂BK120).

K. Deubiquitylation

In budding yeast, two enzymes (Ubp8 and Ubp10) have been identified that antagonize ubiquitylation of H₂BK123. The Ubp8 enzyme (subunit of the SAGA acetyltransferase complex) is required for activation of transcription, indicating that both the addition and removal of ubiquition is necessary for stimulation of transcription. The Ubp10 deubiquitylase functions in transcriptional silencing at heterochromatic sites in budding yeast (Emre et al., 2005, Gardner et al., 2005).

L. Proline Isomerization

Prolines exist in either a cis or trans conformation. These conformational changes can severely distort the polypeptide backbone. An enzyme, FPR4, has been identified in budding yeast that can isomerize prolines in the tail of H3 (Nelson et al., 2006). FPR4 isomerizes H3P38 and thereby regulates the levels of methylation at H3K36. The appropriate proline isomer is likely to be necessary for the recognition and methylation of H3K36 by the Set2 methyltranferase. In addition, demethylation of H3K36 is also affected by isomerization at H3P38 (Chen et al., 2006). The catalytic cleft of the JMJD2 demethylase is very deep and may necessitate a bend in the polypeptide (mediated by proline isomerization) to accommodate the methyl group at H3K36.

M. Deimination

Deimination involves the conversion of an arginine to a citrulline. Arginines in H3 and H4 can be converted to citrullines by the PADI4 enzyme. Deimination antagonizes the activating effect of arginine methylation since citrulline prevents arginines from being methylated (Cuthbert et al., 2004, Wang et al., 2004). In addition, in vivo data demonstrate that mono- (but not di-) methylated arginines can be deiminated (Wang et al., 2004). In vitro analysis of the PADI4 enzyme suggests that the reversal of monomethyl arginine to citrulline is not carried out by the recombinant enzyme when methylated peptides are used as substrates, suggesting that a cofactor may be necessary in vivo (Hidaka et al., 2005). Converting citrulline to arginine has not been described, although citrulline is cyclic on the pS2 promoter, so reversal may be possible (Bannister et al., 2005).

N. Sumoylation

Like ubiquitylation, sumoylation is a very large modification and shows some low similarity to ubiquitylation. This modification has been shown to take place on all four core histones, and specific sites have been identified on H4, H2A, and H₂B (Nathan et al., 2006). Sumoylation antagonizes both acetylation and ubiquitylation, which occur on the same lysine residue, and consequently this modification is a repressive one for transcription.

O. ADP Ribosylation

This histone modification is ill defined with respect to function. ADP ribosylation can be mono- or poly-, and the enzymes that mediate it are MARTs (Mono-ADP-ribosyltransferases) or PARPs (poly-ADP-ribose polymerases), respectively (Hassa et al., 2006). In addition, the Sir family of NAD-dependent histone deacetylases have been shown to have low levels of this activity, so they may represent another class of this family. There are many reports of ADP ribosylation of histones, for example, one site, H2BE2ar1, has been definitively mapped. The function of the enzymes has often been linked to transcription. Recently a role for PARP-1 activity in transcription has been demonstrated under conditions where DNA repair is induced. Double-strand breaks mediated by Topoisomerase II β activate the PARP-1 enzyme, which then directs chromatin changes to the estrogen-regulated PS2 gene (Ju et al., 2006).

P. Epigenetics and Pluripotency Factors

Both pluripotency factors and epigenetic regulators provide fundamental mechanisms underlying pluripotency. Both pathways also engage in cross-talk with one another in order to maintain pluripotency. First, pluripotency factors regulate genes encoding epigenetic control factors. It has been shown that Oct-3/4, Sox-2 and Nanog co-regulate certain genes encoding components of chromatin remodeling and histone modifying complexes, such as SMARCAD1, MYS3 and SET (Boyer L. A., et al., Core transcriptional regulatory circuitry in human embryonic stem cells. Cell (2005) 122:947-956). Second, pluripotency factors also interact with histone modifying enzymes and chromatin remodeling complexes. Nanog and Oct-3/4 interact directly or indirectly with the histone deacetylase NuRD (P66b and HDAC2), polycomb group (YY1, Rnf2 and Rybp) and SWI/SNF chromatin remodeling (BAF155) complexes (Wang J., et al., A protein interaction network for pluripotency of embryonic stem cells. Nature (2006) 444:364-368). Finally, the genes of pluripotency factors are subjected to epigenetic regulation. Good examples of this are two histone demethylase genes, Jmjd1a and Jmjd2c, which are downstream targets of Oct-3/4 (Loh Y. H., et al., The Oct-3/4 and Nanog transcription network regulates pluripotency in mouse embryonic stem cells. Nat. Genet. (2006) 38:431-440 and Loh Y. H., et al., Jmjd1a and Jmjd2c histone H3 Lys 9 demethylases regulate self-renewal in embryonic stem cells. Genes Dev. (2007) 21:2545-2557). Jmjd1a acts as a positive regulator of the pluripotency-associated genes, Tcl1, Tcfcf2l1 and Zfp57, by demethylating H3K9Me2 at the promoters. Jmjd2c removes H3K9Me3 marks at the Nanog promoter to positively regulate Nanog expression (Loh et al., 2007)

### V. Pluripotency Factors

As used herein the term “pluripotency gene”, refers to a gene that is associated with pluripotency. A pluripotency factor corresponds to a gene product (i.e., a polypeptide) that is associated with pluripotency. The expression of a pluripotency gene is typically restricted to pluripotent stem cells, and is crucial for the functional identity of pluripotent stem cells. The transcription factor Oct-4 (also called Pou5fl, Oct-3, Oct3/4) is an example of a pluripotency factor. Oct-4 has been shown to be required for establishing and maintaining the undifferentiated phenotype of ES cells and plays a major role in determining early events in embryogenesis and cellular-differentiation (Nichols et al., 1998, Cell 95:379-391; Niwa et al., 2000, Nature Genet. 24:372-376). Oct-4 is down-regulated as stem cells differentiate into specialised cells. Other exemplary pluripotency genes include, but are not limited to Nanog, Sox2, cMyc, Klf-4, and Lin-28, among others.

The present invention contemplates, in part, methods to reprogram and program cells comprising contacting the cells with a composition comprising at least one repressor and/or activator, in any number or combination, to modulate a component of a cellular potency pathway and thereby reprogram or program the cell. In various embodiments, a component of the cellular potency pathway is a pluripotency factor selected from the group consisting of: Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281.

In particular embodiments, the component of the cellular potency pathway is a pluripotency factor selected from the group consisting of: Oct-4, Nanog, Sox-2, cMyc, Klf-4, Lin-28, Stat-3, and Tcf-3.

In other related embodiments, the component of the cellular potency pathway is a pluripotency factor selected from the group consisting of: Oct-3/4, Sox-2, Nanog, Lin-28, c-Myc, and Klf-4.

In certain related embodiments, the component is a pluripotency factor selected from the group consisting of: Oct-3/4, Sox-2, and Nanog.

In one embodiment, the component is the pluripotency factor Oct-3/4.

In other embodiments, one or more components of one of more cellular potency pathways are modulated to alter the potency of a cell. Thus, any number and/or combination of the components of a cellular pathway associated with a developmental potency of a cell as discussed herein, supra or infra, is suitable to modulate the potency of a cell. For example, in some embodiments, a cell is contacted with a composition comprising 1, 2, 3, 4, 5, 6, 7, 8, 9, 10 or more repressors and/or activators in any number or combination that modulates 1, 2, 3, 4, 5, 6, 7, 8, 9, 10 or more components of a cellular potency pathway, including any number or combination of pluripotency factors.

In another embodiment, a cell is contacted with a composition comprising one or more repressors and/or activators that modulates at least 1, at least 2, at least 3, at least 4, at least 5, at least 6, at least 7, at least 8, at least 9, or at least 10 or more components of a cellular potency pathway, including any number or combination of pluripotency factors.

In other related embodiments, the component of the cellular potency pathway is a pluripotency factor selected from the group consisting of: Oct-3/4, Sox-2, Nanog, Lin-28, c-Myc, Klf-4, or hTERT.

In certain related embodiments, the component is a pluripotency factor selected from the group consisting of: Oct-3/4, Sox-2, or Nanog.

In one embodiment, the component is the pluripotency factor Oct-3/4.

In yet other related embodiments, the one or more activators and/or repressors are themselves pluripotency factors or components of a pathway associated with the potency of a cell.

In related embodiments, the repressors and/or activators are transcription factors that either increase or decrease expression of a component of a cellular pathway associated with cell potency (e.g., a pluripotency factor), and thereby alter the potency of the cell.

Illustrative pluripotency factors are described in further detail below. However, one having ordinary skill in the art would recognize that pluripotency factors of the present invention are not limited by the description below, but instead, pluripotency factors of the present invention encompass all pluripotency factors.

In certain embodiment, pluripotency factors are also illustrative repressors and/or activators suitable for use in the methods of reprogramming and programming cells of the present invention, as they are known to both positively and negatively regulate the expression of many genes involved in cellular pathways associated with the potency of a cell.

A. Oct Family

Oct-3/4 was identified as a novel Oct family protein specifically expressed in EC cells, early embryos, and germ cells (Okamoto et al., 1990, Rosner et al., 1990, Scholer et al., 1990). The octamer (“Oct”) family of transcription factors contains the POU domain, a 150 amino acid sequence conserved among Pit-I, Oct-1, Oct-2, and uric-86. Oct-3/4 and other POU proteins bind to the octamer transcription factor binding site sequence (ATTA/TGCAT). Expression of Oct-3/4 is restricted to the blastomeres of the developing mouse embryo, the ICM of blastocysts, the epiblast, and germ cells. Oct-3/4 is also expressed in pluripotent stem cells, including ESCs, EG cells, EC cells, and mGS cells and plays a role in establishing and maintaining pluripotency.

Oct-3/4 null embryos die in utero during the peri-implantation stages of development (Nichols et al., 1998). Although these embryos are able to reach the blastocyst stage, in vitro culture of the ICM of homozygous mutant blastocysts produces only trophoblast lineages. ESCs can not be derived from Oct-3/4 null blastocysts. Suppression of Oct-3/4 resulted in spontaneous differentiation into the trophoblast lineages in both mouse (Niwa et al., 2000) and human ESCs (Zaehres et al., 2005). These data demonstrate the essential roles of Oct-3/4 in the establishment and maintenance of pluripotency.

Oct-3/4 also plays important roles in promoting differentiation. Only a 50% increase in the Oct-3/4 protein in ESCs resulted in spontaneous differentiation into primitive endoderm and mesoderm (Niwa et al., 2000), which is consistent with the transient increase in Oct-3/4 expression during the initial stage of primitive endoderm differentiation from ICM. Oct-3/4 also plays a role in the neural (Shimozaki et al., 2003) and cardiac (Zeineddine et al., 2006) differentiation from ESCs. Thus, Oct-3/4 expression levels are an important determinant of the cell fate in ESCs.

The activation of Oct-3/4 in gastric epithelial tissues results in dysplastic growth that is dependent on continuous transgene expression (Hochedlinger et al., 2005). Dysplastic lesions show an expansion of progenitor cells and an increased β-catenin transcriptional activity. In the intestine, Oct-3/4 expression causes dysplasia by inhibiting cellular differentiation. These data indicate that specific adult progenitors may remain competent to respond to key embryonic signals, and they might also be a driving force in tumorigenesis.

Various other genes in the “Oct” family, including Oct-3/4's close relatives, Oct1 and Oct6, fail to elicit induction of pluripotency, thus demonstrating the exclusiveness of Oct-3/4 to the induction process.

Illustrative members of the Oct family of transcription factors include, but are not limited to: Oct-1, Oct-2, Oct-3/4, Oct-6, Oct-7, Oct-8, Oct-9, and Oct-11.

B. Sox Family

Sox-2 was identified as a Sox (SRY-related HMG box) protein expressed in EC cells (Yuan et al., 1995). The high mobility group (HMG) domain is a DNA binding domain conserved in abundant chromosomal proteins, including, but not limited to HMG1 and HMG2, which bind DNA with little or no sequence specificity. The HMG domain is also present in sequence-specific transcription factors, including, but not limited to SRY, SOX, and LEF-1. The SOX family of transcription factors appears to recognize a similar binding motif, A/TA/TCAAA/TG. Like Oct-3/4, Sox-2 also marks the pluripotent lineage of the early mouse embryo; it is expressed in the ICM, epiblast, and germ cells. Unlike Oct-3/4, however, Sox-2 is also expressed by the multipotential cells of the extraembryonic ectoderm (Avilion et al., 2003). In addition, Sox-2 expression is associated with uncommitted dividing stem and precursor cells of the developing central nervous system (CNS), and it can be used to isolate such cells (Li et al., 1998, Zappone et al., 2000).

Sox-2 null embryos die at the time of implantation due to a failure of epiblast (primitive ectoderm) development (Avilion et al., 2003). Homozygous mutant blastocysts appear morphologically normal, but undifferentiated cells fail to proliferate when blastocysts are cultured in vitro, and only trophectoderm and primitive endoderm-like cells are produced. The deletion of Sox-2 in ESCs results in trophectoderm differentiation (Masui et al., 2007). Therefore, Sox-2, like Oct-3/4, is essential for the establishment and maintenance of pluripotency.

Sox proteins, in general, regulate their target genes by associating with specific partner factors (Kamachi et al., 2000, Wilson et al., 2002). Sox-2 forms a heterodimer with Oct-3/4 and synergistically regulates Fgf4 (Yuan et al., 1995), UTF1 (Nishimoto et al., 2003), and Fbx15 (Tokuzawa et al., 2003). In addition, similar coregulation by Sox-2 and Oct-3/4 has been reported in the regulation of Sox-2 and Oct-3/4 themselves (Chew et al., 2005, Okumura-Nakanishi et al., 2005, Tomioka et al., 2002), as well as Nanog (Kuroda et al., 2005, Rodda et al., 2005). Genome-wide chromatin immunoprecipitation analyses demonstrated that Oct-3/4, Sox-2, and Nanog share many target genes in both mouse and human ESCs (Boyer et al., 2005, Loh et al., 2006). Surprisingly, Sox-2 deletion in mouse ESCs is rescued by the cDNA introduction of not only Sox-2 but also Oct-3/4, thus suggesting that the primary function of Sox-2 might be to maintain Oct-3/4 expression (Masui et al., 2007).

Other genes in the Sox family have been found to work as well in the induction process. For example, Sox1 yields induced pluripotent stem cells (iPS cells) with a similar efficiency as Sox-2, and genes Sox3, Sox15, and Sox18 also generate iPS cells, although with somewhat less efficiently.

Illustrative members of the Sox family of transcription factors include, but are not limited to: Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30.

C. Klf Family

Klf-4 belongs to Krüppel-like factors (KLFs), zinc-finger proteins that contain amino acid sequences resembling those of the Drosophila embryonic pattern regulator Krüppel (Schuh et al., 1986). Klf-4 is highly expressed in differentiated, postmitotic epithelial cells of the skin and the gastrointestinal tract. Klf-4 is expressed in fibroblasts including MEF and NIH3T3 cells (Garrett-Sinha et al., 1996, Shields et al., 1996). Shields et al., found that, in NIH3T3 cells, Klf-4 mRNA is found in high levels in cells during growth arrest and is nearly undetectable in cells that are in the exponential phase of proliferation (Shields et al., 1996). In addition, Klf-4 is highly expressed in undifferentiated mouse ESCs.

Klf-4 can function both as a tumor suppressor and an oncogene. In cultured cells, the forced expression of Klf-4 results in the inhibition of DNA synthesis and cell cycle progression (Chen et al., 2001, Shields et al., 1996). Klf-4 null embryos develop normally, but newborn mice die within 15 hr and show an impaired differentiation in the skin (Segre et al., 1999) and in the colon (Katz et al., 2002), thus indicating that this Klf transcription factor plays a role as a switch from proliferation to differentiation. A conditional knockout mouse model suggests that Klf-4 plays a role as a tumor suppressor in gastrointestinal cancers (Katz et al., 2005). Klf-4, however, is overexpressed in squamous cell carcinomas and breast cancers (Foster et al., 2000, Foster et al., 1999). Moreover, the induction of Klf-4 in basal keratinocytes blocks the proliferation-differentiation switch and initiates squamous epithelial dysplasia (Foster et al., 2005). Therefore, Klf-4 is associated with both tumor suppression and oncogenesis.

The inactivation of STAT3 in mouse ESCs markedly decreases Klf-4 expression, and forced expression of Klf-4 enables LIF-independent self-renewal. A positive effect of Klf-4 in self-renewal of mouse ESCs has also been reported (Li et al., 2005). In addition, Klf-4 cooperates with Oct-3/4 and Sox-2 to activate the Lefty1 core promoter in mouse ESCs (Nakatake et al., 2006).

Klf-4 was identified as a pluripotency factor for the generation of mouse and human iPS cells. However, it was later reported that Klf-4 was not required for the generation of human iPS cells. Klf2 and Klf-4 were found to be factors capable of generating iPS cells in mice, and related genes Klf1 and Klf5 did as well, although with reduced efficiency.

Illustrative members of the Sox family of transcription factors include: Klf1, Klf2, Klf3, Klf-4, Klf5, Klf6, Klf7, Klf8, Klf9, Klf10, Klf11, Klf12, Klf13, Klf14, Klf15, Klf16, and Klf17.

D. Myc Family

c-Myc is one of the first proto-oncogenes discovered in human cancers (Dalla-Favera et al., 1982). The N terminus of Myc binds to several proteins, including TRRAP, which are components of the TIP60 and GCN5 histone acetyltransferase complexes, and TIP48 and TIP49, which contain ATPase domains (Adhikary et al., 2005). The C terminus of the Myc protein contains the basic region/helix-loop-helix/leucine zipper (BR/HLH/LZ) domain, through which Myc binds to a partner protein, Max. Myc-Max dimers bind to a DNA sequence (CACA/GTG), which is a subset of the general E box sequence (CANNTG) bound by all bHLH transcription factors. In addition to binding to DNA, the C terminus of Myc is also involved in transactivation through binding to CBP and p300, which have histone acetylase activities.

Mouse embryos homozygous for a c-Myc deletion die between 9.5 and 10.5 days of gestation (Davis et al., 1993). Pathologic abnormalities include the heart, pericardium, neural tube, and delay or failure in turning of the embryo. The lethality of c-Myc^{-/-} embryos is also associated with profound defects in vasculogenesis and primitive erythropoiesis (Baudino et al., 2002). In addition, c-Myc^{-/-} ESCs are defective in vascular differentiation. However, earlier-stage embryos are apparently normal despite the deficiency of c-Myc, and c-Myc^{-/-} ESCs show normal proliferation and self-renewal. In contrast, the dominant-negative form of c-Myc induces differentiation in mouse ESCs (Cartwright et al., 2005), thus suggesting that the c-Myc deficiency might be compensated by the related proteins N-Myc and L-Myc.

The most surprising new finding is that there are as many as 25,000 Myc binding sites in vivo in the human genome (Cawley et al., 2004, Fernandez et al., 2003, Li et al., 2003). These studies revealed that only a minority portion of the in vivo binding sites of Myc-Max have the consensus CACA/GTG sequence. The direct binding of the Myc-Max dimer to noncanonical sequences is observed in the human Werner syndrome gene, WRN (Grandori et al., 2003). Alternatively, the Myc-Max dimer is recruited to nonconsensus binding sites through an interaction with other transcription factors, such as Miz1 (Peukert et al., 1997). By binding to numerous sites in genome, c-Myc may modify the chromatin structure (Knoepfler et al., 2006) and regulate the expression of noncoding RNAs (O'Donnell et al., 2005).

Several groups have demonstrated that c-Myc is a factor implicated in the generation of mouse and human iPS cells. However, it was later reported that c-myc was unnecessary for generation of human iPS cells. Usage of the “myc” family of genes in induction of iPS cells is troubling for the eventuality of iPS cells as clinical therapies, as 25% of mice transplanted with c-myc-induced iPS cells developed lethal teratomas. N-myc and L-myc have been identified to induce in the stead of c-myc with similar efficiency.

E. Nanog

Nanog, an NK-2 type homeodomain protein, was identified as a gene that is specifically expressed in mouse ESCs and preimplantation embryos and has been proposed to play a key role in maintaining stem cell pluripotency presumably by regulating the expression of genes critical to stem cell renewal and differentiation. Mouse ICMs deficient in Nanog failed to generate epiblast and only produced parietal endoderm-like cells, while mouse ESCs deficient in Nanog lost pluripotency and differentiated into cells of the extraembryonic endoderm lineage. These observations demonstrated that Nanog is a factor underlying the establishment and/or maintenance of pluripotency in both ICM and ESCs (Mitsui et al., 2003).

Nanog was also found to direct the propagation of undifferentiated ESCs. Nanog mRNA was present in pluripotent mouse and human cell lines and was absent from differentiated cells. In preimplantation embryos, Nanog was restricted to founder cells from which ESCs could be derived. Endogenous Nanog was found to act in parallel with cytokine stimulation of Stat3 to drive ESC self-renewal. Elevated Nanog expression from transgene constructs was sufficient for clonal expansion of ESCs, bypassing Stat3 and maintaining Oct-3/4 levels. Cytokine dependence, multilineage differentiation, and embryo colonization capacity were fully restored upon transgene excision. These findings established a central role for Nanog in the transcription factor hierarchy that defines ESC identity (Chambers et al., 2003).

In fusions between ESCs and neural stem (NS) cells, increased levels of Nanog stimulated pluripotent gene activation from the somatic cell genome and enabled an up to 200-fold increase in the recovery of hybrid colonies, all of which showed ESC characteristics (Silva et al., 2006). Nanog also improved hybrid yield when thymocytes or fibroblasts were fused to ESCs; however, fewer colonies were obtained than from ES×NS cell fusions, consistent with a hierarchical susceptibility to reprogramming among somatic cell types. Notably, for NS×ESC fusions, elevated Nanog enabled primary hybrids to develop into ESC colonies with identical frequency to homotypic ES×ES fusion products. Thus, without wishing to be bound by any particular theory, increased Nanog expression is sufficient for the NS cell epigenome to be reset completely to a state of pluripotency. Therefore, Nanog can orchestrate ESC machinery to instate pluripotency with an efficiency of up to 100% depending on the differentiation status of the somatic cell.

A protein interaction network has been identified for Nanog. Nanog-associated proteins, include, but are not limited to Dax1, Nac1, Zfp281, and Oct-3/4. The Nanog protein interaction network is highly enriched for nuclear factors that are individually important for the establishment and maintenance of a pluripotent state and functions as a cellular module dedicated to pluripotency. The network is further linked to multiple corepressor pathways and is composed of numerous proteins whose encoding genes are putative direct transcriptional targets of its members (Wang et al., 2006).

F. Lin-28

Lin-28 homolog (C. elegans), also known as Lin28, is a human gene that encodes a cytoplasmic mRNA-binding protein. The Lin28 locus was identified as a binding site for Oct-3/4, Sox-2, and Nanog in a genome-wide location analysis (Boyer et al., 2005), suggesting that these three reprogramming factors might induce its expression and, with appropriate induction levels, allow reprogramming in its absence.

Human Lin28 mRNA was identified as a target of the micro RNAs miR-125b and miRNA-125a. These miRNAs act to reduce the translational efficiency and mRNA abundance of Lin28 (Wu and Belasco 2005). Deletion of the two miRNA-responsive elements (miREs) that mediate repression in the 3-prime UTR of Lin28 reduced the level of miRNA control over Lin28. Lin28 downregulation was found to involve miR-125.

Loss-of-function and gain-of-function assays in cultured myoblasts, showed that expression of Lin28 was essential for skeletal muscle differentiation in mice and that Lin28 binds to polysomes, thereby increasing the efficiency of protein synthesis (Polesskaya et al., 2007). An important target of Lin28 is Igf2, a growth and differentiation factor for muscle tissue. Interaction of Lin28 with translation initiation complexes in skeletal myoblasts and in the embryonic carcinoma cell line P19 was confirmed by localization of Lin28 to the stress granules, temporary structures that contain stalled mRNA-protein translation complexes.

Lin28 was shown to selectively block the processing of Let7 primary (pri-Let7) miRNAs in embryonic stem cells (Viswanathan et al., 2008). Lin28 was also found to be necessary and sufficient for blocking Microprocessor-mediated cleavage of pri-Let7 miRNAs. Lin28 was also identified as a negative regulator of miRNA biogenesis that may play a central role in blocking miRNA-mediated differentiation in stem cells and in certain cancers.

Lin28 is a marker of undifferentiated human embryonic stem cells and has been used to enhance the efficiency of the formation of iPS cells from human fibroblasts. These human iPS cells have normal karyotypes, express telomerase activity, express cell surface markers and genes characteristic of human ESCs, and maintain the developmental potential to differentiate into advanced derivatives of all 3 primary germ layers.

G. Components

As used herein, the terms “component of a cellular pathway associated with potency” and “component of a potency pathway” refer to an endogenous gene or gene product that is important in establishing, determining, maintaining, regulating, or altering the developmental potency of a cell. Pluripotency factors are components of cellular pathways that affect cell potency, as are numerous developmental genes, chromatin remodeling enzymes, and transcription factors as discussed herein throughout. The present invention contemplates, in part, that any transcripional target of a pluripotency factor, or of a component of the present invention may also be a component of the present invention. Without wishing to be bound to a particular theory, it is known in the art that transcriptional circuits that regulate, establish, and/or maintain aspects of potency pathways have multiple layers of regulation (Sharov et al., 2008; Chen and Daley, 2008; Jaenisch and Young, 2008; Marson et al, 2008; and Campbell et al., 2007)

Illustrative components of developmental potency pathways that may either be altered by repression and/or activation include, but are not limited to members of the Hedgehog pathway, components of the Wnt pathway, receptor tyrosine kinases, non-receptor tyrosine kinases, TGF family members, BMP family members, Jak/Stat family members, Hox family members, Sox family members, Klf family members, Myc family members, Oct family members, components of a chromatin modulation pathway, components of a histone modulation pathway, miRNAs regulated by pluripotency factors, miRNAs that regulate pluripotency factors and/or components of cellular pathway associated with the developmental potency of a cell, members of the NuRD complex, Polycomb group proteins, SWI/SNF chromatin remodeling enzymes, Ac133, Alp, Atbf1, Axin2, BAF155, bFgf, Bmi1, Boc, C/EBPβ, CD9, Cdon, Cdx-2, c-Kit, c-Myc, Coup-Tf1, Coup-Tf2, Csl, Ctbp, Dax1, Dnmt3A, Dnmt3B, Dnmt3L, Dppa2, Dppa4, Dppa5, Ecat1, Ecat8, Eomes, Eras, Esg1, Esrrb, Fbx15, Fgf2, Fgf4, Flt3, Foxc1, Foxd3, Fzd9, Gbx2, Gcnf, Gdf10, Gdf3, GdfS, Grb2, Groucho, Gsh1, Hand1, Hdac1, Hdac2, HesX1, Hic-5, HoxA10, HoxA11, HoxB1, HP1α, HP1β, HPV16 E6, HPV16 E7, Irx2, Isl1, Jarid2, Jmjd1a, Jmjd2c, Klf-3, Klf-4, Klf-5, Left, Lefty-1, Lefty-2, Lif, Lin-28, Mad1, Mad3, Mad4, Mafa, Mbd3, Meis1, MeI-18, Meox2, Mta1, Mxi1, Myf5, Myst3, Nac1, Nanog, Neurog2, Ngn3, Nkx2.2, Nodal, Oct-4, Olig2, Onecut, Otx1, Oxt2, Pax5, Pax6, Pdx1, Pias1, Pias2, Pias3, Piasy, REST, Rex-1, Rfx4, Rif1, Rnf2, Rybp, Sal1l4, Sal1l1, Scf, Scgf, Set, Sip1, Skil, Smarcad1, Sox-15, Sox-2, Sox-6, Ssea-1, Ssea-2, Ssea-4, Stat3, Stella, SV40 large T antigen, Tbx3, Tcf1, Tcf2, Tcf3, Tcf4, Tcf-7, Tcf7l1, Tcl1, Tdgf-1, Terf, hTert, Tif1, Tra-1-60, Tra-1-81, Uff-1, Wnt3a, Wnt8a, YY1, Zeb2, Zfhx1b, Zfp281, Zfp57, Zic3, β-catenin, histone acetylases, histone de-acetylases, histone methyltransferases, histone demethylases or substrates, cofactors, co-activators, co-repressors and/or a downstream effectors thereof.

Illustrative examples of the accession numbers for polynucleotide and polypeptide sequences of the foregoing factors include, but are not limited to: Ac133 (e.g., NM_{—}001145852, NM_{—}001145851, NM_{—}001145850, NM_{—}001145849, NM_{—}001145848, NM_{—}001145847, NM_{—}006017, NP_{—}001139324, NP_{—}001139323, NP_{—}001139322, NP_{—}001139321, NP_{—}001139320, NP_{—}001139319, NP_{—}006008); Alp (e.g., NM_{—}207303 and NP_{—}997186); Atbf1 (e.g., NM_{—}006885 and NP_{—}008816); Axin2 (e.g., NM_{—}004655 and NP_{—}004646); BAF155 (e.g., NM_{—}003074 and NP_{—}003065); bFgf (e.g., NM_{—}002006 and NP_{—}001997); Bmi1 (e.g., NM_{—}005180 and NP_{—}005171); Boc (e.g., NM_{—}033254, NP_{—}150279); C/EBPβ (e.g., NM_{—}005194 and NP_{—}005185); CD9 (e.g., NM_{—}001769 and NP_{—}001760); Cdon (e.g., NM_{—}016952 and NP_{—}058648); Cdx-2 (e.g., NM_{—}001265 and NP_{—}001256); c-Kit (e.g., NM_{—}000222, NM_{—}001093772, NP_{—}001087241, and NP_{—}000213); c-Myc (e.g., NM_{—}002467 and NP_{—}002458); Coup-Tf1 (e.g., NM_{—}005654 and NP_{—}005645); Csl (e.g., NM_{—}022579, NM_{—}022580, NM_{—}022581, NM_{—}001318, NP_{—}001309, NP_{—}072103, NP_{—}072102, and NP_{—}072101); Ctbp (e.g., NP_{—}203292, NP_{—}203291, NM_{—}002894, NP_{—}976037, NP_{—}976036, and NP_{—}002885); Dax1 (e.g., NM_{—}000475 and NP_{—}000466); Dnmt3A (e.g., NM_{—}175630, NM_{—}175629, NM_{—}153759, NM_{—}022552, NP_{—}715640, NP_{—}783329, NP_{—}783328, and NP_{—}072046); Dnmt3B (e.g., NM_{—}175850, NM_{—}175849, NM_{—}175848, NM_{—}006892, NP_{—}787046, NP_{—}787045, NP_{—}787044, and NP_{—}008823); Dnmt3L (e.g., NM_{—}175867, NM_{—}013369, NP_{—}787063, and NP_{—}037501); Dppa2 (e.g., NM_{—}138815 and NP_{—}620170); Dppa4 (e.g., NM_{—}018189 and NP_{—}060659); Dppa5 (e.g., NM_{—}001025290 and NP_{—}001020461); Ecat1 (e.g., NM_{—}001017361 and NP_{—}001017361); Ecat8 (e.g., NM_{—}001110822 and NP_{—}001104292); Eomes (e.g., NM_{—}005442 and NP_{—}005433); Eras (e.g., NM_{—}181532 and NP_{—}853510); Esg1 (e.g., NM_{—}005077 and NP_{—}005068); Esrrb (e.g., NM_{—}004452 and NP_{—}004443); Fbx15 (e.g., NM_{—}001142958, NM_{—}152676, NP_{—}001136430, and NP_{—}689889); Fgf4 (e.g., NM_{—}002007 and NP_{—}001998); Flt3 (e.g., NM_{—}004119 and NP_{—}004110); Foxc1 ((e.g., NM_{—}001453 and NP_{—}001444); Foxd3 (e.g., NM_{—}012183 and NP_{—}036315); Fzd9 (e.g., NM_{—}003508 and NP_{—}003499); Gbx2 (e.g., NM_{—}001485 and NP_{—}001476); Gcnf (e.g., NM_{—}033334, NM_{—}001489, NP_{—}001480, and NP_{—}201591); Gdf10 (e.g., NM_{—}004962 and NP_{—}004953); Gdf3 (e.g., NM_{—}020634 and NP_{—}065685); GdfS (e.g., NM_{—}000557 and NP_{—}000548); Grb2 (e.g., NM_{—}203506, NM_{—}002086, NP_{—}002077, and NP_{—}987102); Groucho (e.g., NM_{—}005077, NP_{—}005068, NM_{—}007005, NP_{—}008936, NM_{—}001105192, NM_{—}020908, NM_{—}005078, NP_{—}001098662, NP_{—}065959, NP_{—}005069, NM_{—}001144762, NM_{—}001144761, NM_{—}003260, NP_{—}001138234, NP_{—}001138233, and NP_{—}003251); Gsh1 (e.g., NM_{—}145657 and NP_{—}663632); Hand1 (e.g., NM_{—}004821 and NP_{—}004812, Hdac1 (e.g., NM_{—}004964 and NP_{—}004955); Hdac2 (e.g., NM_{—}001527.2 and NP_{—}001518.2); HesX1 (e.g., NM_{—}003865 and NP_{—}003856); Hic-5 (e.g., NM_{—}001042454, NM_{—}015927, NP_{—}001035919, and NP_{—}057011); HoxA10 (e.g., NM_{—}018951.3 and NP_{—}061824.3); HoxA11 (e.g., NM_{—}005523.5 and NP_{—}005514.1); HoxB1 (e.g., NM_{—}002144 and NP_{—}002135); HP1a (e.g., NM_{—}001127322, NM_{—}001127321, NM_{—}012117, NP_{—}001120794, NP_{—}001120793, and NP_{—}036249); HP1β (e.g., NM_{—}006807, NM_{—}001127228, NP_{—}001120700, and NP_{—}006798); Irx2 (e.g., NM_{—}001134222, NM_{—}033267, NP_{—}150366, and NP_{—}001127694); Isl1 (e.g., NM_{—}002202 and NP_{—}002193); Jarid2 (e.g., NM_{—}004973 and NP_{—}004964); Jmjd1a (e.g., NM_{—}001146688, NM_{—}018433, NP_{—}001140160, and NP_{—}060903); Jmjd2c (e.g., NM_{—}001146696, NM_{—}001146695, NM_{—}001146694, NM_{—}01506, NP_{—}001140168, NP_{—}001140167, NP_{—}001140166, and NP_{—}055876); Klf-3 (e.g., NM_{—}016531 and NP_{—}057615); Klf-4 (e.g., NM_{—}004235 and NP_{—}004226); Klf-5 (e.g., NM_{—}001730 and NP_{—}001721); Lef1 (e.g., NM_{—}001130714, NM_{—}001130713, NM_{—}016269, NP_{—}001124186, NP_{—}001124185, NP_{—}057353); Lefty-1 (e.g., NM_{—}020997 and NP_{—}066277); Lefty-2 (e.g., NM_{—}003240 and NP_{—}003231); Lif (e.g., NM_{—}002309 and NP_{—}002300); Lin-28 (e.g., NM_{—}024674 and NP_{—}078950); Mad1 (e.g., NM_{—}001013837, NM_{—}001013836, NM_{—}003550, NP_{—}001013859, NP_{—}001013858, and NP_{—}003541); Mad3 (e.g., NM_{—}001142935, NM_{—}031300, NP_{—}001136407, and NP_{—}112590); Mad4 (e.g., NM_{—}006454 and NP_{—}006445); Mafa (e.g., NM_{—}201589 and NP_{—}963883); Mbd3 (e.g., NM_{—}003926 and NP_{—}003917); Meis1 (e.g., NM_{—}002398 and NP_{—}002389); MeI-18 (e.g., NM_{—}007144 and NP_{—}009075); Meox2 (e.g., NM_{—}005924 and NP_{—}005915); Mta1 (e.g., NM_{—}004689 and NP_{—}004680); Mxi1 (e.g., NM_{—}001008541, NM_{—}005962, NM_{—}130439, NP_{—}001008541, NP_{—}005953, and NP_{—}569157); Myf5 (e.g., NM_{—}005593 and NP_{—}005584); Myst3 (e.g., NM_{—}001099413, NM_{—}006766, NM_{—}001099412, NP_{—}001092883, NP_{—}006757, and NP_{—}001092882); Nac1 (e.g., NM_{—}052876, and NP_{—}443108); Nanog (e.g., NM_{—}024865 and NP_{—}079141); Neurog2 (e.g., NM_{—}024019 and NP_{—}076924); Ngn3 (e.g., NM_{—}020999 and NP_{—}066279); Nkx2.2 (e.g., NM_{—}002509 and NP_{—}002500); Nodal (e.g., NM_{—}018055 and NP_{—}060525); Oct-4 (e.g., NM_{—}203289, NM_{—}002701, NP_{—}976034, and NP_{—}002692); Olig2 (e.g., NM_{—}005806 and NP_{—}005797); Onecut (e.g., NM_{—}004852 and NP_{—}004843); Otx1 (e.g., NM_{—}014562 and NP_{—}055377); Otx2 (e.g., NM_{—}172337, NM_{—}021728, NP_{—}758840, and NP_{—}068374); Pax5 (e.g., NM_{—}016734 and NP_{—}057953); Pax6 (e.g., NM_{—}001127612, NM_{—}001604, NM_{—}000280, NP_{—}001121084, NP_{—}001595, NP_{—}000271); Pdx1 (e.g., NM_{—}000209 and NP_{—}000200); Pias1 (e.g., NM_{—}016166 and NP_{—}057250); Pias2 (e.g., NM_{—}173206, NM_{—}004671, NP_{—}004662, and NP_{—}775298); Pias3 (e.g., NM_{—}006099 and NP_{—}006090); Piasy (e.g., NM_{—}015897 and NP_{—}056981); REST (e.g., NM_{—}005612 and NP_{—}005603); Rex-1 (e.g., NM_{—}174900 and NP_{—}777560); Rfx4 (e.g., NM_{—}213594, NM_{—}002920, NM_{—}032491, NP_{—}998759, NP_{—}115880, and NP_{—}002911); Rif1 (e.g., NM_{—}018151 and NP_{—}060621); Rnf2 (e.g., NM_{—}007212, NP_{—}009143); Rybp (e.g., NM_{—}012234 and NP_{—}036366); Sall4 (e.g., NM_{—}020436.3 and NP_{—}065169.1); Sall2 (e.g., NM_{—}005407.1 and NP_{—}005398.1); Sall1 (e.g., NM_{—}001127892.1, NM_{—}002968.2, NP_{—}001121364.1, NP_{—}002959.2); Scf (e.g., NP_{—}003985, NP_{—}000890, NM_{—}003994, and NM_{—}000899); Scgf (e.g., NM_{—}002975 and NP_{—}002966); Set (e.g., NM_{—}001122821, NM_{—}003011, NP_{—}001116293, and NP_{—}003002); Sip1 (e.g., NM_{—}001009183, NM_{—}001009182, NM_{—}003616, NP_{—}001009183, NP_{—}001009182, NP_{—}003607); Skil (e.g., NM_{—}001145098, NM_{—}001145097, NM_{—}005414, NP_{—}001138570, NP_{—}001138569, and NP_{—}005405); Smarcad1 (e.g., NM_{—}001128430, NM_{—}020159, NM_{—}001128429, NP_{—}001121902, NP_{—}064544, and NP_{—}001121901), Sox-15 (e.g., NM_{—}006942 and NP_{—}008873); Sox-2 (e.g., NM_{—}003106 and NP_{—}003097); Sox-6 (e.g., NM_{—}001145819, NM_{—}001145811, NM_{—}017508, NM_{—}033326, NP_{—}001139291, NP_{—}001139283, NP_{—}201583, and NP_{—}059978); Ssea-1 (e.g., NM_{—}002033 and NP_{—}002024); Stat3 (e.g., NM_{—}213662, NM_{—}003150, NM_{—}139276, NP_{—}998827, NP_{—}644805, and NP_{—}003141); Stella (e.g., NM_{—}199286 and NP_{—}954980); Tbx3 (e.g., NM_{—}016569, NM_{—}005996, NP_{—}057653, and NP_{—}005987); Tcf1 (e.g., NM_{—}000545 and NP_{—}000536); Tcf2 (e.g., NM_{—}000458 and NP_{—}000449); Tcf3 (e.g., NM_{—}001136139, NM_{—}003200, NP_{—}001129611, and NP_{—}003191); Tcf4 (e.g., NM_{—}003199, NM_{—}001083962, NP_{—}001077431, and NP_{—}003190); Tcf7 (e.g., NM_{—}201632, NM_{—}003202, NM_{—}213648, NM_{—}201634, NM_{—}001134852, NM_{—}001134851, NP_{—}201633, NP_{—}001128324, NP_{—}001128323, NP_{—}998813, NP_{—}963965, NP_{—}003193, NP_{—}963963, and NP_{—}963964); Tcf7l1 (e.g., NM_{—}031283 and NP_{—}112573); Tcl1 (e.g., NM_{—}001098725, NM_{—}021966, NP_{—}001092195, and NP_{—}068801); Tdgf-1 (e.g., NM_{—}003212 and NP_{—}003203); Terf (e.g., NM_{—}001134855, NM_{—}001024941, NM_{—}001024940m NM_{—}016102m NP_{—}01128327, NP_{—}001020112, NP_{—}001020111, and NP_{—}057186); hTert (e.g., NM_{—}198253, NM_{—}198255, NP_{—}937983, NP_{—}937986); Tif1 (e.g., NM_{—}015905, NM_{—}003852, NP_{—}056989, and NP_{—}003843); Tra-1-60 (e.g., NM_{—}001018111, NM_{—}005397, NP_{—}005388, and NP_{—}001018121); Utf-1 (e.g., NM_{—}003577 and NP_{—}003568); Wnt3a (e.g., NM_{—}033131 and NP_{—}149122); Wnt8a (e.g., NM_{—}058244 and NP_{—}490645); YY1 (e.g., NM_{—}003403 and NP_{—}003394); Zeb2 (e.g., NM_{—}014795 and NP_{—}055610); Zfp57 (e.g., NM_{—}001109809 and NP_{—}001103279); Zic3 (e.g., NM_{—}003413 and NP_{—}003404); B-catenin (e.g., NM_{—}001098209, NM_{—}001904, NM_{—}001098210, NP_{—}001091679, NP_{—}001091680, and NP_{—}001895); Coup-Tf2 (e.g., NM_{—}009697, NM_{—}183261, NP_{—}899084, and NP_{—}033827); Zfp281 (e.g., NM_{—}001160251, NM_{—}177643, NP_{—}001153723, and NP_{—}808311); HPV16 E6 (e.g., NP_{—}041325); and HPV16 E7 (e.g., NP_{—}041326), all of which are herein incorporated by reference in their entirety.

The present invention contemplates, in part, methods of altering the developmental potency of a cell, such as to increase the potency of a cell relative to the initial developmental potency of the cell. Also contemplated by the present invention are methods to alter the developmental potency of a cell, such as to decrease the potency of a cell relative to the initial developmental potency of the cell.

Certain embodiments of altering cellular potency may comprise contacting the cell with one or more repressors and/or activators, or a composition comprising the same, to modulate a component of a cellular potency pathway and thereby program or reprogram the cell. In certain embodiments, the component of the cellular pathway associated with the developmental potency of the cells comprises one or more transcription factors. In certain embodiments, components of a cellular pathway associated with the potency of a cell comprise pluripotency factors that are general transcription factors, or basal transcription factors. In other embodiments, the pluripotency factors may comprise the major transcription factors active in a given cell population. Still, in other embodiments, one or more repressors and/or activators, or a composition comprising the same, comprises any number or combination of the pluripotency factors, including, but not limited to any transcription factors described supra or infra. Thus, the exemplary components of cellular potency pathways described elsewhere herein and below, are also illustrative repressors and/or activators suitable for use in the methods of reprogramming and programming cells of the present invention.

Eukaryotic basal transcription regulation involves an important class of transcription factors called general transcription factors, which are necessary to initiate and maintain transcriptional activity. The general transcription factors are typically defined as the minimal complement of proteins necessary to reconstitute accurate transcription from a minimal promoter (such as a TATA element or initiator sequence). Many general transcription factors do not bind DNA, but are part of the large transcription preinitiation complex that interacts directly with RNA polymerase. The most common general transcription factors are TFIIA, TFIIB, TFIID (see also TATA binding protein), TFIIE, TFIIF, TFIIH, and TFIIK.

For example, TATA binding protein (TBP) binds to the TATAA box (T=Thymine, A=Adenine), a nucleic acid motif that resides directly upstream of the coding region in all genes. TBP is responsible for the recruitment of the RNA Pol II holoenzyme, the final event in transcription initiation. This ubiquitious protein interacts with the core promoter region of DNA, which contains the transcription start site(s) of all class II genes.

Other general transcription factors play a role in elongation, the second general step in transcription. For example, members of the FACT complex (SUPT16H/SSRP1 in humans) facilitate the rapid movement of RNA Pol II over the encoding region of genes. This is accomplished by moving the histone octamer out of the way of an active polymerase and thereby decondensing the chromatin.

In certain embodiments, the transcription factors are the major transcription factors active in given cell or cell population. These transcription factors may vary depending on the starting cell or cell population, and may be determined according to routine techniques known in the art. In addition, transcription factor databases may be used to predict the major transcription factors active in a given cell or cell population. For example, in certain embodiments, the major transcription factors active in a cell include transcription factors that comprise DNA-binding domains from the superclass of basic domains, the superclass of zinc-coordinating binding domains, the superclass of helix-turn-helix domain, the superclass of β-scaffold domains with minor groove contact, and the superclass of “other” domains.

Exemplary transcription factors comprising the superclass of basic domains are characterized by a large excess of positive charges, preventing them from being structured when free in solution, but becoming α-helically folded when interacting with DNA. Basic domains typically appear in tight connection with a dimerization domain, a leucine zipper, a helix-loop-helix, or a helix-span-helix domain. Examples of classes of transcription factors having a basic domain include leucine zipper factors, helix-loop-helix factors, helix-loop-helix/leucine zipper factors, NF-1 factors, RF-X factors, and helix-span-helix factors.

Examples of leucine zipper factors include, but are not limited to, the Jun subfamily (e.g., XBP-1, v-Jun, c-Jun), the Fos subfamily (e.g., v-Fos, c-Fos, FosB, Fra-1, Fra-2), the Maf subfamily (e.g., v-Maf, c-Maf, NRL), the NF-E2 subfamily (e.g., NF-ED p45, Nrf1 long form, Nrf1 short form, Nrf2), the CRE-BP/ATF subfamily (e.g., CREB-2, ATF-3, CRE-BP1, CRE-BPa, ATF-a, ATF-aDelta), CREB (e.g., CREB-341), ATF-1, CREM (e.g., ICERA, ICER-IIgamma), dCREB2, the C/EBP-like factor family (e.g., C/EBPalpha, CEBPbeta), the bZIP/PAR family (e.g., HIf), and the ZIP only family (e.g., GCF).

Examples of helix-loop-helix factors include, but are not limited to, ubiquitous (class A) factors (e.g., E2a, E47, ITF-1, ITF-2/SEF2-1B, SEF2-1A, HEB/SCBP), myogenic transcription factors (e.g., MyoD, Myogenin, Myf-5, MRF4, MASH-1), Tal/Twist/Atonal/Hen factors (e.g., lymphoid factors Tal-1, p42Tal-1, Tal-2, Lyl-1; mesodermal Twist-like factors like bHLH-EC2; Hen factors HEN1 and HEN2; Atonal factors like NeuroD/BETA2; and pancreatic factors like INSAF), Hairy factors, factors with PAS domain (e.g., Ahr, Arnt), and HLH domain only factors (e.g., Id1, Id2, Id3, Id4).

Examples of helix-loop-helix/leucine zipper factors include, but are not limited to, ubiquitous bHLH-ZIP factors (e.g., TFE3, TFE3-L, TFEB, Mi, USF, USF2, USF2a, USF2b, SREBP, SREBP-1a, SREBP-1b, SREBP-1c, SREBP-2, AP-4), cell-cycle controlling factors (e.g., c-Myc, N-Myc, L-Myc, Max, Max1, Max2, DeltaMax, Mad1, Mxi1, Mxi1-WR).

Examples of NF-1 factors include, but are not limited to, NF-1, NF-1A, NF-1B, NF-1C, NF-1C2/CTF-2, CTF-3, CTF-4, CTF-5, CTF-6, and CTF-7. Examples of RF-X factors include, but are not limited to, RF-X1, RF-X2, RF-X3, and RF-X5. Examples of helix-span-helix factors include, but are not limited to, AP-2, AP-2alpha, AP-2beta, and AP-2gamma.

Transcription factors comprising the superclass of zinc-coordinating DNA-binding domains include various classes, which classifications have undergone various changes over time, but which have been, or may be referred to as Cys 4 zinc finger of nuclear receptor types, diverse Cys4 zinc fingers, Cys2His zinc finger domains, and Cys6 cystein-zinc clusters, or nuclear receptors, C6 zinc clusters, DM, GCM and WRKY transcription factor classes. Examples include steroid hormone receptors (e.g., corticoid receptorslike GR, GRa, GRb, and MR; progesterone receptors like PR, PR-A, PR-B; andogen receptors like AR, AR-A, AR-B; estrogen receptors like ER, ER-A, ER-B), thyroid hormone receptor-like factors (e.g., retinoic acid receptors like RAR-alpha1, RAR-beta2, RAR-gamma, RAR-gamma1, RAR-delta; retinoid X receptors like RXR-alpha, RXR-beta, RXR-beta1; thyroid hormone receptors like T3R-alpha, T3R alpha-1, T3R alpha-2, T3R beta, T3R-beta1, T3R-beta2; vitamin D receptor; NGFI-B; FTZ-F1 factors like SF-1, FTZ-F1-like, ELP, FTZ-F1; PPAR; EcR factors like EcR A, EcR B1, EcR B2; ROR factors like HR3, RORalpha/RZRalpha, RORalpha1, RORalpha2, RORalpha3, RZRbeta, RORgamma; TII/COUP factors like TR2, TR2-11, TR2-5, TR2-9, TR2-7, TR4, COUP-TFI, ARP/COUP-TFII; HNF-4 factors like HNF-4-alpha, HNF-4-alpha1, HNF-4-alpha2, HNF-4-alpha3, HNF-4-alpha4, HNF-4-alpha5, HNF-4-alpha6, HNF-4-gamma), among others.

Additional examples of zinc-coordinating DNA-binding domain classes of transcription factors include, but are not limited to, diverse Cys4 zinc fingers such as GATA-factors (e.g., GATA-1, GATA-2, GATA-3, GATA-4), Cys2His2 zinc finger domains such as ubiquitous factors (e.g., TFIIIA, Sp1, Sp3, Sp4, YY1), developmental/cell cycle regulators (e.g., Egr/Krox factors like Egr-1, Egr-2, Egr-3, MZF-1, NRSF, GLI, GLI3, WT1+KTS, WT1-KTS, WT1 I, WT1 I-KTS, WT1-del2, WT1-del2 I), and large factors with NF-6B-like binding properties (e.g., HIV-EP1, HIV-EP2, MBP-2, KBP-1), among others.

Transcription factors comprising the superclass of helix-turn-helix domains include, for example, members of the classes referred to as homeobox domain, paired box, Forkhead-winged helix, heat shock factors, tryptophan clusters, and TEA domain. Examples of homeobox domain transcription factors include homeodomain only family members AbdB (e.g., HOXA9, HOXB9, HOXD9, PL1, PL2, HOXC10, HOXD10) Antp (e.g., HOXB3, HOXA4, BOXB4, HOXD4, HOXAS, HOXBS, HOXCS, HOXB6, HOXC6, HOXA7, HOXB7), Cad, Cut (e.g., CDP), DII, Ems (e.g., EMX1, EMX2), En (e.g., EN-1, En-2), Eve (e.g., Evx-1), Prd (e.g., Alx3, K-2, Otx1, Otx2, Unc-4), HD-ZIP, H2.0 (e.g., HB24, Hox11/Hlx), HNF1 (e.g., HNF-1A, HNF-1B, HNF-1C, vHNF-1A, vHNF-1B, vHNF-1C), Lab (e.g., HOXA1, HOXB1), Msh (e.g., Msx-1, Msx-2), NK-2 (e.g., NK-2, NK-3, NK-4, Nkx-6.1, Tinman, TTF-1), Bcd, XANF, and PBC (e.g., Pbx1a, Pbx1b, Pbx2, Pbx3), Prh, Hat24, HB9, Unc-30, BarH1, BarH2, Aalpha Y1, Aalpha Y2, Aalpha Y3, alpha2-1, beta2-1, d1-1). Additional examples of Examples of homeobox domain transcription factors include POU family members, such as Pit-1, Pit1b, Oct-1, Oct-2.1, Oct-2.2/Oct-2A, Oct-2.5/Oct2B, N-Oct-3, N-Oct-SA, N-Oct-SB, Oct-6, Brn-4, Brn-3a(s), Brn-3b, Oct-3b, TCFbeta1, in addition to homeodomain with LIM region family members, such as Lim-1, LH-2, LIM-only transcription factor family members, and homeo domain plus zinc finger motif family members, such as ATBF1-A and ATBF1-B, among others.

Examples of paired box class members include, but are not limited to, paired plus homeo domain family members such as Pax-3, Pax-6, Pax-5/Pd-5, Pax-7, in addition to paired domain only family members such as Paz-1, Pax-5, Pax-8a, Pax-8b, Pax-8c, and Pax-8d. Examples of Forkhead/winged helix class members include, but are not limited to, developmental regulators (e.g., BF-1), tissue specific regulators (e.g., HNF-3alpha, HNF-3beta, HNF-3gamma), cell cycle controlling factors (e.g., E2f, E2F-1, E2F-2, E2F-3, E2F-4, E2F-5, DP, DP-1, DP-2), and other regulators (e.g., ILF, FKHR, HTLF, FD1, FD2, FD3, FD4, FD5, HFH-1, HFH-2, HFH-3, HFH-4, HFH-5, HFH-6, HFH-7, HFH-B2, HFH-B3, Fkh-1, Fkh-2, Fkh-3, Fkh-4, Fkh-5, Fkh-6, BF-2). Examples of heat shock factor class members include, but are not limited to, HSF, HSF1, HSF1(long), HSF1(short), and HSF2, among others.

Examples of tryptophan cluster class members include, but are not limited to, Myb family members (e.g., c-Myb, A-Myb, B-Myb, v-Myb), Ets-type family members (e.g., c-ETS-1, c-ETS-1 p54, Ets-1 DeltaVII, Ets-2, v-Ets, PEA3, Elk-1, SAP-1, SAP-1a, SAP-1b, SAP-2, Erg-1, Erg-2, p38erg, p55erg, p49erg, Fli-1, Spi-B, E4TF1-60/GABP-alpha, Elf-1, Tel), interferon-regulating factors (e.g., IRF-1, IRF-2, ISGF-3gamma). Examples of TEA domain class members include TEF-1, among others.

Transcription factors comprising the superclass of β-scaffold domains with minor groove contact include, but are not limited to, Rel homology region (RHR), STAT, p53-like, MADS, β-barrel α-helix factors, TATA-binding proteins, HMG, heteromeric CCAAT factors, Grainyhead factors, cold-shock domain factors, Runt factors, SMAD/NF-1, and T-box domain factors. Examples of RHR class members include, but are not limited to, Rel/ankyrin factors (e.g., NF-kappaB1, p105, p50; NF-kappaB2, p100, p52, p49; RelA, p65, p65Delta; RelB; c-Rel), ankyrin only factors (e.g., IkappaBalpha, IkappaBbeta, IkappaBgamma, IkappaBR, BcI-3), and NF-AT factors (e.g., NF-ATc, NF-ATp, NF-ATx). Examples of STAT class members include, but are not limited to, STAT1, p91, p84, STAT2, STAT3, STAT4, STATS, STATE). Examples of MADS box class members include, but are not limited to, regulators of differentiation such as MEF-2 (e.g., MEF-2A, aMEF-2, RSRFC4, RSRFC9, MEG-2B1, MEF-2C, MEF-2C/Delta8, MEF-2c/Delta 32, MEF-2C/Delta8, Delta32, MEF-2D, MEF-2AB, MEF-2A′B, MEF-2DOB, MEF-2DAO, MEF-2D00), homeotic genes (e.g., PI, PMADS3, Fbp2, Fbp3, AGL1, AGL2, AGL3, AGL4, AGLS, AGL6, SQA, O-MADS, TAG1, TDR3, TDR4, TDRS, TDR6, NAG1, Tobmads1, MADS1), and responders to external signals (e.g., SRF), among others.

Examples of HMG class members include, but are not limited to, SRY, Sox-4, Sox-5, Sox-8, Sox-9, TCF-1, TCF-1alpha, TCF-1A, TCF-1B, TCF-1C, TCF-1D, TCF-1E, TCF-1F, TCF-1G, TCF-1P, SSRP1, UBF1, UBF2. Examples of heteromeric CCAAT factor class members include, but are not limited to, CP1A, CP1B, and CBF-C. Examples of Grainyhead class members include CP2 and LBP-1a. Examples of cold-shock domain factors include DbpA, DbpAv, and YB-1/DbpB/EFI. Examples of Runt class members include PEBP2aIphaA, PEBP2aIphaA1/AML-3, PEBP2alphaB/AML1, PEBP2alphaB1/AML1b, AML1a, AML1c, AML1DeltaN, and PEBP2alphaC1/AML2, among others.

Transcription factors comprising the superclass of “other” transcription factors include, but are not limited to, copper fist proteins, HMGI(Y) facors (e.g., HMG I, HMG Y, HMGI-C), and pocket domain factors (e.g., Rb, p107), AP2/EREBP-related factors, and SAND factors. A person skilled in the art will appreciate that the above-described classification of exemplary transcription factors may change over time, as may the designation of certain transcription factors. The transcription factors noted herein are provided as exemplary transcription factors that may be modulated or regulated by the repressors and/or or activators provided herein for the purpose of modulating the developmental potency and/or fate of a cell.

One example of a repressive complex comprising transcription factors and HMTs is the Polycomb Repressive Complex (PRC). Primarily, the PcG proteins comprise two functionally and biochemically distinct multimeric Polycomb repressive complexes (PRCs), 2-5 MDa in size, called PRC1 and PRC2. Biochemical purification of PRC1 from human cells has revealed the presence of a number of subunits including BMI1/MEL18 (vertebrate ortholog of posterior sex combs), RING1A/RING1B/RNF2 (ring finger protein), hPC 1-3 (Polycomb), hPH1-3 (Polyhomeotic), and YY1 (Pleiohomeotic) among others. PRC2 comprises the core components enhancer of zeste-2 (EZH2), suppressor of zeste-12 (SUZ12), and embryonic ectoderm development (Eed). Both the SUZ12 and the Eed are required for complex stability and for the methyltransferase activity of the EZH2. The EZH2-mediated transcriptional silencing depends upon the evolutionarily conserved catalytic SET (Su[VAR]3-9, Ezh2, Trithorax) domain, which imparts histone methyltransferase activity to the complex. Components of PRC1 and PRC2 contain intrinsic histone modifying activities specific for ubiquitination of lysine 119 of histone H2A (H2AK119ub) and trimethylation of lysine residue 27 of histone H3 (denoted as H3K27me3), respectively. Moreover, PRC2 has additional activity in lysine 26 of histone H1 under certain conditions.

As noted above, in various embodiments, the above-described exemplary transcription factors, while being modulated, may themselves be repressors and activators of other components of cell developmental potency pathways.

Other relevant pluripotency factors which may be used, separately or in conjunction with those noted above, can include essentially any other factors known to one having ordinary skill in the art that are capable of modulating components of developmental potency pathways involved in establishment and/or maintenance of pluripotency.

### VI. Pluripotency Pathways

A. Wnt Pathway

Wnt proteins are secreted cystein-rich proteins and about 20 have been identified in mammals. Several pathways exist through which Wnt proteins can elicit cell responses. For example, the Wnt pathway which involves β-catenin has been shown to control the specification, maintenance and activation of stem cells. Further, Wnt signaling pathways have been implicated in the both the establishment and maintenance of ES-cell pluripotency.

For example, Wnt3a activity can contribute to the self-renewal of ESCs, and its activation can sustain the expression of the pluripotent stage-specific transcription factors Oct 4 and Nanog. (J Cell Sci, 120, 55-65 and Stem Cells, 20, 284, 2002). Wnt3a activity also contributes the induced pluripotent cell reprogramming, where it is thought that Wnt activity substitutes for c-Myc activity (Lluis et al., 2008; Marson et al., 2008). Activation of the Wnt pathway leads to inhibition of GSK3, subsequent nuclear accumulation of β-catenin and the expression of target genes. In addition, activation of the Wnt canonical pathway maintains the undifferentiated phenotype in both mouse and human ESCs, and sustains expression of the pluripotent state specific transcription factors Oct-3/4, REX-1 and Nanog (Nature Med, 10, 55-63, 2004).

WNT signaling pathways are key components of the stem cell signaling network. The human WNT gene family consists of 19 members, encoding evolutionarily conserved glycoproteins with 22 or 24 Cys residues. Examples of WNT proteins include Wntl, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, WntlOa, WntlOb, Wntll, Wnt14, Wnt15, or Wntl 6. Wnt signaling pathways have been implicated in the maintenance of ES-cell pluripotency, and can contribute to the self-renewal of ESCs. (J Cell Sci, 120, 55-65 and Stem Cells, 20, 284, 2002). Generally, WNT signals are transduced through the canonical pathways for cell fate determination. For example, activation of the WNT canonical pathway maintains the undifferentiated phenotype in both mouse and human ESCs, and sustains expression of the pluripotent state specific transcription factors Oct-3/4, REX-1 and Nanog (Nature Med, 10, 55-63, 2004)

Canonical, or cell fate determining, WNT signals may be transduced, for example, through Frizzled (FZD) family receptors as well as the LRP5/LRP6 coreceptor to the β-catenin signaling cascade. In the absence of canonical WNT signaling, β-catenin complexed with APC and AXIN may be phosphorylated by casein kinase la (CKIa) and glycogen synthase kinase 3β (GSK3β) in the NH2-terminal degradation box, which may then be polyubiquitinated by βTRCP1 or βTRCP2 complex for subsequent proteasome mediated degradation. In the presence of canonical WNT signaling, Dishevelled (DVL) may be phosphorylated by CKIa for high-affinity binding to FRAT. Because canonical WNT signal induces the assembly of FZD-DVL complex and LRP4/6-AXIN-FRAT complex, β-catenin may be released from phosphorylation by CKIα and GSK3β for stabilization and nuclear accumulation. Nuclear β-catenin may complex with T-cell factor/lymphoid enhancer factor (TCF/LEF) family transcription factors and also with Legless family docking proteins, such as BCL9 and BCL9L, associated with PYGO family coactivators, such as PYGO1 and PYGO2. The TCF/LEF-β-catenin-Legless-PYGO nuclear complex may be the effector of the canonical WNT signaling pathway to activate the transcription of target genes such as FGF20, DKK1, WISP1, Myc, and CCND1.

WNT signaling modulators may include, merely by way of example, secreted-type WNT signaling inhibitors (e.g., repressors) and intracellular-type canonical WNT signaling inhibitors (e.g., repressors). Examples of secreted-type WNT signaling inhibitors (e.g., repressors) include, but are not limited to, SFRP1, SFRP2, SFRP3, SFRP4, SFRP5, WIF1, DKK1, DKK3, and DKK4. SFRP family members and WIF1 represent WNT repressors that inhibit WNT binding to FZD family receptors. DKK family members interact with LRP5/LRP6 coreceptor and trigger its endocytosis to prevent formation of the WNT-FZD-LRP5/LRP6 complex involved in canonical WNT signaling.

Examples of intracellular-type canonical WNT signaling repressors include, but are not limited to, APC, AXIN1, AXIN2, CKIα, GSK3β, NKD1, NKD2, ANKRD6, and NLK. APC, AXIN1, and AXIN2 represent scaffold proteins of the β-catenin destruction complex, whereas CKIα and GSK3β are serine/threonine kinases that phosphorylate β-catenin to trigger degradation.

Additional negative regulators (e.g., repressors) of WNT signaling include, for example, Engrailed-1, which negatively regulates β-catenin transcriptional activity by destabilizing β-catenin via a GSK3β-independent pathway, and protein kinase CK1-mediated steps, which may negatively regulate Wnt signalling by disrupting the lymphocyte enhancer factor-1/β-catenin complex. Idax functions as a negative regulator of the Wnt signaling pathway by directly binding to the PDZ domain of Dvl, which prevents the PDZ domain of Dvl from acting as a positive regulator (e.g., activator) in the Wnt signaling pathway. Accordingly, the PDZ domain of Dvl may activate the WNT pathway.

Duplin is a negative regulator (e.g., repressor) of β-catenin-dependent T-cell factor (Tcf) transcriptional activity in the Wnt signaling pathway, which acts by repressing Tcf-4 and/or STAT3. Suppressor of fused Su(fu) negatively regulates (e.g., represses) β-catenin signaling, and thus, WNT signaling. CRM-1-mediated nuclear export plays a role in regulation by Su(fu).

In addition, Akt participates in the Wnt signaling pathway through Dishevelled. For example, expression of Wnt or Dishevelled (Dvl) increases Akt activity, and activated Akt binds to the Axin-GSK3β complex in the presence of Dvl, phosphorylates GSK3β, and increases free β-catenin levels. Furthermore, in Wnt-overexpressing PC12 cells, dominant-negative Akt decreases free β-catenin and derepresses nerve growth factor-induced differentiation. Therefore, Akt acts in association with Dvl as an important regulator of the Wnt signaling pathway.

B. Hedgehog Pathway

Hedgehog (hh) proteins represent a family of secreted signal proteins responsible for the formation of numerous structures in embryogenesis (see, e.g., Smith, Cell 76 (1994) 193-196; Perrimon, Cell 80 (1995) 517-520; Chiang et al., Nature 83 (1996) 407; Bitgood et al., Curr. Biol. 6 (1996) 298-304; Vortkamp et al., Science 273 (1996) 613; and Lai et al., Development 121 (1995) 2349). During biosynthesis, signal sequence cleavage and autocatalytic cleavage form a 20 kDa N-terminal domain and a 25 kDa C-terminal domain. In its natural form, the N-terminal domain is modified with cholesterol or palmitoyl (see, e.g., Porter et al., Science 274 (1996) 255-259; Pepinski et al., J. Biol. Chem. 273 (1998) 14037-14045). In higher life-forms the Hh family is composed of at least three members, including Sonic, Indian and Desert Hedgehog (Shh, Ihh, Dhh; M. Fietz et al., Development (Suppl.) (1994) 43-51).

Hedgehog (Hh) molecules have been shown to play key roles in a variety of processes including tissue patterning, mitogenesis, morphogenesis, cellular differentiation and embryonic development (Lum et al., Science 304:1755-1759 (2004); and Bijlsma et al., BioEssays 26:387-394 (2004)). In mammals, three members of the Hh family of proteins have been identified, including Sonic Hedgehog (Shh), Indian Hedgehog (Ihh) and Desert Hedgehog (Dhh, mainly present in neural tissues). In addition to its role in embryonic development, Hh signaling plays a crucial role in postnatal development and maintenance of tissue/organ integrity and function. Studies using genetically engineered mice have demonstrated that Hh signaling is critical during skeletogenesis and vasculogenesis, as well as in the development of osteoblasts and endothelial cells in vitro and in vivo (Spinella-Jaegle et al., J Cell Sci 114:2085-2094 (2001); Hilton et al., Development 132:4339-4351 (2005); Chiang et al., Nature 383:407-413 (1996); and St-Jacques et al., Genes Develop 13:2072-2086 (1999)).

Hh signaling involves a very complex network of factors that includes plasma membrane proteins, kinases, phosphatases, and factors that facilitate the shuttling and distribution of Hedgehog molecules. Production of Hh proteins from a subset of producing/signaling cells involves synthesis, auto-processing and lipid modification. Hh signal transduction involves binding of processed Hh proteins to the Hh receptor Patched (Ptch), a 12-pass transmembrane protein that, in the absence of ligand, represses HH pathway activity by inhibiting the activity of the seven-transmembrane domain protein Smoothened (Smo). Binding of Hh protein to Ptch triggers the signaling activity of Smo, which eventually converts the latent GLI zinc finger transcription factors GLI2 and GLI3 into transcriptional activators to control Hh target gene expression. The Ci/Gli transcription factors enter the nucleus from the cytoplasm after a very intricate interaction between the members of a complex of accessory molecules that regulate the localization of Gli. Genes that are targeted by Hh signaling include Gli1, Ptch, bone morphogenetic protein 2 (BMP2), Wnt and homeobox genes. BMPs, Wnts, and homeobox genes are important regulators of osteoblast differentiation and bone formation in the skeleton and in the arterial wall (Hu et al., Development 132:49-60 (2004); and Shao et al., J Clin Invest 115:1210-1220 (2005)).

As noted above, the Ci/Gli family of transcription factors mediate Hedgehog (Hh) signaling in many key developmental processes. As a particular example, an Hh-induced MATH and BTB domain containing protein (HIB) represents a negative regulator (e.g., repressor) of the Hh pathway. Overexpressing HIB down regulates Ci and blocks Hh signaling, whereas inactivating HIB results in Ci accumulation and enhanced pathway activity. HIB binds the N- and C-terminal regions of Ci, both of which mediate Ci degradation. HIB forms a complex with Cul3, a scaffold for modular ubiquitin ligases, and promotes Ci ubiquitination and degradation through Cul3. Furthermore, HIB-mediated Ci degradation is stimulated by Hh and inhibited by Suppressor of Fused (Sufu). The mammalian homolog of HIB, SPOP, can functionally substitute for HIB, and Gli proteins are degraded by HIB/SPOP in Drosophila.

Additional examples of genes that contribute components to the Hh signaling pathway include, for example, the gene microtubule star (mts), which that encodes a subunit of protein phosphatase 2A, and the gene second mitotic wave missing (swm), which is predicted to encode an evolutionarily conserved protein with RNA binding and Zn+ finger domains. It is believed that mts is necessary for full activation of Hh signaling, and that swm is a negative regulator of Hh signaling and is essential for cell polarity.

7-dehydrocholesterol reductase (DHCR7), an enzyme catalyzing the final step of cholesterol biosynthesis, functions as positive regulator (e.g., activator) of Hh signaling that acts to regulate the cholesterol adduction of Hh ligand or to affect Hh signaling in the responding cell. DHCR7 also functions as a negative regulator (e.g., repressor) of Hh signaling at the level or downstream of Smoothened (Smo), and affects intracellular Hh signaling. In addition, the small GTPase Rab23 acts as a repressor of the Hedgehog signaling pathway. Protein kinase A (PKA) also acts in target cells as a common repressor of Hedgehog signaling.

Further examples of gene products that have specific roles in Hh signaling, include CKI a, dally-like (dip), caupolican (caup), and tlxe predicted gene, CG9211. Among them, CKI is a repressor, while dip, caup and CG9211 are all activators of Hh signaling.

C. Notch Pathway

Notch signaling controls selective cell-fate determination in a variety of tissues. The canonical Notch signaling pathway specifically regulates cell-fate decisions through close-range cell-cell interactions, and in both murine somatic and hESCs, the cytoplasmic signals induced by Notch activation are opposed by a control mechanism that involves the p38 mitogen-activated protein kinase (Nature, 442, 823-826, 2006). Repression of MEK/ERK by the MEK inhibitor PD098059 also inhibits differentiation and maintains ES-cell self-renewal in culture. The Notch Signaling Pathway (NSP) is a highly conserved pathway for cell-cell communication. Signals exchanged between neighboring cells through the Notch receptor can amplify and consolidate molecular differences, which eventually dictate cell fates. Notch signals control how cells respond to intrinsic or extrinsic developmental cues that are necessary to carryout specific developmental programs. Notch signaling controls selective cell-fate determination in a variety of tissues. The canonical Notch signaling pathway specifically regulates cell-fate decisions through close-range cell-cell interactions, for example, in both mature somatic cells and embryonic stem cells.

NSP is involved in the regulation of cellular differentiation, proliferation, and specification. For example, the NSP is utilised by continually renewing adult tissues such as blood, skin, and gut epithelium not only to maintain stem cells in a proliferative, pluripotent, and undifferentiated state, but also to direct the cellular progeny to adopt different developmental cell fates. Analogously, it is used during embryonic development to create fine-grained patterns of differentiated cells, notably during neurogenesis where the NSP controls patches such as that of the vertebrate inner ear where individual hair cells are surrounded by supporting cells. The NSP has been adopted by several other biological systems for binary cell fate choice. The Notch signaling pathway begins to inhibit new cell growth during adolescence, and keeps neural networks stable in adulthood.

The Notch receptor is synthesized in the rough endoplasmic reticulum as a single polypeptide precursor. Newly synthesized Notch receptor is proteolytically cleaved in the trans-golgi network, creating a heterodimeric mature receptor comprising of non-covalently associated extracellular and transmembrane subunits. This assembly travels to the cell surface, where it remains ready to interact with specific ligands. Following ligand activation and further proteolytic cleavage, an intracellular domain is released and translocates to the nucleus where it regulates gene expression.

Notch and most of its ligands are transmembrane proteins, so the cells expressing the ligands typically need to be adjacent to the Notch expressing cell for signaling to occur. Similar to Notch itself, Notch ligands are generally single-pass transmembrane proteins, such as members of the Delta/Serrate/LAG-2 (DSL) family of proteins. Mammalian Notch ligands include, for example, multiple Delta, Delta-like, Serrate, and Jagged ligands, as well as a variety of other ligands, such as F3/contactin.

The NSP may also be regulated or modulated by components that post-translational modify a Notch protein. Such components of the NSP include, but are not limited to, for example, Furin, Fringe, and O-FucT-1.

The NSP comprises numerous activators and repressors that are components of the Notch signaling pathway. For example, certain Notch signaling modulators include the ligands mentioned above, in addition to tumor necrosis factor alpha converting enzyme (TACE), Fringe, Deltex, Numb, Dv1, and the γ-secretase complex (comprising PSE2, PSEN, NCSTN, and APH-1). Additional components in the Notch pathway include Mastermind, Enhancer of Split, Hesl, Split, Hairless, Suppressor of Hairless, and RBP-Jk.

The NSP also comprises numerous downstream components, which are activated or repressed by Notch activation, such as through the Notch intracellular domain. Downstream components of the NSP may include, for example, Ras/MAPK and the MAPK signaling pathway. In addition, the downstream cytoplasmic signals induced by Notch activation may be repressed by a control mechanism that involves the p38 mitogen-activated protein kinase (Nature, 442, 823-826, 2006). Merely by way of example, repression of MEK/ERK by the MEK inhibitor PD098059 also inhibits differentiation and maintains ES-cell self-renewal in culture. Additional downstream components may include, for example, the transcription factor CSL, which may be co-activated by MAML and HATs, and which may be further regulated by co-repressors such as SMRT, CIR, CtBP, KyoT2, SHARP, NcoR, and/or HDAC, or protein degradation pathways such as Sel10 and/or CycC:CDK8. Notch activation via the transcription factor CSL may further induce the transcription of other downstream effectors, such as Hes1/5 and PreTα, in addition to other genes involved in modulating the fate of a cell.

D. LIF

Fetal calf serum (FCS) and LIF are generally required for the maintenance of undifferentiated mES-cell lines in vitro (Nature, 1988, 336, 688-690); however, LIF is not necessary for the maintenance of hESCs. LIF is a soluble glycoprotein of the interleukin (IL)-6 family of cytokines and acts via a membrane bound gp130 signaling complex to control signal transduction and activation of transcription (STAT) signaling. One specific phosphorylation target for this signaling cascade is c-Myc, which is critical for LIF regulation of mESCs (Development, 2005, 885-896). In addition to the pathway leading to STAT3 nuclear translocation, the intracellular domains of the LIFR-gp130 heterodimer can, on binding LIF, recruit the non receptor tyrosine kinase Janus (JAK) and the antiphosphotyrosine immunoreactive kinase (TIK) and activate other pathways. The treatment of ESCs with LIF also induces the phosphorylation of extracellular signal-regulated protein kinases, ERK1 and ERK2, and increases mitogen-activated protein kinase (MAPK) activity.

Other members in this family of cytokines, including IL-6, IL-11, oncostatin M, ciliary neurotrophic factor, and cardiotrophin-1, all show similar properties with respect to the maintenance of the pluripotency of mESCs. Importantly, the absence of IL-6 family members, the removal of mouse embryonic fibroblasts (MEFs), or the inactivation of STAT3 (a downstream signaling molecule of the gp130 signaling complex) promote ESCs to differentiate spontaneously in vitro (J Cell Biol, 1997, 138, 1207). LIF, when applied to serum-free ES-cell cultures, is however insufficient to maintain pluripotency, and other factors generally need to also be used in conjunction with LIF.

E. TGF-beta

Members of the transforming growth factor-beta (TGF-β) superfamily play important roles in the biology of epiblasts and ESCs. This family, which is composed of nearly 30 members, including activin, Nodal, and BMPs, elicit their responses through a variety of cell surface receptors that activate Smad protein signaling cascades. In combination with LIF, BMPs sustain self-renewal, multi-lineage differentiation, chimera colonization, and germ-line transmission properties. An important contribution of BMP is to induce the expression of 1d genes via activation of Smads 1, 5, or 8. The forced expression of 1d genes frees ESCs from BMP or serum dependence and allows self-renewal in LIF alone. Blockade of lineage specific transcription factors by Id proteins furthermore permits the self-renewal response to LIF/STAT3 signaling. Activin-Nodal signaling is, however, mediated primarily via Smads 2 and 3, and recent results have suggested that activin-Nodal-TGFβ signaling, but not BMP signaling, is indispensable for ES-cell propagation (Biochem Biophys Res Commun, 343,159-166,2006; Cell research, 2007, 17:42-49).

Members of the transforming growth factor-beta (TGF-β) superfamily play important roles in the biology of epiblasts and ESCs. This family, which is composed of nearly 30 members, including activin, Nodal, growth and differentiation factors (GDFs), and bone morphogenic proteins (BMPs), elicit their responses through a variety of cell surface receptors that activate Smad protein signaling cascades. In combination with LIF, BMPs sustain self-renewal, multi-lineage differentiation, chimera colonization, and germ-line transmission properties. Bone morphogenetic proteins cause the transcription of mRNAs involved in osteogenesis, neurogenesis, and ventral mesoderm specification. An important contribution of BMP is to induce the expression of Id genes via activation of Smads 1, 5, or 8. The forced expression of Id genes frees ESCs from BMP or serum dependence and allows self-renewal in LIF alone. Blockade of lineage specific transcription factors by Id proteins furthermore permits the self-renewal response to LIF/STAT3 signaling. Activin-Nodal signaling is, however, mediated primarily via Smads 2 and 3, and recent results have suggested that activin-Nodal-TGFβ signaling, but not BMP signaling, is indispensable for ES-cell propagation (Biochem Biophys Res Commun, 343,159-166,2006; Cell research, 2007, 17:42-49).

There are at least five receptor regulated SMADs in the TGF-β pathway: SMAD1, SMAD2, SMAD3, SMAD5 and SMAD9. There are essentially two intracellular pathways involving these R-SMADs. TGF beta's, Activins and Nodals may mediated by SMAD2 and SMAD3, while BMPs, GDFs and AMH may mediated by SMAD1, SMAD5 and SMAD9. The binding of the R-SMAD to the type I receptor may be mediated by a zinc double finger FYVE domain containing protein. Two such proteins that mediate the TGF beta pathway include SARA (The SMAD anchor for receptor activation) and HGS (Hepatocyte growth factor-regulated tyrosine kinase substrate).

SARA is present in an early endosome which, by clathrin-mediated endocytosis, internalizes the receptor complex. SARA recruits an R-SMAD. SARA permits the binding of the R-SMAD to the L45 region of the Type I receptor. SARA orients the R-SMAD such that serine residue on its C-terminus faces the catalytic region of the Type I receptor. The Type I receptor phosphorylates the serine residue of the R-SMAD. Phosphorylation induces a conformational change in the MH2 domain of the R-SMAD and its subsequent dissociation from the receptor complex and SARA

Exemplary components of the TGF-β cellular pathway include, for example, TGF-β, latent TGF-β, TGF-βRI, TGF-βRII, SARA, PP2A, SMADs, SMAD2, SMAD3, SMAD4, SMAD6, SMAD7, TAK1, TAB1, Ras, SHC, GRB2, SOS, MKK3, MKK4, JNK, p38, RhoA, PI3K, Cdh1, Akt/PKB, MEKs, ERK1/2, Ski/SnON, ATF2, c-Jun, c-Fos, CBP, p300, and R-SMAD/coSMAD complexes,

F FGF Signaling Pathway

Autocrine FGF signaling has also been shown to be important in human ESCs and these also express FGF2, 13, and 19 which are down-regulated upon induction of differentiation. While other pathways may as well be FGF-dependent in hESCs, FGF2 has been shown to activate the ERK/MAPK signaling cascade (BMC Developmental biology, 2007, 7:46).

The fibroblast growth factor (FGF) gene family is composed of 22 members, FGF-1 through FGF-23 that variously bind to seven FGF receptor isoforms from four FGF receptor genes: FGFR1b; FGFR1c; FGFR2b; FGFR2c; FGFR3b; FGFR3c and FGFR4. The b and c isoforms of FGFR1, FGFR2 and FGFR3 derive from alternative mRNA splicing that specifies the sequence of the carboxy-terminal half of each receptor's Ig-domain III. Many of the FGF gene products also exist in multiple isoforms generated by alternative gene splicing. Fibroblast growth factors have been organized into seven subfamilies based on sequence comparisons: the FGF1 subfamily (FGF1, FGF2) contains the prototype acidic FGF and basic FGF; the FGF4 subfamily (FGF4, FGF6, FGF5); the FGF7 (keratinocyte growth factor, KGF) subfamily (FGF3, FGF7, FGF10, FGF22); the FGF8 subfamily (FGF8, FGF17 and FGF18); the FGF9 subfamily (FGF9, FGF16, FGF29); the FGF11 subfamily (FGF11, FGF12, FGF13 and FGF14), originally the FGF homologous factors (FHF) 1-4 family (FHF1-FHF4) and the FGF19 subfamily (FGF19, FGF,21 and FGF23).

Fibroblast growth factor binding induces receptor tyrosine kinase (RTK) dimerization and activation leading to the activation of a plethora of signaling pathways involved with cell growth, differentiation and functions important for normal development, tissue maintenance and wound repair. Activation of specific cell signaling pathways is dependent upon the interaction of specific FGF ligands and FGF receptors, in addition to cell context. Effective activation of extracellular FGF signaling typically (except the FGF11 subfamily) requires the association of FGF and the FGF receptor with the extracellular matrix through components such as heparan sulfate glycosaminoglycans (HS). In addition to cell surface signaling, some FGF:FGF receptor complexes are translocated to the nucleus where they signal gene expression.

Exemplary components of the FGF signaling pathway include, for example, FRS2, GRB2, SOS, PLCy, Ras, PIP2, DAG, IP3, Rac1, PI3K, Raf1, RalGDS, Ral, MEKKs, MEKs, PKC, RalBP1, PLD, SEK, MKK3/6, JNK, p38, ERK1/2, IP3R, ATF2, and ELK1.

G. PI3K/AKT Signaling Pathway

PI3Ks are a family of lipid kinases, whose products, phosphoinositide 3,4-bisphosphate (P1(3,4)P2) and phosphoinositide 3,4,5-trisphosphate (PI(3,4,5)P3) act as intracellular second messengers. Members of the three distinct classes of PI3Ks have been implicated in the regulation of an array of physiological processes, notably the control of proliferation, cell survival, cell migration, and trafficking. Members of the class IA family of PI3Ks, comprising a regulatory subunit (typically 85 or 55 kDa) and a 110 kDa catalytic subunit are known to be activated via gp130, the signaling component of the LIF receptor. The role of phosphoinositide signaling in ESCs has been shown in reports implicating PI3Ks in the control of ESC proliferation. PI3Ks are also involved in regulation of self-renewal of murine ESCs. Using both pharmacological and molecular tools, it has been demonstrated that PI3K signaling is required for efficient self-renewal in the presence of LIF (J Biol Chem, 279, 46, 2004, 48063). Loss of self renewal upon inhibition of PI3K signaling is associated with an increase in ERK phosphorylation, which appears to play a functional role in this response. Additional evidence further supports the involvement of PI3K (J Biol Chem, 282, 9, 6265, 2007). The downstream molecular mechanisms that contribute to the ability of PI3Ks to regulate pluripotency of mouse ESCs was studied and it was shown that inhibition of PI3K activity with either pharmacological or genetic tools resulted in decreased expression of RNA for the homeodomain transcription factor Nanog and decreased Nanog protein levels.

H. Grb2/MEK Pathway

A sodium vanadate-induced tyrosine phosphorylation signal to repress Nanog in mice is transmitted via Grb2 (Mol Cell Biol, 2006, 26, 20, 7539). Grb2 is an adaptor molecule with an SH2 domain that specifically binds to a peptide motif containing a phosphotyrosine. This motif links Grb2 to downstream signaling cascades, in particular to the Sos/Ras/Raf/Mek/Erk pathway. Among the various kinase inhibitors tested, only the Mek inhibitor selectively blocked the effects of sodium vanadate on Nanog repression.

Moreover, transfection of a constitutively active form of Mek mutant repressed Nanog and led to primitive endoderm differentiation.

Illustrative inhibitors of MEK include flavone, PD98059, PD-325901, ARRY-142886, ARRY-438168, U0126

I. PI3K/AKT;MAPK/ERK

Large-scale transcriptional comparison of the hES-NCL1 line derived from a day 8 embryo with H1 line derived from a day 5 embryo (WiCell Inc.) showed that only 0.52% of the transcripts analysed varied significantly between the two cell lines. This is within the variability range that has been reported when hESC derived from days 5-6 embryos have been compared with each other. This implies that transcriptional differences between the cell lines are likely to reflect their genetic profile rather than the embryonic stage from which they were derived. Bioinformatic analysis of expression changes observed when these cells were induced to differentiate as embryoid bodies suggested that many of the downregulated genes were components of signal transduction networks. Subsequent analysis using western blotting, flow cytometry and antibody arrays implicated components of the PI3K/AKT kinase, MAPK/ERK and NFkb pathways and confirmed that these components are decreased upon differentiation. Disruption of these pathways in isolation using specific inhibitors resulted in loss of pluripotency and/or loss of viability, confirming the importance of such signaling pathways in embryonic stem cells. (Human Molecular Genetics, 2006, 15, 11, 18940).

### VII. Transcriptional Networks Affecting Pluripotency

The gene-expression program of pluripotent stem cells is a product of regulation by specific transcription factors, chromatin-modifying enzymes, regulatory RNA molecules, and signal-transduction pathways. Recent studies have provided new insights into how the key stem cell regulators work together to produce the pluripotent state.

Genetic studies first showed that the homeodomain transcription factors Oct4 and Nanog are essential regulators of early development and ES cell identity (Chambers et al., 2003, Chambers and Smith, 2004, Mitsui et al., 2003,Nichols et al., 1998). These transcription factors are expressed both in pluripotent ES cells and in the inner cell mass (ICM) of the blastocyst from which ES cells are derived. Disruption of Oct4 and Nanog causes loss of pluripotency and inappropriate differentiation of ICM and ES cells to trophectoderm and extraembryonic endoderm, respectively (Chambers et al., 2003, Nichols et al., 1998,Ying et al., 2002). Oct4 can heterodimerize with the HMG-box transcription factor Sox2 in ES cells and Sox2 contributes to pluripotency, at least in part, by regulating Oct4 levels (Masui et al., 2007). Oct4 is rapidly and apparently completely silenced during early cellular differentiation. Oct4, Sox2, and Nanog are central to the transcriptional regulatory hierarchy that specifies embryonic stem cell identity.

Identification of the genes occupied by Oct4, Sox2, and Nanog through genome-wide location analysis has provided insights into the molecular mechanisms by which these transcription factors contribute to pluripotency in human and murine ES cells (Boyer et al., 2005, Loh et al., 2006). These experiments yielded the following observations: (i) Oct4, Sox2, and Nanog bind together at their own promoters to form an interconnected autoregulatory loop, (ii) the three factors often co-occupy their target genes, and (iii) Oct4, Sox2, and Nanog collectively target two sets of genes, one that is actively expressed and another that is silent in ES cells but remains poised for subsequent expression during cellular differentiation (Boyer et al., 2005).

The Oct4, Sox2, and Nanog transcription factors occupy actively transcribed genes, including transcription factors and signaling components necessary to maintain the pluripotent stem cell state. Exemplary genes of this type, include, but are not limited to Oct4, Sox2, Nanog, Klf-4, Lin-28, AASDH, ADD3, ANKRD1, ANKRD15, ATAD2, ATP6V1G1, B3GALT4, BAMBI, BC061909, BMP7, BUB1B, BUB3, C12orf2, C13orf7, C15orf29, C6orf111, C9orf74, CA2, CA4, CABLES1, CACNA2D1, CAPZA2, CDCl₄B, CDC7, CDW92, CDYL, COL12A1, COMMD7, CPT1A, CTGF, DHRS3, DKK1, DPPA4, DPYSL2, DPYSL3, DTNA, DUSP12, DUSP6, EDD, ENPP2, ENST00000298406, EPHA1, EXOSC9, FAM33A, FBXW11, FEZ1, FGF2, FGFR1, FGFR2, FLJ10374, FLJ10652, FLJ10769, FLJ11029, FLJ14936, FRAT2, FUS, FZD10, GJA1, GNG10, GTPBP3, H2AFJ, HAS2, HN1, hSyn, ICMT, IER5L, JMJD1A, JUP, KCNN2, KDR, KIAA1143, KIAA1623, KIF15, KLHL5, LAMA4, LARGE, LEFTY2, LHPP, LOC124491, LRAT, LRFN3, LRRN1, LRRN6A, MAN2C1, MGC14798, MGC40168, MGC4170, MGEA5, MTM1, NAALAD2, NEBL, NID67, NUCKS, OLFML3, ORC1L, PARG, PCTK2, PDCL, PFTK1, PIPDX, PPAP2A, PPP2R1B, PPP2R3A, PRKCDBP, PTPN2, RAB5A, RAD54B, RASGRF2, RBM22, RIF1, RNF24, ROR1, RPS18, RPS3A, SET, SFRP1, SFRP2, SFRS4, SKIL, SMARCAD1, SNRPN, SPAG9, SPRED1, SULF1, TALDO1, TDGF1, TFCP2L3, THBS2, TIMM23, TMEM23, TNC, TNRC6A, TOP2A, TSC22D1, UBE2D3, Ufm1, USP44, USP7, VPS52, WDR36, ZIC2, ARID1B, COMMD3, EOMES, FOXO1A, HESX1, HHEX, HMG20A, IF116, IRX2, JARID2, KLF5, MED12, MLLT10, MSC, MYST3, NFE2L3, PHF17, PHF8, POLR3G, PRDM14, REST, SALL1, STAT3, TAF12, TAL1, TBL1XR1, TCF20, TCF7L1, TIF1, TLE3, TRIM22, ZFHX1B, ZFP36L1, ZIC1, and ZIC3, among others.

The three regulators also occupy the promoter regions of silent genes encoding transcription factors that, if expressed, would promote other more programmed or differentiated cell states. At these “stalled” set of genes, RNA polymerase II (POL2) initiates transcription but does not produce complete transcripts due to the repressive action of PcG proteins. The PcG proteins prevent RNA polymerase from transitioning into a fully modified transcription elongation apparatus, and thus, these differentiation genes are kept silent while the cells are maintained in a pluripotent state. Exemplary genes of this type include, but are not limited to: ACCN4, ADAMTS16, ADAMTSL1, ADRA1A, APOBEC3G, ARSD, BC020923, BC026345, BDH, BHLHB5, C7orf16, C7orf33, CCL2, CD82, CD99L2, CEI, CHRNA1, CPS1, CSAD, CSMD3, DBCCR1L, DEPDC2, DKFZp667B0210, ENST00000246083, ENST00000291982, ENST00000296508, ENST00000308142, ENST00000309467, ENST00000319884, ENST00000331014, ENST00000333380, ENST00000334440, EPHA4, FERD3L, FGF1, FIBL-6, FLJ14816, FLJ23263, FLJ25369, FLJ25791, FLJ32447, FLJ33167, FLJ35409, FLJ39779, FLJ43582, FLJ45187, FLJ46347, FTLL1, GAD2, GALNT3, GALNT8, GCNT2, GOLGA6, GRAP2, HBG1, HBG2, HIST1H1B, HIST1H1D, H1ST1H2AM, HIST1H₂BE, HIST1H2BF, HIST1H2BO, HIST1H3D, HIST1H3I, HIST1H3J, HIST1H4E, HIST1H4F, HIST1H4L, HIST2H4, HIST4H4, HSC201FIH1, IL2RG, INHA, KIAA1919, KITLG, LBP, LGI1, LOC153364, LOC169355, LOC283337, LOC349136, LOC440590, LRRC2, LRRTM3, LY96, MAB21L1, ME3, MGC34830, MGC39545, NS3TP2, OLFML2A, OR5AR1, OSR2, PDE10A, PRAC, PTF1A, PTHLH, RHD, RNF127, SEMA3A, SESN3, SHC3, ShrmL, SLC24A2, SLC24A3, SLC30A1, SPAG6, ST6GAL2, STEAP2, SYNPR, TRIMS, UNQ1940, WDR49, XCL1, ZIC4, ZICS, ZNF312, ATBF1, BC069363, DACH1, DLX1, DLX4, DLX5, DMRT1, EN1, ESX1L, FLI1, FLJ20097, FOXA2, FOXB1, FOXD3, GBX2, GLI3, GSC, GSH-2, HAND1, HAND2, HOP, HOXB1, HOXB3, HOXC4, INSM1, IPF1, ISL1, LBX1, LHX2, LHX5, MEIS1, MYF5, NEUROG1, NFIA, NFIX, NKX2-2, NKX2-3, NPAS3, NR2E1, NR4A2, NR6A1, OLIG3, ONECUT1, OTP, OTX1, PAX6, PCGF4, PROX1, RORB, SOX5, SPIC, TBX5, TFAP2C, TITF1, and YAF2.

Oct4, Sox2, and Nanog all autoregulatory (i.e., bind to and regulate their own promoters), as well as regulating the promoters of the genes encoding the two other factors (Boyer et al., 2005). This autoregulatory circuitry suggests that the three factors function collaboratively to maintain their own expression. Autoregulation is thought to enhance the stability of gene expression (Alon, 2007), which facilitates the maintenance of the pluripotent state. Autoregulatory loops appear to be a general feature of master regulators of cell state (Odom et al., 2006). Functional studies have confirmed that Oct4 and Sox2 co-occupy and activate the Oct4 and Nanog genes (Kuroda et al., 2005, Okumura-Nakanishi et al., 2005), and experiments with an inducible Sox2 null murine embyronic stem cell line have provided compelling evidence for the existence of this interconnected autoregulatory loop and its role in the maintenance of pluripotency (Masui et al., 2007).

The interconnected autoregulatory loop formed by Oct4, Sox2, and Nanog also suggests how the core regulatory circuitry of induced pluripotent cells might be jump-started when Oct4, Sox2, and other transcription factors are overexpressed in fibroblasts (Maherali et al., 2007, Okita et al., 2007, Takahashi and Yamanaka, 2006, Wernig et al., 2007). When these factors are exogenously overexpressed, they may contribute directly to the activation of endogenous Oct4, Sox2, and Nanog, the products of which in turn contribute to the maintenance of their own gene expression.

Oct4, Sox2, and Nanog co-occupy several hundred genes, often at apparently overlapping genomic sites (Boyer et al., 2005, Loh et al., 2006). This is evidence that these pluripotency factors generally do not control their target genes independently, but rather act coordinately to maintain the transcriptional program required for pluripotency. A large multiprotein complex containing Oct4 and Nanog can be obtained by iterative immunoprecipitation in pluripotent stem cells, providing further evidence that multiple interacting proteins coordinately control pluripotency (Wang et al., 2006). The possibility that multiple pluripotency factors function in a complex to coordinately control their target genes may help explain why efficient somatic cell reprogramming appears to require the combinatorial overexpression of multiple transcription factors. Not all components of this putative complex are required to initiate the process of reprogramming, however, because exogenous Nanog is not necessary for generation the generation of pluriptent cells by somatic cell reprogramming. It seems likely that exogenous Oct4 and other factors induce expression of endogenous Nanog to levels sufficient to accomplish full reprogramming.

The master regulators of pluripotency occupy the promoters of active genes encoding transcription factors, signal transduction components, and chromatin-modifying enzymes that promote pluripotent stem cell self-renewal (Boyer et al., 2005, Loh et al., 2006). However, these transcriptionally active genes account for only about half of the targets of Oct4, Sox2, and Nanog in ES cells. These master regulators also co-occupy the promoters of a large set of developmental transcription factors that are silent in pluripotent stem cells, but whose expression is associated with lineage commitment and cellular differentiation. Silencing of these developmental regulators is an important feature of pluripotency, because expression of these developmental factors is associated with commitment to particular lineages. For example, MyoD is a transcription factor capable of inducing a muscle gene expression program in a variety of cells (Davis et al., 1987). Therefore Oct4, Sox2, and Nanog help maintain the undifferentiated state of pluripotent stem cells by contributing to repression of lineage specification factors.

Most of the transcriptionally silent developmental regulators targeted by Oct4, Sox2, and Nanog are also occupied by the Polycomb group (PcG) proteins (Bernstein et al., 2006, Boyer et al., 2006, Lee et al., 2006), which are epigenetic regulators that facilitate maintenance of cell state through gene silencing. The PcG proteins form multiple polycomb repressive complexes (PRCs), the components of which are conserved from Drosophila to humans (Schuettengruber et al., 2007). PRC2 catalyzes histone H3 lysine-27 (H3K27) methylation, an enzymatic activity required for PRC2-mediated epigenetic gene silencing. H3K27 methylation provides a binding surface for PRC1, which facilitates oligomerization, condensation of chromatin structure, and inhibition of chromatin remodeling activity in order to maintain silencing. PRC1 also contains a histone ubiquitin ligase, Ring1b, whose activity contributes to silencing in ES cells (Stock et al., 2007).

Recent studies revealed that the silent developmental genes that are occupied by Oct4, Sox2, and Nanog and PcG proteins experience an unusual form of transcriptional regulation (Guenther et al., 2007). These genes undergo transcription initiation but not productive transcript elongation in ES cells. The transcription initiation apparatus is recruited to the promoters of genes encoding developmental regulators, where histone modifications associated with transcription initiation and the initial step of elongation (such as H3K4 methylation) are found, but RNA polymerase is incapable of fully transcribing these genes, presumably because of repression mediated by the PcG proteins. These observations explain why the silent genes encoding developmental regulators are generally organized in bivalent domains that are occupied by nucleosomes with histone H3K4me3, which is associated with gene activity, and by nucleosomes with histone H3K27me3, which is associated with repression (Azuara et al., 2006, Bernstein et al., 2006, Guenther et al., 2007).

The presence of RNA polymerase at the promoters of genes encoding developmental regulators (Guenther et al., 2007) may explain why these genes are especially poised for transcription activation during differentiation (Boyer et al., 2006, Lee et al., 2006). Polycomb complexes and associated proteins may serve to pause RNA polymerase machinery at key regulators of development in pluripotent cells and in lineages where they are not expressed. At genes that are activated in a given cell type, PcG proteins and nucleosomes with H3K27 methylation are lost (Bernstein et al., 2006, Boyer et al., 2006, Lee et al., 2006, Mikkelsen et al., 2007), allowing the transcription apparatus to fully transcribe these genes. The mechanisms that lead to selective activation of genes encoding specific developmental regulators involve signals brought to the genome by signal transduction pathways and likely involve H3K27 demethylation by enzymes such as the JmjC-domain-containing UTX and JMJD3 proteins (Lan et al., 2007).

The Oct4/Sox2/Nanog/Tcf3 complex regulates at least two groups of miRNAs: one group of miRNAs that is preferentially expressed in pluripotent cells and a second, Polycomb-occupied group that is silenced in pluripotent stem cells and is poised to contribute to cell-fate decisions during mammalian development.

Several miRNA polycistrons, which encode the most abundant miRNAs in pluripotent stem cells and which are silenced during early cellular differentiation (Houbaviy et al., 2003,Houbaviy et al., 2005,Suh et al., 2004), were occupied at their promoters by Oct4, Sox2, Nanog, and Tcf3. The most abundant in murine pluripotent stem cells was the mir-290-295 cluster, which contains multiple mature miRNAs with seed sequences similar or identical to those of the miRNAs in the mir-302 cluster and the mir-17-92 cluster. miRNAs with the same seed sequence also predominate in human embryonic stem cells (Laurent et al., 2008). miRNAs in this family have been implicated in cell proliferation (O'Donnell et al., 2005, He et al., 2005, Voorhoeve et al., 2006), consistent with the impaired self-renewal phenotype observed in miRNA-deficient ES cells (Kanellopoulou et al., 2005, Murchison et al., 2005, Wang et al., 2007). Additionally, the zebrafish homolog of this miRNA family, miR-430, contributes to the rapid degradation of maternal transcripts in early zygotic development (Giraldez et al., 2006), and mRNA expression data suggest that this miRNA family also promotes the clearance of transcripts in early mammalian development (Farh et al., 2005).

In addition to promoting the rapid clearance of transcripts as cells transition from one state to another during development, miRNAs also contribute to the control of cell identity by fine-tuning the expression of genes. miR-430, the zebrafish homolog of the mammalian mir-290295 family, serves to precisely tune the levels of Nodal antagonists Lefty1 and Lefty 2 relative to Nodal, a subtle modulation of protein levels that has pronounced effects on embryonic development (Choi et al., 2007). Recently, a list of 250 murine pluripotent stem cell mRNAs that appear to be under the control of miRNAs in the miR-290-295 cluster was reported (Sinkkonen et al., 2008). This study reports that Lefty1 and Lefty2 are evolutionarily conserved targets of the miR-290-295 miRNA family. These miRNAs also maintain the expression of de novo DNA methyltransferases 3a and 3b (Dnmt3a and Dnmt3b), by dampening the expression of the transcriptional repressor Rbl2, helping to poise pluripotent stem cells for efficient methylation of Oct4 and other pluripotency genes during differentiation.

Lefty1 and Lefty2, both actively expressed in pluripotent stem cells, are directly occupied at their promoters by Oct4/Sox2/Nanog/Tcf3. mir-290-295, which is also directly occupied by Oct4/Sox2/Nanog/Tcf3, depends on Oct4 for proper expression. Therefore, core pluripotent stem cell transcription factors appear to promote the active expression of Lefty1 and Lefty2 but also fine-tune the expression of these important signaling proteins by activating a family of miRNAs that target the Lefty1 and Lefty2 3′UTRs. This network motif whereby a regulator exerts both positive and negative effects on its target, termed incoherent feed-forwardregulation (Alon, 2007), provides a mechanism to fine-tune the steady-state level or kinetics of a target's activation. Over a quarter of the proposed targets of the miR-290-295 miRNAs (Sinkkonen et al., 2008) are predicted to be under the direct transcriptional control of Oct4/Sox2/Nanog/Tcf3 based binding site maps, suggesting that these miRNAs could participate broadly in tuning the effects of pluripotent stem cell transcription factors.

The miRNA expression program directly downstream of Oct4/Sox2/Nanog/Tcf3 help to poise pluripotent stem cells for rapid and efficient differentiation, consistent with the phenotype of miRNA-deficient cells (Kanellopoulou et al., 2005, Murchison et al., 2005, Wang et al., 2007). Oct4/Sox2/Nanog/Tcf3 contributes to this poising, in part, by their occupancy of the Let-7g promoter. Mature Let-7 transcripts are scarce in ES cells but were among the most abundant miRNAs in more differentiated cells such as MEFs and NPCs. Primary pri-Let-7g transcript is abundant in ES cells, but its maturation is blocked by Lin28 (Viswanathan et al., 2008). The promoters of both Let-7g and Lin28 are occupied by Oct4/Sox2/Nanog/Tcf3, suggesting that the core ES cell transcription factors promote the transcription of both primary pri-Let-7g and Lin28, which blocks the maturation of Let-7g. Indeed, proper expression of pri-Let-7g is dependent on Oct4. In this way Let-7 and Lin28 participate in an incoherent feed-forward circuit downstream of Oct4/Sox2/Nanog/Tcf3 to contribute to rapid cellular differentiation. Notably, ectopic expression of Lin28 in human fibroblasts promotes the induction of pluripotency (Yu et al., 2007), suggesting that blocked maturation of pri-Let-7 transcripts plays an important role in the pluripotent state. Additionally, Dnmt3a and Dnmt3b, which are indirectly upregulated by the miR-290295 miRNAs (Sinkkonen et al., 2008), are also occupied at their promoters by Oct4/Sox2/Nanog/Tcf3, providing examples of coherent regulation of important target genes by pluripotent stem cell transcription factors and the pluripotent stem cell miRNAs maintained by those transcription factors.

As noted above, pluripotent embryonic stem cells can be maintained in an undifferentiated state in culture, but are poised to rapidly differentiate. Extracellular signals have been identified that contribute to the maintenance of ES cell pluripotency or that stimulate differentiation down defined lineages. One such signaling molecule is LIF, which can help maintain murine pluripotent stem cells in an undifferentiated state in vitro, although it is not necessary for pluripotency in vivo (Smith et al., 1988). Other soluble factors, including Wnt, activin/nodal, and bFGF, have also been shown to contribute to maintenance of pluripotency, at least under certain culture conditions (Ogawa et al., 2006). Furthermore, human ES cells and the human fibroblasts on which pluripotent stem cells were plated have been reported to send reciprocal paracrine signals of FGF and IGF, respectively, sufficient to maintain the pluripotency of the ES cells (Bendall et al., 2007). These findings suggest that various signals help to establish a local microenvironment in vitro and presumably in vivo that helps to maintain pluripotency (see Essays by J. Rossant and J. Silva and A. Smith, and Review by C.E. Murry and G. Keller).

Signaling pathways also play key roles in promoting directed cellular differentiation. For example, activation of the Notch and BMP4 pathways can promote differentiation of ES cells (Chambers and Smith, 2004, Lowell et al., 2006). The Notch pathway has been shown to promote neural differentiation in both human and mouse embryonic stem cells. BMP4, on the other hand, can under certain conditions prevent neural cell differentiation while inducing differentiation into other cell types (Chambers and Smith, 2004).

When cell lineage commitment occurs, Oct4 is rapidly silenced and the appropriate regulators of development lose Polycomb-mediated repression and are activated. Oct4 and other regulators of pluripotency are highly restricted in their expression pattern to pluripotent stem cells, cells of the inner cell mass, and to cells of the germ line (Lengner et al., 2007). Ectopic expression of Oct4 has been shown to lead to rapid and massive expansion of poorly differentiated cells, especially in the intestine, and rapid fatality, highlighting the strong evolutionary pressure to ensure complete silencing of pluripotency regulators in somatic cells (Hochedlinger et al., 2005). Retinoic acid, a particularly well-characterized inducer of differentiation, has been shown to directly contribute to silencing of the Oct4 locus (Okamoto et al., 1990, Pikarsky et al., 1994). In addition, a set of nuclear repressors has been identified that are induced in differentiating cells and are required for proper silencing of Oct4, including ARP-1, COUP-TF1, and GCNF (also referred to as Nr6a1) (Ben-Shushan et al., 1995, Fuhrmann et al., 2001, Gu et al., 2005, Gu et al., 2006). Histone modifications associated with gene activity, including H3K4me3 and H3K7 and H3K9 acetylation, are lost at Oct4. Histone modifications associated with heterochromatin, H3K9me2 and me3, are gained in a G9a histone methyltransferase-dependent manner (Feldman et al., 2006). Finally, in a process dependent on de novo DNA methyltransferases DNMT3a/3b, which are recruited directly or indirectly by G9a, the Oct4 promoter undergoes CpG DNA methylation. Thus Oct4 and other pluripotent stem cell-specific genes, including Rex1, but not Nanog or Sox2, undergo a multistep, tightly regulated form of silencing, during which they adopt an epigenetic state characteristic of heterochromatin (Feldman et al., 2006). These epigenetic changes appear to enforce a more stable form of silencing compared to the more labile epigenetic silencing associated with H3K27 methylation at genes that must be dynamically regulated during development. As discussed below, these multilayered marks of epigenetic silencing, including H3K9 methylation and DNA methylation, must be progressively removed in the process of generating pluripotent cells by reprogramming somatic cells.

Thus, in various embodiments, the present invention contemplates, in part, to contact a population of somatic cells with one or more repressors and/or activators, to modulate one or more components of a cellular potency pathway(s) in order to reprogram the cells by activating the endogenous potency pathways of the cell, as described above and herein throughout. In one embodiment, it is preferred to mimic the endogenous cellular processes of reprogramming in order to reprogram a somatic cell to a multipotent, pluripotent, or totipotent state.

In particular embodiments, a method of reprogramming a cell, modulates a component of a potency pathway by altering the epigenetic state, chromatin structure, transcription, mRNA splicing, post-transcriptional modification, mRNA stability and/or half-life, translation, post-translational modification, protein stability and/or half-life and/or protein activity to facilitate reprogramming.

In certain embodiments, the components are transcriptionally activated to facilitate reprogramming. In other embodiments, the components are transcriptionally silenced (e.g., stalled or silenced epigenetically) to facilitate reprogramming, in part, by preventing cellular differentiation.

In certain embodiments, the components are transcriptionally repressed to facilitate programming. In other embodiments, the components are transcriptionally activated to facilitate programming, in part, by activating genes involved in cellular differentiation.

In another embodiment, factors upstream of pluripotency factors are used as repressors and/or activators in order to modulate a component (e.g., one or more pluripotency factors) of a cellular pathway associated with the developmenta potency of a cell. Such regulators are discussed elsewhere herein, for example in the section “Repressors and Activators”.

### VIII. Methods to Assess Pluripotency

The compositions and methods of the present invention provide, in part, reprogrammed pluripotent stem cells. In various embodiments, the pluripotency of a stem cell may be measured by any suitable method known to those having ordinary skill in the art, including, but not limited to: i) pluripotent stem cell morphology; ii) expression of pluripotent stem cell markers; iii) ability of pluripotent stem cells to contribute to germline transmission in mouse chimeras; iv) ability of plurpotent stem cells to contribute to the embryo proper using tetraploid embryo complementation assays; v) teratoma formation of pluripotent stem cells; vi) formation of embryoid bodies: and vii) inactive X chromosome reactivation.

The suitability of reprogrammed and/or programmed cells of the invention for use in methods and compositions of the present invention can also undergo karyotyping. That is, analysis of the chromosomal number and architecture is preferred in particular embodiments of the invention. Normal karyotyps in reprogrammed and/or programmed cells of the present invention would indicate that preferential use of these cells over others bearing abnormal karyotypes. Such abnormal karyotypes are indicators of genomic instability and often lead to disease processes, including, but not limited to, various forms of cancer.

### IX. Repressors and Activators

According to the present invention, a method of altering the potency of a cell (either by reprogramming or programming) comprises contacting a cell in an initial state of potency, with one or more repressors and/or activators, wherein the one or more repressors and/or activators modulates one or more components of a cellular pathway associated with the potency of a cell, thereby altering the initial state of potency to a less potent (e.g., programming) or more potent (e.g., reprogramming) state.

As noted above, a repressor can be an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

Also noted above, an activator can be an antibody or an antibody fragment, an mRNA, a bifunctional antisense oligonucleotide, a dsDNA, a polypeptide or an active fragment thereof, a peptidomimetic, a peptoid, or a small organic molecule.

In particular embodiments, any number and/or combination of these repressors or activators is suitable to formulate in a reprogramming or programming composition for use in the methods of the present invention as described elsewhere herein.

In certain embodiments, a repressor or activator is itself a component of a cellular developmental potency pathway, including, but not limited to a pluripotency factor, a transcription factor (including transcriptional activators and transcriptional repressors), a chromatin remodeling enzyme, and the like.

In other embodiments, the repressor or activator is a transcriptional repressor, a transcriptional activator, or an artificial transcription factor (either a repressor or activator), and the like. Other illustrative repressors and activators are described below.

A. DNAzymes

DNA enzymes (DNAzymes or deoxyribozymes), like ribozymes, may be perceived as gene-specific molecular scissors. Catalytic DNA has not been observed in nature, and all existing molecules have been derived by in vitro selection processes similar to those used to identify aptamers. The most well characterized DNAzyme is the “10-23” subtype comprising a cation-dependent catalytic core of 15 deoxyribonucleotides that binds to and cleaves its target RNA between an unpaired purine and paired pyrimidine through a de-esterification reaction, producing a 2′,3′-cyclic phosphate terminus and a 5′-hydroxyl terminus. Sequence conservation in the border regions of the catalytic core is important for the maintenance of catalytic activity. This core is flanked by complementary binding arms of 6 to 12 nucleotides in length that confer target mRNA specificity.

DNAzymes recognize the complementary mRNA sequence of its hybridizing arms via Watson-Crick base pairing and catalyze degradation of the target mRNA, producing two products, one containing a 2′,3′-cyclic phosphate terminus and the other a 5′-hydroxyl terminus.

The 10-23 DNAzyme, named by virtue of its selection process in vitro, catalyzes sequence-specific RNA cleavage in a manner akin to the hammerhead ribozyme and hence has substantial utility as a gene-silencing agent. In vitro cleavage experiments have shown that the 10-23 DNAzyme is highly specific and sensitive to small changes in target sequence. DNAzyme activity is dependent on the prevailing secondary structure of long-target RNA at the cleavage site. Thus, it is merely routine for one having skill in the art to test a range of molecules in order to identify those that display a high level of activity against biologically relevant target molecules. In terms of biological specificity, an important control in the assessment of DNAzyme antigene efficacy and specificity is the “scrambled DNAzyme,” wherein the sequence of nucleotides in the binding arms of the DNAzyme is randomly assembled while the catalytic core is preserved. This produces a molecule of identical size, the same percentage composition of nucleic acids, and the same net charge with a binding sequence that is not matched to the target gene. DNAzymes with nonsense or mismatch sequences in the binding arms or with point mutations in the catalytic core that render the DNAzyme enzymatically inactive can serve as additional controls.

A number of structural modifications have been used to enhance the stability and to improve the potency of DNAzymes. An important, commonly used modification is the incorporation of a 3′-3′ inverted nucleotide at the 3′ end of the DNAzyme to prevent exonuclease degradation. This can dramatically increase stability of the molecule, extending the half-life from 70 minutes to >21 hours in human serum. In addition, DNAzymes with this modification can remain functionally intact for at least 24 to 48 hours after exposure to serum compared with its unmodified counterpart with little change in the kinetics. Phosphorothioate (PS) linkages, which enhance stability by rendering the oligonucleotide more resistant to endogenous nucleases, have been used with DNAzymes. The introduction of PS modifications may affect cleavage efficiency and has been associated with toxicity, immunological responsiveness, and increased affinity for cellular proteins, resulting in sequence-independent effects.

Locked nucleic acids (LNAs), more recently, have been attractive monomers for modifying oligonucleotides and DNAzymes, in an attempt to increase binding affinity. LNA bases comprise a 2′-O 4-C methylene bridge that locks in a C3′-endo conformation, which places constraint on the ribose ring, thereby increasing affinity for complementary sequences. The advantages of LNAs include, but are not limited to increased thermal stability of duplexes toward complementary DNA or RNA, stability toward 3′-exonucleolytic degradation, solubility due to structural similarities to nucleic acids, easy automated synthesis with complete modified LNA or chimeric (LNA/DNA or LNA/RNA) oligonucleotides, and straightforward cellular delivery using standard transfection reagents. LNA incorporation into DNAzymes may influence catalytic activity under single-turnover conditions and biological potency. DNAzymes with an inverted nucleotide at the 3′ end are catalytically more efficient compared with their LNA-modified counterparts because of a slower product release rate.

Accumulating evidence indicates the utility, efficacy, and potency of DNAzymes in a variety of animal models of disease, allowing characterization of key molecular pathways underlying pathogenesis and use as a therapeutic agent. For instance, DNAzymes targeting the “master-regulator” zinc finger transcription factor Egr-1 have shown promise in experimental models of restenosis via inhibition of smooth muscle cell hyperplasia. Inhibition of neointima formation in the rat carotid artery after both balloon injury (first demonstration of DNAzyme efficacy in an animal model) and carotid artery ligation has also been demonstrated. Furthermore, intracoronary administration of DNAzymes targeting human Egr-1 reduced neointima formation in porcine coronary arteries after stent implantation. Likewise, Egr-1 DNAzymes attenuated neointima formation in human internal mammary arteries ex vivo.

Thus, in particular embodiments, the present invention provides a method to alter the potency of a cell, comprising contacting the cell with one or more repressors or a composition comprising the one or more repressors, wherein the one or more repressors includes a DNAzyme or combination of DNAzymes, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more DNAzymes, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one DNAzyme, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more DNAzymes; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one DNAzyme, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

B. RNA Interference

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

Elbashir et al., 2001, Nature, 411, 494 and Tuschl et al., International PCT Publication No. WO 01/75164, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877 and Tuschl et al., International PCT Publication No. WO 01/75164) has revealed certain requirements for siRNA length, structure, chemical composition, and sequence that are essential to mediate efficient RNAi activity. These studies have shown that 21-nucleotide siRNA duplexes are most active when containing 3″-terminal dinucleotide overhangs.

Furthermore, complete substitution of one or both siRNA strands with 2″-deoxy (2″-H) or 2″-O-methyl nucleotides abolishes RNAi activity, whereas substitution of the 3″-terminal siRNA overhang nucleotides with 2″-deoxy nucleotides (2″-H) was shown to be tolerated. Single mismatch sequences in the center of the siRNA duplex were also shown to abolish RNAi activity. In addition, these studies also indicate that the position of the cleavage site in the target RNA is defined by the 5″-end of the siRNA guide sequence rather than the 3″-end of the guide sequence (Elbashir et al., 2001, EMBO J, 20, 6877). Other studies have indicated that a 5″-phosphate on the target-complementary strand of a siRNA duplex is required for siRNA activity and that ATP is utilized to maintain the 5″-phosphate moiety on the siRNA (Nykanen et al., 2001, Cell, 107, 309).

The use of longer dsRNA has been described. For example, Tuschl et al., International PCT Publication No. WO 01/75164, describe a Drosophila in vitro RNAi system and the use of specific siRNA molecules for certain functional genomic and certain therapeutic applications. Fire et al., International PCT Publication No. WO 99/32619, describe particular methods for introducing certain long dsRNA molecules into cells for use in inhibiting gene expression in nematodes. Mello et al., International PCT Publication No. WO 01/29058, describe the identification of specific genes involved in dsRNA-mediated RNAi. Driscoll et al., International PCT Publication No. WO 01/49844, describe specific DNA expression constructs for use in facilitating gene silencing in targeted organisms. Fire et al., U.S. Pat. No. 6,506,559, describe certain methods for inhibiting gene expression in vitro using certain long dsRNA (299 bp-1033 bp) constructs that mediate RNAi.

Illustrative mechanisms of RNA interference, include, but are not limited to post transcriptional gene silencing, translational inhibition, transcriptional inhibition, or epigenetic RNAi. For example, siRNA molecules of the invention can be used to epigenetically silence genes at both the post-transcriptional level or the pre-transcriptional level. In a non-limiting example, epigenetic modulation of gene expression by siRNA molecules of the invention can result from siRNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siRNA molecules of the invention can result from siRNA mediated cleavage of RNA (either coding or non-coding RNA) via RISC, or alternately, translational inhibition as is known in the art. In a further non-limiting example embodiment, modulation of gene expression by siRNA molecules of the invention can result from transcriptional inhibition (see for example Janowski et al., 2005, Nature Chemical Biology, 1, 216-222).

In certain embodiments, a repressor, or RNAi oligonucleotide, is single stranded. In other embodiments, the repressor, or RNAi oligonucleotide, is double stranded. Certain embodiments may also employ short-interfering RNAs (siRNA). In certain embodiments, the first strand of the double-stranded oligonucleotide contains two more nucleoside residues than the second strand. In other embodiments, the first strand and the second strand have the same number of nucleosides; however, the first and second strands are offset such that the two terminal nucleosides on the first and second strands are not paired with a residue on the complimentary strand. In certain instances, the two nucleosides that are not paired are thymidine resides.

In instances when the repressor comprises siRNA, the agent should include a region of sufficient homology to the target gene, and be of sufficient length in terms of nucleotides, such that the siRNA agent, or a fragment thereof, can mediate down regulation of the target gene. Thus, an siRNA is or includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the over all double-strand character of the molecule.

In addition, an siRNA may be modified or include nucleoside analogs. Single stranded regions of an siRNA may be modified or include nucleoside analogs, e.g., the unpaired region or regions of a hairpin structure, e.g., a region which links two complementary regions, can have modifications or nucleoside analogs. Modification to stabilize one or more 3′- or 5′-terminus of an siRNA, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

Thus, in particular embodiments, the present invention provides a method to alter the potency of a cell, comprising contacting the cell with one or more repressors or a composition comprising the one or more repressors, wherein the one or more repressors includes an siRNA or combination of siRNAs, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more siRNAs, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one sRNA, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more siRNAs; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one sRNA, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

C. MicroRNAs (miRNAs)

MicroRNAs (miRNAs) are small non-coding RNAs of 20-22 nucleotides, typically excised from ˜70 nucleotide foldback RNA precursor structures known as pre-miRNAs. miRNAs constitute a recently discovered class of gene regulators that are found in both plants and animals. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. Briefly, mRNA transcripts are cleaved by ribonucleases in the miRNA-associated, multiprotein RNA-induced-silencing complex (miRISC), which results in the degradation of target mRNAs. This mechanism of miRNA-mediated gene silencing is commonly found in plants, but miRNA-directed mRNA cleavage has also been shown to occur in mammals.

However, most animal miRNAs are thought to use a second mechanism of gene regulation that does not involve the cleavage of their mRNA targets. These miRNAs exert their regulatory effects by binding to imperfect complementary sites within the 3′ untranslated regions (UTRs) of their mRNA targets, and they repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. However, recent findings indicate that miRNAs that share only partial complementarity with their targets can also induce mRNA degradation.

The biogenesis of miRNAs has only recently been elucidated. miRNAs, which generally seem to be transcribed by RNA polymerase II, are initially made as large RNA precursors that are called pri-miRNAs. The pri-miRNAs are processed in the nucleus by the RNase III enzyme, Drosha, and the double-stranded-RNA-binding protein, Pasha (also known as DGCR8), into 70-120 nucleotide pre-miRNAs, which fold into imperfect stem-loop structures. The pre-miRNAs are then exported into the cytoplasm by the RAN GTP-dependent transporter exportin 5 and undergo an additional processing step in which a double-stranded RNA of 20-22 nucleotides in length, referred to as the miRNA:miRNA duplex, is excised from the pre-miRNA hairpin by another RNAse III enzyme, Dicer. Subsequently, the miRNA:miRNA duplex is incorporated into the miRISC complex. The mature miRNA strand is preferentially retained in the functional miRISC complex and negatively regulates its target genes.

MicroRNAs have diverse functions of in animal development and disease. MicroRNA expression profiles in both human and mouse ESCs revealed that ESCs express a unique set of miRNAs, and that these miRNAs are down-regulated as ESCs differentiate into embryoid bodies. Some of these miRNAs are conserved between human and mouse and are clustered in the genome (Suh M. R. et al., Human embryonic stem cells express a unique set of microRNAs. Dev. Biol. (2004) 270:488-498 and Houbaviy H. B., et al., Embryonic stem cell-specific MicroRNAs. Dev. Cell (2003) 5:351-358).

MiRNAs play a role in the maintenance of pluripotency. Loss of DGCR8, an RNA-binding protein that assists the RNase III enzyme Drosha in the processing of miRNA, results in a complete absence of mature miRNAs, though the RNAi pathway is not affected. DGCR8-deficient ESCs fail to fully down-regulate pluripotency markers during differentiation and retain an ESC colony morphology. Nevertheless, they do express some markers of differentiation, confirming the specific role of miRNAs in ESC differentiation (Wang Y., et al., DGCR8 is essential for microRNA biogenesis and silencing of embryonic stem cell self-renewal. Nat. Genet. (2007) 39:380-385).

MicroRNAs facilitate differentiation by down-regulation of pluripotency-associated genes. It has been shown that the microRNA miR-134 promotes ESC differentiation into the ectodermal lineage, partly due to its direct translational attenuation of Nanog and LRH1 (Tay Y. M., et al., MicroRNA-134 modulates the differentiation of mouse embryonic stem cells where it causes post-transcriptional attenuation of Nanog and LRH1. Stem Cells (2008) 26:17-29).

High-resolution, genome-wide maps of core ESC transcription factors, have identified promoter regions for most miRNA genes, and deduced the association of the ESC transcription factors with these miRNA genes. Transcriptional regulators in ESCs collectively occupied the promoters of many of the miRNAs that are most abundant in ESCs, including those that are downregulated as ESCs differentiate. In addition, these factors also occupy the promoters of a second, smaller set of miRNAs that are repressed in ESCs and are selectively expressed in specific differentiated cell types. In ESCs, this second group of miRNAs are co-occupied by Polycomb group proteins, which are also known to silence key lineage-specific, protein-coding developmental regulators. Thus, two key groups of miRNAs are direct targets of Oct-3/4/Sox-2/Nanog/Tcf3: one group of miRNAs that is preferentially expressed in pluripotent cells and a second, Polycomb-occupied group that is silenced in ESCs and is poised to contribute to cell-fate decisions during mammalian development.

Several miRNA polycistrons, which encode the most abundant miRNAs in ESCs and which are silenced during early cellular differentiation (Houbaviy et al., 2003, Houbaviy et al., 2005, Suh et al., 2004), are occupied at their promoters by Oct-3/4, Sox-2, Nanog, and Tcf3. The most abundant miRNAs in murine ESCs was the mir-290-295 cluster, which contains multiple mature miRNAs with seed sequences similar or identical to those of the miRNAs in the mir-302 cluster and the mir-17-92 cluster. mRNAs with the same seed sequence also predominate in human embryonic stem cells (Laurent et al., 2008). mRNAs in this family have been implicated in cell proliferation (O'Donnell et al., 2005, He et al., 2005, Voorhoeve et al., 2006), consistent with the impaired self-renewal phenotype observed in miRNA-deficient ESCs (Kanellopoulou et al., 2005, Murchison et al., 2005,Wang et al., 2007).

In addition to promoting the rapid clearance of transcripts as cells transition from one state to another during development, miRNAs also likely contribute to the control of cell identity by fine-tuning the expression of genes. miR-430, the zebrafish homolog of the mammalian mir-290-295 family, serves to precisely tune the levels of Nodal antagonists Lefty1 and Lefty 2 relative to Nodal, a subtle modulation of protein levels that has pronounced effects on embryonic development (Choi et al., 2007). Recently, a list of 250 murine ESC mRNAs that appear to be under the control of miRNAs in the miR-290-295 cluster was reported (Sinkkonen et al., 2008). This study reports that Lefty1 and Lefty2 are evolutionarily conserved targets of the miR-290-295 miRNA family. These miRNAs also maintain the expression of de novo DNA methyltransferases 3a and 3b (Dnmt3a and Dnmt3b), perhaps by dampening the expression of the transcriptional repressor Rbl2, helping to poise ESCs for efficient methylation of Oct-3/4 and other pluripotency genes during differentiation.

The core transcriptional circuitry of ESCs connects to both miRNAs and protein-coding genes and reveals recognizable network motifs downstream of Oct-3/4/Sox-2/Nanog/Tcf3, involving both transcriptional and posttranscriptional regulation, that provide new insights into how this circuitry controls ESC identity. Lefty1 and Lefty2, both actively expressed in ESCs, are directly occupied at their promoters by Oct-3/4/Sox-2/Nanog/Tcf3. mir-290-295, which is also directly occupied by Oct-3/4/Sox-2/Nanog/Tcf3, depends on Oct-3/4 for proper expression. Therefore, core ESC transcription factors promote the active expression of Lefty1 and Lefty2 but also fine-tune the expression of these important signaling proteins by activating a family of miRNAs that target the Lefty1 and Lefty2 3′UTRs. This network motif whereby a regulator exerts both positive and negative effects on its target, termed “incoherent feed-forward” regulation (Alon 2007), provides a mechanism to fine-tune the steady-state level or kinetics of a target's activation. Over a quarter of the proposed targets of the miR-290-295 miRNAs (Sinkkonen et al., 2008) are likely under the direct transcriptional control of Oct-3/4/Sox-2/Nanog/Tcf3 based on transcription factor binding site mapping studies. Thus, these miRNAs can participate broadly in tuning the effects of ESC transcription factors.

The miRNA expression program directly downstream of Oct-3/4/Sox-2/Nanog/Tcf3 prepares ESCs for rapid and efficient differentiation, consistent with the phenotype of miRNA-deficient cells (Kanellopoulou et al., 2005, Murchison et al., 2005,Wang et al., 2007). Oct-3/4/Sox-2/Nanog/Tcf3 likely contributes to this preparation by their occupancy of the Let-7g promoter. Mature Let-7 transcripts are scarce in ESCs but were among the most abundant miRNAs in both MEFs and NPCs. Primary pri-Let-7g transcript is abundant in ESCs, but its maturation is blocked by Lin28 (Viswanathan et al., 2008). The promoters of both Let-7g and Lin28 are occupied by Oct-3/4/Sox-2/Nanog/Tcf3, thus, the core ESC transcription factors promote the transcription of both primary pri-Let-7g and Lin28, which blocks the maturation of Let-7g. Indeed, proper expression of pri-Let-7g is dependent on Oct-3/4. In this way Let-7 and Lin28 participate in an incoherent feed-forward circuit downstream of Oct-3/4/Sox-2/Nanog/Tcf3 to contribute to rapid cellular differentiation. Notably, ectopic expression of Lin28 in human fibroblasts promotes the induction of pluripotency (Yu et al., 2007), thus, blocked maturation of pri-Let-7 transcripts plays an important role in the pluripotent state. Additionally, Dnmt3a and Dnmt3b, which are indirectly upregulated by the miR-290-295 miRNAs (Sinkkonen et al., 2008), are also occupied at their promoters by Oct-3/4/Sox-2/Nanog/Tcf3, providing examples of “coherent” regulation of important target genes by ESC transcription factors and the ESC miRNAs maintained by those transcription factors.

Oct-3/4, Sox-2, Nanog, and Tcf3 occupy the promoters of two key sets of miRNAs, similar to the two sets of protein-coding genes regulated by these factors: one set that is actively expressed in pluripotent ESCs and another that is silenced in these cells by Polycomb group proteins and whose later expression might serve to facilitate establishment or maintenance of differentiated cell states.

The number of human miRNAs reported so far (the April 2008 release of miRBase at the Sanger Institute) is 678, nearly three times as many as initial calculations indicated. Additionally, more than 1,000 predicted miRNA genes are awaiting experimental confirmation.

Illustrative miRNAs that are suitable for use with the present invention include, but are not limited to: hsa-let-7a, hsa-let-7b, hsa-let-7c, hsa-let-7d, hsa-let-7e, hsa-let-7f, 15 hsa-miR-15a, hsa-miR-16, hsa-miR-17-5p, hsa-miR-17-3p, hsa-miR-18a, hsa-miR-19a, hsa-miR-19b, hsa-miR-20a, hsa-miR-21, hsa-miR-22, hsa-miR-23a, hsa-miR-189, hsa-miR-24, hsa-miR-25, hsa-miR-26a, hsa-miR-26b, hsa-nniR-27a, hsa-miR-28, hsa-miR-29a, hsa-miR-30a-5p, hsa-miR-30a-3p, hsa-miR-31, hsa-miR-32, hsa-miR-33, hsa-miR-92, hsa-miR-93, hsa-miR-95, hsa-miR-96, hsa-miR-98, hsa-miR-99a, hsa-miR-100, hsa-miR-20 101, hsa-miR-29b, hsa-miR-103, hsa-miR-105, hsa-miR-106a, hsa-miR-107, hsa-miR-192, hsa-miR-196a, hsa-miR-197, hsa-miR-198, hsa-miR-199a, hsa-miR-199a*, hsa-miR-208, hsa-miR-129, hsa-miR-148a, hsa-miR-30c, hsa-miR-30d, hsa-miR-139, hsa-miR-147, hsa-miR-7, hsa-miR-10a, hsa-miR-10b, hsa-miR-34a, hsa-miR-181a, hsa-miR-181b, hsa-miR-181c, hsa-miR-182, hsa-miR-182*, hsa-miR-183, hsa-miR-187, hsa-miR-199b, hsa-25 miR-203, hsa-miR-204, hsa-miR-205, hsa-miR-210, hsa-miR-211, hsa-miR-212, hsa-miR-181a*, hsa-miR-214, hsa-miR-215, hsa-miR-216, hsa-miR-217, hsa-miR-218, hsa-miR-219, hsa-miR-220, hsa-miR-221, hsa-miR-222, hsa-miR-223, hsa-miR-224, hsa-miR-200b, hsa-let-7g, hsa-let-71, hsa-miR-1, hsa-miR-15b, hsa-miR-23b, hsa-miR-27b, hsa-miR-30b, hsa-miR-122a, hsa-miR-124a, hsa-miR-125b, hsa-miR-128a, hsa-miR-130a, 30 hsa-miR-132, hsa-miR-133a, hsa-miR-135a, hsa-miR-137, hsa-miR-138, hsa-miR-140, hsa-miR-141, hsa-miR-142-5p, hsa-miR-142-3p, hsa-miR-143, hsa-miR-144, hsa-miR-145, hsa-miR-152, hsa-miR-153, hsa-miR-191, hsa-miR-9, hsa-miR-9*, hsa-miR-125a, hsa-miR-126*, hsa-miR-126, hsa-miR-127, hsa-miR-134, hsa-miR-136, hsa-miR-146a, hsa-miR-149, hsa-miR-150, hsa-miR-154, hsa-miR-154*, hsa-miR-184, hsa-miR-185, hsa-miR-186, hsa-miR-188, hsa-miR-190, hsa-miR-193a, hsa-miR-194, hsa-miR-195, hsa-miR-206, hsa-miR-320, hsa-miR-200c, hsa-miR-155, hsa-miR-128b, hsa-miR-106b, hsa-miR-29c, hsa-miR-200a, hsa-miR-302a*, hsa-miR-302a, hsa-miR-34b, hsa-miR-34c, hsa-miR-299-3p, hsa-miR-301, hsa-miR-99b, hsa-miR-296, hsa-miR-130b, hsa-miR-30e-5p, hsa-miR-30e-3p, hsa-miR-361, hsa-miR-362, hsa-miR-363, hsa-miR-365, hsa-miR-302b*, hsa-miR-302b, hsa-miR-302c*, hsa-miR-302c, hsa-miR-302d, hsa-miR-367, hsa-miR-368, hsa-miR-369-3p, hsa-miR-370, hsa-miR-371, hsa-miR-372, hsa-miR-373*, hsa-miR-373, hsa-miR-374, hsa-miR-375, hsa-miR-376a, hsa-miR-377, hsa-miR-378, hsa-miR-422b, hsa-miR-379, hsa-miR-380-5p, hsa-miR-380-3p, hsa-miR-381, hsa-miR-382, hsa-miR-383, hsa-miR-340, hsa-miR-330, hsa-miR-328, hsa-miR-342, hsa-miR-337, hsa-miR-323, hsa-miR-326, hsa-miR-151, hsa-miR-135b, hsa-miR-148b, hsa-miR-331, hsa-miR-324-5p, hsa-miR-324-3p, hsa-miR-338, hsa-miR-339, hsa-miR-335, hsa-miR-133b, hsa-miR-325, hsa-miR-345, hsa-miR-346, ebv-miR-BHRFI-1, ebv-miR-BHRFI-2*, ebv-miR-BHRFI-2, ebv-miR-BHRFI-3, ebv-miR-BARTI-5p, ebv-miR-BART2, hsa-miR-384, hsa-miR-196b, hsa-miR-422a, hsa-miR-423, hsa-miR-424, hsa-miR-425-3p, hsa-miR-18b, hsa-miR-20b, hsa-miR-448, hsa-miR-429, hsa-miR-449, hsa-miR-450, hcmv-miR-UL22A, hcmv-miR-UL22A*, hcmv-miR-UL36, hcmv-miR-UL112, hcmv-miR-UL148D, hcmv-miR-US5-1, hcmv-miR-US5-2, hcmv-miR-US25-I, hcmv-miR-US25-2-5p, hcmv-miR-US25-2-3p, hcmv-miR-US33, hsa-miR-191*, hsa-miR-200a*, hsa-miR-369-5p, hsa-miR-431, hsa-miR-433, hsa-miR-329, hsa-miR-453, hsa-miR-451, hsa-miR-452, hsa-miR-452*, hsa-miR-409-5p, hsa-miR-409-3p, hsa-miR-412, hsa-miR-410, hsa-miR-376b, hsa-miR-483, hsa-miR-484, hsa-miR-485-5p, hsa-miR-485-3p, hsa-miR-486, hsa-miR-487a, kshv-miR-K12-10a, kshv-miR-K12-10b, kshv-miR-K12-II, kshv-miR-K12-1, kshv-miR-K12-2, kshv-miR-K12-9*, kshv-miR-K12-9, kshv-miR-K12-8, kshv-miR-K12-7, kshv-miR-K12-6-5p, kshv-miR-K12-6-3p, kshv-miR-K12-5, kshv-miR-K12-4-5p, kshv-miR-K12-4-3p, kshv-miR-K12-3, kshv-miR-K12-3*, hsa-miR-488, hsa-miR-489, hsa-miR-490, hsa-miR-491, hsa-miR-511, hsa-miR-146b, hsa-miR-202*, hsa-miR-202, hsa-miR-492, hsa-miR-493-5p, hsa-miR-432, hsa-miR-432*, hsa-miR-494, hsa-miR-495, hsa-miR-496, hsa-miR-193b, hsa-miR-497, hsa-miR-181d, hsa-miR-512-5p, hsa-miR-512-3p, hsa-miR-498, hsa-miR-520e, hsa-miR-515-5p, hsa-miR-515-3p, hsa-miR-519e*, hsa-miR-519e, hsa-miR-520f, hsa-miR-526c, hsa-miR-519c, hsa-miR-520a*, hsa-miR-520a, hsa-miR-526b, hsa-miR-526b*, hsa-miR-519b, hsa-miR-525, hsa-miR-525*, hsa-miR-523, hsa-miR-518f*, hsa-miR-518f, hsa-miR-520b, hsa-miR-518b, hsa-miR-526a, hsa-miR-520c, hsa-miR-518c*, hsa-miR-518c, hsa-miR-524*, hsa-miR-524, hsa-miR-517*, hsa-miR-517a, hsa-miR-519d, hsa-miR-521, hsa-miR-520d*, hsa-miR-520d, hsa-miR-517b, hsa-miR-520g, hsa-miR-516-5p, hsa-miR-516-3p, hsa-miR-518e, hsa-miR-527, hsa-miR-518a, hsa-miR-518d, hsa-miR-517c, hsa-miR-520h, hsa-miR-522, hsa-miR-519a, hsa-miR-499, hsa-miR-500, hsa-miR-501, hsa-miR-502, hsa-miR-503, hsa-miR-504, hsa-miR-505, hsa-miR-513, hsa-miR-506, hsa-miR-507, hsa-miR-508, hsa-miR-509, hsa-miR-510, hsa-miR-514, hsa-miR-532, hsa-miR-299-5p, hsa-miR-18a*, hsa-miR-455, hsa-miR-493-3p, hsa-miR-539, hsa-miR-544, hsa-miR-545, hsa-miR-487b, hsa-miR-551a, hsa-miR-552, hsa-miR-553, hsa-miR-554, hsa-miR-92b, hsa-miR-555, hsa-miR-556, hsa-miR-557, hsa-miR-558, hsa-miR-559, hsa-miR-560, hsa-miR-561, hsa-miR-562, hsa-miR-563, hsa-miR-564, hsa-miR-565, hsa-miR-566, hsa-miR-567, hsa-miR-568, hsa-miR-551b, hsa-miR-569, hsa-miR-570, hsa-miR-571, hsa-miR-572, hsa-miR-573, hsa-miR-574, hsa-miR-575, hsa-miR-576, hsa-miR-577, hsa-miR-578, hsa-miR-579, hsa-miR-580, hsa-miR-581, hsa-miR-582, hsa-miR5 583, hsa-miR-584, hsa-miR-585, hsa-miR-548a, hsa-miR-586, hsa-miR-587, hsa-miR-548b, hsa-miR-588, hsa-miR-589, hsa-miR-550, hsa-miR-590, hsa-miR-591, hsa-miR-592, hsa-miR-593, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-10 612, hsa-miR-613, hsa-miR-614, hsa-miR-615, hsa-miR-616, hsa-miR-548c, hsa-miR-617, hsa-miR-618, hsa-miR-619, hsa-miR-620, hsa-miR-621, hsa-miR-622, hsa-miR-623, hsa-miR-624, hsa-miR-625, hsa-miR-626, hsa-miR-627, hsa-miR-628, hsa-miR-629, hsa-miR-630, hsa-miR-631, hsa-miR-33b, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-15 miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-548d, hsa-miR-661, hsa-miR-662, hsa-miR-663, hsa-miR-449b, hsa-miR-653, hsa-miR-411, hsa-miR-654, hsa-miR-655, hsa-miR-656, hsa-miR-549, hsa-miR-657, hsa-miR-658, hsa-miR-659, hsa-miR-660, hsa-miR-421, hsa-miR-542-5p, hcmv-miR-USA, hcmv-miR-UL70-5p, hcmv-20 miR-UL70-3p, hsa-miR-363*, hsa-miR-376a*, hsa-miR-542-3p, ebv-miR-BARTI-3p, hsa-miR-425-5p, ebv-miR-BART3-5p, ebv-miR-BART3-3p, ebv-miR-BART4, ebv-miR-BART5, ebv-miR-BART6-5p, ebv-miR-BART6-3p, ebv-miR-BART7, ebv-miR-BART8-5p, ebv-miR-BART8-3p, ebv-miR-BART9, ebv-miR-BARTIO, ebv-miR-BARTII-5p, ebv-miR-BARTII-3p, ebv-miR-BART12, ebv-miR-BART13, ebv-miR-BART14-5p, ebv-miR-BART14-3p, kshv-miR-25 K12-12, ebv-miR-BART15, ebv-miR-BART16, ebv-miR-BART17-5p, ebv-miR-BART17-3p, ebv-miR-BART18, ebv-miR-BART19, ebv-miR-BART20-5p, ebv-miR-BART20-3p, hsvl-miR-HI, hsa-miR-758, hsa-miR-671, hsa-miR-668, hsa-miR-767-5p, hsa-miR-767-3p, hsa-miR-454-5p, hsa-miR-454-3p, hsa-miR-769-5p, hsa-miR-769-3p, hsa-miR-766, hsa-miR-765, hsa-miR-768-5p, hsa-miR-768-3p, hsa-miR-770-5p, hsa-miR-802, hsa-miR-801, and hsa-30 miR-675.

One having ordinary skill in the art is aware that miRNAs encompass both naturally occurring miRNAs, such as those listed above, as well as artificially designed miRNAs. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Boden, Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

Thus, in particular embodiments, the present invention provides a method to alter the potency of a cell, comprising contacting the cell with one or more repressors or a composition comprising the one or more repressors, wherein the one or more repressors includes an miRNA or combination of miRNAs, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more miRNAs, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one miRNA, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more miRNAs; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one miRNA, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

In yet other related embodiments, the miRNAs are artificially designed miRNAs.

D. Short Hairpin RNAs

A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell. In preferred embodiments, expression of an shRNA is regulated by an RNA polymerase III promoters; such promoters are known to produce efficient silencing. While essentially any PoIII promoters may be used, desirable examples include the human U6 snRNA promoter, the mouse U6 snRNA promoter, the human and mouse H1 RNA promoter and the human tRNA-val promoter. A U6 snRNA leader sequence may be appended to the primary transcript; such leader sequences tend to increase the efficiency of sub-optimal shRNAs while generally having little or no effect on efficient shRNAs. For transcription from a transgene in vivo, a regulatory region (e.g., promoter, enhancer, silencer, splice donor and acceptor, polyadenylation) may be used to regulate expression of the shRNA strand (or strands). Inhibition may be controlled by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. The use and production of an expression construct are known in the art (see also WO 97/32016; U.S. Pat. Nos. 5,593,874, 5,698,425, 5,712,135, 5,789,214, and 5,804,693; and the references cited therein).

In a preferred embodiment, a shRNA construct is designed with 29 by helices following a U6 snRNA leader sequence with the transcript being produced by the human U6 snRNA promoter. This transcription unit may be delivered via a Murine Stem Cell Virus (MSCV)-based retrovirus, with the expression cassette inserted downstream of the packaging signal. Further information on the optimization of shRNA constructs may be found, for example, in the following references: Paddison, P. J., A. A. Gaudy, and G. J. Hannon, Stable suppression of gene expression by RNAi in mammalian cells. Proc Natl Acad Sci USA, 2002. 99(3): p. 1443-8; 13. Brummelkamp, T. R., R. Bemards, and R. Agami, A System for Stable Expression of Short Interfering RNAs in Mammalian Cells. Science, 2002. 21: p. 21; Kawasaki, H. and K. Taira, Short hairpin type of dsRNAs that are controlled by tRNA(Val) promoter significantly induce RNAi-mediated gene silencing in the cytoplasm of human cells. Nucleic Acids Res, 2003. 31(2): p. 700-7, Lee, N. S., et al., Expression of small interfering RNAs targeted against HIV-1 rev transcripts in human cells. Nat Biotechnol, 2002. 20(5): p. 500-5; Miyagishi, M. and K. Taira, U6 promoter-driven siRNAs with four uridine 3′ overhangs efficiently suppress targeted gene expression in mammalian cells. Nat Biotechnol, 2002. 20(5): p. 497-500; Paul, C P., et al., Effective expression of small interfering RNA in human cells. Nat Biotechnol, 2002. 20(5): p. 505-8.

Thus, in particular embodiments, the present invention provides a method to alter the potency of a cell, comprising contacting the cell with one or more repressors or a composition comprising the one or more repressors, wherein the one or more repressors includes an shRNA or combination of shRNAs, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more shRNAs, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one shRNA, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more shRNAs; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one shRNA, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

E. Ribozymes

Ribozymes are catalytically active RNA molecules capable of site-specific cleavage of target mRNA and, unlike DNAzymes, can occur naturally. Like DNAzymes and antisense oligonucleotides (ASOs), ribozymes need access to their binding sites in the target RNA. Several subtypes have been described; those most commonly studied are hammerhead and hairpin ribozymes, which differ in their catalytic response to changes in solvent pH rather than their capacity to bind and ligate cleavage products or reliance on metal ions. Ribozyme catalytic activity and stability can be improved by substituting deoxyribonucleotides for ribonucleotides at noncatalytic bases.

While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

Chimeric DNA-RNA hammerhead ribozymes targeting platelet-derived growth factor A-chain mRNA have been shown to inhibit intimal thickening in balloon-injured rat carotid arteries after local delivery, whereas those targeting transforming growth factor-β protect against renal injury in hypertensive rats after systemic (intraperitoneal) delivery. Clinically, ribozymes have been explored therapeutically in several small trials. Hammerhead anti-HIV ribozymes have been used in T-lymphocyte expansion strategies ex vivo followed by infusion into patients. Hammerhead ribozymes targeting a highly conserved portion of 5′-untranslated region of hepatitis C virus HEPTAZYME showed promise in phase I and II trials. However, because of toxicological concerns, the study was suspended. Ribozymes have also been evaluated as potential adjuncts in cancer therapy. These include the synthetic antiangiogenic ANGIOZYME, which targets the VEGF receptor VEGF R1 (Flt-1) in a variety of solid tumors, and HERzyme, which targets human epidermal growth factor-2 overexpressed in breast and ovarian cell carcinoma.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators, published International patent application No. WO88/04300. The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.). A preferred method of delivery involves uses a DNA construct “encoding” the ribozyme under the control of a strong constitutive pol III or pol II promoter, so that transfected cells will produce sufficient quantities of the ribozyme to destroy targeted messages and inhibit translation. Because ribozymes, unlike antisense molecules, are catalytic, a lower intracellular concentration is required for efficiency.

Thus, in particular embodiments, the present invention provides a method to alter the potency of a cell, comprising contacting the cell with one or more repressors or a composition comprising the one or more repressors, wherein the one or more repressors includes a ribozyme or combination of ribozymes, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more ribozymes, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one ribozyme, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more ribozymes; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one ribozyme, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

In certain embodiments, the ribozyme is a hammerhead ribozyme. In other embodiments, the ribozymes is a Cech-type ribozyme.

F. Antagomirs

An “antagomir” or “oligonucleotide agent” of the present invention refers to a single stranded, double stranded or partially double stranded oligomer or polymer of ribonucleic acid (RNA) or deoxyribonucleic acid (DNA) or both or modifications thereof, which is antisense with respect to its target. Antagomirs include, but are not limited to, oligonucleotides composed of naturally-occurring nucleobases, sugars and covalent internucleoside (backbone) linkages and non-naturally-occurring portions which function similarly.

In some embodiments, modified or substituted oligonucleotides are preferred over native forms because of desirable properties such as, for example, enhanced cellular uptake, enhanced affinity for nucleic acid target and increased stability in the presence of nucleases. In one embodiment, the antagomir does not include a sense strand, and in another preferred embodiment, the antagomir does not self-hybridize to a significant extent. An antagomir featured in the invention can have secondary structure, but it is substantially single-stranded under physiological conditions. An antagomir that is substantially single-stranded is single-stranded to the extent that less than about 50% (e.g., less than about 40%, 30%, 20%, 10%, or 5%) of the antagomir is duplexed with itself.

As used herein, the term “substantially complementary” means that two sequences are substantially complementary that a duplex can be formed between them. The duplex may have one or more mismatches but the region of duplex formation is sufficient to down-regulate expression of the target nucleic acid. The region of substantial complementarity can be perfectly paired. In other embodiments, there will be nucleotide mismatches in the region of substantial complementarity. In a preferred embodiment, the region of substantial complementarity will have no more than 1, 2, 3, 4, or 5 mismatches.

The antagomirs featured in the invention can be about 12 to about 30 nucleotides long, e.g., about 15 to about 25, or about 18 to about 25 nucleotides long (e.g., about 19, 20, 21, 22, 23, 24 nucleotides long). The antagomirs featured in the invention can target RNA, e.g., an endogenous pre-miRNA or miRNA of the subject or an endogenous pre-miRNA or miRNA of a pathogen of the subject. For example, an antagomir of the present invention can target any miRNA of a cell in vivo or ex vivo using the methods described herein.

In a particular embodiment, an antagomir of the present invention can be used to target one or more miRNAs families and/or clusters selected from the group consisting of: Let-7 family, miR-10 family, miR-103, miR-124, miR-130, miR-132, miR-137, miR-15, miR-153, miR-155, miR-16, miR-17-20, miR-17-92, miR-181a/b, miR-182, miR-183, miR-196, miR-21, miR-22, miR-222, miR-23, miR-24, miR-26, miR-26a/b, miR-27, miR-29, the mir-290-295 cluster, miR-301, the miR-302 cluster, miR-375, miR-615, miR-708, miR-9, miR-96, and miR-99a.

Thus, in particular embodiments, the present invention provides a method to alter the potency of a cell, comprising contacting the cell with one or more repressors or a composition comprising the one or more repressors, wherein the one or more repressors includes an antagomir or combination of antagomirs, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more antagomirs, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one antagomir, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more antagomirs; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one atagomir, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

G. Aptamers

An “aptamer” may be a nucleic acid molecule, such as RNA or DNA that is capable of binding to a specific molecule with high affinity and specificity (Ellington et al., Nature 346, 818-22 (1990); and Tuerk et al., Science 249, 505-10 (1990)). Exemplary ligands that bind to an aptamer include, without limitation, small molecules, such as drugs, metabolites, intermediates, cofactors, transition state analogs, ions, metals, nucleic acids, and toxins. Aptamers may also bind natural and synthetic polymers, including proteins, peptides, nucleic acids, polysaccharides, glycoproteins, hormones, receptors and cell surfaces such as cell walls and cell membranes. The binding of a ligand to an aptamer, which is typically RNA, causes a conformational change in the effector domain and alters its ability to interact with its target molecule. Therefore, ligand binding affects the effector domain's ability to mediate gene inactivation, transcription, translation, or otherwise interfere with the normal activity of the target gene or mRNA, for example. An aptamer will most typically have been obtained by in vitro selection for binding of a target molecule. However, in vivo selection of an aptamer is also possible.

Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand as compared to the dissociation constant of the aptamer for other materials in the environment or unrelated molecules in general. A ligand is one which binds to the aptamer with greater affinity than to unrelated material. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 300 nucleotides in length. More commonly, an aptamer will be between about 20 and about 100 nucleotides, between about 30 and about 75 nucleotides, or between about 40 and about 60 nucleotides in length.

In one embodiment, an aptamer-regulated nucleic acid of the invention comprises an aptamer domain and an effector nucleic acid domain. An aptamer-regulated nucleic acid of the invention may comprise DNA or RNA and may be single-stranded or double-stranded. An aptamer-regulated nucleic acid may comprise multiple modular components, e.g., one or more aptamer domains and/or one or more effector domains. Aptamer-regulated nucleic acids may further comprise a functional group or a functional agent, e.g., an intercalator or an alkylating agent. Aptamer-regulated nucleic acids may comprise synthetic or non-natural nucleotides and analogs (e.g., 6-mercaptopurine, 5-fluorouracil, 5-iodo-2′-deoxyuridine and 6-thioguanine) or may include modified nucleic acids. Exemplary modifications include cytosine exocyclic amines, substitution of 5-bromo-uracil, backbone modifications, methylations, and unusual base-pairing combinations. Aptamer-regulated nucleic acids may include labels, such as fluorescent, radioactive, chemical, or enzymatic labels. An aptamer domain responds to ligand binding to induce an allosteric change in the effector domain, and alters the ability of the effector domain to interact with its target molecule. Ligand binding, therefore, switches the effector domain from “off” to “on,” or vice versa. Aptamer-regulated nucleic acids, therefore, act as a switch whose activity is turned “off” and “on” in response to ligand binding. The response of the aptamer domain to the ligand may also depend on the ligand identity and/or the amount or concentration of ligand exposed to the aptamer domain. For example, an aptamer may bind small molecules, such as drugs, metabolites, intermediates, cofactors, transition state analogs, ions, metals, nucleic acids, and toxins. Alternatively, an aptamer may bind natural and synthetic polymers, including proteins, peptides, nucleic acids, polysaccharides, glycoproteins, hormones, receptors and cell surfaces such as cell walls and cell membranes. In certain other embodiments, the aptamer domain of a ligand controlled nucleic acid is responsive to environmental changes. Environmental changes include, but are not limited to changes in pH, temperature, osmolarity, or salt concentration. An effector nucleic acid domain may comprise an antisense nucleic acid or a DNA. An effector nucleic acid domain may also comprise a sequence that can be used as an RNAi sequence, such as a sRNA or miRNA. In preferred embodiments, ligand binding at the aptamer domain mediates a change in the conformational dynamics of these molecules that allows the effector nucleic acid domain to interact with a target nucleic acid, for example, an mRNA.

In one embodiment, the effector domain of an aptamer-regulated nucleic acid interacts with a target gene by nucleic acid hybridization. For instance, an aptamer-regulated nucleic acid may comprise an effector domain that comprises a hybridization sequence that hybridizes to a target sequence of a gene and an aptamer domain that binds to a ligand. The binding of the ligand to the aptamer domain causes a conformational change in the aptamer-regulated nucleic acid that alters the ability (such as availability and/or Tm) of the hybridization sequence of the effector domain to hybridize to a target sequence. Furthermore, an effector domain may modulate the expression or activity of its target by any method known in the art. In one embodiment, the effector domain of an aptamer-regulated nucleic acid comprises an effector domain that comprises an antisense sequence and acts through an antisense mechanism in modulating expression of a target gene. For instance, an aptamer-regulated nucleic acid may comprise an effector domain that comprises an antisense sequence for inhibiting expression of a target gene and an aptamer domain that binds to a ligand. The binding of the ligand to the aptamer domain causes a conformational change in the aptamer-regulated nucleic acid that alters the ability of the antisense sequence of the effector domain to inhibit expression of the target sequence.

In another embodiment, the effector domain of an aptamer-regulated nucleic acid comprises an effector domain that comprises an RNAi sequence and acts through an RNAi or miRNA mechanism in modulating expression of a target gene. For example, an aptamer-regulated nucleic acid may comprise an effector domain that comprises a miRNA or sRNA sequence for inhibiting expression of a target gene and an aptamer domain that binds to a ligand. The binding of the ligand to the aptamer domain causes a conformational change in the aptamer-regulated nucleic acid that alters the ability of the miRNA or sRNA sequence of the effector domain to inhibit expression of the target sequence.

In one embodiment, an effector domain comprises a miRNA or sRNA sequence that is between about 19 nucleotides and about 35 nucleotides in length, or preferably between about 25 nucleotides and about 35 nucleotides. In certain embodiments, the effector domain is a hairpin loop that may be processed by RNAse enzymes (e.g., Drosha and Dicer). RNA-mediated silencing mechanisms include inhibition of mRNA translation and directed cleavage of targeted mRNAs. Recent evidence has suggested that certain RNAi constructs may also act through chromosomal silencing, i.e. at the genomic level, rather than, or in addition to, the mRNA level. Thus, the sequence targeted by the effector domain can also be selected from untranscribed sequences that regulate transcription of a target gene of the genomic level.

Thus, in particular embodiments, the present invention provides a method to alter the potency of a cell, comprising contacting the cell with one or more repressors or a composition comprising the one or more repressors, wherein the one or more repressors includes an aptamer or combination of aptamers, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more aptamers, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one aptamer, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more aptamers; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one aptamer, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

H. Antisense Oligonucleotides

For purposes of the invention, the term “oligonucleotide” includes polymers of two or more deoxyribonucleosides, ribonucleosides, or 2′-O-substituted ribonucleoside residues, or any combination thereof. Preferably, such oligonucleotides have from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines, and adamantane. The term oligonucleotide also encompasses such polymers as PNA and LNA. For purposes of the invention the term “2′-O-substituted” means substitution of the 2′ position of the pentose moiety with an —O-lower alkyl group containing 1-6 saturated or unsaturated carbon atoms, or with an —O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al., U.S. Pat. Nos. 5,635,377 and 5,366,878). Preferably, such chimeric oligonucleotides contain at least one, at least two, at least three, or at least four consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide will contain at least one, at least two, at least three, or at least four consecutive deoxyribonucleosides and will also contain ribonucleosides, 2′-O-substituted ribonucleosides, or combinations thereof (see e.g., Metelev and Agrawal, U.S. Pat. Nos. 5,652,355 and 5,652,356).

Antisense oligonucleotides utilized in the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Methods in Molec. Biol. 20: 465-496, 1993).

Antisense approaches involve the design of oligonucleotides (either DNA or RNA) that are complementary to mRNA encoding a component of a cellular pathway associated with the pluripotency of a cell. On the basis of mechanism of action, two classes of antisense oligonucleotide can be discerned: (a) the RNase H-dependent oligonucleotides, which induce the degradation of mRNA; and (b) the steric-blocker oligonucleotides, which are RNAse H inactive because they lack phosphorothioate groups, are believed to function by sterically blocking target RNA formation, nucleocytoplasmic transport or translation. This steric-blocker class of oligonucleotides includes, for example, methylphosphonates, morpholino oligonucleotides, peptide nucleic acids (PNA's), 2′-O-allyl or 2′-O-alkyl modified oligonucleotides, and N3′->P5′phosphoramidates.

The majority of the antisense drugs investigated in the clinic function via an RNase H-dependent mechanism. RNase H is a ubiquitous enzyme that hydrolyzes the RNA strand of an RNA/DNA duplex. Oligonucleotide-assisted RNase H-dependent reduction of targeted RNA expression can be quite efficient, reaching 80-99% down-regulation of protein and mRNA expression. Furthermore, in contrast to the steric-blocker oligonucleotides, RNase H-dependent oligonucleotides can inhibit protein expression when targeted to virtually any region of the mRNA. Thus, whereas most steric-blocker oligonucleotides are efficient only when targeted to the 5′- or AUG initiation codon region, phosphorothioate oligonucleotides, e.g., can inhibit protein expression when targeted to widely separated areas in the coding region.

The importance of RNase H-induced cleavage of mRNA has been demonstrated in at least four systems, including wheat germ extract rabbit reticulocyte lysate, Xenopus oocytes, and human leukemia cells. RNase H competent backbones include oligodeoxynucleotide phosphodiesters and phosphorothioates. 2′-fluorooligodeoxynucleotides are also RNase H competent. Other modifications, including methylphosphonates, 2′-O-methyloligoribonucleotides, PNAs, and morpholino oligonucleotides, are not RNase H competent. Using chimeric oligonucleotides in which 2′-O-methyloligoribonucleotide phosphorothioates are placed at the 3′ and 5′ termini of the oligonucleotide, while the central region remains phosphorothioate oligodeoxyribonucleotide, it has been demonstrated that a 5-bp region of homology is sufficient to induce RNase H activity.

Other oligonucleotide modifications (2′-O-alkyl, PNA, and morpholinos) may use different mechanisms to inhibit protein expression, e.g., they can inhibit intron excision, a key step in the processing of mRNA. Splicing occurs during the maturation step and can be inhibited by the hybridization of an oligonucleotide to the 5′ and 3′ regions involved in this process. Such inhibition can lead to the lack of expression of a mature protein or, as numerous reports have shown, to the correction of aberrant splicing and the restoration of a functional protein. This approach has been also developed in mice. Most of the oligonucleotides capable of inhibiting splicing are non RNase H dependent.

Numerous reports in the literature also demonstrate that oligonucleotides can efficiently inhibit mRNA translation. This inhibition is attributable to the disruption of the ribosomes and/or by physically blocking the initiation or elongation steps of protein translation. Steric blockade of translation can be demonstrated by the arrest of the polypeptide chain elongation, as shown by Dias et al. 1999.

Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs are also effective at inhibiting translation of mRNAs. Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation, but could also be used in accordance with the invention. Whether designed to hybridize to the 5′, 3′ or coding region of mRNA, antisense nucleic acids should be at least 6, at least 8, at least 10, at least 12, at least 14, at least 15, at least 16, at least 17, at least 18, at least 19, at least 20, at least 21, at least 22, at least 23, at least 24, or at least 25 nucleotides in length, and are preferably less that about 100, about 90, about 80, about 70, about 60, about 50, about 40, about 30, about 25, about 20, about 18, about 16, about 12, or about 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., PCT Publication No. WO88/09810) or the blood-brain barrier (see, e.g., PCT Publication No. WO89/10134), hybridization-triggered cleavage agents or intercalating agents. To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are known in the art. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The present invention also contemplates, in part, one or more antisense oligonucleotides comprising “locked nucleic acids” (LNAs), which are novel conformationally restricted oligonucleotide analogues containing a methylene bridge that connects the 2′-O of ribose with the 4′-C (see, Singh et al, Chem. Commun., 1998, 4:455-456).

In yet a further embodiment, the antisense oligonucleotide is an anomeric oligonucleotide. An anomeric oligonucleotide forms specific double-stranded hybrids with complementary RNA in which, contrary to the usual units, the strands run parallel to each other. The oligonucleotide is a 2′-O-methylribonucleotide, or a chimeric RNA-DNA analogue.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., methylphosphonate oligonucleotides can be prepared by use of controlled pore glass polymer supports.

While antisense nucleotides complementary to the coding region of an mRNA sequence can be used, those complementary to the transcribed untranslated region and to the region comprising the initiating methionine are preferred in some embodiments.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigen expressed on the target cell surface) can be administered systematically.

Another approach utilizes a recombinant DNA construct in which the antisense oligonucleotide is placed under the control of a strong pol m or pol II promoter. The use of such a construct to transfect target cells in the patient will result in the transcription of sufficient amounts of single stranded RNAs that will form complementary base pairs with the endogenous transcripts and thereby prevent translation. For example, a vector can be introduced in vivo such that it is taken up by a cell and directs the transcription of an antisense RNA. Such a vector can remain episomal. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include, but are not limited to the SV40 early promoter region, the promoter contained in the 3′ long terminal repeat of Rous sarcoma virus, the herpes thymidine kinase promoter, the regulatory sequences of the metallothionein gene (Brinster et al., 1982, Nature 296:3942), etc.

Thus, in particular embodiments, the present invention provides a method to alter the potency of a cell, comprising contacting the cell with one or more repressors or a composition comprising the one or more repressors, wherein the one or more repressors includes an antisense oligonucleotide or combination of antisense oligonucleotides, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more antisense oligonucleotides, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one antisense oligonucleotide, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more antisense oligonucleotides; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one antisense oligonucleotide, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

I. Bifunctional Antisense Oligonucleotides

Alternative pre-mRNA splicing is a fundamental mechanism for regulating the expression of a multitude of eukaryotic genes. The basic splicing signals, which include the 5′ splice site, branch site, and polypyrimidine tract-AG, are initially recognized by the U1 small nuclear ribonucleoprotein (snRNP), U2 snRNP, U2 snRNP auxiliary factor (U2AF), respectively, and a number of other proteins. These basic splicing signals tend to be degenerate in higher eukaryotes and cannot alone confer the specificity required to achieve accurate splice site selection. Various types of exonic and intronic elements that can modulate the use of nearby splice sites have now been identified. Among the best known examples of such elements are the exonic splicing enhancers, i.e., sequences naturally present in pre-mRNA that stimulate the splicing of pre-mRNA transcripts to form mature mRNAs (Cartegni, L. et al. (2002) Nat. Rev. Genet. 3(4), 285-298, PMID: 11967553; Cáceres, J. F. and Kornblihtt, A. R. (2002) Trends Genet. 18(4), 186-193, PMID: 11932019). The definition of “enhancer” is functional, and includes sequences within exons that are not located at the splice sites and are not universally obligatory but do stimulate splicing at least in the gene in which they were identified. Enhancers are commonly thought of as elements in alternatively spliced exons that compensate in part for weak canonical splicing signals. However, it has been shown recently that even constitutive exons can contain several enhancer sequences. The majority of enhancer sequences identified are rich in purines, although recent selection strategies have shown that more diverse classes of sequence are also functional. In a number of cases, it has been shown that these sequences are recognised directly by specific SR (for serine and arginine-rich) proteins. These RNA-binding proteins play a critical role in initiating complex assembly on pre-mRNA, and are essential fox constitutive splicing and also affect alternative splicing both in vivo and in vitro. It is very likely that other proteins, such as Tra2α or β or hnRNP G also play a role in enhancer sequence recognition and/or processing.

Pre-mRNA molecules may also contain cryptic or mutant splice sites, especially 5′ splice sites. The 5′ splice site is defined by a poorly conserved short sequence around a highly conserved GU (guanine-uracil) dinucleotide. In most cases, there are many similar sequences in the adjacent intron and exon, but the correct site is chosen as a result of a combination of influences: the extent to which the sequences fit the consensus, the positions of exon elements and other splice sites, and the concentration of the various factors that affect 5′ splice sites. Numerous genetic diseases result from mutations at the 5′ splice site, the consequences of which are either skipping of the exon or the use of some of the other candidate sites (cryptic splice sites). Enhancer defects are difficult to assign and have only recently entered the broader consciousness as possible explanations for the effects of mutations. Well-known examples of genetic diseases that arise from mutations affecting splicing include thalassaemias (e.g. OMIM #141900 for haemoglobin-beta locus), muscular dystrophies (e.g. OMIM #310200), collagen defects (van Leusden, M. R. et al. (2001) Lab Invest. 81(6), 887-894, PMID: 11406649), and proximal spinal muscular atrophy (SMA) (Monani, U. R., et al. (1999) Hum. Mol. Genet. 8, 1177-1183, PMID: 10369862; Lorson, C L., et al. (1999) Proc. Natl. Acad. Sci. USA 96, 6307-6311, PMID: 1 0339583).

Thus, according to one embodiment of the present invention, a nucleic acid molecule is provided comprising a first and a second domain, the first domain being capable of forming a first specific binding pair with a target sequence of a target RNA species, and the second domain consisting of a sequence which forms a second specific binding pair with at least one RNA processing or translation factor.

The nucleic acid molecule may be considered to be a gene-specific trans-acting enhancer of RNA processing or translation. Thus, the first domain of the nucleic acid molecule is an RNA binding domain and the second domain is an RNA factor binding domain.

The first domain of the nucleic acid molecule is designed to bind to the target sequence on the target RNA species sufficiently close to am RNA processing or translation site in the target RNA species for processing or translation at the site to be enhanced by the action of the second domain, i.e., by the binding of the second domain to the RNA processing or translation factor, thus recruiting the factor to the RNA processing or translation site.

One having ordinary skill in the art would readily appreciate that there are practical constraints on the size of the first domain of the nucleic acid molecule. If it is too short, the binding to the target sequence would be unstable; if it is too long there is an increased possibility that part of the first domain will anneal to other targets. Thus, in a preferred embodiment, the full length of the first domain anneals to the target region of the target RNA species to maximize specificity of binding. In a related embodiment, the first domain of the nucleic acid molecule is from 8 to 50 nucleotides in length. In a particular embodiment, the first domain is about 8, or 9, or 10, or 11, or 12, or 13, or 14, or 15, or 16, or 17, or 18, or 19, or 20 to 25, or 26 to 30, or 31 to 40, or 41 to 50 nucleotides in length. Preferably, it is between 10 to 25 nucleotides in length.

Typically, the first domain of the nucleic acid molecule binds to the target sequence on the target RNA species by complementary base pairing. Preferably, the first domain has at least 90% sequence identity with the target sequence, more preferably at least 95% or at least 99% sequence identity. It is most preferred if the first domain has 100% sequence identity with the target sequence. When the first domain is between 10 to 25 nucleotides in length, it requires a higher level of sequence identity with the target sequence, and preferably having only a single mismatch or none at all. However, with a longer first domain, such as 50 nucleotides or more, a lower level of sequence identity with the target sequence may be acceptable.

It is preferred if the target sequence occurs only once in the target RNA species. It is also preferred if the target sequence only occurs once in the genome of the organism from which the target RNA is expressed.

Typically, the nucleic acid molecule is arranged such that upon formation of a first specific binding pair with said target sequence, the at least one RNA processing or translation factor interacts with the RNA target species at the RNA processing or translation site to effect RNA processing or translation at the RNA processing or translation site.

It is appreciated that the second domain of the nucleic acid molecule can form a second specific binding pair with the RNA processing or translation factor before, after or substantially simultaneously with the formation of the first specific binding pair. The second domain of the nucleic acid molecule should not be complementary to the RNA target species, so that it is available for the binding of RNA processing factors.

Typically, the second domain of the nucleic acid molecule is typically from 5 to 50 nucleotides in length, and may be longer. Thus, the second domain can be 5, or 6, or 7, or 8, or 9, or 10, or 11, or 12, or 13, or 14, or 15, or 16, or 17, or 18, or 19, or 20, to 25, or 26 to 30, or 31 to 40, or 41 to 50 or more nucleotides in length. The minimum binding site for an RNA processing or translation factor is three nucleotides although to allow accessibility to the factors, a minimum size for this domain would be around 5 nucleotides. However, the optimal size is typically higher. The length of the second domain may be increased by including tandem repeats or arrays of recognition motifs for the RNA processing or translation factor, to minimise spurious binding.

Thus, the entire nucleic acid molecule is typically from 13 to 100 nucleotides or more in length. Preferably, the entire nucleic acid molecule is from 15 to 50 nucleotides in length, and can be, for example, 15 or 16, or 17, or 18, or 19, or 20, or 21, or 22, or 23, or 24, or 25, or 26, or 27, or 28, or 29, or 30, or 31 to 40, or 41 to 50 or more nucleotides in length.

Thus the invention includes a nucleic acid molecule comprising first and second domains, said first domain being capable of forming a first specific binding pair with a target sequence of a target RNA species, said second domain consisting of a sequence which forms a second specific binding pair with at least one RNA processing or translation factor.

Regarding the proximity of the target sequence to the RNA processing or translation site on the target RNA species, as used herein, the terms “sufficiently close”, “near to” and “close to” may mean between 0 and 1,000 nucleotides, more preferably between 0 and 500 nucleotides, still more preferably between 0 and 200 nucleotides, and yet more preferably between 0 and 100 nucleotides. For example, the target sequence may be 0, 1, 2, 3, 4, or 5, 6, 7, 8, 9, or 10, 15, 20, 25, 30, 35, 40, 45, 50, 55, 60, 65, 70, 75, 80, 85, 90, 95, or 100 nucleotides from the RNA processing or translation site. However, RNA is known to form a range of secondary structures which may bring the target sequence on the target RNA species sufficiently close to the RNA processing or translation site for processing or translation at the site to be enhanced by the action of the factor bound to the second domain, even if the target sequence and the RNA processing or translation site are separated by many kilobases apart on the target RNA species.

Preferably, the second domain of the nucleic acid molecule has a sequence binding motif that is recognised by the RNA processing or translation factor allowing the formation of the second specific binding pair with the factor.

RNA processing factors may be any RNA or protein that stimulates splicing activity or translation when recruited to the RNA target species at the RNA processing or translation site. Illustrative RNA processing factors include, but are not limited to RNA molecules, RNA structural molecules, RNA stability molecules, splicing factors, polyadenylation factors, transcription factors, and translation factors. These factors may include cellular proteins, nucleic acids, ribonucleoprotein complexes, and combinations thereof.

RNA splicing factors may comprise any one of the group of proteins that influence the site or efficiency of splicing, such as SR proteins, SR-related proteins (Graveley, B. R. (2000) RNA 6(9): p 1197-1211, PMID: 10999598), or hnRNP proteins (Krecic, A. M. and Swanson, M. S. (1999) Curr. Opin. Cell Bio. 11(3): p 363-371, PMID: 10395553). The RNA sequence binding motifs associated with these proteins are well characterised and are known to a person skilled in the art. Further splicing enhancer sequences known in the prior art (supra) may also be utilised.

In addition to SR-dependent enhancers, numerous sequences in introns or exons have been shown to affect splice site selection or exon incorporation. In some cases, these affect the processing of specific target gene transcripts in precise ways (reviewed by Smith & Valcarcel, Trends Biochem Sci 25, 381-388 (2000)). However, many of them are bound by hnRNP proteins, which are known to bind nascent transcripts, to be at least reasonably abundant and, often, to be expressed ubiquitously (Krecic & Swanson, Curr Opin Cell Biol 11, 363-371 (1999)), leading to the supposition that they will in fact recognise sequences in numerous transcripts and influence splicing rather widely. Other sequence elements defined recently include (A+C)-rich enhancers, found recently to be recognised by the protein YB-1 52 (Stickeler et al., Embo J 20, 3821-3830. (2001); intronic GGG triplets, recognised by U1 snRNA (McCullough & Berget, Mol Cell Biol 20, 9225-9235. (2000)); GGGGCUG sequences that are recognised by mBBP (Carlo et al, Mol Cell Biol 20, 3988-3995. (2000)); and purine-rich sequences recognised by T-STAR, a possible mediator of signalling responses identified by this laboratory (Venables et al., Hum Mol Genet. 8, 959-969 (1999)) and then shown to affect splicing (Stoss et al., J Biol Chem 276, 8665-8673. (2001)). RNA splicing factors also include STAR proteins, CELF proteins, peliotropic proteins such as YB1, nuclear scaffold proteins and helicases.

It is appreciated that the second domain may contain sequence binding motifs that are known to enhance RNA processing or translation, such as splicing, even if the RNA processing or translation factor which recognises these motifs has not yet been identified. For example, Fairbrother et al, (2002, Science 297 (5583): 1007-1013) identified ten exonic splicing enhancer sequence motifs in human genes, each of which may be suitable for inclusion in the second domain.

A useful motif for the second domain of the nucleic acid molecule is CAGGUAAGU which is the binding site for the U1 snRP. In other embodiments, the second domain may contain other GGA repeat motifs which may act as a recognition site for the SF2/ASF factor.

The nucleic acid molecule may contain multiple functional domains, for example, it may contain binding sites for one or more RNA processing or translation factor such as an SRor SR-related protein (see, for example, Hertel & Maniatis (1998), “The function of multisite splicing enhancers” Molecular Cell 1(3): 449-55).

Typically and preferably, the nucleic acid molecule is an RNA molecule, i.e., it is an oligoribonucleotide. Preferably, the nucleic acid molecule is not DNA as this would trigger ribonuclease H degradation of the target RNA species. The nucleic acid molecule may include phosphoramidate linkages which improve stability, the free energy of annealing and resistance to degradation (Faria et al, 2001, Nature Biotechnol. 19(1): 40-44); or locked nucleic acids (LNA, Kurreck et al, 2002, Nucleic Acids Res. 30(9): 1911-8), or peptide nucleic acids (PNA).

Thus, in particular embodiments, the present invention provides a method to alter the potency of a cell, comprising contacting the cell with one or more activators or a composition comprising the one or more activators, wherein the one or more activators includes a bifunctional antisense oligonucleotide or combination of bifunctional antisense oligonucleotides, and wherein the one or more activators modulate a component of a cellular pathway associated with cell potency.

In related embodiments, a method of reprogramming a cell comprises contacting the cell with one or more activators or a composition comprising the one or more activators, wherein the one or more activators comprises one or more bifunctional antisense oligonucleotides, and wherein the one or more activators modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises at least one bifunctional antisense oligonucleotide, and wherein the one or more activators modulates a component of a cellular pathway associated with cell potency, thereby programming the cell.

In a particular related embodiment, a method of reprogramming or programming a cell comprise contacting the cell with: i) one or more activators, wherein the one or more activators comprises one or more bifunctional antisense oligonucleotides; and ii) at least one repressor, wherein the one or more activators and repressor(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators comprises at least one bifunctional antisense oligonucleotide, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

J. Locked Nucleic Acids

The present invention contemplates, in part, that any nucleic acid (e.g., repressors and activators) of the present invention may comprise one or more “locked nucleic acids” (LNAs), which are novel conformationally restricted oligonucleotide analogues containing a methylene bridge that connects the 2′-O of ribose with the 4′-C (see, Singh et al, Chem. Commun., 1998, 4:455-456). LNA oligonucleotides contain one or more nucleotide building blocks in which an extra methylene bridge, as noted above, that fixes the ribose moiety either in the C3′-endo (β-D-LNA) or C2′-endo (α-L-LNA) conformation.

LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mismatched sequences show that LNA obey the Watson-Crick base pairing rules with generally improved selectivity compared to the corresponding unmodified reference strands. Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

The one or more antisense agents comprising LNAs can be designed as “gapmers” in which the oligonucleotide comprises a stretch of LNAs at the 5′ end, followed by a “gap” of DNA nucleotides, then a second stretch of LNAs at the 3′ end.

In one embodiment, an antisense nucleic acid of the invention comprises LNAs. In another embodiment, an antisense nucleic acid of the invention comprises β-D-LNAs. In a related embodiment, an antisense nucleic acid of the invention is an LNA gapmer, as described above.

K. Peptide Nucleic Acids

The present invention contemplates, in part, that any nucleic acid (e.g., repressors and activators) of the present invention may comprise one or more “peptide nucleic acids” (PNAs), which are nucleic acid mimics (e.g., DNA mimics), wherein the deoxyribose phosphate backbone is replaced by a pseudopeptide backbone and only the four natural nucleobases are retained. The neutral backbone of PNAs allows for specific hybridization to DNA and RNA under conditions of low ionic strength.

PNAs can be used as antisense or antigene agents for sequence-specific modulation of gene expression by inducing transcription or translation arrest or inhibiting replication. PNAs may also be used in the analysis of single base pair mutations (e.g., PNA directed PCR clamping; as artificial restriction enzymes when used in combination with other enzymes, e.g., 51 nucleases (Hyrup and Nielsen, 1996); or as probes or primers for DNA sequence and hybridization (Hyrup and Nielsen, 1996; Perry-O'Keefe et al., 1996).

PNAs can be modified to enhance their stability or cellular uptake. Lipophilic or other helper groups may be attached to PNAs or PNA-DNA dimers. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996).

The synthesis of PNA-DNA chimeras can be performed (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989) or PCT Publication No. WO88/09810) or the blood-brain barrier (e.g., PCT Publication No. WO 89/10134). In addition, oligonucleotides can be modified with hybridization-triggered cleavage agents (van der Krol et al., 1988a) or intercalating agents (Zon, 1988). The oligonucleotide may be conjugated to another molecule, e.g., a peptide, a hybridization triggered cross-linking agent, a transport agent, a hybridization-triggered cleavage agent, and the like.

L. Artificial Transcription Factors

The present invention further contemplates, in part, the use of transcription factors in a method to alter the potency of the cell. In addition to the natural transcription factors that are described elsewhere herein, artificially designed transcription factors are also suitable for use in the methods of the present invention. The artificial transcription factors (ATFs) can be either transcriptional repressors or activators depending on the context in which they are used.

The ATFs are engineered zinc finger proteins that are capable precisely regulating gene expression at any given locus. In the methods of the present invention, one or more ATFs are designed so as to bind to and modulate the transcription of the genetic locus of a component of a cellular pathway associated with cell potency. It will be apparent to one of skill in the art that ATF(s) can be used facilitate the modulation of any component of a cellular potency pathway, and thus, alter the potency of a cell, either by reprogramming or programming the cell.

As used herein, the term “binding protein” “or binding domain” is a protein or polypeptide that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

As used herein, the term “artificial transcription factor” is an engineered zinc finger protein or or fusion protein that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” An ATF of the present invention, has a ZFP DNA binding domain comprising at least one finger, typically two fingers, three fingers, or six fingers. Each-finger binds from two to four base pairs of DNA, typically three or four base pairs of DNA. An ATF binds to a nucleic acid sequence called a target site or target segment. Each finger typically comprises an approximately 30 amino acid, zinc-chelating, DNA-binding subdomain. An exemplary motif characterizing one class of these proteins (C2H2 class) is -Cys-(X)₂₋₄-Cys-(X)₁₂-His-(X)₃₋₅-His (where X is any amino acid). Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

An “artificial transcription factor” is a protein or fusion protein not occurring in nature whose structure and composition result principally from rational criteria. Rational criteria for design include application of substitution rules and computerized algorithms for processing information in a database storing information of existing ZFP designs and binding data, for example as described in WO 00/42219, U.S. Pat. No. 5,789,538; U.S. Pat. No. 6,007,988; U.S. Pat. No. 6,013,453; WO 95/19431; WO 96/06166 and WO 98/54311.

Target sequences can be nucleotide sequences (either DNA or RNA) or amino acid sequences. A single target site typically has about four to about ten base pairs. Typically, an ATF comprising two zinc fingers recognizes a four to seven base pair target site, an ATF comprising three zinc fingers recognizes a six to ten base pair target site, and an ATF comprising six zinc fingers recognizes two adjacent nine to ten base pair target sites. By way of a non-limiting example, a DNA target sequence for an ATF comprising three zinc fingers is generally either 9 or 10 nucleotides in length, depending upon the presence and/or nature of cross-strand interactions between the zinc fingers and the target sequence. Target sequences can be found in any DNA or RNA sequence, including regulatory sequences, exons, introns, or any non-coding sequence.

To determine the level of gene expression modulation by an ATF, cells contacted with ATFs are compared to control cells, e.g., without the ATF, to examine the extent of repression or activation. Control samples are assigned a relative gene expression activity value of 100%. In one embodiment, modulation/repression of gene expression is achieved when the gene expression activity value relative to the control is about 95%, 90%, 85%, 80%, 75%, 70%, 65%, 60%, 55%, 50%, 45%, 40%, 35%, 30%, 25%, 20%, 15%, 10%, 5%, 1% or 0%.

In a related embodiment, modulation/activation of gene expression is achieved when the gene expression activity value relative to the control is 110%, 120%, 130%, 140%, 150%, 160%, 170%, 180%, 190%, 200%, 250%, 300%, 350%, 400%, 450%, 500%, 600%, 700%, 800%, 900%, 1000%, 1500%, or 2000% or more.

As noted above, transcriptional activators and transcriptional repressors or functional fragments thereof, have the ability to modulate transcription, as described above. Such proteins include, in addition to those mentioned elsewhere herein, transcription factors and co-factors (e.g., KRAB, MAD, ERD, SID, nuclear factor kappa B subunit p65, early growth response factor 1, and nuclear hormone receptors, VP16, VP64), endonucleases, integrases, recombinases, methyltransferases, histone acetyltransferases, histone deacetylases etc. Activators and repressors further include co-activators and co-repressors (see, e.g., Utley et al., Nature 394:498-502 (1998)), and the like.

As used herein, the term, “regulatory domain” or “functional domain” refers to a protein or a polypeptide sequence that has transcriptional modulation activity, or that is capable of interacting with proteins and/or protein domains that have transcriptional modulation activity. Typically, a functional domain is covalently or non-covalently linked to a DNA-binding domain (e.g., one or more zinc fingers) to modulate transcription of a component of a cellular potency pathway. Alternatively, an ATP comprising one or more zinc fingers can act, in the absence of a functional domain, to modulate transcription. Furthermore, transcription of a component of a cellular potency pathway can be modulated by an ATF comprising one or more zinc fingers linked to multiple functional domains.

According to the present invention, a functional fragment of an ATF protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. An ATF functional fragment can possess more, fewer, or the same number of residues as the corresponding native molecule, and/or can contain one or more amino acid or nucleotide substitutions. Methods for determining the function of an ATF nucleic acid (e.g., coding function, ability to hybridize to another nucleic acid) are well-known in the art. Similarly, methods for determining ATF protein functions are well-known. For example, the DNA-binding function of an ATF polypeptide can be determined, for example, by filter-binding, electrophoretic mobility-shift, or immunoprecipitation assays. See Ausubel et al., supra. The ability of an ATF protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

As used herein, the term “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and a transcriptional activation domain) and fusion nucleic acids (for example, a nucleic acid encoding the fusion polypeptide described herein). Examples of the second type of fusion molecule include, but are not limited to, a fusion between a triplex-forming nucleic acid and a polypeptide, and a fusion between a minor groove binder and a nucleic acid.

As used herein, the term “heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. In contrast, a naturally translocated piece of chromosome would not be considered heterologous in the context of this patent application, as it comprises an endogenous nucleic acid sequence that is native to the mutated cell.

Similarly, a heterologous protein indicates that the protein comprises two or more subsequences that are not found in the same relationship to each other in nature (e.g., a “fusion protein,” where the two subsequences are encoded by a single nucleic acid sequence). See, e.g., Ausubel, supra, for an introduction to recombinant techniques.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

As used herein, the terms “operative linkage” and “operatively linked” are used with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. An operatively linked transcriptional regulatory sequence is generally joined in cis with a coding sequence, but need not be directly adjacent to it. For example, an enhancer can constitute a transcriptional regulatory sequence that is operatively-linked to a coding sequence, even though they are not contiguous.

The engineering of novel DNA binding proteins (e.g., ATFs) that selectively regulate the expression of a gene at its endogenous locus (i.e., genes as they occur in the context of their natural chromosomal structure) has been described. See, for example, WO 00/41566 and WO 00/42219. This approach provides a unique capacity to selectively turn on or turn off endogenous gene expression in the cell and thus affect fundamental mechanisms of regulating cell potency.

The present invention contemplates, in part, to engineer ATFs to recognize a selected target site in a component of a cellular pathway associated with the potency of a cell. A suitable ATF scaffold comprises any suitable C₂H₂ ZFP, such as SP-1, SP-1C, or ZIF268 (see, e.g., Jacobs, EMBO J. 11:4507 (1992); Desjarlais & Berg, PNAS 90:2256-2260 (1993)). A number of methods are known in the art that can then be used to design and/or select an ATF comprising one or more zinc fingers that has a high affinity for its target (e.g., preferably with a K_{d} of less than about 25 nM). As described above, an ATF comprising a ZFP DNA binding domain can be designed or selected to bind to any suitable target site in the genetic locus of a component of a cellular pathway associated with cell potency with high affinity. WO 00/42219 comprehensively describes methods for design, construction, and expression of ATPs comprising ZFP DNA binding domains for selected target sites.

Any suitable method known in the art can be used to design and construct nucleic acids encoding ZFPs, e.g., phage display, random mutagenesis, combinatorial libraries, computer/rational design, affinity selection, PCR, cloning from cDNA or genomic libraries, synthetic construction and the like. (see, e.g., U.S. Pat. No. 5,786,538; Wu et al., PNAS 92:344-348 (1995); Jamieson et al., Biochemistry 33:5689-5695 (1994); Rebar & Pabo, Science 263:671-673 (1994); Choo & Klug, PNAS 91:11163-11167 (1994); Choo & Klug, PNAS 91: 11168-11172 (1994); Desjarlais & Berg, PNAS 90:2256-2260 (1993); Desjarlais & Berg, PNAS 89:7345-7349 (1992); Pomerantz et al., Science 267:93-96 (1995); Pomerantz et al., PNAS 92:9752-9756 (1995); Liu et al., PNAS 94:5525-5530 (1997); Griesman & Pabo, Science 275:657-661 (1997); Desjarlais & Berg, PNAS 91:11-99-11103 (1994)).

Thus, these methods work by selecting a target gene, and systematically searching within every possible subsequence of 9 or 10 contiguous bases on either strand of a potential target gene is evaluated to determine whether it contains putative target sites, as described, e.g., in U.S. Pat. No. 6,453,242. Typically, such a comparison is performed by computer, and a list of target sites is output.

The target sites identified by the above methods can be subject to further evaluation by other criteria or can be used directly for design or selection (if needed) and production of an ATF comprising zinc finger domains specific for such a site. A further criterion for evaluating potential target sites is their proximity to particular regions within a gene. If an ATF is to be used to repress a cellular gene on its own (e.g., without linking the ATF to a repressing moiety), then the optimal location appears to be at, or within 50 by upstream or downstream of the site of transcription initiation, to interfere with the formation of the transcription complex (Kim & Pabo, J. Biol. Chem. 272:29795-296800 (1997)) or compete for an essential enhancer binding protein. If, however, an ATF comprising a ZFP DNA binding domain is fused to a functional domain such as the KRAB repressor domain or the VP16 activator domain, the location of the binding site is considerably more flexible and can be outside known regulatory regions. For example, a KRAB domain can repress transcription at a promoter up to at least 3 kilobases from where KRAB is bound (Margolin et al., PNAS 91:4509-4513 (1994)). Thus, target sites can be selected that do not necessarily include or overlap segments of demonstrable biological significance with target genes, such as regulatory sequences.

After a target segment has been selected, an ATF comprising a ZFP DNA binding domain that binds to the segment can be provided by a variety of approaches. The simplest of approaches is to provide a precharacterized ZFP from an existing collection that is already known to bind to the target site. However, in many instances, such ZFPs do not exist. An alternative approach can also be used to design new ATFs comprising new ZFP DNA binding domains, which uses the information in a database of existing DNA binding domains of ZFPs and their respective binding affinities. A further approach is to design an ATF with a ZFP DNA binding domain based on substitution rules. See, e.g., WO 96/06166; WO 98/53058; WO 98/53059 and WO 98/53060. A still further alternative is to select an ATF with a ZFP DNA binding domain having specificity for a given target by an empirical process such as phage display. See, e.g., WO 98/53057. In some such methods, each component finger of a ZFP DNA binding domain is designed or selected independently of other component fingers. For example, each finger can be obtained from a different preexisting ZFP DNA binding domain or each finger can be subject to separate randomization and selection.

ATF polypeptides and nucleic acids can be made using routine techniques in the field of recombinant genetics. Basic texts disclosing the general methods of use in the field include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). In addition, essentially any nucleic acid can be custom ordered from any of a variety of commercial sources. Similarly, peptides and antibodies can be custom ordered from any of a variety of commercial sources.

Any suitable method of protein purification known to those of skill in the art can be used to purify ATFs (see Ausubel, supra, Sambrook, supra). In addition, any suitable host can be used, e.g., bacterial cells, insect cells, yeast cells, mammalian cells, and the like.

ATF binding domains (e.g., ZFP DNA binding domains) can optionally be associated with regulatory domains (e.g., functional domains) for modulation of gene expression. The ATF comprising one or more ZFP DNA binding domains can be covalently or non-covalently associated with one or more regulatory domains, alternatively two or more regulatory domains, with the two or more domains being two copies of the same domain, or two different domains. The regulatory domains can be covalently linked to the ZFP DNA binding domain, e.g., via an amino acid linker, as part of a fusion protein. The ZFP DNA binding domains can also be associated with a regulatory domain via a non-covalent dimerization domain, e.g., a leucine zipper, a STAT protein N terminal domain, or an FK506 binding protein (see, e.g., O'Shea, Science 254: 539 (1991), Barahmand-Pour et al., Curr. Top. Microbiol. Immunol. 211:121-128 (1996); Klemm et al., Annu. Rev. Immunol. 16:569-592 (1998); Klemm et al., Annu. Rev. Immunol. 16:569-592 (1998); Ho et al., Nature 382:822-826 (1996); and Pomeranz et al., Biochem. 37:965 (1998)). The regulatory domain can be associated with the ZFP DNA binding domain at any suitable position, including the C- or N-terminus.

Common regulatory domains suitable for use in the ATFs of the present invention include, but are not limited to effector domains from transcription factors (activators, repressors, co-activators, co-repressors), silencers, nuclear hormone receptors, oncogene transcription factors (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases, topoisomerases, helicases, ligases, kinases, phosphatases, polymerases, endonucleases) and their associated factors and modifiers.

Transcription factor polypeptides from which one can obtain a regulatory domain also include those that are involved in regulated and basal transcription. Such polypeptides include, but are not limited to transcription factors, their effector domains, coactivators, silencers, nuclear hormone receptors (see, e.g., Goodrich et al., Cell 84:825-30 (1996) for a review of proteins and nucleic acid elements involved in transcription; transcription factors in general are reviewed in Barnes & Adcock, Clin. Exp. Allergy 25 Suppl. 2:46-9 (1995) and Roeder, Methods Enzymol. 273:165-71 (1996)). Databases dedicated to transcription factors are known (see, e.g. Science 269:630 (1995)). Nuclear hormone receptor transcription factors are described in, for example, Rosen et al., J. Med. Chem. 38:4855-74 (1995). The C/EBP family of transcription factors are reviewed in Wedel et al., Immunobiology 193:171-85 (1995). Coactivators and co-repressors that mediate transcription regulation by nuclear hormone receptors are reviewed in, for example, Meier, Eur. J. Endocrinol. 134(2):158-9 (1996); Kaiser et al., Trends Biochem. Sci. 21:342-5 (1996); and Utley et al., Nature 394:498-502 (1998)). GATA transcription factors, which are involved in regulation of hematopoiesis, are described in, for example, Simon, Nat. Genet. 11:9-11 (1995); Weiss et al., Exp. Hematol. 23:99-107. TATA box binding protein (TBP) and its associated TAF polypeptides (which include TAF30, TAF55, TAF80, TAF110, TAF150, and TAF250) are described in Goodrich & Tijan, Curr. Opin. Cell Biol. 6:403-9 (1994) and Hurley, Curr. Opin. Struct. Biol. 6:69-75 (1996). The STAT family of transcription factors are reviewed in, for example, Barahmand-Pour et al., Curr. Top. Microbiol. Immunol. 211:121-8 (1996). Transcription factors involved in disease are reviewed in Aso et al., J. Clin. Invest. 97:1561-9 (1996).

In one embodiment, a method of altering the potency of a cell comprises contacting the cell with a composition comprising one or more repressors, said one or more repressors comprising an ATF having the KRAB repression domain from the human KOX-1 protein (Thiesen et al., New Biologist 2:363-374 (1990); Margolin et al., PNAS 91:4509-4513 (1994); Pengue et al., Nucl. Acids Res. 22:2908-2914 (1994); Witzgall et al., PNAS 91:4514-4518 (1994)).

In another embodiment, the composition further comprises KAP-1, a KRAB co-repressor, is used with KRAB (Friedman et al., Genes Dev. 10:2067-2078 (1996)).

In related embodiment, an ATF that acts as a repressor comprises transcriptional repressor domains from transcription factors such as MAD (see, e.g., Sommer et al., J. Biol. Chem. 273:6632-6642 (1998); Gupta et al., Oncogene 16:1149-1159 (1998); Queva et al., Oncogene 16:967-977 (1998); Larsson et al., Oncogene 15:737-748 (1997); Laherty et al., Cell 89:349-356 (1997); and Cultraro et al., Mol. Cell. Biol. 17:2353-2359 (19977)); FKHR (forkhead in rhapdosarcoma gene; Ginsberg et al., Cancer Res. 15:3542-3546 (1998); Epstein et al., Mol. Cell. Biol. 18:4118-4130 (1998)); EGR-1 (early growth response gene product-1; Yan et al., PNAS 95:8298-8303 (1998); and Liu et al., Cancer Gene Ther. 5:3-28 (1998)); the ets2 repressor factor repressor domain (ERD; Sgouras et al., EMBO J. 14:4781-4793 ((19095)); and the MAD smSIN3 interaction domain (SID; Ayer et al., Mol. Cell. Biol. 16:5772-5781 (1996)).

In another embodiment, a method of altering the potency of a cell comprises contacting the cell with a composition comprising one or more activators, said one or more activators comprising an ATF having the HSV VP16 activation domain (see, e.g., Hagmann et al., J. Virol. 71:5952-5962 (1997)); the VP64 activation domain (Seipel et al., EMBO J. 11:4961-4968 (1996)); a nuclear hormone receptors activation domain (see, e.g., Torchia et al., Curr. Opin. Cell. Biol. 10:373-383 (1998)); the activation domain from the p65 subunit of nuclear factor kappa B (Bitko & Barik, J. Virol. 72:5610-5618 (1998) and Doyle & Hunt, Neuroreport 8:2937-2942 (1997)); and the EGR-1 activation domain (early growth response gene product-1; Yan et al., PNAS 95:8298-8303 (1998); and Liu et al., Cancer Gene Ther. 5:3-28 (1998)).

As described, useful domains can also be obtained from the gene products of oncogenes (e.g., myc, jun, fos, myb, max, mad, rel, ets, bcl, myb, mos family members) and their associated factors and modifiers. Oncogenes are described in, for example, Cooper, Oncogenes, 2nd ed., The Jones and Bartlett Series in Biology, Boston, Mass., Jones and Bartlett Publishers, 1995. The ets transcription factors are reviewed in Waslylk et al., Eur. J. Biochem. 211:7-18 (1993) and Crepieux et al., Crit. Rev. Oncog. 5:615-38 (1994). Myc oncogenes are reviewed in, for example, Ryan et al., Biochem. J. 314:713-21 (1996). The jun and fos transcription factors are described in, for example, The Fos and Jun Families of Transcription Factors, Angel & Herrlich, eds. (1994). The max oncogene is reviewed in Hurlin et al., Cold Spring Harb. Symp. Quant. Biol. 59:109-16. The myb gene family is reviewed in Kanei-Ishii et al., Curr. Top. Microbiol. Immunol. 211:89-98 (1996). The mos family is reviewed in Yew et al., Curr. Opin. Genet. Dev. 3:19-25 (1993).

ATFs can further comprise regulatory domains obtained from DNA repair enzymes and their associated factors and modifiers. DNA repair systems are reviewed in, for example, Vos, Curr. Opin. Cell Biol. 4:385-95 (1992); Sancar, Ann. Rev. Genet. 29:69-105 (1995); Lehmann, Genet. Eng. 17:1-19 (1995); and Wood, Ann. Rev. Biochem. 65:135-67 (1996). DNA rearrangement enzymes and their associated factors and modifiers can also be used as regulatory domains (see, e.g., Gangloff et al., Experienitia 50:261-9 (1994); Sadowski, FASEB J. 7:760-7 (1993)).

Similarly, regulatory domains can be derived from DNA modifying enzymes (e.g., DNA methyltransferases, topoisomerases, helicases, ligases, kinases, phosphatases, polymerases) and their associated factors and modifiers. Helicases are reviewed in Matson et al., Bioessays, 16:13-22 (1994), and methyltransferases are described in Cheng, Curr. Opin. Struct. Biol. 5:4-10 (1995). Chromatin associated proteins and their modifiers (e.g., kinases, acetylases and deacetylases), such as histone deacetylase (Wolffe, Science 272:371-2 (1996)) are also useful as domains for addition to an ATF that modulates one or more components of a cellular pathway associated the potency of a cell.

In one preferred embodiment, the regulatory domain is a DNA methyl transferase that acts as a transcriptional repressor (see, e.g. Van den Wyngaert et al., FEBS Lett. 426:283-289 (1998); Flynn et al., J. Mol. Biol. 279:101-116 (1998); Okano et al., Nucleic Acids Res. 26:2536-2540 (1998); and Zardo & Caiafa, J. Biol. Chem. 273:16517-16520 (1998)). In another embodiment, the regulatory domain is a DNA demethylase that acts as a transcriptional activator. In another preferred embodiment, endonucleases such as Fok1 are used as transcriptional repressors, which act via gene cleavage (see, e.g., WO 95/09233; and PCT/US94/01201).

In one embodiment, histone acetyltransferase is used as a transcriptional activator (see, e.g., Jin & Scotto, Mol. Cell. Biol. 18:4377-4384 (1998); Wolffe, Science 272:371-372 (1996); Taunton et al., Science 272:408-411 (1996); and Hassig et al., PNAS 95:3519-3524 (1998)). In another embodiment, histone deacetylase is used as a transcriptional repressor (see, e.g., Jin & Scotto, Mol. Cell. Biol. 18:4377-4384 (1998); Syntichaki & Thireos, J. Biol. Chem. 273:24414-24419 (1998); Sakaguchi et al., Genes Dev. 12:2831-2841 (1998); and Martinez et al., J. Biol. Chem. 273:23781-23785 (1998)).

Additional exemplary repression domains include those derived from histone deacetylases (HDACs, e.g., Class I HDACs, Class II HDACs, SIR-2 homologues), HDAC-interacting proteins (e.g., SIN3, SAP30, SAP15, NCoR, SMRT, RB, p107, p130, RBAP46/48, MTA, Mi-2, Brg1, Brm), DNA-cytosine methyltransferases (e.g., Dnmt1, Dnmt3a, Dnmt3b), proteins that bind methylated DNA (e.g., MBD1, MBD2, MBD3, MBD4, MeCP2, DMAP1), protein methyltransferases (e.g., lysine and arginine methylases, SuVar homologues such as Suv39H1), polycomb-type repressors (e.g., Bmi-1, eed1, RING1, RYBP, E2F6, Mell8, YY1 and CtBP), viral repressors (e.g., adenovirus E1b 55K protein, cytomegalovirus UL34 protein, viral oncogenes such as v-erbA), hormone receptors (e.g. Dax-1, estrogen receptor, thyroid hormone receptor), and repression domains associated with naturally-occurring zinc finger proteins (e.g., WT1, KAP1). Further exemplary repression domains include members of the polycomb complex and their homologues, HPH1, HPH2, HPC2, NC2, groucho, Eve, tramtrak, mHP1, SIP1, ZEB1, ZEB2, and Enx1/Ezh2. In all of these cases, either the full-length protein or a functional fragment can be used as a repression domain for fusion to a zinc finger binding domain. Furthermore, any homologues of the aforementioned proteins can also be used as repression domains, as can proteins (or their functional fragments) that interact with any of the aforementioned proteins.

It will be clear to those of skill in the art that, in the formation of a fusion protein (e.g., an ATF) (or a nucleic acid encoding same) between a zinc finger binding domain and a functional domain, either a repressor or a molecule that interacts with a repressor is suitable as a functional domain. Essentially any molecule capable of recruiting a repressive complex and/or repressive activity (such as, for example, histone deacetylation) to the target gene is useful as a repression domain of a fusion protein.

Additional exemplary activation domains include, but are not limited to, p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, API, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

It will be clear to those of skill in the art that, in the formation of a fusion protein (e.g., an ATF) (or a nucleic acid encoding same) between a zinc finger binding domain and a functional domain, either an activator or a molecule that interacts with an activator is suitable as a functional domain. Essentially any molecule capable of recruiting an activating complex and/or activating activity (such as, for example, histone acetylation) to the target gene is useful as an activating domain of a fusion protein.

Insulator domains, chromatin remodeling proteins such as ISWI-containing domains and/or methyl binding domain proteins suitable for use as functional domains in fusion molecules are described, for example, in PCT application US01/40616 and U.S. Patent applications 60/236,409; 60/236,884; and 60/253,678.

In a further embodiment, a DNA-binding domain (e.g., a zinc finger domain) is fused to a bifunctional domain (BFD). A bifunctional domain is a transcriptional regulatory domain whose activity depends upon interaction of the BFD with a second molecule. The second molecule can be any type of molecule capable of influencing the functional properties of the BFD including, but not limited to, a compound, a small molecule, a peptide, a protein, a polysaccharide or a nucleic acid. An exemplary BFD is the ligand binding domain of the estrogen receptor (ER). In the presence of estradiol, the ER ligand binding domain acts as a transcriptional activator; while, in the absence of estradiol and the presence of tamoxifen or 4-hydroxy-tamoxifen, it acts as a transcriptional repressor. Another example of a BFD is the thyroid hormone receptor (TR) ligand binding domain which, in the absence of ligand, acts as a transcriptional repressor and in the presence of thyroid hormone (T3), acts as a transcriptional activator. An additional BFD is the glucocorticoid receptor (GR) ligand binding domain. In the presence of dexamethasone, this domain acts as a transcriptional activator; while, in the presence of RU486, it acts as a transcriptional repressor. An additional exemplary BFD is the ligand binding domain of the retinoic acid receptor. In the presence of its ligand all-trans-retinoic acid, the retinoic acid receptor recruits a number of co-activator complexes and activates transcription. In the absence of ligand, the retinoic acid receptor is not capable of recruiting transcriptional co-activators. Additional BFDs are known to those of skill in the art. See, for example, U.S. Pat. Nos. 5,834,266 and 5,994,313 and PCT WO 99/10508.

Linker domains between polypeptide domains, e.g., between two ATFs or between a ZFP DNA binding domain and a regulatory domain, can be included. Such linkers are typically polypeptide sequences, such as poly gly sequences of between about 5 and 200 amino acids. Preferred linkers are typically flexible amino acid subsequences which are synthesized as part of a recombinant fusion protein. For example, in one embodiment, the linker DGGGS is used to link two ATFs. In another embodiment, the flexible linker linking two ATFs is an amino acid subsequence comprising the sequence TGEKP (see, e.g., Liu et al., PNAS 5525-5530 (1997)). In another embodiment, the linker LRQKDGERP is used to link two ATFs. In another embodiment, the following linkers are used to link two ATFs: GGRR (Pomerantz et al. 1995, supra), (G4S)ₙ (Kim et al., PNAS 93, 1156-1160 (1996.); and GGRRGGGS; LRQRDGERP; LRQKDGGGSERP; LRQKd(G3S)₂ ERP. Alternatively, flexible linkers can be rationally designed using computer program capable of modeling both DNA-binding sites and the peptides themselves (Desjarlais & Berg, PNAS 90:2256-2260 (1993), PNAS 91:11099-11103 (1994) or by phage display methods.

In other embodiments, a chemical linker is used to connect synthetically or recombinantly produced domain sequences. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages. In addition to covalent linkage of ZFPs to regulatory domains, non-covalent methods can be used to produce molecules with ZFPs associated with regulatory domains.

In addition to regulatory domains, often the ZFP is expressed as a fusion protein such as maltose binding protein (“MBP”), glutathione S transferase (GST), hexahistidine, c-myc, and the FLAG epitope, for ease of purification, monitoring expression, or monitoring cellular and subcellular localization.

Thus, in particular embodiments, the present invention provides a method to alter the potency of a cell, comprising contacting the cell with one or more activators or a composition comprising the one or more activators, wherein the one or more activators includes an ATF or combination of ATFs, and wherein the one or more activators modulate a component of a cellular pathway associated with cell potency. In related particular embodiments, the present invention provides a method to alter the potency of a cell, comprising contacting the cell with one or more repressors or a composition comprising the one or more repressors, wherein the one or more repressors includes an ATF or combination of ATFs, and wherein the one or more repressors modulate a component of a cellular pathway associated with cell potency.

In related embodiments, a method of reprogramming a cell comprises contacting the cell with one or more activators or a composition comprising the one or more activators, wherein the one or more activators comprises one or more ATFs, and wherein the one or more activators modulates a component of a cellular pathway associated with cell potency, thereby reprogramming the cell. In particular related embodiments, a method of reprogramming a cell comprises contacting the cell with one or more repressors or a composition comprising the one or more repressors, wherein the one or more repressors comprises one or more ATFs, and wherein the one or more repressors modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises at least one ATF, and wherein the one or more activators modulates a component of a cellular pathway associated with cell potency, thereby programming the cell. In other related embodiments, a method of programming a cell comprises contacting the cell with one or more repressors or a composition comprising the one or more repressors, wherein the one or more repressors comprises at least one ATF, and wherein the one or more repressors modulates a component of a cellular pathway associated with cell potency, thereby programming the cell.

In a particular related embodiment, a method of reprogramming or programming a cell comprise contacting the cell with: i) one or more activators, wherein the one or more activators comprises one or more ATFs; and ii) at least one repressor, wherein the one or more activators and repressor(s) modulate a component of a cellular pathway associated with cell potency, thereby reprogramming or programming the cell. In a particular related embodiment, a method of reprogramming or programming a cell comprise contacting the cell with: i) one or more repressors, wherein the one or more repressors comprises one or more ATFs; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators and/or repressors comprises at least one ATF, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

In particular embodiments, an ATF comprises at least one, at least two, at least three, at least four, at least five, or at least six or more ZFP DNA binding domains. In related embodiments, wherein the ATF further comprises a transcriptional repression domain, the repressor modulates the transcription of at least one component of a cellular pathway, said modulation comprising repression of gene expression relative to a control of about 95%, 90%, 85%, 80%, 75%, 70%, 65%, 60%, 55%, 50%, 45%, 40%, 35%, 30%, 25%, 20%, 15%, 10%, 5%, 1% or 0%.

In a further related embodiment, wherein the ATF further comprises a transcriptional activation domain, the activator modulates the transcription of at least one component of a cellular pathway, said modulation comprising activation of gene expression relative to a control of about 110%, 120%, 130%, 140%, 150%, 160%, 170%, 180%, 190%, 200%, 250%, 300%, 350%, 400%, 450%, 500%, 600%, 700%, 800%, 900%, 1000%, 1500%, or 2000% or more.

M. Hormone Binding Domain-Transcription Factor Fusion Proteins

The present invention further contemplates, in part, the use of temporally controlled transcription factors in a method to alter the potency of the cell. In particular illustrative embodiments, the natural transcription factors and artificial transcription factors that are described elsewhere herein further comprise a hormone binding domain (HBD) suitable to regulate the activity of the transcription factor. HBD-transcription factor fusion proteins can be either transcriptional repressors or activators depending on the context in which they are used.

The ATFs are engineered zinc finger proteins that are capable precisely regulating gene expression at any given locus. In the methods of the present invention, one or more ATFs are designed so as to bind to and modulate the transcription of the genetic locus of a component of a cellular pathway associated with cell potency. It will be apparent to one of skill in the art that ATF(s) or any other transcription factors described herein may be fused to a hormone binding domain in order to facilitate the temporal control of transcription factor activity.

Ectopic expression of transcription factors in a temporally controlled manner is useful for regulation of gene expression of one or more components of a cell potency pathway. Such precise control offers numerous advantages to reprogramming and/or programming cells of the present invention in in vivo and/or ex vivo methods of cell, tissue, and/or organ regenerative therapy.

In particular illustrative embodiments, a steroid hormone-inducible system allows high levels of expression, in addition to temporal control of protein activity. The temporally controlled activity can be transcriptional repression or transcriptional activation.

In particular illustrative embodiments, a steroid hormone inducible system utilizes fusions between the hormone-binding domain (HBD) of a steroid receptor and a heterologous protein (reviewed in (Mattioni et al., 1994)).

Without wishing to be bound to any particular theory, in the absence of hormone, the HBD-fusion protein is held in an inactive state, presumably due to complex formation with hsp 90 (Scherrer et al., 1993). Addition of hormone causes a conformational change that dissociates hsp90, resulting in the rapid activation of the fusion protein (Tsai and O'Malley, 1994).

One having ordinary skill in the art would recognize that there are several advantages to the precise hormonal control of transcription factors. For example, the hormone ligand binding domain can stabilize the protein relative to the wild type protein (Kolm and Sive, 1995; Tada et al., 1997), allowing activation for a prolonged period of time. Further, steriod hormones are small lipophilic molecules that can diffuse through various cells and tissues. The steroid hormones or suitable analogs (e.g., dexamethasone, RU486, tamoxifen, etc.) may be administered by any of the techniques described herein. It would further be clear to one having ordinary skill in the art that various mutated HBDs may be fused to transcription factors of the present invention.

In certain illustrative embodiments, HBDs are preferred and often advantageous as they can be made insensitive to endogenous hormones, and highly sensistive to various hormone analogs (Feil R, Wagner J, Metzger D, and Chambon P. Regulation of Cre recombinase activity by mutated estrogen receptor ligand-binding domains.Biochem Biophys Res Commun. 1997 Aug. 28; 237(3):752-7).

Additionally, hormone administration rapidly activates the HBD transcription factor, so that increases or descreases in the levels of downstream targets can be seen in a relatively short time. This makes hormone inducible proteins ideal for the control of downstream targets of transcription factors (Braselmann et al, 1992). In particular embodiments, homone inducible transcription factors of the present invention are ideal for methods of reprogramming and/or programming cells of the present invention, as described herein throughout.

A wide variety of different types of HBD fusion polypeptides have been reported, including a number of types of DNA binding proteins, RNA binding proteins, kinases, and enzymes. One having ordinary skill in the art would understand that one concern is that the fusing a HBD to a transcription factor alters the function of the transcription factor. However, the skilled artisan routinely uses in vitro transcriptional activation assays of the transcription factor with and without the HBD fusion. In this way, the skilled artisan ensures that the HBD fusion polypeptide is suitable for use in particular methods and compositions of the present invention.

Hormone binding domains from both the steroid and thyroid hormone families of receptors can be used to regulate protein function. As noted above, there are a number of HBDs with point mutations that specifically bind synthetic hormones, rather than the normal endogenous ligand.

Illustrative examples of HBD mutants include, but are not limited to, a mutant estrogen receptor that specifically binds tamoxifen and a mutant progesterone receptor specifically binds RU486. Additionally, tissue culture data suggests that the Drosophila ecdysone recptor (EcR) HBD may be used to make myristerone-inducible proteins (Christopherson et al., 1992; No et al., 1996).

Without wishing to be bound by any particular theory, maximal temporal regulation of an HBD transcription factor fusion polypeptide is achieved when the HBD is fusion relatively close to the functional domain to be regulated (Mattioni et al., 1994; Picard D, Salser S J, and Yamamoto K R. Cell. 1988 Sep. 23; 54(7):1073-80; Godowski P J, Picard D, and Yamamoto KR. Science. 1988 Aug. 12; 241(4867):812-6). For example, the HBD may be fused about 1 amino acid, about 2 amino acids, about 3 amino acids, about 4 amino acids, about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 100 amino acids, or more from the domain wherein the hormone inducible regulation is desired.

It has been demonstrated that removal of hormone from the medium can reverse the activity of HBD fusion proteins (Jackson et al., 1993; Mattioni et al., 1994; Spitkovsky et al., 1994).

Thus, in certain embodiments, the hormone or analog thereof is administered to a subject in an amount and for a duration sufficient to induce the desired therapy. In a related embodiment, termination of the therapy may be accomplished by further administering to the patient, one or more antagonists of the hormone or analog thereof.

Thus, in particular embodiments, the present invention provides a method to alter the potency of a cell, comprising contacting the cell with one or more activators or a composition comprising the one or more activators, wherein the one or more activators includes an HBD domain, fragment, and/or variant thereof, and wherein the one or more activators modulate a component of a cellular pathway associated with cell potency. In related particular embodiments, the present invention provides a method to alter the potency of a cell, comprising contacting the cell with one or more repressors or a composition comprising the one or more repressors, wherein the one or more repressors includes an HBD domain, fragment, and/or variant thereof, and wherein the one or more repressors modulate a component of a cellular pathway associated with cell potency.

In particular embodiments, the HBD is selected from the group consisting of: the ER hormone binding domain, the PR hormone binding domain, the GR hormone binding domain, and the ecdysone receptor hormone binding domain or hormone binding fragments thereof. In certain embodiments, the HBD is mutated to increase hormone ligand specificity.

In related embodiments, a method of reprogramming a cell comprises contacting the cell with one or more activators or a composition comprising the one or more activators, wherein the one or more activators comprises an HBD domain, fragment, and/or variant thereof, and wherein the one or more activators modulates a component of a cellular pathway associated with cell potency, thereby reprogramming the cell. In particular related embodiments, a method of reprogramming a cell comprises contacting the cell with one or more repressors or a composition comprising the one or more repressors, wherein the one or more repressors comprises an HBD domain, fragment, and/or variant thereof, and wherein the one or more repressors modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises an HBD domain, fragment, and/or variant thereof, and wherein the one or more activators modulates a component of a cellular pathway associated with cell potency, thereby programming the cell. In other related embodiments, a method of programming a cell comprises contacting the cell with one or more repressors or a composition comprising the one or more repressors, wherein the one or more repressors comprises an HBD domain, fragment, and/or variant thereof, and wherein the one or more repressors modulates a component of a cellular pathway associated with cell potency, thereby programming the cell.

In a particular related embodiment, a method of reprogramming or programming a cell comprise contacting the cell with: i) one or more activators, wherein the one or more activators comprises an HBD domain, fragment, and/or variant thereof; and ii) at least one repressor, wherein the one or more activators and repressor(s) modulate a component of a cellular pathway associated with cell potency, thereby reprogramming or programming the cell. In a particular related embodiment, a method of reprogramming or programming a cell comprise contacting the cell with: i) one or more repressors, wherein the one or more repressors comprises an HBD domain, fragment, and/or variant thereof; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators and/or repressors comprises an HBD domain, fragment, and/or variant thereof, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

In particular embodiments, an HBD fusion polypeptide further comprises at least one, at least two, at least three, at least four, at least five, or at least six or more ZFP DNA binding domains. In related embodiments, wherein the HBD fusion polypeptide further comprises a transcriptional repression domain, the repressor modulates the transcription of at least one component of a cellular pathway, said modulation comprising repression of gene expression relative to a control of about 95%, 90%, 85%, 80%, 75%, 70%, 65%, 60%, 55%, 50%, 45%, 40%, 35%, 30%, 25%, 20%, 15%, 10%, 5%, 1% or 0%.

In a further related embodiment, wherein the HBD fusion polypeptide further comprises a transcriptional activation domain, the activator modulates the transcription of at least one component of a cellular pathway, said modulation comprising activation of gene expression relative to a control of about 110%, 120%, 130%, 140%, 150%, 160%, 170%, 180%, 190%, 200%, 250%, 300%, 350%, 400%, 450%, 500%, 600%, 700%, 800%, 900%, 1000%, 1500%, or 2000% or more.

N. Peptidomimetics

In addition to peptides consisting only of naturally-occurring amino acids, peptidomimetics or peptide analogs are also provided. Peptide analogs are commonly used in the pharmaceutical industry as non-peptide drugs with properties analogous to those of the template peptide. These types of non-peptide compound are termed “peptide mimetics” or “peptidomimetics” (Luthman, et al., A Textbook of Drug Design and Development, 14:386-406, 2nd Ed., Harwood Academic Publishers (1996); Joachim Grante, Angew. Chem. Int. Ed. Engl., 33:1699-1720 (1994); Fauchere, J., Adv. Drug Res., 15:29 (1986); Veber and Freidinger TINS, p. 392 (1985); and Evans, et al., J. Med. Chem. 30:229 (1987), which are incorporated herein by reference). A peptidomimetic is a molecule that mimics the biological activity of a peptide but is no longer peptidic in chemical nature. Peptidomimetic compounds are known in the art and are described, for example, in U.S. Pat. No. 6,245,886.

In some embodiments, the use of peptidomimetics may be preferred over unmodified polypeptides, because they have more economical production, greater chemical stability, enhanced pharmacological properties (half-life, absorption, potency, efficacy, etc.), altered specificity (e.g., a broad-spectrum of biological activities), reduced antigenicity, and others.

Thus, in particular embodiments, the present invention provides a method to alter the potency of a cell, comprising contacting the cell with one or more activators or a composition comprising the one or more activators, wherein the one or more activators includes a peptidomimetic or combination of peptidomimetics, and wherein the one or more activators modulate a component of a cellular pathway associated with cell potency. In related particular embodiments, the present invention provides a method to alter the potency of a cell, comprising contacting the cell with one or more repressors or a composition comprising the one or more repressors, wherein the one or more repressors includes a peptidomimetic or combination of peptidomimetics, and wherein the one or more repressors modulate a component of a cellular pathway associated with cell potency.

In related embodiments, a method of reprogramming a cell comprises contacting the cell with one or more activators or a composition comprising the one or more activators, wherein the one or more activators comprises one or more peptidomimetics, and wherein the one or more activators modulates a component of a cellular pathway associated with cell potency, thereby reprogramming the cell. In particular related embodiments, a method of reprogramming a cell comprises contacting the cell with one or more repressors or a composition comprising the one or more repressors, wherein the one or more repressors comprises one or more peptidomimetics, and wherein the one or more repressors modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises at least one peptidomimetic, and wherein the one or more activators modulates a component of a cellular pathway associated with cell potency, thereby programming the cell. In other related embodiments, a method of programming a cell comprises contacting the cell with one or more repressors or a composition comprising the one or more repressors, wherein the one or more repressors comprises at least one peptidomimetic, and wherein the one or more repressors modulates a component of a cellular pathway associated with cell potency, thereby programming the cell.

In a particular related embodiment, a method of reprogramming or programming a cell comprise contacting the cell with: i) one or more activators, wherein the one or more activators comprises one or more peptidomimetics; and ii) at least one repressor, wherein the one or more activators and repressor(s) modulate a component of a cellular pathway associated with cell potency, thereby reprogramming or programming the cell. In a particular related embodiment, a method of reprogramming or programming a cell comprise contacting the cell with: i) one or more repressors, wherein the one or more repressors comprises one or more peptidomimetics; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators and/or repressors comprises at least one peptidomimetic, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

O. Peptoids

The present invention also provides peptoids. Peptoid derivatives of peptides represent another form of modified peptides that retain the important structural determinants for biological activity, yet eliminate the peptide bonds, thereby conferring resistance to proteolysis (Simon, et al., 1992, Proc. Natl. Acad. Sci. US., 89:9367-9371 and incorporated herein by reference). Peptoids are oligomers of N-substituted glycines. A number of N-alkyl groups have been described, each corresponding to the side chain of a natural amino acid. The peptidomimetics of the present invention include compounds in which at least one amino acid, a few amino acids or all amino acid residues are replaced by the corresponding N-substituted glycines.

Thus, in particular embodiments, the present invention provides a method to alter the potency of a cell, comprising contacting the cell with one or more activators or a composition comprising the one or more activators, wherein the one or more activators includes a peptoid or combination of peptoids, and wherein the one or more activators modulate a component of a cellular pathway associated with cell potency. In related particular embodiments, the present invention provides a method to alter the potency of a cell, comprising contacting the cell with one or more repressors or a composition comprising the one or more repressors, wherein the one or more repressors includes a peptoid or combination of peptoids, and wherein the one or more repressors modulate a component of a cellular pathway associated with cell potency.

In related embodiments, a method of reprogramming a cell comprises contacting the cell with one or more activators or a composition comprising the one or more activators, wherein the one or more activators comprises one or more peptoids, and wherein the one or more activators modulates a component of a cellular pathway associated with cell potency, thereby reprogramming the cell. In particular related embodiments, a method of reprogramming a cell comprises contacting the cell with one or more repressors or a composition comprising the one or more repressors, wherein the one or more repressors comprises one or more peptoids, and wherein the one or more repressors modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises at least one peptoid, and wherein the one or more activators modulates a component of a cellular pathway associated with cell potency, thereby programming the cell. In other related embodiments, a method of programming a cell comprises contacting the cell with one or more repressors or a composition comprising the one or more repressors, wherein the one or more repressors comprises at least one peptoid, and wherein the one or more repressors modulates a component of a cellular pathway associated with cell potency, thereby programming the cell.

In a particular related embodiment, a method of reprogramming or programming a cell comprise contacting the cell with: i) one or more activators, wherein the one or more activators comprises one or more peptoids; and ii) at least one repressor, wherein the one or more activators and repressor(s) modulate a component of a cellular pathway associated with cell potency, thereby reprogramming or programming the cell. In a particular related embodiment, a method of reprogramming or programming a cell comprise contacting the cell with: i) one or more repressors, wherein the one or more repressors comprises one or more peptoids; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators and/or repressors comprises at least one peptoid, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

P. Intrabodies

The present invention contemplates, in part, to use single chain variable fragment (scFv) antibodies within the cell to directly modulate one or more components of a cellular pathway that affects the potency of a cell. Such antibodies are commonly referred to as an intrabodies. The high specificity and affinity of intrabodies to target antigens is well-established and intrabodies possess a much longer active half-life compared to reagents, such as siRNA. When the active half-life of the intracellular target molecule is long, the effects of intrabody expression are nearly instantaneous. Further, it is possible to design intrabodies to block certain binding interactions of a particular target molecule, while sparing others.

Thus, in particular embodiments, the present invention provides a method to alter the potency of a cell, comprising contacting the cell with one or more repressors or a composition comprising the one or more repressors, wherein the one or more repressors includes a intrabody or combination of intrabodies, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more intrabodies, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one intrabody, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more intrabodies; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one intrabody, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

Q. Transbodies

The present invention also contemplates, in part, to provide intrabodies that are fused to membrane translocation peptides or protein transduction domains (PTD), to create a ‘cell-permeable’ intrabody, which is known as a transbody. Membrane translocation peptides are short peptide sequences that enable proteins to translocate across the cell membrane and be internalized within the cytosol, through atypical secretory and internalization pathways. There are a number of distinct advantages that transbodies possess. For example correct conformational folding and disulfide bond formation can take place prior to introduction into the target cell. The use of cell-permeable antibodies or transbodies would also avoid the overwhelming safety and ethical concerns surrounding the direct application of recombinant DNA technology in human clinical therapy. Transbodies introduced into the cell would possess only a limited active half-life, without resulting in any permanent genetic alteration.

Thus, in particular embodiments, the present invention provides a method to alter the potency of a cell, comprising contacting the cell with one or more repressors or a composition comprising the one or more repressors, wherein the one or more repressors includes a transbody or combination of transbodies, and wherein the one or more repressors modulate a component of a cellular pathway associated with cell potency.

In related embodiments, a method of reprogramming a cell comprises contacting the cell with one or more repressors or a composition comprising the one or more repressors, wherein the one or more repressors comprises one or more transbodies, and wherein the one or more repressors modulates a component of a cellular pathway associated with cell potency, thereby reprogramming the cell.

In other related embodiments, a method of programming a cell comprises contacting the cell with one or more repressors or a composition comprising the one or more repressors, wherein the one or more repressors comprises at least one transbody, and wherein the one or more repressors modulates a component of a cellular pathway associated with cell potency, thereby programming the cell.

In a particular related embodiment, a method of reprogramming or programming a cell comprise contacting the cell with: i) one or more repressors, wherein the one or more repressors comprises one or more transbodies; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more repressors and/or activators that modulates a component of a cellular pathway associated with cell potency and wherein the one or more repressors comprises at least one transbody, thereby reprogramming the cell to a more potent state; and ii) contacting the cell with a second composition comprising one or more repressors and/or activators to modulate the same or a different component of a cellular pathway associated with cell potency, thereby programming the cell to a less potent state.

In particular embodiments, the transbody will target a repressor of one or more pluripotency factors in a cell. In related embodiments, the transbody will target multiple repressors of one or more pluripotency factors. Without wishing to be bound to any particular theory, relieving the repression of one or more pluripotent factors will lead to establishing a pluripotent state in the cell.

R. Small Molecules

The present invention also provides compositions and methods directed to the use of small molecules. A “small molecule” refers to a composition that has a molecular weight of less than about 5 kD, less than about 4 kD, less than about 3 kD, less than about 2 kD, less than about 1 kD, or less than about 0.5 kD. Small molecules can be nucleic acids, peptides, polypeptides, peptidomimetics, peptoids, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

A cell-free assay comprises contacting a cell with one or more test compounds, and determining the ability of the test compound to alter the potency of the cell, where determining the ability of the test compound to alter the potency of the cell comprises determining developmental potential of the cell, by methods known to those of skill in the art, and as described elsehere, herein. The invention disclosed herein encompasses the use of different libraries for the identification of small molecule modulators of one or more components of a cellular pathway associated with cell potency. Libraries useful for the purposes of the invention include, but are not limited to, (1) chemical libraries, (2) natural product libraries, and (3) combinatorial libraries comprised of random peptides, oligonucleotides and/or organic molecules.

Exemplary small molecules suitable for use in the compositions and methods of the present invention include, but are not limited to IBMV, TSA, VPA, SB203580, Hh-Ag1.3, cyclopamine, valproic acid, purmorphamine, forskolin, TWS119, BIO, cardigiol C, reversine, rosiglitasone, PD98059, WHI-P131, DAPT, 5-aza-C, all-trans RA, and ascorbic acid (Vitamin C), and the like, as described elsewhere herein.

Thus, in particular embodiments, the present invention provides a method to alter the potency of a cell, comprising contacting the cell with one or more activators or a composition comprising the one or more activators, wherein the one or more activators includes a small molecule or combination of small molecules, and wherein the one or more activators modulate a component of a cellular pathway associated with cell potency. In related particular embodiments, the present invention provides a method to alter the potency of a cell, comprising contacting the cell with one or more repressors or a composition comprising the one or more repressors, wherein the one or more repressors includes a small molecule or combination of small molecules, and wherein the one or more repressors modulate a component of a cellular pathway associated with cell potency.

In related embodiments, a method of reprogramming a cell comprises contacting the cell with one or more activators or a composition comprising the one or more activators, wherein the one or more activators comprises one or more small molecules, and wherein the one or more activators modulates a component of a cellular pathway associated with cell potency, thereby reprogramming the cell. In particular related embodiments, a method of reprogramming a cell comprises contacting the cell with one or more repressors or a composition comprising the one or more repressors, wherein the one or more repressors comprises one or more small molecules, and wherein the one or more repressors modulates a component of a cellular pathway associated with cell potency, thereby reprogramming the cell.

In other related embodiments, a method of programming a cell comprises contacting the cell with one or more activators or a composition comprising the one or more activators, wherein the one or more activators comprises at least one small molecule, and wherein the one or more activators modulates a component of a cellular pathway associated with cell potency, thereby programming the cell. In other related embodiments, a method of programming a cell comprises contacting the cell with one or more repressors or a composition comprising the one or more repressors, wherein the one or more repressors comprises at least one small molecule, and wherein the one or more repressors modulates a component of a cellular pathway associated with cell potency, thereby programming the cell.

In a particular related embodiment, a method of reprogramming or programming a cell comprise contacting the cell with: i) one or more activators, wherein the one or more activators comprises one or more small molecules; and ii) at least one repressor, wherein the one or more activators and repressor(s) modulate a component of a cellular pathway associated with cell potency, thereby reprogramming or programming the cell. In a particular related embodiment, a method of reprogramming or programming a cell comprise contacting the cell with: i) one or more repressors, wherein the one or more repressors comprises one or more small molecules; and ii) at least one activator, wherein the one or more repressors and activator(s) modulate a component of a cellular pathway associated with cell potency, thereby reprogramming or programming the cell.

In another particular related embodiment, a method of reprogramming and subsequently programming a cell comprises i) contacting the cell with a first composition comprising one or more activators and/or repressors that modulates a component of a cellular pathway associated with cell potency and wherein the one or more activators and/or repressors comprises at least one small molecule, thereby reprogramming the cell to a more potent state; and ii) contacting the cell with a second composition comprising one or more activators and/or repressors to modulate the same or a different component of a cellular pathway associated with cell potency, thereby programming the cell to a less potent state.

S. Other Repressors and Activators

The present invention contemplates, in part, methods of reprogramming and/or programming cells comprising contacting the cells with one or more activators and/or repressors, or a composition comprising the same, in order to modulate one of more components of a cellular potency pathway and thereby reprogram and/or program the cell.

In particular embodiments, polypeptide-based repressors and or activators are preferred. In certain embodiments, these polypeptide-based repressors and activators are transcription factors. In certain particular embodiments that transcription factors are transcriptional activators, and in other embodiments the transcription factors are transcriptional repressors.

In related embodiments, the transcription factors are fusion polypeptides, comprising one or more membrane translocating polypeptides. In further related embodiments, the transcription factors are Artificial Transcription Factors, as described elsewhere herein. In certain embodiments, the ATFs are fusion polypeptides comprising one or more membrance translocating polypeptides.

In further embodiments, miRNAs are used to regulate one or more pluripotency factors in order to program cells. In other embodiments, miRNAs are used to relieve repression of pluripotency factors by targeting the repressors; thus, resulting in the establishment of a pluripotent state.

In various embodiments, a repressor of the present invention will target repressors of pluripotent genes; thus, establishing or contributing to a pluripotent state.

In preferred embodiments, the repressors and activators are delivered in a cell specific manner (i.e., targeted to a specific cell type), as described elsewhere herein.

1. Repressors and Activators of Sox2

In one embodiment a repressor of the invention will target miR-134, which binds to and leads to the degradation of Sox2 mRNA. Such repression may be achieve with various repressors of the present invention, including, but not limited to antagomirs, antisense oligonucleotides, siRNAs, ribozymes, small molecules, aptamers, and the like.

Using retroviral-mediated transgene delivery, Lmx1a (LIM homeobox transcription factor 1, alpha), Ngn2 (neurogenin 2), or Pitx3 (paired-like homeodomain transcription factor 3) was overexpressed in neurospheres derived from embryonic day 14.5 rat ventral mesencephalic progenitors. Lmx1a, Ngn2, and Pitx3 downregulated the expression of Sox2 in these multipotent progenitor cells.

In one embodiment, a repressor of the invention will target Lmx1a, Ngn2, and/or Pitx3 in order to relieve repression of Sox2, and thus, facilitate the reprogramming or dedifferentiation of a cell to a more potent state. Suitable repressors for use in targeting Lmx1a, Ngn2, and/or Pitx3, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Lmx1a, Ngn2, and/or Pitx3 in order to relieve repression of Sox2, and thus, facilitate the reprogramming or dedifferentiation of a cell to a more potent state.

In another embodiment, the repressor is a transbody that binds to Lmx1a, Ngn2, or Pitx3 in order to prevent or suppress transcriptional repression of Sox2 and thereby facilitate cellular reprogramming or dedifferentiation.

HP1α is a transcriptional repressor, which binds directly to Brahma-related proteins at a highly conserved site and which is also ubiquitously expressed in early embryos. Consistent with this, overexpression of HP1α in the neural plate represses Sox2. A dominant-negative form of HP1α (ΔHP1α) consisting of its isolated chromoshadow domain (which can bind to Brahma-related proteins but lacks repressor activity) fails to repress Sox2.

In one embodiment, a repressor of the invention will target HP1a in order to relieve repression of Sox2, and thus, facilitate the reprogramming or dedifferentiation of a cell to a more potent state. Suitable repressors for use in targeting HP1a, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of HP1a in order to relieve repression of Sox2, and thus, facilitate the reprogramming or dedifferentiation of a cell to a more potent state.

In another embodiment, the repressor is a transbody that binds to HP1α in order to prevent or suppress transcriptional repression of Sox2 and thereby facilitate cellular reprogramming or dedifferentiation.

BMP4 does not induce trophoblast differentiation in monkey pluripotent stem cells, but instead induces primitive endoderm differentiation. Prominent downregulation of Sox2, which plays a pivotal role not only in pluripotency but also placenta development, was observed in cells treated with BMP4.

In one embodiment, a repressor of the invention will target BMP4 in order to relieve repression of Sox2, and thus, facilitate the reprogramming or dedifferentiation of a cell to a more potent state. Suitable repressors for use in targeting BMP4, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of BMP4 in order to relieve repression of Sox2, and thus, facilitate the reprogramming or dedifferentiation of a cell to a more potent state.

In another embodiment, the repressor is a transbody that binds to BMP4 in order to prevent or suppress transcriptional repression of Sox2 and thereby facilitate cellular reprogramming or dedifferentiation.

Other exemplary repressors of Sox2, include, but are not limited to Zfp281, HP1γ, Cdx2, SIP1, Zfhx1b, Zeb2, p300, and pCAF, among others.

Sip1 (Zfhx1b/Zeb2) belongs to the Zfhx1 family of multi-domain transcriptional repressors characterized by a homeodomain-like domain and by two zinc finger clusters each of which binds with high affinity to CACCTG and CACANNTG binding sites and can form complexes with Smads (Remacle et al., 1999 and Verschueren et al., 1999), the co-repressor CtBP (C-terminal binding protein) (Postigo and Dean, 2000 A. A. Postigo and D. C. Dean, Differential expression and function of members of the zfh-1 family of zinc finger/homeodomain repressors, Proc. Natl. Acad. Sci. U.S.A. 97 (2000), pp. 6391-6396. View Record in Scopus I Cited By in Scopus (46) Postigo and Dean, 2000 and van Grunsven et al., 2003), and the co-activators p300 and pCAF (p300/CBP associated factor) (van Grunsven et al., 2006).

In one embodiment, a repressor of the invention will target Zfp281, HP1γ, Cdx2, SIP1, Zfhx1b, Zeb2, p300, and pCAF in order to relieve repression of Sox2, and thus, facilitate the reprogramming or dedifferentiation of a cell to a more potent state. Suitable repressors for use in targeting Zfp281, HP1γ, Cdx2, SIP1, Zfhx1b, Zeb2, p300, and pCAF, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Zfp281, HP1γ, Cdx2, SIP1, Zfhx1b, Zeb2, p300, and pCAF in order to relieve repression of Sox2, and thus, facilitate the reprogramming or dedifferentiation of a cell to a more potent state.

In another embodiment, the repressor is a transbody that binds to Zfp281, HP1γ, Cdx2, SIP1, Zfhx1b, Zeb2, p300, and pCAF in order to prevent or suppress transcriptional repression of Sox2 and thereby facilitate cellular reprogramming or dedifferentiation.

STAT3 is a member of the signal transducer and activator or transcription (STAT) family of proteins. In a novel signaling pathway activated during early neural development STAT3 directly regulates the Sox2 promoter leading to Sox2 expression.

In one embodiment, an activator of the invention is a polypeptide or fusion polypeptide that comprises the full-length STAT3 or a functional fragment thereof that activates the expression of Sox2 (e.g., transcriptional activation); thus, facilitating the reprogramming or dedifferentiation of a cell to a more potent state. Suitable STAT3 based activators can be a STAT3 mRNA, a STAT3 specific bifunctional antisense oligonucleotide, a dsDNA comprising STAT3, a STAT3 polypeptide or an active fragment thereof, a peptidomimetics of STAT3, peptoids of STAT3, or a small organic molecule that mimics the transcriptional activity of STAT3.

In particular embodiments, an artificial transcription factor comprises the STAT3 polypeptide or a functional fragment thereof. In certain embodiments, the artificial transcription optionally comprises a membrane translocation peptide. A STAT3 based activator of the present invention increase or upregulates expression of Sox2 (e.g., by transcriptional activation) thus, facilitate the reprogramming or dedifferentiation of a cell to a more potent state.

Gli2 binds to an enhancer that is vital for sox2 expression in telencephalic neuroepithelial (NE) cells, which consist of NSCs and neural precursor cells. Overexpression of a truncated form of Gli2 (Gli2DeltaC) or Gli2-specific shRNA in NE cells in vivo and in vitro inhibits cell proliferation and the expression of Sox2 and other NSC markers, including Hes1, Hes5, Notch1, CD133 and Bmi1.

In one embodiment, an activator of the invention is a polypeptide or fusion polypeptide that comprises the full-length GLI2 or a functional fragment thereof that activates the expression of Sox2 (e.g., transcriptional activation); thus, facilitating the reprogramming or dedifferentiation of a cell to a more potent state. Suitable GLI2 based activators can be a GLI2 mRNA, a GLI2 specific bifunctional antisense oligonucleotide, a dsDNA comprising GLI2, a GLI2 polypeptide or an active fragment thereof, a peptidomimetics of GLI2, peptoids of GLI2, or a small organic molecule that mimics the transcriptional activity of GLI2.

In particular embodiments, an artificial transcription factor comprises the GLI2 polypeptide or a functional fragment thereof. In certain embodiments, the artificial transcription optionally comprises a membrane translocation peptide. A GLI2 based activator of the present invention increase or upregulates expression of Sox2 (e.g., by transcriptional activation) thus, facilitate the reprogramming or dedifferentiation of a cell to a more potent state.

Glycoprotein M6A (GPM6A) is known as a transmembrane protein and an abundant cell surface protein on neurons in the central nervous system (CNS). Expression of shRNA against GPM6A markedly reduced the expression of neuroectodermal-associated genes (OTX1, Lmx1b, En1, Pax2, Pax5, Sox1, Sox2, and Wnt1).

In one embodiment, an activator of the invention is a polypeptide or fusion polypeptide that comprises the full-length GPM6A or a functional fragment thereof that activates the expression of Sox2 (e.g., by cell signaling cascade); thus, facilitating the reprogramming or dedifferentiation of a cell to a more potent state. Suitable GPM6A based activators can be a GPM6A mRNA, a GPM6A specific bifunctional antisense oligonucleotide, a dsDNA comprising GPM6A, a GPM6A polypeptide or an active fragment thereof, a peptidomimetics of GPM6A, peptoids of GPM6A, or a small organic molecule that mimics the transcriptional activity of GPM6A.

In particular embodiments, an artificial transcription factor comprises the GPM6A polypeptide or a functional fragment thereof. In certain embodiments, the artificial transcription optionally comprises a membrane translocation peptide. A GPM6A based activator of the present invention increase or upregulates expression of Sox2 (e.g., by cell signaling cascade); thus, facilitate the reprogramming or dedifferentiation of a cell to a more potent state.

Rat oligodendrocyte precursor cells (OPCs) can be induced by extracellular signals to convert to multipotent neural stem-like cells (NSLCs), which can then generate both neurons and glial cells. The conversion of OPCS to NSLCs depends on the reactivation of the Sox2 gene, which in turn depends on the recruitment of the tumor suppressor protein Brca1 and the chromatin-remodeling protein Brahma (Brm) to an enhancer in the Sox2 promoter. Moreover, the conversion is associated with the modification of Lys 4 and Lys 9 of histone H3 at the same enhancer.

In one embodiment, an activator of the invention is a polypeptide or fusion polypeptide that comprises the full-length BRM and/or Brca1 or a functional fragment thereof that activates the expression of Sox2 (e.g., by chromatin remodeling); thus, facilitating the reprogramming or dedifferentiation of a cell to a more potent state. Suitable BRM and/or Brca1 based activators can be a BRM and/or Brca1 mRNA, a BRM and/or Brca1 specific bifunctional antisense oligonucleotide, a dsDNA comprising BRM and/or Brca1, a BRM and/or Brca1 polypeptide or an active fragment thereof, a peptidomimetics of BRM and/or Brca1, peptoids of BRM and/or Brca1, or a small organic molecule that mimics the chromatin remodeling activity of BRM and/or Brca1.

In particular embodiments, an artificial transcription factor comprises the BRM and/or Brca1 polypeptide or a functional fragment thereof. In certain embodiments, the artificial transcription optionally comprises a membrane translocation peptide. A BRM and/or Brca1 based activator of the present invention increase or upregulates expression of Sox2 (e.g., by chromatin remodeling) thus, facilitate the reprogramming or dedifferentiation of a cell to a more potent state.

BAF250a deficiency compromises stem cell pluripotency, severely inhibits self-renewal, and promotes differentiation into primitive endoderm-like cells under normal feeder-free culture conditions. DNA microarray, immunostaining, and RNA analyses revealed that BAF250a-mediated chromatin remodeling contributes to the proper expression of numerous genes involved in ES cell self-renewal, including Sox2, Utf1, and Oct4.

In one embodiment, an activator of the invention is a polypeptide or fusion polypeptide that comprises the full-length BAF250a or a functional fragment thereof that activates the expression of Sox2 and/or Oct4 (e.g., by chromatin remodeling); thus, facilitating the reprogramming or dedifferentiation of a cell to a more potent state. Suitable BAF250a based activators can be a BAF250a mRNA, a BAF250a specific bifunctional antisense oligonucleotide, a dsDNA comprising BAF250a, a BAF250a polypeptide or an active fragment thereof, a peptidomimetics of BAF250a, peptoids of BAF250a, or a small organic molecule that mimics the chromatin remodeling activity of BAF250a.

In particular embodiments, an artificial transcription factor comprises the BAF250a polypeptide or a functional fragment thereof. In certain embodiments, the artificial transcription optionally comprises a membrane translocation peptide. A BAF250a based activator of the present invention increase or upregulates expression of Sox2 and/or Oct4 (e.g., by chromatin remodeling) thus, facilitate the reprogramming or dedifferentiation of a cell to a more potent state.

Pax6 is a key regulator in the neuronal fate determination as well as the proliferation of neural stem cells. Pax6 regulates the proliferation of neural progenitor cells of cortical subventricular zone, through transcriptional activation of Sox2. Pax6 binds to the Sox2 promoter by chromatin immunoprecipitation assay and activates Sox2 expression in a luciferase reporter gene assay.

In one embodiment, an activator of the invention is a polypeptide or fusion polypeptide that comprises the full-length Pax6 or a functional fragment thereof that activates the expression of Sox2 (e.g., transcriptional activation); thus, facilitating the reprogramming or dedifferentiation of a cell to a more potent state. Suitable Pax6 based activators can be a Pax6 mRNA, a Pax6 specific bifunctional antisense oligonucleotide, a dsDNA comprising Pax6, a Pax6 polypeptide or an active fragment thereof, a peptidomimetics of Pax6, peptoids of Pax6, or a small organic molecule that mimics the transcriptional activity of Pax6.

In particular embodiments, an artificial transcription factor comprises the Pax6 polypeptide or a functional fragment thereof. In certain embodiments, the artificial transcription optionally comprises a membrane translocation peptide. A Pax6 based activator of the present invention increase or upregulates expression of Sox2 (e.g., by transcriptional activation) thus, facilitate the reprogramming or dedifferentiation of a cell to a more potent state.

Another exemplary activator of Sox2 is Notch.

In one embodiment, an activator of the invention is a polypeptide or fusion polypeptide that comprises the full-length NOTCH or a functional fragment thereof that activates the expression of Sox2 (e.g., transcriptional activation); thus, facilitating the reprogramming or dedifferentiation of a cell to a more potent state. Suitable NOTCH based activators can be a NOTCH mRNA, a NOTCH specific bifunctional antisense oligonucleotide, a dsDNA comprising NOTCH, a NOTCH polypeptide or an active fragment thereof, a peptidomimetics of NOTCH, peptoids of NOTCH, or a small organic molecule that mimics the transcriptional activity of NOTCH.

In particular embodiments, an artificial transcription factor comprises the NOTCH polypeptide or a functional fragment thereof. In certain embodiments, the artificial transcription optionally comprises a membrane translocation peptide. A NOTCH based activator of the present invention increase or upregulates expression of Sox2 (e.g., by transcriptional activation) thus, facilitate the reprogramming or dedifferentiation of a cell to a more potent state.

2. Repressors and Activators of Nanog

In one embodiment a repressor of the invention will target miR-296 miR-470, which bind to and lead to the degradation of Nanog mRNA. Such repression may be achieve with various repressors of the present invention, including, but not limited to antagomirs, antisense oligonucleotides, siRNAs, ribozymes, small molecules, aptamers, and the like.

Tcf3 acts broadly on a genome-wide scale to reduce the levels of several promoters of self-renewal (Nanog, Tcl1, Tbx3, Esrrb) while not affecting other ESC genes (Oct4, Sox2, Fgf4). Comparing effects of Tcf3 ablation with Oct4 or Nanog knockdown revealed that Tcf3 counteracted effects of both Nanog and Oct4.

In one embodiment, a repressor of the invention will target Tcf3 in order to relieve repression of Nanog and/or Oct-4, and thus, facilitate the reprogramming or dedifferentiation of a cell to a more potent state. Suitable repressors for use in targeting Tcf3, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Tcf3 in order to relieve repression of Nanog and/or Oct-4, and thus, facilitate the reprogramming or dedifferentiation of a cell to a more potent state.

In another embodiment, the repressor is a transbody that binds to Tcf3 in order to prevent or suppress transcriptional repression of Nanog and/or Oct-4 and thereby facilitate cellular reprogramming or dedifferentiation.

Slug (approved gene symbol Snail2), a member of the Snail gene family of zinc-finger transcription factors, is believed to function in the maintenance of the nonepithelial phenotype. Slug target genes validated by real-time PCR or Western analyses include self-renewal genes (Bmi1, Nanog, Gfi1), epithelial-mesenchymal genes (Tcfe2a, Ctnb1, Sin3a, Hdac1, Hdac2, Muc1, Cldn11), survival genes (Bcl2, Bbc3), and cell cycle/damage genes (Cdkn1a, Rbl1, Mdm2).

In one embodiment, a repressor of the invention will target Slug in order to relieve repression of Nanog, and thus, facilitate the reprogramming or dedifferentiation of a cell to a more potent state. Suitable repressors for use in targeting Slug, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Slug in order to relieve repression of Nanog, and thus, facilitate the reprogramming or dedifferentiation of a cell to a more potent state.

In another embodiment, the repressor is a transbody that binds to Slug in order to prevent or suppress transcriptional repression of Nanog, and thereby facilitate cellular reprogramming or dedifferentiation.

Nuclear tumor suppressor p53 transactivates proapoptotic genes or antioxidant genes depending on stress severity, while cytoplasmic p53 induces mitochondrial-dependent apoptosis without gene transactivation. Although SIRT1 is a p53 deacetylase, it inhibits p53-mediated transactivation. SIRT1 blocks nuclear translocation of cytoplasmic p53 in response to endogenous reactive oxygen species (ROS) and triggers mitochondrial-dependent apoptosis in mouse embryonic stem (mES) cells. ROS generated by antioxidant-free culture caused p53 translocation into mitochondria in wild-type mES cells but induced p53 translocation into the nucleus in SIRT1(−/−) mES cells. Endogenous ROS triggered apoptosis of wild-type mES through mitochondrial translocation of p53 and BAX but inhibited Nanog expression of SIRT1(−/−) mES, indicating that SIRT1 makes mES cells sensitive to ROS and inhibits p53-mediated suppression of Nanog expression.

In one embodiment, a repressor of the invention will target p53 in order to relieve repression of Nanog, and thus, facilitate the reprogramming or dedifferentiation of a cell to a more potent state. Suitable repressors for use in targeting p53, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of p53 in order to relieve repression of Nanog, and thus, facilitate the reprogramming or dedifferentiation of a cell to a more potent state.

In another embodiment, the repressor is a transbody that binds to p53 in order to prevent or suppress transcriptional repression of Nanog, and thereby facilitate cellular reprogramming or dedifferentiation.

In one embodiment, an activator of the invention is a polypeptide or fusion polypeptide that comprises the full-length SIRT1 or a functional fragment thereof that activates the expression of Nanog (e.g., transcriptional activation); thus, facilitating the reprogramming or dedifferentiation of a cell to a more potent state. Suitable SIRT1 based activators can be a SIRT1 mRNA, a SIRT1 specific bifunctional antisense oligonucleotide, a dsDNA comprising SIRT1, a SIRT1 polypeptide or an active fragment thereof, a peptidomimetics of SIRT1, peptoids of SIRT1, or a small organic molecule that mimics the transcriptional activity of SIRT1.

In particular embodiments, an artificial transcription factor comprises the SIRT1 polypeptide or a functional fragment thereof. In certain embodiments, the artificial transcription optionally comprises a membrane translocation peptide. A SIRT1 based activator of the present invention increase or upregulates expression of Nanog (e.g., by transcriptional activation) thus, facilitate the reprogramming or dedifferentiation of a cell to a more potent state.

Nanog, Oct4 and the repressor proteins, including the NuRD, Sin3A and Pml complexes, co-occupy Nanog-target genes in mouse ES cells, suggesting that Nanog and Oct4 together may communicate with distinct repression complexes to control gene transcription. Of the various core components in the NuRD complex with which Nanog and Oct4 interact, Mta1 was preferred, whereas Mbd3 and Rbbp7 were either absent or present at sub-stoichiometric levels. This unique Hdac1/2- and Mta1/2-containing complex is named NODE (for Nanog and Oct4 associated deacetylase). Other illustrative repressors of Nanog and/or Oct-4 are Sin3A and Pml1.

In one embodiment, a repressor of the invention will target a member of the NuRD complex, Sin3A, Pml1, HDAC1/2, and MTA1/2 in order to relieve repression of Nanog and/or Oct-4, and thus, facilitate the reprogramming or dedifferentiation of a cell to a more potent state. Suitable repressors for use in targeting a member of the NuRD complex, Sin3A, Pml1, HDAC1/2, and MTA1/2, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of a member of the NuRD complex, Sin3A, Pml1, HDAC1/2, and MTA1/2 in order to relieve repression of Nanog and/or Oct-4, and thus, facilitate the reprogramming or dedifferentiation of a cell to a more potent state.

In another embodiment, the repressor is a transbody that binds to a member of the NuRD complex, Sin3A, Pml1, HDAC1/2, and MTA1/2 in order to prevent or suppress transcriptional repression of Nanog and/or Oct-4, and thereby facilitate cellular reprogramming or dedifferentiation.

Other illustrative repressors included, but are not limited to Zfp281, TCF 1, 3, 4, and 7, Groucho, CtBP, Hic-5, and Lef1.

These Tcf proteins are the DNA-binding transcriptional regulators of the canonical Wnt signaling pathway. Through a highly conserved HMG domain and an amino-terminal β-catenin interaction domain, each Tcf protein can promote transcription of downstream targets when Wnt-stabilized β-catenin accumulates intracellularly. In the absence of stabilized β-catenin, Tcf proteins have been shown to function as transcriptional repressors by interacting with corepressor proteins, such as Groucho, CtBP, and HIC-5. Direct relationships between the biochemical properties of Tcf proteins and their physiological effects have been demonstrated by several studies expressing mutated forms of the proteins in model organisms.

In one embodiment, a repressor of the invention will target Zfp281, TCF 1, 3, 4, and 7, Groucho, CtBP, Hic-5, and/or Lef1 in order to relieve repression of Nanog and/or Oct-4, and thus, facilitate the reprogramming or dedifferentiation of a cell to a more potent state. Suitable repressors for use in targeting Zfp281, TCF 1, 3, 4, and 7, Groucho, CtBP, Hic-5, and/or Lef1, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Zfp281, TCF 1, 3, 4, and 7, Groucho, CtBP, Hic-5, and/or Lef1 in order to relieve repression of Nanog and/or Oct-4, and thus, facilitate the reprogramming or dedifferentiation of a cell to a more potent state.

In another embodiment, the repressor is a transbody that binds to Slug in order to prevent or suppress transcriptional repression of Nanog and/or Oct-4, and thereby facilitate cellular reprogramming or dedifferentiation.

FoxD3 behaves as a positive activator of Nanog to counter the repressive effect of Oct4. The expression of Oct4 is activated by FoxD3 and Nanog but repressed by Oct4 itself, thus, exerting an important negative feedback loop to limit its own activity. Indeed, overexpression of either FoxD3 or Nanog in ES cells failed to increase the concentration of Oct4 beyond the steady-state concentration, whereas knocking down either FoxD3 or Nanog reduces the expression of Oct4 in ES cells.

In one embodiment, an activator of the invention is a polypeptide or fusion polypeptide that comprises the full-length Foxd3 or a functional fragment thereof that activates the expression of Nanog (e.g., transcriptional activation); thus, facilitating the reprogramming or dedifferentiation of a cell to a more potent state. Suitable Foxd3 based activators can be a Foxd3 mRNA, a Foxd3 specific bifunctional antisense oligonucleotide, a dsDNA comprising Foxd3, a Foxd3 polypeptide or an active fragment thereof, a peptidomimetics of Foxd3, peptoids of Foxd3, or a small organic molecule that mimics the transcriptional activity of Foxd3.

In particular embodiments, an artificial transcription factor comprises the Foxd3 polypeptide or a functional fragment thereof. In certain embodiments, the artificial transcription optionally comprises a membrane translocation peptide. A Foxd3 based activator of the present invention increase or upregulates expression of Nanog (e.g., by transcriptional activation) thus, facilitate the reprogramming or dedifferentiation of a cell to a more potent state.

TGFbeta- and BMP-responsive SMADs can bind with the NANOG proximal promoter. NANOG promoter activity is enhanced by TGFbeta/Activin and FGF signaling and is decreased by BMP signaling

In one embodiment, an activator of the invention is a polypeptide or fusion polypeptide that comprises the full-length TGFbeta/Activin and/or FGFor a functional fragment thereof that activates the expression of Nanog (e.g., cell signaling pathways); thus, facilitating the reprogramming or dedifferentiation of a cell to a more potent state. Suitable TGFbeta/Activin and/or FGFbased activators can be a TGFbeta/Activin and/or FGFmRNA, a TGFbeta/Activin and/or FGFspecific bifunctional antisense oligonucleotide, a dsDNA comprising TGFbeta/Activin and/or FGF, a TGFbeta/Activin and/or FGFpolypeptide or an active fragment thereof, a peptidomimetics of TGFbeta/Activin and/or FGF, peptoids of TGFbeta/Activin and/or FGF, or a small organic molecule that mimics the transcriptional activity of TGFbeta/Activin and/or FGF.

In particular embodiments, an artificial transcription factor comprises the TGFbeta/Activin and/or FGFpolypeptide or a functional fragment thereof. In certain embodiments, the artificial transcription optionally comprises a membrane translocation peptide. A TGFbeta/Activin and/or FGFbased activator of the present invention increase or upregulates expression of Nanog (e.g., by cell signaling pathways) thus, facilitate the reprogramming or dedifferentiation of a cell to a more potent state.

Esrrb can interact with Oct4 independently of DNA. Esrrb is recruited near the Oct-Sox element in the Nanog proximal promoter, where it positively regulates Nanog expression. Esrrb recruitment to the Nanog promoter requires both the presence of Oct4 and a degenerate estrogen-related receptor DNA element. Consistent with its role in Nanog regulation, expression of the Esrrb protein within the Oct4-positive ES cell population is mosaic and correlates with the mosaic expression of the Nanog protein.

In one embodiment, an activator of the invention is a polypeptide or fusion polypeptide that comprises the full-length Esrrb or a functional fragment thereof that activates the expression of Nanog (e.g., transcriptional activation); thus, facilitating the reprogramming or dedifferentiation of a cell to a more potent state. Suitable Esrrb based activators can be a Esrrb mRNA, a Esrrb specific bifunctional antisense oligonucleotide, a dsDNA comprising Esrrb, a Esrrb polypeptide or an active fragment thereof, a peptidomimetics of Esrrb, peptoids of Esrrb, or a small organic molecule that mimics the transcriptional activity of Esrrb.

In particular embodiments, an artificial transcription factor comprises the Esrrb polypeptide or a functional fragment thereof. In certain embodiments, the artificial transcription optionally comprises a membrane translocation peptide. A Esrrb based activator of the present invention increase or upregulates expression of Nanog (e.g., by transcriptional activation) thus, facilitate the reprogramming or dedifferentiation of a cell to a more potent state.

A further illustrative example of an activator of Nanog are the transcription factors Klf-2, Klf-4, and Klf-5, among others.

In one embodiment, an activator of the invention is a polypeptide or fusion polypeptide that comprises the full-length Klf-2, Klf-4, and/or Klf-5 or a functional fragment thereof that activates the expression of Nanog (e.g., transcriptional activation); thus, facilitating the reprogramming or dedifferentiation of a cell to a more potent state. Suitable Klf-2, Klf-4, and/or Klf-5 based activators can be Klf-2, Klf-4, and/or Klf-5 mRNAs, Klf-2, Klf-4, and/or Klf-5 specific bifunctional antisense oligonucleotides, a dsDNA comprising Klf-5, Klf-2, Klf-4, and/or Klf-5 polypeptides or an active fragment thereof, a peptidomimetics of Klf-2, Klf-4, and/or Klf-5, peptoids of Klf-2, Klf-4, and/or Klf-5, or a small organic molecule that mimics the transcriptional activity of Klf-2, Klf-4, and/or Klf-5.

In particular embodiments, an artificial transcription factor comprises a Klf-2, Klf-4, and/or Klf-5 polypeptide or a functional fragment thereof. In certain embodiments, the artificial transcription optionally comprises a membrane translocation peptide. A Klf-2, Klf-4, and/or Klf-5 based activator of the present invention increase or upregulates expression of Nanog (e.g., by transcriptional activation) thus, facilitate the reprogramming or dedifferentiation of a cell to a more potent state.

3. Repressors and Activators of Oct-4

In one embodiment a repressor of the invention will target miR-470, which bind to and lead to the degradation of Oct-4 mRNA. Such repression may be achieve with various repressors of the present invention, including, but not limited to antagomirs, antisense oligonucleotides, siRNAs, ribozymes, small molecules, aptamers, and the like.

The pluripotency-determining gene Oct3/4 (also called Pou5f1) undergoes postimplantation silencing in a process mediated by the histone methyltransferase G9a. Microarray analysis shows that this enzyme may operate as a master regulator that inactivates numerous early-embryonic genes by bringing about heterochromatinization of methylated histone H3K9 and de novo DNA methylation. Genetic studies in differentiating embryonic stem cells demonstrate that a point mutation in the G9a SET domain prevents heterochromatinization but still allows de novo methylation, whereas biochemical and functional studies indicate that G9a itself is capable of bringing about de novo methylation through its ankyrin domain, by recruiting Dnmt3a and Dnmt3b independently of its histone methyltransferase activity.

In one embodiment, a repressor of the invention will target G9a, Dnmt3a, and/or Dnmt3b in order to relieve repression of Oct-4, and thus, facilitate the reprogramming or dedifferentiation of a cell to a more potent state. Suitable repressors for use in targeting G9a, Dnmt3a, and/or Dnmt3b, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of G9a, Dnmt3a, and/or Dnmt3b in order to relieve repression of Oct-4, and thus, facilitate the reprogramming or dedifferentiation of a cell to a more potent state.

In another embodiment, the repressor is a transbody that binds to G9a, Dnmt3a, and/or Dnmt3b in order to prevent or suppress transcriptional repression of Oct-4 and thereby facilitate cellular reprogramming or dedifferentiation.

In addition, Oct-4 is heavily regulated by nuclear hormone receptors, including retinoic acid based heterodimers, by both repression and activation.

Illustrative examples of repressors of Oct-4, include, but are not limited to Cdx2, GCNF, PIASy, PIAS1, 2, and 3, Nr2f2, Eomes, Esx1, CoupTF1, CoupTFII, COUTR1, Cdx-2, RARβ/RXRα, RARα/RXRα, and/or Zfp281, among others.

In one embodiment, a repressor of the invention will target Cdx2, GCNF, PIASy, PIAS1, 2, and 3, Nr2f2, Eomes, Esx1, CoupTF1, CoupTFII, COUTR1, Cdx-2, RARβ/RXRα, RARα/RXRα, and/or Zfp281 in order to relieve repression of Oct-4, and thus, facilitate the reprogramming or dedifferentiation of a cell to a more potent state. Suitable repressors for use in targeting Cdx2, GCNF, PIASy, PIAS1, 2, and 3, Nr2f2, Eomes, Esx1, CoupTF1, CoupTFII, COUTR1, Cdx-2, RARβ/RXRα, RARα/RXRα, and/or Zfp281, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Cdx2, GCNF, PIASy, PIAS1, 2, and 3, Nr2f2, Eomes, Esx1, CoupTF1, CoupTFII, COUTR1, Cdx-2, RARβ/RXRα, RARα/RXRα, and/or Zfp281 in order to relieve repression of Oct-4, and thus, facilitate the reprogramming or dedifferentiation of a cell to a more potent state.

In another embodiment, the repressor is a transbody that binds to Cdx2, GCNF, PIASy, PIAS1, 2, and 3, Nr2f2, Eomes, Esx1, CoupTF1, CoupTFII, COUTR1, Cdx-2, RARβ/RXRα, RARα/RXRα, and/or Zfp281 in order to prevent or suppress transcriptional repression of Oct-4 and thereby facilitate cellular reprogramming or dedifferentiation.

Illustrative activators of Oct-4 gene expression include, but are not limited to RARβ/RXRβ, SF1, Nr5a2, GABPα, Esrrb, Klf-5, BAF250a, and/or Sox2, among others.

In one embodiment, an activator of the invention is a polypeptide or fusion polypeptide that comprises the full-length RARβ/RXRβ, SF1, Nr5a2, GABPα, Esrrb, Klf-5, BAF250a, and/or Sox2 or a functional fragment thereof that activates the expression of Oct-4 (e.g., transcriptional activation); thus, facilitating the reprogramming or dedifferentiation of a cell to a more potent state. Suitable RARβ/RXRβ, SF1, Nr5a2, GABPα, Esrrb, Klf-5, BAF250a, and/or Sox2 based activators can be a RARβ/RXRβ, SF1, Nr5a2, GABPα, Esrrb, Klf-5, BAF250a, and/or Sox2 mRNA, a RARβ/RXRβ, SF1, Nr5a2, GABPα, Esrrb, Klf-5, BAF250a, and/or Sox2 specific bifunctional antisense oligonucleotide, a dsDNA comprising RARβ/RXRβ, SF1, Nr5a2, GABPα, Esrrb, Klf-5, BAF250a, and/or Sox2, a RARβ/RXRβ, SF1, Nr5a2, GABPα, Esrrb, Klf-5, BAF250a, and/or Sox2 polypeptide or an active fragment thereof, a peptidomimetics of RARβ/RXRβ, SF1, Nr5a2, GABPα, Esrrb, Klf-5, BAF250a, and/or Sox2, peptoids of RARβ/RXRβ, SF1, Nr5a2, GABPα, Esrrb, Klf-5, BAF250a, and/or Sox2, or a small organic molecule that mimics the transcriptional activity of RARβ/RXRβ, SF1, Nr5a2, GABPα, Esrrb, Klf-5, BAF250a, and/or Sox2.

In particular embodiments, an artificial transcription factor comprises the RARβ/RXRβ, SF1, Nr5a2, GABPα, Esrrb, Klf-5, BAF250a, and/or Sox2 polypeptide or a functional fragment thereof. In certain embodiments, the artificial transcription optionally comprises a membrane translocation peptide. A RARβ/RXRβ, SF1, Nr5a2, GABPα, Esrrb, Klf-5, BAF250a, and/or Sox2 based activator of the present invention increase or upregulates expression of Oct-4 (e.g., by transcriptional activation) thus, facilitate the reprogramming or dedifferentiation of a cell to a more potent state.

4. Repressors and Activators of Klf4

Lower levels of KLF4 expression in the proliferative compartment of the intestinal epithelium are regulated by the transcription factors TCF4 and SOX9, an effector and a target, respectively, of beta-catenin/Tcf signaling, and independently of CDX2. Thus, reduced levels of KLF4 tumor suppressor activity in colon tumors may be driven by elevated beta-catenin/Tcf signaling.

In one embodiment, a repressor of the invention will target TCF4 and/or SOX9 in order to relieve repression of Klf-4, and thus, facilitate the reprogramming or dedifferentiation of a cell to a more potent state. Suitable repressors for use in targeting TCF4 and/or SOX9, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of TCF4 and/or SOX9 in order to relieve repression of Klf-4, and thus, facilitate the reprogramming or dedifferentiation of a cell to a more potent state.

In another embodiment, the repressor is a transbody that binds to TCF4 and/or SOX9 in order to prevent or suppress transcriptional repression of Klf-4 and thereby facilitate cellular reprogramming or dedifferentiation.

PIAS1 regulates the function of KLF4 for SMC gene expression. PIAS1 interacted with KLF4 in mammalian two-hybrid and coimmunoprecipitation assays, and overexpression of PIAS1 inhibited KLF4-repression of SM alpha-actin promoter activity. Moreover, PIAS1 promoted degradation of KLF4 through sumoylation.

In one embodiment, a repressor of the invention will target PIASy, PIAS1, PIAS 2, and/or PIAS 3 in order to relieve repression of Klf-4, and thus, facilitate the reprogramming or dedifferentiation of a cell to a more potent state. Suitable repressors for use in targeting PIASy, PIAS1, PIAS 2, and/or PIAS, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of PIASy, PIAS1, PIAS 2, and/or PIAS in order to relieve repression of Klf-4, and thus, facilitate the reprogramming or dedifferentiation of a cell to a more potent state.

In another embodiment, the repressor is a transbody that binds to PIASy, PIAS1, PIAS 2, and/or PIAS in order to prevent or suppress transcriptional repression of Klf-4 and thereby facilitate cellular reprogramming or dedifferentiation.

C/EBPbeta knockdown increases levels of KLF4 and Krox20, suggesting that C/EBPbeta normally suppresses Krox20 and KLF4 expression via a tightly controlled negative feedback loop. KLF4 is specifically induced in response to cAMP, which by itself can partially activate adipogenesis. These data suggest that KLF4 functions as an immediate early regulator of adipogenesis to induce C/EBPbeta.

In one embodiment, a repressor of the invention will target C/EBPbeta in order to relieve repression of Klf-4, and thus, facilitate the reprogramming or dedifferentiation of a cell to a more potent state. Suitable repressors for use in targeting C/EBPbeta, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of C/EBPbeta in order to relieve repression of Klf-4, and thus, facilitate the reprogramming or dedifferentiation of a cell to a more potent state.

In another embodiment, the repressor is a transbody that binds to C/EBPbeta in order to prevent or suppress transcriptional repression of Klf-4 and thereby facilitate cellular reprogramming or dedifferentiation.

The ERK transcription factor represses the level of KLF4 gene expression. Transfection of GT1-7 cells with ERK inhibited KLF4 gene expression, as did treating the cells with the peptide enterostatin. The later effect was blocked by the ERK inhibitor, U0126, suggesting that ERK was mediating the effect of enterostatin (Park M, Oh H, and York DA 2009. Enterostatin affects cyclic AMP and ERK signaling pathways to regulate Agouti-related protein (AgRP) expression. Peptides 30:181-190).

In one embodiment, a repressor of the invention will target ERK in order to relieve repression of Klf-4, and thus, facilitate the reprogramming or dedifferentiation of a cell to a more potent state. Suitable repressors for use in targeting ERK, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of ERK in order to relieve repression of Klf-4, and thus, facilitate the reprogramming or dedifferentiation of a cell to a more potent state.

In another embodiment, the repressor is a transbody that binds to ERK in order to prevent or suppress transcriptional repression of Klf-4 and thereby facilitate cellular reprogramming or dedifferentiation.

HT29 human colon cancer cells treated with the gamma-secretase inhibitor dibenzazepine to inhibit Notch signaling or small interfering RNA directed against Notch increased KLF4 levels. Conversely, overexpression of Notch in HT29 cells reduced KLF4 levels and suppressed KLF4 promoter activity. HES1 binding sites are present in the KLF4 promoter. Overexpression of HES1, or Notch, an upstream activator of HES1, inhibited KLF4 promoter activity (Ghaleb A M, Aggarwal G, Bialkowska A B, Nandan M O, Yang V W 2008. Notch inhibits expression of the Krüppel-like factor 4 tumor suppressor in the intestinal epithelium. Mol Cancer Res 6(12):1920-1927).

In one embodiment, a repressor of the invention will target Notch and/or HES1 in order to relieve repression of Klf-4, and thus, facilitate the reprogramming or dedifferentiation of a cell to a more potent state. Suitable repressors for use in targeting Notch and/or HES1, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Notch and/or HES1 in order to relieve repression of Klf-4, and thus, facilitate the reprogramming or dedifferentiation of a cell to a more potent state.

In another embodiment, the repressor is a transbody that binds to Notch and/or HES1 in order to prevent or suppress transcriptional repression of Klf-4 and thereby facilitate cellular reprogramming or dedifferentiation.

Heat stress up-regulated KLF4 messenger RNA and protein levels in a time-dependent manner in vivo and in four cell lines. Moreover, a study with heat shock transcription factor 1 (Hsf1) gene knockout mice indicated that the induction of KLF4 in response to heat stress was mediated by Hsf1. This process occurred rapidly, indicating that KLF4 is an immediate early response gene of heat stress (Liu Y, Wang J, Yi Y, Zhang H, Liu J, Liu M, Yuan C, Tang D, Benjamin I J, Xiao X 2006. Induction of KLF4 in response to heat stress. Cell Stress & Chaperones 11(4):379-389).

In one embodiment, an activator of the invention is a polypeptide or fusion polypeptide that comprises the full-length HSF-1 or a functional fragment thereof that activates the expression of Klf-4 (e.g., transcriptional activation); thus, facilitating the reprogramming or dedifferentiation of a cell to a more potent state. Suitable HSF-1 based activators can be a HSF-1 mRNA, a HSF-1 specific bifunctional antisense oligonucleotide, a dsDNA comprising HSF-1, a HSF-1 polypeptide or an active fragment thereof, a peptidomimetics of HSF-1, peptoids of HSF-1, or a small organic molecule that mimics the transcriptional activity of HSF-1.

In particular embodiments, an artificial transcription factor comprises the HSF-1 polypeptide or a functional fragment thereof. In certain embodiments, the artificial transcription optionally comprises a membrane translocation peptide. A HSF-1 based activator of the present invention increase or upregulates expression of Klf-4 (e.g., by transcriptional activation) thus, facilitate the reprogramming or dedifferentiation of a cell to a more potent state.

The 5′-flanking region of the mouse Klf-4 transcription unit was sequenced and found to contain multiple cis-elements homologous to the binding sites of several established transcription factors including Sp1, AP-1, Cdx, LATA, and USF. In co-transfection experiments, Sp1, Sp3 and Cdx-2 transactivated a reporter gene linked to the Klf-4 1 kb 5′-flanking region (Mahatan C S, Kaestner K H, Geiman D E, Yang V W 1999. Characterization of the structure and regulation of the murine gene encoding gut-enriched Krüppel-like factor (Krüppel-like factor 4). Nucleic Acids Res 27(23):4562-4569).

In one embodiment, an activator of the invention is a polypeptide or fusion polypeptide that comprises the full-length Sp1, Sp3 and/or Cdx-2 or a functional fragment thereof that activates the expression of Klf-4 (e.g., transcriptional activation); thus, facilitating the reprogramming or dedifferentiation of a cell to a more potent state. Suitable Sp1, Sp3 and/or Cdx-2 based activators can be a Sp1, Sp3 and/or Cdx-2 mRNA, a Sp1, Sp3 and/or Cdx-2 specific bifunctional antisense oligonucleotide, a dsDNA comprising Sp1, Sp3 and/or Cdx-2, a Sp1, Sp3 and/or Cdx-2 polypeptide or an active fragment thereof, a peptidomimetics of Sp1, Sp3 and/or Cdx-2, peptoids of Sp1, Sp3 and/or Cdx-2, or a small organic molecule that mimics the transcriptional activity of Sp1, Sp3 and/or Cdx-2.

In particular embodiments, an artificial transcription factor comprises the Sp1, Sp3 or Cdx-2 polypeptide or a functional fragment thereof. In certain embodiments, the artificial transcription optionally comprises a membrane translocation peptide. A Sp1, Sp3 or Cdx-2 based activator of the present invention increase or upregulates expression of Klf-4 (e.g., by transcriptional activation) thus, facilitate the reprogramming or dedifferentiation of a cell to a more potent state.

The KLF9-induced mRNAs encode proteins which participate in: regulation and function of the actin cytoskeleton (COTL1, FSCN1, FXYD5, MYO10); cell adhesion, extracellular matrix and basement membrane formation (e.g., AMIGO2, COL4A1, COL4A2, LAMC2, NID2); transport (CLIC4); cellular signaling (e.g., BCAR3, MAPKAPK3); and transcriptional regulation (e.g., KLF4).

In one embodiment, an activator of the invention is a polypeptide or fusion polypeptide that comprises the full-length Klf-9 or a functional fragment thereof that activates the expression of Klf-4 (e.g., transcriptional activation); thus, facilitating the reprogramming or dedifferentiation of a cell to a more potent state. Suitable Klf-9 based activators can be a Klf-9 mRNA, a Klf-9 specific bifunctional antisense oligonucleotide, a dsDNA comprising Klf-9, a Klf-9 polypeptide or an active fragment thereof, a peptidomimetics of Klf-9, peptoids of Klf-9, or a small organic molecule that mimics the transcriptional activity of Klf-9.

In particular embodiments, an artificial transcription factor comprises the Klf-9 polypeptide or a functional fragment thereof. In certain embodiments, the artificial transcription optionally comprises a membrane translocation peptide. A Klf-9 based activator of the present invention increase or upregulates expression of Klf-4 (e.g., by transcriptional activation) thus, facilitate the reprogramming or dedifferentiation of a cell to a more potent state.

Lymphocytes circulate in a quiescent (G(0)) state until they encounter specific antigens. In T cells, quiescence is programmed by transcription factors of the forkhead box O (FOXO) and Krüppel-like factor (KLF) families. KLF4 is a candidate tumor suppressor gene in B lymphocytes, and thus a likely candidate for regulating B cell homeostasis. RNA and protein expression of murine KLF4 decreases following B cell activation. Forced expression of KLF4 in proliferating B cell blasts causes a G(1) cell cycle arrest. This effect requires the DNA binding and transactivation domains of KLF4 and correlates with changes in the expression of known KLF target genes. Klf4 is a target gene for FOXO transcription factors, which also suppress B cell proliferation.

In one embodiment, an activator of the invention is a polypeptide or fusion polypeptide that comprises the full-length FOXO or a functional fragment thereof that activates the expression of Klf-4 (e.g., transcriptional activation); thus, facilitating the reprogramming or dedifferentiation of a cell to a more potent state. Suitable FOXO based activators can be a FOXO mRNA, a FOXO specific bifunctional antisense oligonucleotide, a dsDNA comprising FOXO, a FOXO polypeptide or an active fragment thereof, a peptidomimetics of FOXO, peptoids of FOXO, or a small organic molecule that mimics the transcriptional activity of FOXO.

In particular embodiments, an artificial transcription factor comprises the FOXO polypeptide or a functional fragment thereof. In certain embodiments, the artificial transcription optionally comprises a membrane translocation peptide. A FOXO based activator of the present invention increase or upregulates expression of Klf-4 (e.g., by transcriptional activation) thus, facilitate the reprogramming or dedifferentiation of a cell to a more potent state.

To evaluate the effect of STAT1 on Klf-4 gene expression, a 2622-bp mouse Klf-4 promoter was isolated from a liver genomic library. In a transient transfection system, IFN-gamma treatment increased Klf-4 promoter activity by 3.5-fold. Sequential deletion and mutation analysis of the Klf-4 promoter has identified the sequence between −1675 and -1580, a region containing a GAS element, to be essential for IFN-gamma function. By electrophoretic mobility gel shift assay, nuclear extracts from IFN-gamma-stimulated HT-29 cells were found to bind to the GAS motif on the Klf-4promoter and this protein-DNA complex was supershifted by the STAT1 antiserum. These results indicate that IFN-gamma-induced Klf-4 expression required phosphorylated STAT1 and that these effects were mediated, in part, through interaction of STAT1 with the GAS element on the Klf-4 promoter.

In one embodiment, an activator of the invention is a polypeptide or fusion polypeptide that comprises the full-length STAT1 or a functional fragment thereof that activates the expression of Klf-4 (e.g., transcriptional activation); thus, facilitating the reprogramming or dedifferentiation of a cell to a more potent state. Suitable STAT1 based activators can be a STAT1 mRNA, a STAT1 specific bifunctional antisense oligonucleotide, a dsDNA comprising STAT1, a STAT1 polypeptide or an active fragment thereof, a peptidomimetics of STAT1, peptoids of STAT1, or a small organic molecule that mimics the transcriptional activity of STAT1.

In particular embodiments, an artificial transcription factor comprises the STAT1 polypeptide or a functional fragment thereof. In certain embodiments, the artificial transcription optionally comprises a membrane translocation peptide. A STAT1 based activator of the present invention increase or upregulates expression of Klf-4 (e.g., by transcriptional activation) thus, facilitate the reprogramming or dedifferentiation of a cell to a more potent state.

KLF4 (Krüppel-like factor 4 or gut-enriched Krüppel-like factor, GKLF) and KLF5 (Krüppel-like factor 5 or intestinal-enriched Krüppel-like factor, IKLF) are two closely related members of the zinc finger-containing Krüppel-like factor family of transcription factors. Although both genes are expressed in the intestinal epithelium, their distributions are different: Klf4 is primarily expressed in the terminally differentiated villus cells while Klf5 is primarily in the proliferating crypt cells. Previous studies show that Klf4 is a negative regulator of cell proliferation and Klf5 is a positive regulator of cell proliferation. In this study, we demonstrate that Klf5 binds to a number of cis-DNA elements that have previously been shown to bind to Klf4. However, while Klf4 activates the promoter of its own gene, Klf5 suppresses the Klf4 promoter. Moreover, Klf5 abrogates the activating effect of Klf4 on the Klf4 promoter and Klf4 abrogates the inhibitory effect of Klf5 on the same promoter. An explanation of this competing effect is due to physical competition of the two proteins for binding to cognate DNA sequence. The complementary tissue localization of expression of Klf4 and Klf5 and the opposing effect of the two Klfs on the Klf4 promoter activity may provide a basis for the coordinated regulation of expression of the Klf4 gene in the intestinal epithelium.

In one embodiment, an activator of the invention is a polypeptide or fusion polypeptide that comprises the full-length Klf4 and/or Klf-5 or a functional fragment thereof that activates the expression of Klf-4 (e.g., transcriptional activation); thus, facilitating the reprogramming or dedifferentiation of a cell to a more potent state. Suitable Klf4 and/or Klf-5 based activators can be a Klf4 and/or Klf-5 mRNA, a Klf4 and/or Klf-5 specific bifunctional antisense oligonucleotide, a dsDNA comprising Klf4 and/or Klf-5, a Klf4 and/or Klf-5 polypeptide or an active fragment thereof, a peptidomimetics of Klf4 and/or Klf-5, peptoids of Klf4 and/or Klf-5, or a small organic molecule that mimics the transcriptional activity of Klf4 and/or Klf-5.

In particular embodiments, an artificial transcription factor comprises the Klf4 and/or Klf-5 polypeptide or a functional fragment thereof. In certain embodiments, the artificial transcription optionally comprises a membrane translocation peptide. A Klf4 and/or Klf-5 based activator of the present invention increase or upregulates expression of Klf-4 (e.g., by transcriptional activation) thus, facilitate the reprogramming or dedifferentiation of a cell to a more potent state.

Illustrative examples of further activators of Klf-4 include, but are note limited to, cAMP, Mtf-1, PPARgamma, and Cdx2, among others.

In one embodiment, an activator of the invention is a polypeptide or fusion polypeptide that comprises the full-length cAMP, Mtf-1, PPARgamma, and/or Cdx2 or a functional fragment thereof that activates the expression of Klf-4 (e.g., transcriptional activation); thus, facilitating the reprogramming or dedifferentiation of a cell to a more potent state. Suitable cAMP, Mtf-1, PPARgamma, and/or Cdx2 based activators can be a cAMP, Mtf-1, PPARgamma, and/or Cdx2 mRNA, a cAMP, Mtf-1, PPARgamma, and/or Cdx2 specific bifunctional antisense oligonucleotide, a dsDNA comprising cAMP, Mff-1, PPARgamma, and/or Cdx2, a cAMP, Mtf-1, PPARgamma, and/or Cdx2 polypeptide or an active fragment thereof, a peptidomimetics of cAMP, Mtf-1, PPARgamma, and/or Cdx2, peptoids of cAMP, Mtf-1, PPARgamma, and/or Cdx2, or a small organic molecule that mimics the transcriptional activity of cAMP, Mtf-1, PPARgamma, and/or Cdx2.

In particular embodiments, an artificial transcription factor comprises the cAMP, Mtf-1, PPARgamma, and/or Cdx2 polypeptide or a functional fragment thereof. In certain embodiments, the artificial transcription optionally comprises a membrane translocation peptide. A cAMP, Mtf-1, PPARgamma, and/or Cdx2 based activator of the present invention increase or upregulates expression of Klf-4 (e.g., by transcriptional activation) thus, facilitate the reprogramming or dedifferentiation of a cell to a more potent state.

5. Repressors and Activators of Myc

Illustrative repressors of cMyc include, but are not limited to, APC, the Mad family of transcription factors, Mxi1, Mel18, Bmi1, and HIV1-TAT, among others.

In one embodiment, a repressor of the invention will target Mxi1, Mel18, Bmi1, and/or HIV1-TAT in order to relieve repression of Oct-4, and thus, facilitate the reprogramming or dedifferentiation of a cell to a more potent state. Suitable repressors for use in targeting Mxi1, Mel18, Bmi1, and/or HIV1-TAT, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Mxi1, Mel18, Bmi1, and/or HIV1-TAT in order to relieve repression of Oct-4, and thus, facilitate the reprogramming or dedifferentiation of a cell to a more potent state.

In another embodiment, the repressor is a transbody that binds to Mxi1, Mel18, Bmi1, and/or HIV1-TAT in order to prevent or suppress transcriptional repression of Oct-4 and thereby facilitate cellular reprogramming or dedifferentiation.

6. Exemplary Indirect Repressors and Activators

Pluri-potent bone marrow stromal cells (MSCs) provide an attractive opportunity to generate unlimited glucose-responsive insulin-producing cells for the treatment of diabetes. Two HMSC lines were transfected with three genes: PDX-1, NeuroD1 and Ngn3 without subsequent selection, followed by differentiation induction in vitro and transplantation into diabetic mice. Human MSCs expressed mRNAs of the archetypal stem cell markers: Sox2, Oct4, Nanog and CD34, and the endocrine cell markers: PDX-1, NeuroD1, Ngn3, and Nkx6.1.

In one embodiment, an activator of the invention is a polypeptide or fusion polypeptide that comprises the full-length PDX-1, NeuroD1, and/or Ngn3 or a functional fragment thereof that activates the expression of Sox2, Oct4, and Nanog (e.g., transcriptional activation); thus, facilitating the reprogramming or dedifferentiation of a cell to a more potent state. Suitable PDX-1, NeuroD1, and/or Ngn3 based activators can be a PDX-1, NeuroD1, and/or Ngn3 mRNA, a PDX-1, NeuroD1, and/or Ngn3 specific bifunctional antisense oligonucleotide, a dsDNA comprising PDX-1, NeuroD1, and/or Ngn3, a PDX-1, NeuroD1, and/or Ngn3 polypeptide or an active fragment thereof, a peptidomimetics of PDX-1, NeuroD1, and/or Ngn3, peptoids of PDX-1, NeuroD1, and/or Ngn3, or a small organic molecule that mimics the transcriptional activity of PDX-1, NeuroD1, and/or Ngn3.

In particular embodiments, an artificial transcription factor comprises the PDX-1, NeuroD1, and/or Ngn3 polypeptide or a functional fragment thereof. In certain embodiments, the artificial transcription optionally comprises a membrane translocation peptide. A PDX-1, NeuroD1, and/or Ngn3 based activator of the present invention increase or upregulates expression of Sox2, Oct4, and Nanog (e.g., by transcriptional activation) thus, facilitate the reprogramming or dedifferentiation of a cell to a more potent state.

Oct4, Sox2, and Nanog are key components of a core transcriptional regulatory network that controls the ability of embryonic stem cells to differentiate into all cell types. Here we show that Zfp281, a zinc finger transcription factor, is a key component of the network and that it is required to maintain pluripotency. Zfp281 was shown to directly activate Nanog expression by binding to a site in the promoter in very close proximity to the Oct4 and Sox2 binding sites. We present data showing that Zfp281 physically interacts with Oct4, Sox2, and Nanog. Chromatin immunoprecipitation experiments identified 2,417 genes that are direct targets for regulation by Zfp281, including several transcription factors that are known regulators of pluripotency, such as Oct4, Sox2, and Nanog. Gene expression microarray analysis indicated that some Zfp281 target genes were activated, whereas others were repressed, upon knockdown of Zfp281. The identification of both activation and repression domains within Zfp281 suggests that this transcription factor plays bifunctional roles in regulating gene expression within the network.

In one embodiment, a repressor of the invention will target Zfp281 in order to relieve repression of Oct4, Sox2, and Nanog, and thus, facilitate the reprogramming or dedifferentiation of a cell to a more potent state. Suitable repressors for use in targeting Zfp281, include but are not limited to an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule.

In particular embodiments, the repressor is an artificial transcription factor. In certain embodiments, the artificial transcription factor is a transcriptional repressor, optionally comprising a membrane translocation peptide that decreases, down-regulates, suppresses, and/or inhibits the transcription of Zfp281 in order to relieve repression of Oct4, Sox2, and Nanog, and thus, facilitate the reprogramming or dedifferentiation of a cell to a more potent state.

In another embodiment, the repressor is a transbody that binds to Zfp281 in order to prevent or suppress transcriptional repression of Oct4, Sox2, and Nanog and thereby facilitate cellular reprogramming or dedifferentiation.

One having ordinary skill in the art would recognize these examples are applicable to any of the transcription factors that act to increase transcriptional activation of a pluripotent gene or component of a cellular potency pathway as well as to those that increase transcriptional repression of a pluripotent gene or component of a cellular potency pathway.

### X. Polynucleotides

The present invention also provides isolated polynucleotides that encode a polypeptide of the invention and that are employed in the modulation, establishment and/or maintenance of pluripotency as described elsewhere herein (e.g., Sox-2, c-Myc, Oct3/4, Klf-4, Lin28, Nanog, hTERT etc., or a substrate, cofactor and/or downstream effector thereof), as well as compositions comprising such polynucleotides. Fusion polynucleotides that encode fusion polypeptides are also included in the present invention, as described elsewhere herein.

Nucleic acids can be synthesized using protocols known in the art as described in Caruthers et al., 1992, Methods in Enzymology 211, 3-19; Thompson et al., International PCT Publication No. WO 99/54459; Wincott et al., 1995, Nucleic Acids Res. 23, 2677-2684; Wincott et al., 1997, Methods Mol. Bio., 74, 59-68; Brennan et al., 1998, Biotechnol Bioeng., 61, 33-45; and Brennan, U.S. Pat. No. 6,001,311).

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other (see for example, Usman and McSwiggen, supra; Eckstein et al., International PCT Publication No. WO 92/07065; Usman et al., International PCT Publication No. WO 93/15187; Uhlman & Peyman, supra). There are several examples of modified nucleic acid bases known in the art as summarized by Limbach et al., (1994, Nucleic Acids Res. 22, 2183-2196).

Exemplary chemically modified and other natural nucleic acid bases that can be introduced into nucleic acids include, for example, inosine, purine, pyridin-4-one, pyridin-2-one, phenyl, pseudouracil, 2,4,6-trime115thoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5″-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, β-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine, thymine, and uracil at 1″ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1″ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other (see for example, Usman and McSwiggen, supra; Eckstein et al., International PCT Publication No. WO 92/07065; Usman et al., International PCT Publication No. WO 93/15187; Uhlman & Peyman). There are several examples of modified nucleic acid bases known in the art as summarized by Limbach et al., (1994, Nucleic Acids Res. 22, 2183-2196). Exemplary chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, β-D-mannosylqueosine, uridine-5-oxyacetic acid, 2-thiocytidine, threonine derivatives and others (Burgin et al., 1996, Biochemistry, 35, 14090-14097; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides, and the like. Such segments may be naturally isolated, recombinant, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide of the invention or a portion thereof) or may comprise a variant, or a biological functional equivalent of such a sequence. Polynucleotide variants may contain one or more substitutions, additions, deletions and/or insertions, as further described below, preferably such that the reprogramming or programming or potency modulating activity of the encoded polypeptide is not substantially diminished relative to the unmodified polypeptide.

As used herein, the term “homolog” means a gene related to a second gene by descent from a common ancestral DNA sequence. The term “homolog” may apply to the relationship between genes separated by speciation (e.g., ortholog), or to the relationship between genes originating via genetic duplication (e.g., paralog).

As used herein, the term “ortholog” refers to genes in different species that have evolved from a common ancestral gene via speciation. Orthologs often (but certainly not always) retain the same function(s) during the course of evolution. Thus, functions may be lost or gained when comparing a pair of orthologs.

As used herein, the term “paralogs” refers to genes produced via gene duplication within a genome. Paralogs typically evolve new functions or else eventually become pseudogenes.

Also included are polynucleotides that hybridize to polynucleotides that encode a polypeptide of the invention. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

High stringency hybridization conditions are conditions that enable a probe, primer or oligonucleotide to hybridize only to its target sequence. Stringent conditions are sequence-dependent and will differ. Stringent conditions comprise: (1) low ionic strength and high temperature washes (e.g. 15 mM sodium chloride, 1.5 mM sodium citrate, 0.1% sodium dodecyl sulfate at 50° C.); (2) a denaturing agent during hybridization (e.g. 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, Sonicated salmon sperm DNA (50 .mu.g/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% identical to each other typically remain hybridized to each other. These conditions are presented as examples and are not meant to be limiting.

Moderately stringent conditionsare conditions that use washing solutions and hybridization conditions that are less stringent (Sambrook, 1989) than thos for high stringency, such that a polynucleotide will hybridize to the entire, fragments, derivatives or analogs of nucleic acids of the present invention. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described in (Ausubel et al., 1987; Kriegler, 1990).

Low stringent conditions are conditions that use washing solutions and hybridization conditions that are less stringent than those for moderate stringency (Sambrook, 1989), such that a polynucleotide will hybridize to the entire, fragments, derivatives or analogs of nucleic acids of the present invention. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described in (Ausubel et al., 1987; Kriegler, 1990; Shilo and Weinberg, 1981).

In additional embodiments, the present invention provides isolated polynucleotides comprising various lengths of contiguous stretches of sequence identical to or complementary to a polynucleotide encoding a polypeptide as described herein. For example, polynucleotides are provided by this invention that encode at least about 5, 10, 25, 50, 100, 150, 200, 250, 300, 350, 400, 500, 1000 or more contiguous amino acid residues of a polypeptide of the invention, as well as all intermediate lengths. It will be readily understood that “intermediate lengths”, in this context, means any length between the quoted values, such as 6, 7, 8, 9, etc., 101, 102, 103, etc.; 151, 152, 153, etc.; 201, 202, 203, etc.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. Moreover, it will be appreciated by those of ordinary skill in the art that, as a result of the degeneracy of the genetic code, there are many nucleotide sequences that encode a polypeptide as described herein, including polynucleotides that are optimized for human and/or primate codon selection. Further, alleles of the genes comprising the polynucleotide sequences provided herein may also be used.

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells. Amplificaiton techniques are routine in the art.

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. The vector components generally include, but are not limited to, one or more of the following: a signal sequence, an origin of replication, one or more marker genes, an enhancer element, a promoter that is recognized by the host organism, and a transcription termination sequence. Specific initiation signals may also be used to achieve more efficient translation of sequences encoding a polypeptide of interest.

A polypeptide of the invention may be produced recombinantly not only directly, but also as a fusion polypeptide with a heterologous polypeptide, which is preferably a signal sequence or other polypeptide having a specific cleavage site at the N-terminus of the mature protein or polypeptide.

Expression and cloning vectors may contain a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli.

For long-term, high-yield production of recombinant proteins, stable expression is generally preferred. Resistant clones of stably transformed cells may be proliferated using tissue culture techniques appropriate to the cell type.

Host cell strains may be chosen for their ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### XI. Polypeptides

As noted above, the present invention, in certain aspects, provides methods for inducing, modulating and/or maintaining pluripotency by administering polypeptide-based pluripotency factors (e.g., Sox-2, c-Myc, Oct3/4, Klf-4, Lin28, Nanog, hTERT, etc.), or by administering polynucleotides encoding such polypeptides, using techniques known and available in the art.

As used herein, the terms “polypeptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. Polypeptides of the invention may be prepared using any of a variety of well known recombinant and/or synthetic techniques, illustrative examples of which are further discussed below.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R.§§1.821-1.822, abbreviations for amino acid residues are shown in Table 1:

TABLE 1
Table of Amino Acid Nomenclature

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

A polypeptide variant may differ from a naturally occurring polypeptide in one or more substitutions, deletions, additions and/or insertions. Such variants may be naturally occurring or may be synthetically generated, for example, by modifying one or more of the above polypeptide sequences used in the methods of the invention and evaluating their effects using any of a number of techniques well known in the art.

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with an ability to modulate, induce and/or maintain pluripotency as described herein. One skilled in the art, for example, can change one or more of the codons of the encoding DNA sequence, e.g., according to Table 2.

TABLE 2
Amino Acid Codons

| Amino Acids | Codons | | | | | |
|---|---|---|---|---|---|---|
| Alanine | GCA | GCC | GCG | GCU | | |
| Cysteine | UGC | UGU | | | | |
| Aspartic acid | GAC | GAU | | | | |
| Glutamic acid | GAA | GAG | | | | |
| Phenylalanine | UUC | UUU | | | | |
| Glycine | GGA | GGC | GGG | GGU | | |
| Histidine | CAC | CAU | | | | |
| Isoleucine | AUA | AUC | AUU | | | |
| Lysine | AAA | AAG | | | | |
| Leucine | UUA | UUG | CUA | CUC | CUG | CUU |
| Methionine | AUG | | | | | |
| Asparagine | AAC | AAU | | | | |
| Proline | CCA | CCC | CCG | CCU | | |
| Glutamine | CAA | CAG | | | | |
| Arginine | AGA | AGG | CGA | CGC | CGG | CGU |
| Serine | AGC | AGU | UCA | UCC | UCG | UCU |
| Threonine | ACA | ACC | ACG | ACU | | |
| Valine | GUA | GUC | GUG | GUU | | |
| Tryptophan | UGG | | | | | |
| Tyrosine | UAC | UAU | | | | |

Guidance in determining which amino acid residues can be substituted, inserted, or deleted without abolishing biological or immunological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

Such substitutions may be made in accordance with those set forth in TABLE 3 as follows:

TABLE 3
Conservative Amino Acid Substitutions

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

In making such changes, the hydropathic index of amino acids may be considered. The importance of the hydropathic amino acid index in conferring interactive biologic function on a protein is generally understood in the art (Kyte and Doolittle, 1982, incorporated herein by reference). For example, it is known that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Variants of the polypeptides of the invention include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (Mark et al., U.S. Pat. No. 4,959,314).

Of particular interest are substitutions of charged amino acids with another charged amino acid and with neutral or negatively charged amino acids. The latter results in proteins with reduced positive charge to improve the characteristics of the disclosed proteins. The prevention of aggregation is highly desirable (Pinckard et al., Clin. Exp. Immunol. 2:331-340, 1967; Robbins et al., Diabetes 36:838-845, 1987; Cleland et al., Crit. Rev. Therapeutic Drug Carrier Systems 10:307-377, 1993).

Amino acids in polypeptides of the present invention that are essential for function can be identified by methods known in the art, such as site-directed mutagenesis or alanine-scanning mutagenesis (Cunningham and Wells, Science 244:1081-1085, 1989). Sites that are critical for ligand-receptor binding can also be determined by structural analysis such as crystallization, nuclear magnetic resonance or photoaffinity labeling (Smith et al., J. Mol. Biol. 224:899-904, 1992 and de Vos et al. Science 255:306-312, 1992).

Certain changes do not significantly affect the folding or activity of the protein. The number of amino acid substitutions a skilled artisan would make depends on many factors, including those described above. Generally speaking, the number of substitutions for any given polypeptide will not be more than 50, 40, 30, 25, 20, 15, 10, 5 or 3.

In addition, pegylation of polypeptides and/or muteins is expected to provide improved properties, such as increased half-life, solubility, and protease resistance. Pegylation is well known in the art.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Nat'l Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. The BLAST and BLAST 2.0 algorithms, which are described in Altschul et al. (1977) Nucl. Acids Res. 25:3389-3402 and Altschul et al. (1990) J. Mol. Biol. 215:403-410, respectively.

In certain embodiments of the invention, there are provided fusion polypeptides, and polynucleotides encoding fusion polypeptides. Fusion polypeptide and fusion proteins refer to a polypeptide of the invention that has been covalently linked, either directly or via an amino acid linker, to one or more heterologous polypeptide sequences (fusion partners). The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

In one embodiment, a fusion protein may be designed to encode multiple pluripotency factors as described herein, from a single transcript. In another embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments. Still further fusion partners include affinity tags, which facilitate purification of the protein. Fusion polypeptides of the present invention also include, but are not limited to artificially designed transcription factors, as described elsewhere herein.

Fusion polypeptides may be produced by chemical synthetic methods or by chemical linkage between the two moieties or may generally be prepared using other standard techniques. In particular embodiments, it is preferred that fusion polypeptides are produced by fusion of a coding sequence of a cell-specific targeting moiety and a coding sequence of polypeptide-based repressor and/or activator of the present invention. In certain embodiments, the preferred repressor/activator is a transcription factor. In certain related embodiments, the transcription factor is a transcriptional activator or a transcriptional repressor. In further certain related embodiments, a cell-specific targeting moiety is fused to an artificial transcription factor as described elsewhere herein.

A peptide linker sequence may be employed to separate the first and second polypeptide components by a distance sufficient to ensure that each polypeptide folds into its secondary and tertiary structures, if desired. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. A particular example is the flexible polylinker composed of the pentamer Gly-Gly-Gly-Gly-Ser repeated 1 to 3 times (Bird et al., 1988, Science 242:423-426; Huston et al., 1988, Proc. Natl. Acad. Sci. U.S.A. 85:5979-5883); and (Chaudhary et al., 1990, Proc. Natl. Acad. Sci. U.S.A. 87:1066-1070).

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

The present invention also provides for cell-permeating fusion polypeptides. Proteins, lipids and other compounds, which have the ability to translocate polypeptides across a cell membrane, have been described. For example, “membrane translocation polypeptides” have amphiphilic or hydrophobic amino acid subsequences that have the ability to act as membrane-translocating carriers.

Examples of peptide sequences which can facilitate protein uptake into cells include, but are not limited to: an 11 amino acid peptide of the tat protein of HIV; a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). Other suitable chemical moieties that provide enhanced cellular uptake can also be linked, either covalently or non-covalently, to a polypeptide of the present invention (e.g., peptide, protein, peptidomimetics, peptoids, ATF, and the like).

Toxin molecules also have the ability to transport polypeptides across cell membranes. Often, such molecules (called “binary toxins”) are composed of at least two parts: a translocation or binding domain and a separate toxin domain. Typically, the translocation domain, which can optionally be a polypeptide, binds to a cellular receptor, facilitating transport of the toxin into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193. Such subsequences can be fused to a polypeptide and thereby used to translocate the polypeptide, including the polypeptides disclosed herein, across a cell membrane.

The present invention contemplates, in part, to provide repressors and/or activators as discussed herein throughout to cells ex vivo or in vivo, directly, in order to alter the potency of the cell (i.e., to reprogram and/or program the cell).

Thus, in one embodiment, the present invention provides compositions comprising one or more repressors and/or activators of the present invention as discussed herein throughout, wherein at least one repressor and/or activator is cell permeable (e.g., fused to one or more membrane translocation polypeptides), and that modulates at least one component of a cell potency pathway.

In particular embodiments, the present invention provides a method to alter the potency of a cell (e.g., reprogram or program) comprising contacting the cell with at least one repressor and/or activator, or a composition comprising the same, wherein at least one repressor and/or activator is cell permeable, to modulate at least one component of a pathway(s) associated with the potency of a cell, thereby reprogramming the cell. In particular related embodiments, a method of altering the potency of a cell, wherein the alteration is reprogramming, said method further comprises the step of programming the cell to a desired mature somatic cell.

In certain embodiments, the programming is accomplished by contacting a reprogrammed cell of the present invention with one or more repressors and/or activators, or a composition comprising the same, wherein at least one repressor and/or activator is cell permeable, to modulate at least one component of a pathway(s) associated with the potency of a cell, thereby programming the cell.

### XII. Antibodies

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (K) and lambda (A), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., PNAS USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., PNAS USA, 103:3557-3562 (2006) regarding human antibodies generated via a human B-cell hybridoma technology.

An “antigen” is a predetermined moiety to which an antibody can selectively bind. The target antigen may be polypeptide, carbohydrate, nucleic acid, lipid, hapten or other naturally occurring or synthetic compound. Preferably, the target antigen is a polypeptide.

An “acceptor human framework” for the purposes herein is a framework comprising the amino acid sequence of a VL or VH framework derived from a human immunoglobulin framework, or from a human consensus framework. An acceptor human framework “derived from” a human immunoglobulin framework or human consensus framework may comprise the same amino acid sequence thereof, or may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H and 78H; for instance, the amino acid residues at those positions may be 71A, 73T and/or 78A. In one embodiment, the VL acceptor human framework is identical in sequence to the VL human immunoglobulin framework sequence or human consensus framework sequence. Where pre-existing amino acid changes are present in a VH, preferably those changes are only at three, two or one of positions 71H, 73H and 78H; for instance, the amino acid residues at those positions may be 71A, 73T and/or 78A. In one embodiment, the VL acceptor human framework is identical in sequence to the VL human immunoglobulin framework sequence or human consensus framework sequence.

A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. In one embodiment, for the VL, the subgroup is subgroup kappa I as in Kabat et al. In one embodiment, for the VH, the subgroup is subgroup III as in Kabat et al.

A “VH subgroup III consensus framework” comprises the consensus sequence obtained from the amino acid sequences in variable heavy subgroup III of Kabat et al.

A “VL subgroup I consensus framework” comprises the consensus sequence obtained from the amino acid sequences in variable light kappa subgroup I of Kabat et al.

An “unmodified human framework” is a human framework which has the same amino acid sequence as the acceptor human framework, e.g. lacking human to non-human amino acid substitution(s) in the acceptor human framework.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are HVRs that are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures.

The amino acid position/boundary delineating a hypervariable region of an antibody can vary, depending on the context and the various definitions known in the art (as described below). Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. In one embodiment, these hybrid hypervariable positions include one or more of positions 26-30, 26-35 33-35B, 47-49, 49-65, 57-65, 95-102, 93, 94 and 102 in a heavy chain variable domain. In one embodiment, these hybrid hypervariable positions include one or more of positions 24-29, 24-34, 35-36, 46-49, 50-56, 89-97, 56 and 97 in a light chain variable domain.

As used herein, the HVRs of the light chain are referred to interchangeably as HVR-L1, -L2, or -L3, or HVR1-LC, HVR2-LC or HVR3-LC or other similar designation that indicates that a light chain HVR is referenced. As used herein, the HVRs of the heavy chain are referred to interchangeably as HVR-H1, -H2, or -H3, or HVR1-HC, HVR2-HC, or HVR3-HC, or other similar designation that indicates that a heavy chain HVR is referenced.

Hypervariable regions may comprise “extended hypervariable regions” as follows: 24-36 or 24-34 (L1), 46-56 or 50-56 (L2) and 89-97 (L3) in the VL and 26-35 (H1), 50-65 or 49-65 (H2) and 93-102, 94-102 or 95-102 (H3) in the VH. The variable domain residues are numbered according to Kabat et al., supra for each of these definitions.

An “altered hypervariable region” for the purposes herein is a hypervariable region comprising one or more (e.g. one to about 16) amino acid substitution(s) therein.

An “un-modified hypervariable region” for the purposes herein is a hypervariable region having the same amino acid sequence as a non-human antibody from which it was derived, i.e. one which lacks one or more amino acid substitutions therein.

“Framework” or “FR” residues are those variable domain residues other than the hypervariable region residues as herein defined. As used herein, LC-FR1-4 or FR1-4-LC or similar designation is used interchangeably and refers to framework regions of the light chain. As used herein, HC-FR1-4 or FR1-4-HC or similar designation is used interchangeably and refers to framework region of the heavy chain.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al., Bio/Technology 10:779-783 (1992) describes affinity maturation by VH and VL domain shuffling. Random mutagenesis of CDR and/or framework residues is described by: Barbas et al., PNAS USA 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al, J. Mol. Biol. 226:889-896 (1992).

A “blocking” antibody, an “antagonist” antibody, or a “repressor antibody” is one which represses, inhibits or reduces biological activity of the antigen it binds. For example repressor antibodies directed against a pluripotency factor substantially or completely inhibit the effect of the pluripotency factor.

An “activating” antibody or an “agonist” antibody is one which activates, stimulates, and/or maintains biological activity of the antigen it binds. For example activator antibodies directed against a pluripotency factor substantially or completely stimulate the effect of the pluripotency factor.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd,” “KD,” or “Kd value,” is measured by a radiolabeled antigen binding assay (RIA) performed with the Fab version of an antibody of interest and its antigen as described by the following assay that measures solution binding affinity of Fabs for antigen by equilibrating Fab with a minimal concentration of (125I)-labeled antigen in the presence of a titration series of unlabeled antigen, then capturing bound antigen with an anti-Fab antibody-coated plate (Chen, et al., (1999) J. Mol. Biol 293:865-881).

According to another embodiment the Kd or Kd value is measured by using surface plasmon resonance assays using a BIAcore™-2000 or a BIAcore™-3000 (BIAcore, Inc., Piscataway, N.J.) at 25° C. with immobilized antigen CM5 chips at ^{˜}10 response units (RU). Association rates (kon) and dissociation rates (koff) are calculated using a simple one-to-one Langmuir binding model (BIAcore Evaluation Software version 3.2) by simultaneous fitting the association and dissociation sensorgram. The equilibrium dissociation constant (Kd) is calculated as the ratio koff/kon. See, e.g., Chen et al., J. Mol. Biol 293:865-881 (1999). If the on-rate exceeds 106 M-1S-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco™ spectrophotometer (ThermoSpectronic) with a stirred cuvette.

A. Antibody Fragments

The present invention encompasses antibody fragments. Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to tissues. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10: 163-167 (1992)). According to another approach, F(ab′)2 fragments can be isolated directly from recombinant host cell culture. Fab and F(ab′)2 fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

B. Humanized Antibodies

The invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody. See, e.g., Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies. See, e.g., Carter et al. (1992) PNAS USA, 89:4285; Presta et al. (1993) J. Immunol., 151:2623.

It is further generally desirable that antibodies be humanized with retention of high affinity for the antigen and other favorable biological properties. In general, the hypervariable region residues are directly and most substantially involved in influencing antigen binding.

In some embodiments, the invention provides antibodies that are humanized such that HAMA response is reduced or eliminated. Reduction or elimination of a HAMA response is a significant aspect of clinical development of suitable therapeutic agents. See, e.g., Khaxzaeli et al., J. Natl. Cancer Inst. (1988), 80:937; Jaffers et al., Transplantation (1986), 41:572; Shawler et al., J. Immunol. (1985), 135:1530; Sears et al., J. Biol. Response Mod. (1984), 3:138; Miller et al., Blood (1983), 62:988; Hakimi et al., J. Immunol. (1991), 147:1352; Reichmann et al., Nature (1988), 332:323; Junghans et al., Cancer Res. (1990), 50:1495. Variants of these antibodies can further be obtained using routine methods known in the art, some of which are further described below.

C. Human Antibodies

Human antibodies of the invention can be constructed by combining Fv clone variable domain sequence(s) selected from human-derived phage display libraries with known human constant domain sequences(s) as described above. Alternatively, human monoclonal antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is now possible to produce transgenic animals (e.g. mice) that are capable, upon immunization, of producing a full repertoire of human antibodies in the absence of endogenous immunoglobulin production. See, e.g., Jakobovits et al., PNAS USA, 90: 2551 (1993); Jakobovits et al., Nature, 362: 255 (1993); Bruggermann et al., Year in Immunol., 7: 33 (1993).

Gene shuffling can also be used to derive human antibodies from non-human, e.g., rodent antibodies, where the human antibody has similar affinities and specificities to the starting non-human antibody. (see PCT WO 93/06213 published Apr. 1, 1993). Unlike traditional humanization of non-human antibodies by CDR grafting, this technique provides completely human antibodies, which have no FR or CDR residues of non-human origin.

D. Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody of the invention is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. (1997) TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

For example, antibody variants are provided having a carbohydrate structure that lacks fucose attached (directly or indirectly) to an Fc region. Such variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec 13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, an antibody variant comprises an Fc region with one or more amino acid substitutions which further improve ADCC, for example, substitutions at positions 298, 333, and/or 334 of the Fc region (Eu numbering of residues). Such substitutions may occur in combination with any of the variations described above.

In certain embodiments, the invention contemplates, in part, an antibody variant that possesses some but not all effector functions, which make it a desirable candidate for many applications in which the half life of the antibody in vivo is important yet certain effector functions (such as complement and ADCC) are unnecessary or deleterious. In certain embodiments, the Fc activities of the antibody are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). Non-limiting examples of in vitro assays to assess ADCC activity of a molecule of interest is described in U.S. Pat. No. 5,500,362 (see, e.g. Hellstrom, I., et al. PNAS USA 83:7059-7063 (1986)) and Hellstrom, I et al., PNAS USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS USA 95:652-656 (1998). Cl q binding assays may also be carried out to confirm that the antibody is unable to bind Clq and hence lacks CDC activity. To assess complement activation, a CDC assay may be performed (see, for example, Gazzano-Santoro et al., J. Immunol. Methods 202:163 (1996); Cragg, M. S. et al., Blood 101: 1045-1052 (2003); and Cragg, M. S, and M. J. Glennie, Blood 103:2738-2743 (2004)). FcRn binding and in vivo clearance/half life determinations can also be performed using methods known in the art (see, for example, Petkova, S. B. et al., Intl Immunol. 18(12):1759-1769 (2006)).

Other antibody variants having one or more amino acid substitutions are provided. Sites of interest for substitutional mutagenesis include the hypervariable regions, but FR alterations are also contemplated.

Modifications in the biological properties of an antibody may be accomplished by selecting substitutions that affect (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

Alternatively, naturally occurring residues may be divided into groups based on common side-chain properties, as described elsewhere herein.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g., a humanized or human antibody). Generally, the resulting variant(s) selected for further development will have modified (e.g., improved) biological properties relative to the parent antibody from which they are generated. An exemplary substitutional variant is an affinity matured antibody, which may be conveniently generated using phage display-based affinity maturation techniques. Briefly, several hypervariable region sites (e.g., 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g., binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and variants with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the invention, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions including that of a hinge cysteine.

In accordance with this description and the teachings of the art, it is contemplated that in some embodiments, an antibody of the invention may comprise one or more alterations as compared to the wild type counterpart antibody, e.g., in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter, Nature 322:738-40 (1988); U.S. Pat. No. 5,648,260; U.S. Pat. No. 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

In another aspect, the invention provides antibodies comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

In yet another aspect, it may be desirable to create cysteine engineered antibodies, e.g., “thioMAbs,” and “thioFabs” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. In a preferred embodiment, A118 (EU numbering) of the heavy chain is substituted for cysteine. Cysteine engineered thioMabs and thioFabs are described in further detail herein below.

E. Antibody Derivatives

The antibodies of the present invention can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., PNAS USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32 and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies.

F. Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fusion proteins, and antibody fragments can be produced in bacteria, in particular when glycosylation and Fc effector function are not needed, such as when the therapeutic antibody is conjugated to a cytotoxic agent (e.g., a toxin) that by itself shows effectiveness in tumor cell destruction. For expression of antibody fragments and polypeptides in bacteria, see, e.g., U.S. Pat. No. 5,648,237 (Carter et. al.), U.S. Pat. No. 5,789,199 (Joly et al.), U.S. Pat. No. 5,840,523 (Simmons et al.). See also Charlton, Methods in Molecular Biology, Vol. 248 (B. K. C. Lo, ed., Humana Press, Totowa, N.J., 2003), pp. 245-254, describing expression of antibody fragments in E. coli.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger. For a review discussing the use of yeasts and filamentous fungi for the production of therapeutic proteins, see, e.g., Gerngross, Nat. Biotech. 22:1409-1414 (2004).

Certain fungi and yeast strains may be selected in which glycosylation pathways have been “humanized,” resulting in the production of an antibody with a partially or fully human glycosylation pattern. See, e.g., L1 et al., Nat. Biotech. 24:210-215 (2006) (describing humanization of the glycosylation pathway in Pichia pastoris); and Gerngross et al., supra.

Suitable host cells for the expression of glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, duckweed (Lemnaceae), alfalfa (M. truncatula), and tobacco can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR—CHO cells (Urlaub et al., PNAS USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C Lo, ed., Humana Press, Totowa, N.J., 2003), pp. 255-268.

In one technique, an immunogen comprising the polypeptide is initially injected into any of a wide variety of mammals (e.g., mice, rats, rabbits, sheep or goats). Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include 90Y, 123I, 125I, 131I, 186Re, 188Re, 211At, and 212Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A linker group can function as a spacer to distance an antibody from an agent in order to avoid interference with binding capabilities. A linker group can also serve to increase the chemical reactivity of a substituent on an agent or an antibody, and thus increase the coupling efficiency.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Also included are cleavable linker groups. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

Also provided herein are anti-idiotypic antibodies that mimic an immunogenic portion of a polypeptide of the present invention. Anti-idiotypic antibodies that mimic an immunogenic portion of a polypeptide of the present invention are those antibodies that bind to an antibody, or antigen-binding fragment thereof, that specifically binds to an immunogenic portion of a polypeptide of the present invention, as described herein.

### XIII. Formulations and Pharmaceutical Compositions

The formulations and compositions of the invention may comprise one or more repressors and/or activators comprised of a combination of any number of polypeptides, polynucleotides, cells, and small molecules, as described herein, formulated in pharmaceutically-acceptable or physiologically-acceptable solutions (e.g., culture medium) for administration to a cell or an animal, either alone, or in combination with one or more other modalities of therapy.

As described in detail below, the pharmaceutical compositions of the present invention comprising a combination of one or more of: i) a cell; ii) a repressor; iii) an activator; and iv) a pharmaceutically acceptable cell culture medium; may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

An “effective amount” refers to an amount effective, at dosages and for periods of time necessary, to achieve the desired therapeutic or prophylactic result. A “therapeutically effective amount” of one or more repressors and/or activators of the invention, or a composition comprising the same, may vary according to factors such as the disease state, age, sex, and weight of the individual, and the ability of the one or more repressors and/or activators to elicit a desired response in the individual. A therapeutically effective amount is also one in which any toxic or detrimental effects of the one or more repressors and/or activators are outweighed by the therapeutically beneficial effects. The term “therapeutically effective amount” includes an amount that is effective to “treat” a disease or disorder in a mammal (e.g., a patient).

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount is less than the therapeutically effective amount.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) a pharmaceutically acceptable cell culture medium; and (23) other non-toxic compatible substances employed in pharmaceutical formulations.

Certain embodiments include “pharmaceutically-acceptable salts,” including hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19). Additional examples include base addition salts such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

In another embodiment, the amount of active ingredient in a single dosage from that is required to produce a therapeutic effect is about 0.1% active ingredient, about 1% active ingredient, about 5% active ingredient, about 10% active ingredient, about 15% active ingredient, about 20% active ingredient, about 25% active ingredient, about 30% active ingredient, about 35% active ingredient, about 40% active ingredient, about 45% active ingredient, about 50% active ingredient, about 55% active ingredient, about 60% active ingredient, about 65% active ingredient, about 70% active ingredient, about 75% active ingredient, about 80% active ingredient, about 85% active ingredient, about 90% active ingredient, or about 95% active ingredient or more, including all ranges of such values.

In certain embodiments, a formulation of the present invention comprises an excipient selected from the group consisting of cyclodextrins and derivatives, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable one or more repressors and/or activators of the present invention.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a modulating agent as provided herein include powders, sprays, ointments, pastes, creams, lotions, gels, solutions, patches and inhalants. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Absorption enhancers can also be used to increase the flux of the agent across the skin.

Ophthalmic formulations, eye ointments, powders, solutions and the like, are also contemplated as being within the scope of this invention.

Pharmaceutical compositions of this invention suitable for parenteral administration comprise pharmaceutically-acceptable sterile isotonic aqueous (e.g., pharmaceutically acceptable culture medium) or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Injectable depot forms are made by forming microencapsule matrices of the subject compounds in biodegradable polymers such as polylactide-polyglycolide. Examples of other biodegradable polymers include poly-(orthoesters) and poly-(anhydrides).

In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C, et al., J Pharm Sci 80(7), 712-714, 1991).

The phrases “parenteral administration” and “administered parenterally” as used herein means-modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

In general, a suitable daily dose of a composition comprising one or more of a repressor, activator, or cell of the invention will be that amount of the which is the lowest dose effective to produce a therapeutic effect. Administration of one or more repressors, activators, and/or cells can be performed in a single composition or multiple compositions, separately or at the same time.

An effective dose will generally depend upon the factors described above. Generally, oral, intravenous, intracerebroventricular and subcutaneous doses of the repressors and/or activators of this invention for a patient, will range from about 0.000001 to about 1000 mg per kilogram, about 0.000005 to about 950 mg per kilogram, about 0.00001 to about 850 mg per kilogram, about 0.00005 to about 750 mg per kilogram, about 0.0001 to about 500 mg per kilogram, about 0.0005 to about 250 mg per kilogram, about 0.001 to about 100 mg per kilogram, about 0.001 to about 50 mg per kilogram, about 0.001 to about 25 mg per kilogram, about 0.001 to about 10 mg per kilogram, about 0.001 to about 1 mg per kilogram, about 0.005 to about 100 mg per kilogram, about 0.005 to about 50 mg per kilogram, about 0.005 to about 25 mg per kilogram, about 0.005 to about 10 mg per kilogram, about 0.005 to about 1 mg per kilogram, about 0.01 to about 100 mg per kilogram, about 0.01 to about 50 mg per kilogram, about 0.01 to about 25 mg per kilogram, about 0.01 to about 10 mg per kilogram, about 0.01 to about 1 mg per kilogram, about 0.05 to about 50 mg per kilogram, about 0.05 to about 25 mg per kilogram, about 0.05 to about 10 mg per kilogram, about 0.05 to about 1 mg per kilogram, about 0.1 to about 25 mg per kilogram, about 0.1 to about 10 mg per kilogram, about 0.1 to about 1 mg per kilogram, about 0.1 to about 0.5 mg per kilogram of body weight per day.

In another embodiment, one or more repressors and/or activators is administered orally or parenterally to a subject at a dose of about 0.25 to 3g per kg, about 0.5 to 2.5 g per kg, about 1 to 2g per kg, about 1.25 to 1.75 g per kg or about 1.5 g per kg of bodyweight per day.

In particular embodiments, one or more repressors and/or activators is administered orally or parenterally to a subject at a dose of about 10 g per kg, about 0.25 g per kg, about 0.50 g per kg, about 0.75 g per kg, about 1.0 g per kg, about 1.25 g per kg, about 1.50 g per kg, about 1.75 g per kg, or about 2.00 g per kg of bodyweight per day.

In other related embodiments, one or more repressors and/or activators is administered orally or parenterally to a subject at a dose of about 0.01 μg to 1 mg per kg, about 0.1 to 100 μg per kg, or about 1 to 10 μg per kg or any increment of concentration in between. For example, in particular embodiments, one or more repressors and/or activators is administered orally or parenterally to a subject at a dose of about 1 pg per kg, about 2 pg per kg, about 3 μg per kg, about 4 μg per kg, about 5 μg per kg, about 6 μg per kg, about 7 μg per kg, about 8 μg per kg, about 9 μg per kg, or about 10 μg per kg.

In particular embodiments, one or more repressors and/or activators is administered orally or parenterally to a subject at a dose of about 0.005 μg per kg, about 0.01 μg per kg, about 1.0 μg per kg, about 10 μg per kg, about 50 μg per kg, about 100 μg per kg, about 250 μg per kg, about 500 μg per kg, or about 1000 μg per kg

In certain embodiments of the present invention, compositions comprising reprogrammed or programmed cells and optionally comprising one or more repressors and/or activators can further comprise sterile saline, Ringer's solution, Hanks Balanced Salt Solution (HBSS), or Isolyte S, pH 7.4, serum free cellular media, or another pharmaceutically acceptable medium (e.g., pluripotent stem cell culture medium), as discussed elsewhere herein.

In related embodiments, the number of an effective amount of reprogrammed or programmed cells administered to a mammal in need thereof is between about 1×10⁴ and about 1×10¹³ cells per 100 kg of mammal. In some embodiments, the number of an effective amount of reprogrammed or programmed administered is between about 1×10⁶ and about 1×10⁹ cells per 100 kg or between about 1×10⁸ and about 1×10¹² cells per 100 kg. In some embodiments, the number of an effective amount of reprogrammed or programmed cells administered is between about 1×10⁹ and about 5×10¹¹ cells per 100 kg. In some embodiments, the number of an effective amount of reprogrammed or programmed cells administered is about 5×10¹⁰ cells per 100 kg. In some embodiments, the number of an effective amount of reprogrammed or programmed cells administered is 1×10¹⁰ cells per 100 kg.

In particular related embodiments, the number of an effective amount of reprogrammed or programmed cells administered in combination with one or more repressors and/or activators and/or a pharmaceutically acceptable cell culture medium is about or less than about 1×10¹² cells per 100 kg, about 1×10¹¹ cells per 100 kg, about 1×10¹⁰ cells per 100 kg, about 1×10⁹ cells per 100 kg, about 1×10⁸ cells per 100 kg, about 1×10⁷ cells per 100 kg, about 5×10⁶ cells per 100 kg, about 4×10⁶ cells per 100 kg, about 3×10⁶ cells per 100 kg, about 2×10⁶ cells per 100 kg, about 1×10⁶ cells per 100 kg, about 5×10⁵ cells per 100 kg, about 4×10⁵ cells per 100 kg, about 3×10⁵ cells per 100 kg, about 2×10⁵ cells per 100 kg, about 1×10⁵ cells per 100 kg, about 5×10⁴ cells per 100 kg, about 1×10⁴ cells per 100 kg, or about 1×10³ cells per 100 kg. One of ordinary skill in the art would be able to use routine methods in order to determine the correct dosage of an effective amount of reprogrammed or programmed cells for methods of the present invention.

A composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

### XIV. Methods of Delivery

The present invention contemplates, in part, to reprogram or program cells by contacting said cells with one or more repressors and/or activators, wherein the repressors or activators are nucleic acids, polypeptides, small molecules or any number and combination of the foregoing molecules, wherein the one or more repressors and/or activators modulates a component of a cellular potency pathway, thereby reprogramming or programming the cell. It is contemplated that the cells of the invention may be contacted in vitro, ex vivo, or in vivo.

In one embodiment, cells are contacted with a composition comprising one or more repressors and/or activators, wherein the repressors or activators are nucleic acids, polypeptides, small molecules or any number and combination of the foregoing molecules, wherein the one or more repressors and/or activators modulates a component of a cellular potency pathway, thereby reprogramming or programming the cell. It is contemplated that the cells of the invention may be contacted in vitro, ex vivo, or in vivo.

Once formulated, the compositions of the invention can be administered (as proteins/polypeptides, or in the context of expression vectors for gene therapy) directly to the subject or delivered ex vivo, to cells derived from the subject (e.g., as in ex vivo gene therapy). Direct in vivo delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, for example, International Publication No. WO 93/14778. Examples of cells useful in ex vivo applications include, for example, stem cells, particularly hematopoetic, lymph cells, macrophages, dendritic cells, or tumor cells, pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus (and adeno-associated virus) or alphavirus, all well known in the art.

Illustrative, but non-limiting methods of nucleic acid and polypeptide delivery are further discussed below.

In certain embodiments, it will be preferred to deliver one or more pluripotency factors to a cell using a viral vector or other in vivo polynucleotide delivery technique. In a preferred embodiment, the viral vector is a non-integrating vector. This may be achieved using any of a variety or well-known approaches, several of which are outlined below for purposes of illustration.

A. Adenovirus Vectors

One illustrative method for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992).

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991).

Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993).

B. Retrovirus Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990). In order to construct a retroviral vector, a nucleic acid encoding one or more oligonucleotide or polynucleotide sequences of interest is inserted into the viral genome in the place of certain viral sequences to produce a virus that is replication-defective. Also included are episomal or non-integrating forms of retroviral vectors based on lentiviruses (e.g., a type of retrovirus).

C. Adeno-Associated Virus Vectors

AAV (Ridgeway, 1988; Hermonat & Muzyczka, 1984) is a parovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka & McLaughlin, 1988).

AAV is a good choice of delivery vehicles due to its safety, i.e., gnetically engineered (recombinant) does not integrate into the host genome. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

D. Other Viral Vectors as Expression Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), polioviruses and herpes viruses may be employed. They offer several attractive features for various mammalian cells (Friedmann, 1989; Ridgeway, 1988; Coupar et al., 1988; Horwich et al., 1990). Also included are hepatitis B viruses (Horwich et al., 1990; and Chang et al., 1991).

E. Non-Viral Methods

In order to effect expression of the oligonucleotide or polynucleotide sequences of the present invention, the expression construct must be delivered into a cell. This delivery may be accomplished in vitro, as in laboratory procedures for transforming cells lines, or in vivo or ex vivo, as in the treatment of certain disease states

In certain embodiments of the invention, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane (see Dubensky et al., (1984); and Benvenisty & Reshef (1986)).

Another embodiment of the invention for transferring a naked DNA expression construct into cells may involve particle bombardment. (see Klein et al., 1987; Yang et al., 1990; and Zelenin et al., 1991). The microprojectiles used have consisted of biologically inert substances such as tungsten or gold beads.

In another embodiment, cells of the invention may be microinjected with nucleic acids of the present invention. In one embodiment, DNA microinjection is performed using borosilicate glass microinjection capillaries. In another preferred embodiment, DNA microinjection is accomplished using carbon nanotubes.

In related embodiments, the nucleic acids of the invention are transferred to cells via electroporation. In other related embodiments, liposomes act as gene delivery vehicles and are described in U.S. Pat. No. 5,422,120; WO 95/13796; WO 94/23697; WO 91/14445; and EP 0524968. In certain embodiments, it may be desirable to target a liposome using targeting moieties that are specific to a particular cell type, tissue, and the like. Additional approaches are described in Philip, Mol. Cell. Biol. 14:2411 (1994), and in Woffendin, Proc. Natl. Acad. Sci. (1994) 91:11581-11585.

Further embodiments provide additional non-viral delivery suitable for use in the methods of the present invention, including but not limited to mechanical delivery systems such as the approach described in Woffendin et al., Proc. Natl. Acad. Sci. USA 91(24):11581 (1994); deposition of photopolymerized hydrogel materials or use of ionizing radiation (see, e.g., U.S. Pat. No. 5,206,152 and WO 92/11033); the use of a hand-held gene transfer particle gun (see, e.g., U.S. Pat. No. 5,149,655); and the use of ionizing radiation for activating transferred gene (see, e.g., U.S. Pat. No. 5,206,152 and WO 92/11033).

Delivery devices can also be biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques.

Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

Biodegradable polymeric nanoparticles facilitate nonviral gene transfer to human embryonic stem cells (hESCs). Small (approximately 200 nm), positively charged (approximately 10 mV) particles are formed by the self assembly of cationic, hydrolytically degradable poly(beta-amino esters) and plasmid DNA.

Polynucleotides of the present invention may also be administered to cells by direct microinjection, temporary cell permeabilizations (e.g., co-administration of repressor and/or activator with a cell permeabilizing agent), fusion to membrane translocating peptides, and the like.

F. Electroporation

Electroporation based techniques may also be used to introduce polynucleotides or polypeptides into cells. Electroporation, or electropermeabilization, refers generally to a significant increase in the electrical conductivity and permeability of the cell plasma membrane caused by an externally applied electrical field. This technique is commonly used in molecular biology to introduce some substance into a cell, such as loading it with a molecular probe, a drug or protein that can change the cell's function, a piece of coding DNA, or an RNA interference molecule, among others.

Electroporation is a dynamic phenomenon that depends on the local transmembrane voltage at each cell membrane point. It is generally accepted that for a given pulse duration and shape, a specific transmembrane voltage threshold exists for the manifestation of the electroporation phenomenon (from 0.5V to 1V). This leads to the definition of an electric field magnitude threshold for electroporation (Eₜₕ). Typically, if a second threshold (Eᵢᵣ) is reached or surpassed, electroporation will compromise the viability of the cells, i.e., irreversible electroporation.

In molecular biology, the process of electroporation is often used for the transformation of bacteria, yeast, and plant protoplasts. This procedure is also highly efficient for the introduction of foreign genes or other polynucleotides or polypeptides into mammalian cells. For example, it is used in the process of producing knockout mice, as well as in tumor treatment, gene therapies, and cell-based therapies. The process of introducing foreign DNAs into eukaryotic cells is known generally as transfection.

### XV. Cell Targeting

The present invention contemplates, in part, to provide repressors and/or activators as discussed herein throughout to cells ex vivo or in vivo, directly, in order to alter the potency of the cell (i.e., to reprogram and/or program the cell). For example, in particular embodiments, the desired cell to be reprogrammed or programmed can be in a mixed population of cells, and specific targeting of one or more repressors and/or activators, or a composition comprising the same, to the particular cell will be desirable, and in certain embodiments, preferred.

The present invention, also contemplates, in part, that an in vivo method of altering the potency of a cell, wherein the method comprises administering to a subject, one or more repressors and/or activators, or a composition comprising the same, it would be preferred and in certain preferred embodiments, advantageous to specifically target the one or more repressors and/or activators to the cells that are to be reprogrammed and/or programmed.

Thus, in one embodiment, a polypeptide or fusion polypeptide of the present invention, as discussed herein throughout, comprise a cell-specific targeting moiety. The cell-specific targeting moiety confers cell-type specific binding to the molecule, and it is chosen on the basis of the particular cell population to be targeted. A wide variety of proteins are suitable for use as cell-specific targeting moieties, including but not limited to, ligands for receptors such as growth factors, hormones and cytokines, and antibodies or antigen-binding fragments thereof.

Since a large number of cell surface receptors have been identified in the various cell lineages within the human body, ligands or antibodies specific for these receptors may be used as cell-specific targeting moieties. For example, any of the various cell markers that are cell surface receptors, as discussed elsewhere herein would be suitable for use in a method of the present invention, wherein it is desirable to target a particular type of cell in a population of cells, ex vivo or in vivo.

Ligands which may be used to target specific cell subsets include the interleukins (IL1-IL15), granulocyte-colony stimulating factor, macrophage-colony stimulating factor, granulocyte-macrophage colony stimulating factor, leukemia inhibitory factor, tumor necrosis factor, transforming growth factor, epidermal growth factor, hepatocyte growth factor, nerve growth factor, BDNF, CTNF, platelet derived growth factors, insulin-like growth factors, fibroblast growth factor, chemokines, hormones, neurotransmitters, TGFs, BMPs, Wnts, Hedgehogs, Notch ligands, and the like.

Additionally, certain cell surface molecules are highly expressed in tumor cells, including hormone receptors such as human chorionic gonadotropin receptor and gonadotropin releasing hormone receptor (Nechushtan et al., 1997, J. Biol. Chem. 272:11597). Therefore, the corresponding hormones may be used as the cell-specific targeting moieties in cancer therapy.

Antibodies are the most versatile cell-specific targeting moieties because they can be generated against any cell surface antigen of interest. Monoclonal antibodies have been generated against cell surface receptors, tumor-associated antigens, and leukocyte lineage-specific markers such as CD antigens. In certain embodiments, a single chain antibody (e.g., scFv) can be used as a cell-specific targeting moiety in the present invention. In a nOn-limiting example, scFv are used, which are comprised of VH and VL domains linked into a single polypeptide chain by a flexible linker peptide. Furthermore, the Fc portion of the heavy chain of an antibody may be used to target Fc receptor-expressing cells such as the use of the Fc portion of an IgE antibody to target mast cells and basophils. The specific targeting of these cell types is useful for treating IgE-mediated hypersensitivity in humans and animals (Helm et al., 1988, Nature 331:180-183; PCT/IL96/00181).

In the present invention, a “targeting complex” comprises a ligand and means for delivering a component of the effector system to a target cell through cooperation between a marker on the surface of the target cell and the ligand.

For example, a ligand-targeted liposome could be used to deliver proteins and other small molecules, as well as nucleic acids, to a target cell type. Alternatively, proteins may be coupled to ligands by techniques known in the art, or may comprise natural or artificially incorporated ligands within their structure, such as for example carbohydrate groups.

The means for delivery of the component of the effector system can be a DNA vector, in linear or circular form, which comprises the essential functional elements of the invention and may comprise additional sequences as necessary for particular applications.

The targeting complex may also include means for facilitating internalization of the effector system component and means for promoting the functional deployment thereof. Such means may include peptides known to cooperate in as yet ill-defined intracellular mechanisms which permit escape of viral particles to the cytoplasm from endosomal/lysosomal pathways (Wiley and Skehel, Ann. Rev. Biochem. (1987), 56, p. 365-394), and nuclear localisation signals which enhance uptake of the effector component by the nucleus (Picard et al., Cell (1988) 54, 1073-1080).

The constituent components of the targeting complex may be held in association by a variety of means. Any attachment techniques, including the engineering of a ligand into the effector system component, may be employed. For example, proteinaceous components of the effector system may be engineered to comprise peptides or other groups which act as ligands in the process of the invention. Alternative methods of attaching components of the effector system to ligands will be apparent to those skilled in the art.

In the present invention a “ligand” is any entity capable of specific binding to the surface of a cell. For example, any molecule for which a cellular receptor exists could be used as a ligand. Such substances comprise proteins, nucleic acids, carbohydrates, sugars and metal ions. The use of altered ligand molecules having engineered specificities, including a plurality of specificities, is also contemplated. Especially preferred are antibodies and antibody fragments, such as Fab, F(ab′)₂, and Fv fragments.

The present invention contemplates, in part, to provide repressors and/or activators as discussed herein throughout to cells ex vivo or in vivo, directly, in order to alter the potency of the cell (i.e., to reprogram and/or program the cell).

Thus, in one embodiment, the present invention provides compositions comprising one or more repressors and/or activators of the present invention as discussed herein throughout, wherein at least one repressor and/or activator is cell permeable (e.g., fused to a cell permeable peptide), and that modulates at least one component of a cell potency pathway.

In particular embodiments, the present invention provides a method to alter the potency of a cell (e.g., reprogram or program) comprising contacting the cell with at least one repressor and/or activator, or a composition comprising the same, wherein at least one repressor and/or activator is cell permeable, to modulate at least one component of a pathway(s) associated with the potency of a cell, thereby reprogramming the cell. In particular related embodiments, a method of altering the potency of a cell, wherein the alteration is reprogramming, said method further comprises the step of programming the cell to a desired mature somatic cell.

In certain embodiments, the programming is accomplished by contacting a reprogrammed cell of the present invention with one or more repressors and/or activators, or a composition comprising the same, wherein at least one repressor and/or activator is cell permeable, to modulate at least one component of a pathway(s) associated with the potency of a cell, thereby programming the cell.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “immunoliposome” refers to a liposome bearing an antibody or antibody fragment that acts as a targeting moiety enabling the liposome to specifically bind to a particular “target” molecule that may exist in solution or may be bound to the surface of a cell. Where the target molecule is one that is typically found in relative excess (e.g., 10-fold) and in association with a particular cell type or alternatively in a multiplicity of cell types all expressing a particular physiological condition the target molecule is said to be a “characteristic marker” of that cell type or that physiological condition.

A “hydrophilic polymer” as used herein refers to long chain highly hydrated flexible neutral polymers attached to lipid molecules. Examples include, but are not limited to polyethylene glycol-, or polypropylene glycol-modified lipids PI or CS, or ganglioside GM1.

In one embodiment, the present invention provides immunoliposomes for selective delivery of therapeutic agents to specific tissues in a host and methods of use for those liposomes. The liposomes of this invention employ a composition that optimizes internalization of the liposome into the cytoplasm of the cells of the target tissue. The phrase “optimizes internalization” or “optimal internalization” is used to refer to the delivery of liposome contents such that it achieves maximum delivery to the cytoplasm of the target cell and therefore maximum therapeutic effect. It is recognized that internalization of an immunoliposome into the cytoplasm of a cell is a function of the blood half-life of the liposome, the ability of the liposome to recognize and bind to its target cell, and the uptake of the liposome into the cytoplasm of the target cell. It is well known that addition of a hydrophilic polymer to liposomes increases serum half-life by decreasing both liposome agglomeration (aggregation) and liposome uptake by the RES. Without being bound to a particular theory, it is believed that hydrophilic polymers at high concentrations interfere with recognition and binding by the targeting moiety or ligand and with subsequent uptake by the target cell, thereby decreasing the internalization of the liposome contents by the target cell. Optimal internalization into the cytoplasm of the cell refers to that condition in which maximal uptake into the cytoplasm of the target cell is achieved while still maintaining a blood half-life significantly greater than the blood half-life of liposomes lacking any hydrophilic polymer and adequate for targeting purposes.

For example, a liposome comprising a hydrophilic polymer (e.g., PEG-modified lipid) in an amount up to about 3.6 mole percent of total (vesicle-forming) lipid demonstrates a high rate of internalization into the cytoplasm of the target cell while retaining a blood half-life substantially greater than that seen in liposomes lacking a hydrophilic polymer. This is particularly true where the immunoliposome is targeted with Fab′ fragments conjugated to one or more lipid constituents of the liposome.

In addition, liposomes comprising up to 3.6 mole percent of a hydrophilic polymer conjugated with a Fab′ fragment as a targeting moiety show a high degree of cellular specificity and a binding affinity greater than that of the Fab′ fragments alone. In fact, the binding specificity achieved by such immunoliposomes is comparable to the binding specificity of the intact antibody.

While the Fab′ fragment can be conjugated to any portion of the liposome, in particular embodiments, the Fab′ fragment is attached to the distal ends of the hydrophilic polymer (e.g., polyethylene glycol). High levels of internalization of the liposome by the target cell are achieved when even high levels of hydrophilic polymer are present (e.g., up to 15 mole percent of total phospholipid, more preferably from about 10 to 12 mole percent of total phospholipid). Thus, in one preferred embodiment, the present invention provides for a liposome that is internalized by a target cell, where the liposome includes a Fab′ fragment attached to the distal ends of a hydrophilic polymer, e.g., polyethylene glycol. The Fab′ fragment is preferably not attached to even the majority of hydrophilic polymer. Typically, the Fab′ will be attached to only about 1 to about 20% of the hydrophylic polymer, more preferably about 4 to about 10 mole percent of the hydrophilic polymer and most preferably about 6 to about 10 mole percent of the hydrophilic polymer. The hydrophilic polymer bearing Fab′ fragments (e.g., PEG-Fab′) thus are present at about 0.1 to 2.0 mole percent of the total phospholipid, more preferably at about 0.4 to about 1.0 mole percent, and most preferably about 0.6 to about 1.0 mole percent of total phospholipid.

The immunoliposomes of this invention optimize delivery of one or more repressors and/or activators of the present invention, or a composition comprising the same, to the cytoplasm of the target cell by maintaining an elevated blood half-life, by maintaining a high degree of target specificity, and by effective internalization of the liposome itself (carrying therapeutic agent) thereby avoiding considerable loss of the of one or more repressors and/or activators in solution or degradation of the of one or more repressors and/or activators in the endosomic/lysosomic pathway. The liposomes of the present invention are thus particularly useful as vehicles for the delivery of one or more repressors and/or activators to specific target cells.

### XVI. Implants

Compositions comprising one or more of reprogrammed and/or programmed cells, one or more repressors and/or activators, and a pharmaceutically acceptable carrier or diluent (e.g., a pharmaceutically acceptable cell culture medium) as well as other compositions, described herein can be employed as cell-based therapies in animals, for example, in the repair, regeneration, or replacement of a cell, tissue, or organ.

Generally, such methods involve transferring the cell- or cell-culture based compositions to the desired depot. The cell- or cell-culture based compositions are transferred to the desired tissue by any method appropriate, which generally varies according to the tissue type. For example, cell- or cell-culture based compositions can be transferred to a graft by bathing the graft or infusing it with a pharmaceutically acceptable culture medium containing the cells. Alternatively, the cell- or cell culture-based compositions are provided at the desired site within the tissue to establish a population. Cell- or cell culture-based compositions can be transferred to sites in vivo using devices well know to those skilled in the art, including, but not limited to catheters, trocars, cannulae, or stents seeded with the cell- or cell culture-based compositions.

A number of techniques have been reported recently to culture cells in vitro or ex vivo and implant resulting cultured tissues in a patient. The cells may not be cultured alone, but in many cases, the cells are seeded and cultured on a biocompatible material, such as a carrier (base material for tissue regeneration) used as a scaffold of cell proliferation. The carrier can be molded into any suitable form and has especially important roles to prepare tissues in a three-dimensional shape having a certain depth or height.

In one embodiment, a method of cell, tissue and/or organ repair or regeneration comprises: i) implanting a biocompatible material (e.g., a carrier or base material) functioning as a scaffold of tissue regeneration; ii) administering a cell- or cell culture-based composition having an affinity for the implanted carrier or base material; and iii) reproducing the tissues in vivo. This technique is called regenerative medicine or tissue engineering.

Biomaterial science is an established and evolving field (Takayama et al, Principles of Tissue Engineering, Second Edition edit Lanza R P, Langer R, Vacanti J. Academic Press, San Diego, 2000, pg 209-218; Saltmann, et al, Principles of Tissue Engineering, Second Edition edit Lanza R P, Langer R, Vacanti J. Academic Press, San Diego, 2000, p 221-236; Hubbell, et al, Principles of Tissue Engineering, Second Edition edit Lanza R P, Langer R, Vacanti J. Academic Press, San Diego, 2000, p 237-250; Thomson, et al, Principles of Tissue Engineering, Second Edition edit Lanza R P, Langer R, Vacanti J. Academic Press, San Diego, 2000, p 251-262; Pachence, et al, Principles of Tissue Engineering, Second Edition edit Lanza R P, Langer R, Vacanti J. Academic Press, San Diego, 2000, p 263-278). Chemists have developed methods to synthesize biocompatible polymers to direct and modulate cell growth in vitro, ex vivo, and in vivo. The physical properties of the polymers can be modulated to create solid and liquid matrices of specific strengths and viscosities. Some polymers are stable in vivo and will remain in a patient's body for up to 1, 2, 3, 4, 5, 10, 15 or more years. Other polymers are also biodegradable, resorbing at a fixed rate over time to allow replacement by newly synthesized extracellular matrix proteins. Resorption can occur within days to weeks or months following implantation (Pachence, et al, Principles of Tissue Engineering, Second Edition edit Lanza R P, Langer R, Vacanti J. Academic Press, San Diego, 2000, p 263-278).

The biocompatible material also includes bioabsorbable material. A porous carrier is preferably made of one component or a combination of multiple components selected from the group consisting of collagen, collagen derivatives, hyaluronic acid, hyaluronates, chitosan, chitosan derivatives, polyrotaxane, polyrotaxane derivatives, chitin, chitin derivatives, gelatin, fibronectin, heparin, laminin, and calcium alginate, and said support member is made of one component or a combination of multiple components selected from the group consisting of polylactic acid, polyglycolic acid, polycaprolactone, polylactic acid-polyglycolic acid copolymer, polylactic acid-polycaprolactone copolymer, and polyglycolic acid-polycaprolactone copolymer. Metals like titanium, titanium alloys, stainless steels, cobalt-chromium alloys, and cobalt-chromium-molybdenum alloys, ceramics like alumina ceramics, carbon ceramics, zirconia ceramics, silicon carbide ceramics, silicon nitride ceramics, and glass ceramics, and other bioinert materials are also applicable to the material of the support matrix or lattice. Bioactive matrix materials like hydroxyapatite, calcium phosphate, calcium carbonate, and bioglass are further applicable to the material of the support matrix or lattice.

The cell- or cell culture-based compositions of the present invention can also be combined with a viscous, biocompatible liquid material. The biocompatible liquid is capable of gelling at body temperature and is selected from the group consisting of alginate, collagen, fibrin, hyaline, or plasma. The cells can also be combined with a malleable, three dimensional matrix capable of filling an irregular tissue defect. The matrix is a material including, but not limited to, polyglycolic-polylactic acid, poly-glycolic acid, poly-lactic acid, or suture-like material.

The invention also includes a cell, tissue or organ repairing composition comprising an isolated reprogrammed or programmed cell implanted into a subject or patient, in combination with a malleable, three dimensional matrix capable of filling an irregular cell, tissue and/or organ defect and a solid phase, biocompatible material of sufficient structural integrity to serve as an anchor within the matrix underlying the cell, tissue and/or organ defect.

In various embodiments, a biocompatible matrix or scaffold (e.g., material) is implanted in a patient or subject, and the cells, which have an affinity for the matrix, are administered to the patient or subject, in vivo and subsequently reprogrammed and/or programmed as desired to effect therapy.

In particular embodiments, the reprogrammed cells are induced to differentiate ex vivo and expand into tissue prior to implantation into an animal. As such, the cells are cultured on substrates that facilitate formation into three-dimensional structures conducive for tissue development. Thus, for example, the cells are cultured or seeded onto a biocompatible lattice (e.g., material), such as one that includes extracellular matrix material, synthetic polymers, cytokines, growth factors, etc. Such a lattice can be molded into desired shapes for facilitating the development of tissue types. The lattice can be formed from polymeric material, having fibers as a mesh or sponge. Such a structure provides sufficient area on which the cells can grow and proliferate. Desirably, the lattice is biodegradable over time, so that it will be absorbed into the animal matter as it develops. Suitable polymers can be formed from monomers such as glycolic acid, lactic acid, propyl fumarate, caprolactone, and the like. Other polymeric material can include a protein, polysaccharide, polyhydroxy acid, polyorthoester, polyanhydride, polyphosphozene, or a synthetic polymer, particularly a biodegradable polymer, or any combination thereof. Also, the lattice can include hormones, such as growth factors, cytokines, morphogens (e.g. retinoic acid etc), desired extracellular matrix materials (e.g. fibronectin, laminin, collagen etc) or other materials (e.g. DNA, viruses, other cell types etc) as desired.

The cell- or cell culture-based compositions of the present invention are introduced into the lattice such that they permeate into interstitial spaces therein. For example, the matrix can be soaked into a solution or suspension containing the cell- or cell-based compositions, or they can be infused or injected in the matrix. Preferably, a hydrogel formed by cross-linking of a suspension including the polymer and also having the inventive cells dispersed therein is used. This method of formation permits the cells to be dispersed throughout the lattice, facilitating more even permeation of the lattice with the cells. Of course, the composition also can include support cells that supply factors to the cells of the invention. Support cells include other cell types which will promote the differentiation, growth and maintenance of the reprogrammed or programmed cells of the invention.

Those skilled in the art will appreciate that lattices suitable for inclusion into the implanted material can be derived from any suitable source, e.g. Matrigel™, and can of course include commercial sources for suitable lattices. Another suitable lattice can be derived from the acellular portion of adipose tissue, muscle tissue, nervous system tissue, bone marrow tissue, and the like (i.e., other human tissue acellular matrices). Typically such lattices include proteins such as proteoglycans, glycoproteins, hyaluronin, fibronectins, collagens, and the like, all of which serve as excellent substrates for cell growth. Additionally, lattices can include hormones, cytokine, growth factors, and the like.

These techniques involve the seeding and implanting of cells onto a matrix to form tissue and structural components which can additionally provide controlled release of bioactive agents. The matrix is characterized by a network of lumens functionally equivalent to the; naturally occurring vasculature of the tissue formed by the implanted cells and which is further lined with endothelial cells. The matrix is further coupled to blood vessels or other ducts at the time of implantation to form a vascular or ductile network throughout the matrix. The free-form fabrication techniques refer to any technique know in the art that builds a complex 3-dimensional object as a series of 2-dimensional layers. The methods can be adapted for use with a variety of polymeric, inorganic and composite materials to create structures with defined compositions, strengths and densities. Thus, utilizing such methods, precise channels and pores can be created within the matrix to control subsequent cell growth and proliferation within the matrix of one or more cells types having a defined function. In such a way, differentiated adipose-derived cells, corresponding to the various types of a particular organ's cells can be combined to form a partial or whole joint. Such cells are combined in the matrix to provide a vascular network lined with endothelial cells interspersed throughout the cells.

The cell- and cell culture-based compositions, biocompatible materials, matrices, lattices, and compositions used in the methods of the present invention are used in tissue engineering and regeneration in a patient or subject. Thus, the invention pertains to the use of an implantable structure incorporating any of the disclosed features. The exact nature of the implant will vary according to the use desired. The implant can comprise mature tissue or can include immature tissue or the lattice or matrix. Thus, for example, an implant can comprise a population of reprogrammed and/or programmed cells that are optionally seeded within a lattice of a suitable size and dimension. Such an implant is injected or engrafted within a patient or subject to encourage the generation or regeneration of mature tissue within the patient or subject.

In particular embodiments, a cell used in an implant of the present invention is an adult stem cell or progenitor cell. In another embodiment the cell is an adult somatic cell.

In certain embodiments, an implant of the present invention comprises a somatic cell reprogrammed from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an epithelial cell, an immune cell or an endothelial cell. The reprogramming can be accomplished ex vivo or in vivo.

In certain related embodiments, cells reprogrammed ex vivo are subsequently programmed, either ex vivo or in vivo to a cell and/or tissue selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In certain other embodiments, cells reprogrammed in vivo are subsequently programmed, in vivo to a cell and/or tissue selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In particular embodiments, the reprogrammed and/or programmed cells are in contact with a biocompatible material (e.g., and implant).

The cells can be reprogrammed and/or programmed ex vivo in the presence of the biocompatible material, and subsequently, an implant comprising the biocompatible material and the reprogrammed or programmed cells is administered to a subject or patient (e.g., surgically).

In addition, the cells can be reprogrammed and/or programmed ex vivo in the absence of the biocompatible material, and subsequently, the reprogrammed or programmed cells are administered to a subject or patient and target themselves to the implant comprising the biocompatible material that had been previously implanted in the subject or patient (e.g., surgically).

In one embodiment, cells suitable for implants of the present invention are generated by a method comprising altering the potency of a cell, which further comprises contacting a cell ex vivo with the one or more repressors and/or activators, in order to modulate at least one component of a cellular pathway associated with cell potency, wherein the method further comprises the step of administering the reprogrammed or programmed cell to a subject or patient. The cell can be administered in combination with the implant; or alternatively, the implant can be surgically located prior to the administration of cells to the subject. In this case, the cells target themselves to the implant comprising the biocompatible material using cell targeting means. The source of the cells can be allogenic, syngenic, autogenic or xenogenic in nature.

In related embodiments, altering the potency of a cell is conducted in vivo, by administering to the subject or patient a composition comprising one or more repressors and/or activators wherein the one or more repressors and/or activators contacts the cell in a cell specific manner, e.g., by cell specific targeting of a therapeutic composition, as described elsewhere herein. As noted above, the cells can be administered in combination with the implant or alternatively, the implant can be surgically located prior to the administration of cells to the subject, in which case the cells target themselves to the implant comprising the biocompatible material using cell targeting means.

In further related embodiments, a method of in vivo cell-based therapy comprises: i) administering to the subject or patient an implant in combination with one or more cells; ii) administering to the subject or patient a composition comprising one or more repressors and/or activators wherein the one or more repressors and/or activators contacts the cell in a cell specific manner, e.g., by cell specific targeting of a therapeutic composition, as described elsewhere herein; and iii) modulating at least one component of a cellular pathway associated with cell potency with the composition.

In certain embodiments, the cells are administered surgically in combination with an implant. In other embodiments, the implant is surgically implanted prior to administration of cells and the cells target themselves to the implant comprising the biocompatible material using cell targeting means. The source of the cells can be allogenic, syngenic, autogenic or xenogenic in nature.

In further embodiments, a method of ex vivo cell-based therapy comprises: i) contacting a cell with one or more repressors and/or activators, ex vivo; ii) modulating at least one component of a cellular pathway associated with cell potency with the one or more repressors and/or activators; iii) reprogramming and or programming the cells; and iv) administering to the subject or patient the reprogrammed and/or programmed cells.

In a related embodiment, a method of ex vivo cell-based therapy comprises: i) contacting a cell with one or more repressors and/or activators, ex vivo, wherein the cells are in contact with a biocompatible material (e.g., an implant); ii) modulating at least one component of a cellular pathway associated with cell potency with the one or more repressors and/or activators; iii) reprogramming and or programming the cells; and iv) administering to the subject or patient the composition of the biocompatible material and reprogrammed and/or programmed cells.

In various embodiments, a subject is suffering from cancer and/or a disease, disorder, or condition associated with pancreatic tissue, neural tissue, cardiac tissue, bone marrow, muscle tissue, bone tissue, skin tissue, liver tissue, hair follicles, vascular tissue, adipose tissue, lung tissue, or kidney tissue.

In particular embodiments, the subject is about to undergo, is undergoing, or has undergone a surgical procedure or a tissue or organ transplant procedure.

In certain embodiments, the tissue or organ transplant procedure is selected from a liver transplant, heart transplant, neural tissue transplant, kidney transplant, bone marrow transplant, stem cell transplant, skin transplant, lung transplant.

### XVII. Cell Cultures and Cell Culture Compositions

The compositions and methods of the present invention require, in some embodiments, the culture of cells and repressors/activators of the present invention. As discussed herein throughout, the present compositions and methods are useful for ex vivo and in vivo cell-based therapies, which in some embodiments require cell cultures, i.e., culturing the cells to be reprogrammed and/or programmed with one or more repressors and/or activators in a cell culture medium, e.g., a pharmaceutically acceptable cell culture medium. a culture, cell culture, culture system, or cell culture compositions can be administered separately by enteral or parenteral administration methods or in combination with other suitable compounds to effect the desired treatment goals. In particular embodiments, a culture, cell culture, culture system, or cell culture composition of the present invention is administered in an implant or along with a biocompatible material, while in other embodiments, the biocompatible material or implant is surgically located in a subject and then a culture, cell culture, culture system, or cell culture composition is directed administered to the location of the implant.

A. Mouse Embryonic Stem Cell Culture

Mitotically inactivated cell feeder layers were first used to support difficult-to-culture epithelial cells (Puck et al., 1956) and were later successfully adapted for the culture of mouse EC cells (Martin and Evans 1975) and mouse ESCs (Evans and Kaufman 1981). Medium that is “conditioned” by coculture with various cells was found to be able to sustain ESCs in the absence of feeders, and fractionation of conditioned medium led to the identification of leukemia inhibitory factor (LIF), a cytokine that sustains ESCs (Smith et al., 1988; Williams et al., 1988). LIF and its related cytokines act via the gp130 receptor (Yoshida et al., 1994). Binding of LIF induces dimerization of LIFR/gp130 receptors, which in turn activates the Janus-associated tyrosine kinases (JAK)/the latent signal transducer and activator of transcription factor (STAT3) (Yoshida et al., 1994), and Shp2/ERK mitogen-activated protein kinase (MAPK) cascade (Takahashi-Tezuka et al., 1998). STAT3 activation alone is sufficient for LIF-mediated self-renewal of mouse ESCs in the presence of serum (Matsuda et al., 1999). Activation of ERK, however, appears to impair mouse ESC proliferation. In contrast, suppression of the ERK pathway by the addition of MEK inhibitor PD098059 promotes ESC self-renewal (Burdon et al., 1999). Thus, the proliferative effect of LIF on mouse ESCs requires a finely tuned balance between positive and negative effectors.

In serum-free medium, LIF alone is insufficient to prevent mouse ESC differentiation, but in combination with BMP (bone morphogenetic protein, a member of the TGFβ superfamily), mouse ESCs are sustained (Ying et al., 2003). BMPs induce the expression of 1d (inhibitor of differentiation) proteins through the Smad pathway. The overexpression of 1d could indeed promote mouse ESC proliferation in the presence of LIF alone without the need for either BMPs or serum. However, BMPs might also act through inhibition of the MAPK pathways independent of Smads. The latter is supported by the facts that ESCs can be derived from blastocysts lacking Smad4 (the common partner for all Smads) (Sirard et al., 1998) and that inhibition of p38 MAPK by SB203580 allowed derivation of ESCs from blastocysts lacking BMP type I receptor Alk-3, which were previously refractory to ESC derivation (Qi et al., 2004). In normal development, however, there is no apparent requirement for LIF, gp130 or STAT3 prior to gastrulation (Escary et al., 1993; Yoshida et al., 1996; Takeda et al., 1997), and homozygous Alk-3 mutant mouse embryos can develop normally to early post-implantation stage (Mishina et al., 1995).

B. Human Embryonic Stem Cell Culture

Mitotically inactivated fibroblast feeder layers and serum-containing medium were used in the initial derivation of human ESCs, essentially the same conditions used for the derivation of mouse ESCs prior to the identification of LIF (Thomson et al., 1998). However, the specific factors used to sustain mouse ESCs do not support human ESCs. LIF and its related cytokines fail to support human or nonhuman primate ESCs in serum-containing media that supports mouse ESCs (Thomson et al., 1998; Daheron et al., 2004; Humphrey et al., 2004). Consistent with this observation, human ESCs and other pluripotent stem cells do not express or express at very low levels of critical components of the LIF pathway—LIFR, gp130, and JAK 1 and 2 (Brandenberger et al., 2004), and in conditions that do support human ESCs, STAT3 is minimally activated (Daheron et al., 2004). Components of the BMP pathway are all present in human ESCs (Rho et al., 2006) and other pluripotent stem cells, but unlike mouse ESCs, BMPs added to human ESCs in conditions that would otherwise support self-renewal, cause rapid differentiation (Xu et al., 2002).

Basic FGF (bFGF) allows the clonal growth of human ESCs and other pluripotent stem cells on fibroblasts in the presence of a commercially available serum replacement (Amit et al., 2000). At higher concentrations, bFGF allows feeder independent growth of human ESCs and other pluripotent stem cells cultured in the same serum replacement (Wang et al., 2005;C Xu et al., 2005; R. H. Xu et al., 2005; Levenstein et al., 2006). The mechanism through which these high concentrations of bFGF exert their functions is incompletely known, although one of the effects is suppression of BMP signaling (R. H. Xu et al., 2005). Serum and a widely used commercially available serum replacement have significant BMP-like activity, which is sufficient to induce differentiation of human ESCs and other pluripotent stem cells, and conditioning this medium on fibroblasts reduces this activity. At moderate concentrations of bFGF (40 ng/mL), the addition of noggin or other inhibitors of BMP signaling significantly decreases background differentiation of human ESCs and other pluripotent stem cells. At higher concentrations (100 ng/mL), bFGF itself suppresses BMP signaling in human ESCs to levels comparable with those observed in fibroblast-conditioned medium, and the addition of noggin no longer has a significant effect. Suppression of BMP activity by itself is insufficient to maintain human ESCs (R. H. Xu et al., 2005) and other pluripotent stem cells, thus additional roles for bFGF signaling exist. Evidence suggests that bFGF up-regulates the expression of TGFβ ligands in both feeder cells and human ESCs, which, in turn, could promote human ESC self-renewal (Greber et al., 2007). Human ESCs themselves and other pluripotent stem cells produce FGFs, which appear insufficient for low-density cell culture but can maintain high-density cultures for variable periods. Inhibition of FGFRs by SU5402 causes differentiation of human ESCs (Dvorak et al., 2005), suggesting the involvement of FGFRs. The required downstream events, however, are still not well understood, but some evidence implicates activation of the ERK and PI3K pathways (Kang et al., 2005; Li et al., 2007).

Both Activin and TGFβ have strong positive effects on undifferentiated proliferation of human ESCs and other pluripotent stem cells in the presence of low or modest concentrations of FGFs, and based on inhibitor studies, it has been suggested that TGFβ/Activin signaling is essential for human ESC self-renewal (Beattie et al., 2005; James et al., 2005; Vallier et al., 2005). However, when TGFβ/Activin signaling is inhibited with SB431542, there is a concomitant rise in BMP signaling activity (Beattie et al., 2005; James et al., 2005; Vallier et al., 2005), so it has been unclear whether signaling through TGFβ/Activin is merely acting to inhibit the sister BMP pathway, or whether TGFβ/Activin signaling has other, independent roles. Recent studies have revealed multiple interactions between the FGF, TGFβ, and BMP pathways in human ESCs and other pluripotent stem cells. Activin induces bFGF expression (Xiao et al., 2006), and bFGF induces Tgfβ1/TGFβ and Grem1/GREM1 (a BMP antagonist) expression and inhibits Bmp4/BMP4 expression in both fibroblast feeders and in human ESCs (Greber et al., 2007). This reciprocity of induction between the FGF and TGFβ/Activin pathways likely explains why at high doses of bFGF, exogenous TGFβ or Activin has only very modest effects on undifferentiated human ESC proliferation (Ludwig et al., 2006) and, similarly, at sufficient doses of Activin, the beneficial dose of exogenous FGF is greatly reduced (Vallier et al., 2005; Xiao et al., 2006).

Other growth factors have been reported to have a positive effect on human ESC growth and the growth of other pluripotent stem cells including, but not limited to, Wnt (Sato et al., 2004), IGF1 (Bendall et al., 2007), heregulin (Wang et al., 2007), pleiotrophin (Soh et al., 2007), sphingosine-1-phosphate (S1 P), and PDGF (Pebay et al., 2005). Additional compounds have been found that also increase the efficiency of clonal human ESC culture such as the Rock inhibitor Y-27632 (Watanabe et al., 2007), such efficiencies for low passage cells, nonetheless, remain poor.

C. Increasing Efficiency of Stem Cell Cloning

As described above, a difficulty in growing pluripotent cells, like ESCs, in culture is their low cloning efficiency. As used herein, “cloning efficiency” means a number of cells individualized by trypsin that form new ESC colonies divided by the number of individual cells plated in a well of a culture dish. It is known that growing human ESCs in defined and animal-free conditions on a matrix (e.g., Matrigel®), results in a low cloning efficiency (i.e., less than 0.1%). This contrasts with using culture systems based on medium conditioned by exposure to fibroblasts, where the cloning efficiency, while still low (i.e., less than 2%), is high enough to initiate clonal ESC colonies. As disclosed herein, the addition of a small molecule to the culture medium in which human ESCs and other pluripotent stem cells are grown permits the stem cell cultures to be clonally cultivated in a manner that is extremely difficult without the addition of the small molecule.

As a point of clarification, there is a difference between “passaging” human ESCs and initiating clonal colonies. In typical practice in ESC and other pluripotent stem cells cultivation, when a culture container is full, the colony is split into aggregates, which are then placed into new culture containers. These aggregates typically contain 100 to 1,000 cells, which readily initiate growth in culture. In contrast, initiating clonal colonies requires growing human pluripotent stem cell colonies from single individual pluripotent stem cells.

The small molecules used to increase the cloning efficiency of ESCs and other pluripotent stem cells effectively increased the cloning efficiency of the culture of ESC and other pluripotent stem cells, even in different culture conditions.

One preferred small molecule is (S)-(+)-2-methyl-1-[(4-methyl-5-isoquinolinyl)sulfonyl]homopiperazine dihydrochloride (informal name: H-1152). Another preferred small molecule is 1-(5-isoquinolinesulfonyl)piperazine hydrochloride (informal name: HA-100). Although both appear equally effective in facilitating clonal growth, H-1152 can be used at ten times lower working concentrations than HA-100. Other related small molecules that are also effective included the following: 1-(5-isoquinolinesulfonyl)-2-methylpiperazine (informal name: H-7), 1-(5-isoquinolinesulfonyl)-3-methylpiperazine (informal name: iso H-7), N-2-(methylamino)ethyl-5-isoquinoline-sulfonamide dihydrochloride (informal name: H-8), N-(2-aminoethyl)-5-isoquinolinesulphonamide dihydrochloride (informal name: H-9), N-[2-p-bromo-cinnamylamino)ethyl]-5-isoquinolinesulfonamide dihydrochloride (informal name: H-89), N-(2-guanidinoethyl)-5-isoquinolinesulfonamide hydrochloride (informal name: HA-1004), 1-(5-isoquinolinesulfonyl)homopiperazine dihydrochloride (informal name: HA-1077), (S)-(+)-2-Methyl-4-glycyl-1-(4-methylisoquinolinyl-5-sulfonyl)homopiperaz-ine dihydrochloride (informal name: glycyl H-1152) and (+)-(R)-trans-4-(1-aminoethyl)-N-(4-pyridyl)cyclohexanecarboxamide dihydrochloride (informal name: Y-27632). Each small molecule has been reported to support a cloning efficiency >1% in a defined medium, such as TeSR™ medium, on Matrigel®-coated culture dishes. The full constituents and methods of use of the TeSR™1 medium are described in Ludwig et al.

The effect conditioned by these small molecules is not limited to the use of TeSR™1 medium. These small molecules also increased cloning efficiency of pluripotent stem cell cultures grown on conditioned medium, which is medium that has been exposed to fibroblasts. It is thus believed that these small molecules increase the cloning efficiency of any pluripotent stem cell culture medium in which pluripotent stem cells can effectively be grown.

A class of small molecules effective for increasing the cloning efficiency of a ESC culture medium are inhibitors of kinase enzymes, including protein kinase A (PKA), protein kinase C (PKC), protein kinase G (PKG) and Rho-associated kinase (ROCK).

Of particular interest herein are ROCKs. ROCKs are serine/threonine kinases that serve as target proteins for Rho (of which three isoforms exist—RhoA, RhoB and RhoC). Theses kinases were initially characterized as mediators of the formation of RhoA-induced stress fibers and focal adhesions. The two ROCK isoforms—ROCK1 (p160ROCK, also called ROKβ) and ROCK2 (ROKa)—are comprised of a N-terminal kinase domain, followed by a coiled-coil domain containing a Rho-binding domain and a pleckstrin-homology domain (PH). Both ROCKs are cytoskeletal regulators, mediating RhoA effects on stress fiber formation, smooth muscle contraction, cell adhesion, membrane ruffling and cell motility. ROCKs exert their biogical activity by targeting downstream molecules, such as myosin light chain (MLC), MLC phosphatase (MLCP) and the phosphatase and tensin homolog (PTEN).

An exemplary ROCK inhibitor is Y-27632, which selectively targets ROCK1 (but also inhibits ROCK2), as well as inhibits TNF-α and IL-1β. It is cell permeable and inhibits ROCK1/ROCK2 (IC.sub.50=800 nM) by competing with ATP. Ishizaki T, et al., Mol. Pharmacol. 57:976-983 (2000), incorporated herein by reference as if set forth in its entirety. Other ROCK inhibitors include, e.g., H-1152, Y-30141, Wf-536, HA-1077, hydroxyl-HA-1077, GSK269962A and SB-772077-B. Doe C, et al., J. Pharmacol. Exp. Ther. 32:89-98 (2007); Ishizaki et al., supra; Nakajima M, et al., Cancer Chemother. Pharmacol. 52:319-324 (2003); and Sasaki Y, et al., Pharmacol. Ther. 93:225-232 (2002).

The small molecules identified herein have at least a pyridine as a common structural element. As such, other small molecules useful herein include, e.g., N-(4-Pyridyl)-N′-(2,4,6-trichlorophenyl)urea, 3-(4-Pyridyl)-1H-indole and (R)-(+)-trans-N-(4-Pyridyl)-4-(1-aminoethyl)-cyclohexanecarboxamide.

D. Medium Formulations

Pluripotent stem cells can be cultured in any medium used to support growth of pluripotent stem cells. Exemplary culture media include, but are not limited to, a defined medium, such as TeSR™ (StemCell Technologies, Inc.; Vancouver, Canada), mTeSR™ (StemCell Technologies, Inc.) and StemLine® serum-free medium (Sigma; St. Louis, Mo.), as well as conditioned medium, such as mouse embryonic fibroblast (MEF)-conditioned medium. As used herein, a “defined medium” refers to a biochemically defined formulation comprised solely of biochemically-defined constituents. A defined medium may also include solely constituents having known chemical compositions. A defined medium may further include constituents derived from known sources. As used herein, “conditioned medium” refers to a growth medium that is further supplemented with soluble factors from cells cultured in the medium. Alternatively, cells can be maintained on MEFs in culture medium.

A defined and humanized medium for the culture and proliferation of human ESCs typically includes salts, vitamins, a source of glucose, minerals and amino acids. To supplement the medium and supply conditions to support cell growth, initially stem cell media included serum from one source or another. Also previously, it has been reported that the addition of fibroblast growth factor plus a serum replacement additive will permit the cultivation of human ESCs without serum. The serum replacement can be a commercially available product sold for that purpose or can be a formulated mixture of protein, such as serum albumin, vitamins, salts, minerals, a transferrin or transferrin substitute, and insulin or an insulin substitute. This serum replacement component may also be supplemented with selenium. It is preferred here that a defined serum replacement be used in lieu of serum from any source in culturing human pluripotent stem cells, in order to avoid the issues of variation in serum constituents and to use media that are as defined as possible. TeSR1 medium is comprised of a DMEM/DF12 base, supplemented with human serum albumin, vitamins, antioxidants, trace minerals, specific lipids, and cloned growth factors.

As one example, the combination of the use of higher concentrations of any one or more of FGF (10 to 1000 ng/ml) together with the use of GABA (gamma aminobutyric acid), pipecholic acid (PA), lithium (LiCl) and transforming growth factor beta (TGFβ), enables a medium to support undifferentiated stem cell growth. Other combinations thereof are included.

Several factors have positive effects on undifferentiated proliferation. Of these, bFGF, LiCl, .gamma.-aminobutyric acid (GABA), pipecholic acid, and TGFβ are ultimately included in TeSR1. For each of the four cell lines tested, the proliferation rate and the percentage of cells maintaining expression of characteristic human pluripotent stem cell markers was higher in TeSR1 than in control cells cultured in fibroblast-conditioned medium, and removal of any one of these five factors decreased culture performance.

It is also helpful to note the culture conditions for the human pluripotent stem cells in a biological matrix in the culture vessel. One such material that has been used is Matrigel™, which is an artificial basement membrane of mouse cell origin, which is supplied as a commercial product free of mouse cells. Another material of human origin also known now to serve a similar purpose is fibronectin, a human glycoprotein which is used in its insoluble form to create a fiber matrix also to serve as a basement membrane for pluripotent stem cell culture.

The present invention also contemplates, in part, the use of a pharmaceutically acceptable cell-culture medium in particular compositions and/or cultures of the present invention. Such compositions are suitable for administration to human subjects. In particular embodiments, the pharmaceutically acceptable cell culture medium is a serum free medium. An illustrative example of a pharmaceutically acceptable cell culture medium follows. Said medium includes, but is not limited to Calcium Chloride Anhydrous CaCl₃ (158.695 mg/L); Cupric Sulfate CuSO₄ 5H₂O (0.000654 mg/L); Ferric Nitrate Fe(NO₃) 9H₂O (0.0751 mg/L); Ferric Sulfate FeSO₄7H₂O (0.0209 mg/L); Potassium Chloride KCl (306.969 mg/L); Magnesium Chloride MgCl₂ (14.418 mg/L); Magnesium Sulfate MgSO₄ (63.237 mg/L); Sodium Chloride NaCl (5021.73 mg/L); Sodium Bicarbonate NaHCO₄ (1100 mg/L); Sodium Phosphate Monobasic NaH₂PO₄H₂O (93.964 mg/L); Sodium Phosphate dibasic Na₂HPO₄ 7H₂O (35.753 mg/L); Zinc Sulfate ZnSO₄ 7H₂O (0.217 mg/L); D-Glucose (Dexrose) (3836.3 mg/L); Phenol Red (8.127 mg/L); HEPES (3099.505 mg/L); Na Hypoxanthine (1.203 mg/L); Linoleic acid (0.0211 mg/L); DL-68-Thioctic Acid (0.0528 mg/L); Sodium Putrescine 2HCl (0.0407 mg/L); Putrescine 8 Sodium Selenite (2.5×10⁻⁶ mg/L); Sodium Pyruvate (40.1885 mg/L); Alanine (3.24 mg/L); Arginine HCl (116.255 mg/L); Asparagine (4.19 mg/L); Aspartic acid (3.347 mg/L); Cysteine H₂O (9.445 mg/L); Cystine 2HCl (15.752 mg/L); Glutamic acid (3.7 Glutamine (293.55 mg/L); Glycine (24.439 mg/L); Histidine HCl H₂O (36.847 mg/L); Isoleucine (79.921 mg/L); Leucine (82.227 mg/L); Lysine HCl (118.937 mg/L); Methionine (23.679 mg/L); Phenylalanine (50.861 mg/L); Proline (12.564 mg/L); Serine (34.214 mg/L); Threonine (74.408 mg/L); Tryptophan (12.54 mg/L); Tyrosine 2Na⁺ 2 H₂O (64.086 mg/L); Valine (73.606 mg/L); Biotin (0.00176 mg/L); D-Calcium panthenate (3.127 mg/L); Choline chloride (6.52 mg/L); Folic acid (3.334 mg/L); i-Inositol (9.904 mg/L); Niacinamide (3.079 mg/L);Pyridoxine HCl (3.022 mg/L); Riboflavine (0.31 mg/L); Thiamine HCl (3.092 mg/L); Thymidine (0.183 mg/L); Vitamin B12 (0.512 mg/L); PROTEINS Human recombinant Insulin (12.5 mg/L); Human ApoTransferrin (50 mg/L); Progesterone (0.0099 mg/L); Recombinant Human Serum Albumin (0.18 mg/L); β-mercaptoethanol (7.868 mg/L); and Human recombinant bFGF (0.04 mg/L). The osmolarity of said media should be ˜265 milli-osmoles.

The present invention, also provides, in part, a culture, cell culture, culture system, or cell culture composition comprising: (i) a cell; (ii) a composition comprising one or more repressors in contact with the cell; and (iii) a pharmaceutically acceptable culture medium wherein the one or more repressors modulates at least one component of a cellular pathway associated with the pluripotency of the cell.

In particular embodiments, a culture, cell culture, culture system, or cell culture composition comprises: (a) a cell; (b) a composition comprising one or more activators in contact with the cell; and (c) a pharmaceutically acceptable culture medium wherein the one or more activators modulates at least component of a cellular pathway associated with the pluripotency of the cell.

In particular embodiments, the pharmaceutically acceptable cell culture medium is a serum free medium.

### XVIII. Methods of Use

In one embodiment of the invention, the developmental potency is altered such that the cell becomes a pluripotent stem cell. Such reprogrammed pluripotent stem cells are important for numerous areas of therapy according to the present invention.

Reprogrammed pluripotent stem cells have the ability to divide without limit and give rise to many specialized cells in an organism. There are several reasons why human pluripotent stem cells may be important to cancer research and reducing the cancer burden. First, reprogrammed pluripotent stem cells may be used to treat the tissue toxicity brought on by cancer therapy. Bone marrow and peripheral blood multipotent stem cells (which are more committed stem cells) are used already to restore patients' hematopoietic and immune systems after high dose chemotherapy. However, reprogrammed pluripotent stem cells have greater potential for returning the complete repertoire of immune response to patients undergoing bone marrow transplantation, thus contributing to the development of other treatments such as immune/vaccine therapy. Other tissues damaged by cancer therapy can also benefit by replenishing their stem cell pools, e.g., injection of reprogrammed pluripotent stem cells into the heart may permanently reverse cardiomyopathy caused by certain chemotherapeutic agents, injection of reprogrammed pluripotent stem cells that have been differentiated into neural cells may restore brain function after cancer treatment.

Reprogrammed pluripotent stem cells can be used to treat many cardiovascular diseases for which therapy is currently inadequate. For example, reprogrammed pluripotent stem cells could potentially be used to repair the failing heart when it can no longer pump, to generate growth of heart chambers when infants are born with malformed hearts, and to repair vascular damage resulting from high blood pressure and atherosclerosis. Thus, reprogrammed pluripotent stem cells transplanted into the heart successfully repopulate the heart tissue and work together with the host cells.

Reprogrammed pluripotent stem cells can be engineered (e.g., programmed) to specialized cell types such as bone, cartilage and salivary cells, which can be used as replacement for organs damaged by disease or injury. Examples include the treatment of temporomandibular joint disorders (TMDs), the replacement of skeletal elements lacking or damaged in diseases such as fibrous dysplasia of bone using cells grown in special natural or synthetic scaffolding materials, and the replacement of salivary cells damaged by disease (Sjögren's Syndrome) or radiation for head and neck cancer.

Reprogrammed pluripotent stem cells can be differentiated into highly important tissue specific cells. For example, reprogrammed pluripotent stem cells have been differentiated into pancreatic islet beta cells, which is are capable of secreting insulin. Isolated cells of this type are used for transplantation studies and, to a limited extent, in human therapeutic approaches to treat type 1 diabetes. The reprogrammed human pluripotent stem cell could offer an unlimited supply of these cells once the rules of differentiation are known.

Other examples include cellular therapy to replace diseased liver tissue. In this case a reprogrammed pluripotent stem cell is subsequently differentiated or programmed along the cell lineage of a functional liver cell. Other examples could include various forms of kidney cells or potentially bladder cells.

The present invention contemplates, in part, that there are numerous other examples in addition to diabetes, liver failure, kidney failure, and urologic diseases in which reprogrammed human pluripotent stem cells have a major therapeutic role.

Reprogrammed pluripotent stem cells are suitable to treat and/or ameliorate the many diseases that result from the loss of nerve cells, and mature nerve cells that cannot normally divide to replace those that are lost. In Parkinson's disease, nerve cells of the substantia nigra that make the chemical dopamine die. In Alzheimer's disease, cells that make acetylcholine die. In amyotrophic lateral sclerosis the motor nerve cells that activate muscles die. In stroke, brain trauma, and spinal cord injury many types of cells are lost. There are many more disorders that affect both adults and young children in which nerve cells die.

It is important to note that reprogrammed pluripotent stem cells might be used to do very different things to treat different disorders. For example, in some diseases reprogrammed pluripotent stem cells might specialize and replace a particular type of nerve cell—a different kind of nerve cell for Parkinson's than for Alzheimer's than for amyotrophic lateral sclerosis and so on. For other disorders, like multiple sclerosis, it is not nerve cells, but supporting cells, the glial cells that wrap electrical insulation around nerve fibers, that reprogrammed pluripotent stem cells can be programmed to replace. In other neurological insults, for example brain trauma or stroke, reprogrammed pluripotent stem cells can be used to regenerate regions of brain tissue, with many integrated types of brain cells.

Research on human pluripotent stem cells could lead to cures for diseases that require treatment through transplantation, including autoimmune diseases. (Autoimmune diseases include multiple sclerosis, rheumatoid arthritis, systemic lupus erythematosus, and type-I diabetes). The most feasible example over the short term is treatment of type-I diabetes by transplantation of pancreatic islet cells or beta cells produced from autologous human pluripotent stem cells—that is human pluripotent stem cells found in the person who would be receiving the transplant. While much research is needed, including research on whether stems cells can be found in children or adults, the promise is considerable. Gene transfer into pluripotent stem cells could obviate the need for immunosuppressive agents in transplantation and the ensuing susceptibility to other diseases. Moreover, ultimately, human pluripotent stem cells might be used to create transplantable cells, tissues, and organs of any type. In addition to eliminating the need for immunosuppressive drugs, this would address problems ranging from the supply of donor organs to the difficulty of finding matches between donors and recipients.

Reprogrammed human pluripotent stem cells can be used in treatment of virtually all primary immunodeficiencies. There are more than 70 different forms of primary (congenital and inherited) deficiencies of the immune system. Primary immunodeficiency diseases are characterized by an unusual susceptibility to infection and are sometimes associated with anemia, arthritis, malabsorption and diarrhea, and certain malignancies. They can involve considerable pain and suffering, numerous hospitalizations, high medical costs, and even death. Almost all of these diseases are rare. Because these diseases are genetic, gene replacement is an important area of investigation in the search for effective treatment. The transplantation of reprogrammed allogenic human pluripotent stem cells reconstituted comprising a normal gene might result in development of healthy cells of the types affected by the missing or damaged genetic material in the immunodeficiency disease.

Autologous human reprogrammed pluripotent stem cell transplants (transplants to and from the self) can restore immune function and is a viable option for treating HIV disease. Such transplants can regenerate all the components of the immune system that have been damaged by HIV infection.

Reprogrammed human pluripotent stem cells are also useful in applied trauma and burn research, such as research devoted to the development of “artificial skin.” Such a biomaterial is widely applicable in the field of burn therapy. In one embodiment, a biopolymer sponge made of collagen is combined with actual reprogrammed pluripotent cells from burn patients. Reprogrammed human pluripotent stem cells can also be subsequently programmed to skin cells and used in combination with a biopolymer as a source of “skin” to build such a graft, especially for severely burned patients with limiting amounts of remaining intact skin.

Reprogrammed pluripotent stem cell can be subsequently programmed along a certain path to become a liver cell, a blood cell, a brain cell, or any type of cell which then can be used in transplantation and for other purposes, as described throughout herein. For example, programming reprogrammed pluripotent stem cells can be used to replace organs or tissues that are defective as a consequence of birth defects. For example, one such condition is biliary atresia, in which part of the liver does not develop correctly. Thus, in one embodiment, reprogrammed human pluripotent stem cells can be directed to form liver tissue or to replace the damaged organ and save the life of the affected infant.

Reprogrammed pluripotent stem cells can be transplanted into an injury or diseased retina in order to promote repair and/or regeneration of retinal cells. This approach minimizes the immunological rejection and low efficiency associated with other methods presently used in the art to treat retinopathies.

There is also a significant clinical need for improved techniques to promote conjunctival and corneal healing during disease or after injury. Conventional surgery is not consistently successful in treating persistent corneal ulcers, chemical or thermal injury, bullous keratopathy, and various cicatrical diseases. Transplantation with reprogrammed pluripotent stem cells can provide a means of facilitating epithelialization of the ocular surface, reducing inflammation, vascularization, and scarring.

Parkinson's disease, according to most recent findings, has a strong environmental exposure component for one form of the disease. The nature of the agents and the timing of the exposure remain unknown at present. The use of reprogrammed human pluripotent stem cell cultures permits screening for the subtle effects of candidate environmental toxicants and toxicant mixtures on specific cell types in the developmental stages of the cell lineage comprising the nervous system cells and tissue associated with the brain region compromised by the disease. Such explorations yield powerful insight into the biological mechanism(s) underlying human susceptibility to the epigenetic form of this disease with onset after age 50, as well as the genetic-based “early” onset form of the disease.

Thus, in one embodiment, molecular markers or surrogate markers or combinations of these that can be utilized for population-based studies of gene-environment interaction in disease etiology are analyzed. Using the power of reprogrammed human pluripotent stem cell toxicity screening coupled with DNA micro-array technology, one skilled in the art can construct complex matrices of reporter molecules that report a “signature” characteristic of very high risk for the development of a complex source human disease. Applied to newborns and children, the most vulnerable of our population, maximum opportunities for medical health planning for intervention and prevention of disease in sensitive individuals are possible.

Reprogrammed human pluripotent stem cells hold enormous potential for cell replacement or tissue repair therapy in many degenerative diseases of aging. For disorders affecting the nervous system, such as Alzheimer's and Parkinson's diseases, amyotrophic lateral sclerosis, and spinal cord and brain injury, transplantation of neural cell types derived from reprogrammed human pluripotent stem cells provides for replacing cells lost in these conditions and of recovery of function. Reprogrammed human pluripotent stem cells have several critical advantages over stem cells of more mature derivation. The problem of rejection following cell therapy is more easily overcome with pluripotent stem cells than with more mature stem cells. They can differentiate into virtually any cell type in the body and are capable of generating large numbers of cells. In addition, reprogrammed human pluripotent stem cells can provide a model for studying fundamental molecular and cellular processes important in the understanding of aging and age-related diseases.

Because reprogrammed pluripotent stem cells constitute a self-renewing population of cells, they can be cultured to generate greater numbers of bone or cartilage cells than could be obtained from a tissue sample. In one embodiment, a self-renewing population of new stem cells is established in a transplant recipient, said transplant leads to the long-term correction of many diseases and degenerative conditions in which bone or cartilage cells are deficient in numbers or defective in function. This is accomplished by either by transplanting the stem cells from a healthy donor to a recipient, or by genetically modifying a person's own stem cells and returning them to the marrow. Such an approach is an important therapeutic option for genetic disorders of bone and cartilage, such as osteogenesis imperfecta and the various chondrodysplasias. In a somewhat different application, reprogrammed pluripotent stem cells can be stimulated in culture to develop into either bone or cartilage-producing cells. These cells can then be introduced into the damaged areas of joint cartilage in cases of osteoarthritis, or into large gaps in bone that can arise from fractures or surgery. This method of tissue repair would have a number of advantages over the current practice of tissue grafting.

The present invention further contemplated that reprogrammed pluripotent stem cells ca be used to replace the sound-detecting hair cells in the inner ear that are often lost due to genetic, infectious, traumatic, or pharmacologic causes.

There is good evidence that many of the mental and behavioral disorders such as schizophrenia, autism, manic-depressive illness and memory disorders, result from permanent disruption of brain circuitry or brain chemistry. Thus, in one embodiment, reprogrammed pluripotent stem cells can be used to correct such defects and restore mental health to the subject. Similar transplant strategies apply to other severe developmental disorders, such as autism.

Reprogrammed pluripotent stem cells also provide a means of replacing neurons destroyed by drug abuse. This is especially useful for individuals who have abused drugs such as methamphetamine, MDMA (ecstacy) and inhalants which have been shown in animal and some human studies to cause long-term, possibly permanent damage to selected areas of the brain. For example, recent research has shown that methamphetamine can have significant toxic effects on dopaminergic and serotonergic neurons in the brain. This is of particular concern because of the spreading use of this drug and may be related to the dramatic behavioral effects, including the development of psychotic-like behavior patterns that methamphetamine can have in some people. Reprogrammed pluripotent stem cells stimulated to develop into dopaminergic, serotonergic or other types of neurons, can provide a means of replacing neurons destroyed by drug abuse. In this way, we may be able to eventually reverse some of the debilitating behavioral effects of drugs such as methamphetamine.

Alcohol is a major source of damage to organs, such as the liver and brain, which may or may not regain function with abstinence from drinking. Development of medications that accelerate recovery in organs damaged by alcohol would be a major breakthrough. Such an advance would lessen human suffering and the economic burden associated with alcohol-induced organ damage. Reprogrammed human pluripotent stem cell research provide a cost-effective means of discovering mechanisms that underlie alcohol-related pathology and that could be targets for new medications. For cases of irreversible organ damage, reprogrammed human pluripotent stem cell can be used to facilitate generation of new organ tissue.

The epsilon globin gene is expressed only in red blood stem cells. This gene recently has been shown to block the sickling of the sickle cell hemoglobin. Reprogrammed pluripotent stem cell therapy can be used to turn on the epsilon globin gene in adult blood cells and thereby halt the disease process, as the cells used would not contain the sickle cell gene.

As noted herein, the present invention relates generally to methods and compositions for altering the developmental potency of a cell, comprising contacting the cell with one or more repressors and/or activators, or a composition comprising the same, in order to modulate at least one component of a developmental potency pathway, thereby altering the potency of the cell. The methods and compositions provided herein are contemplated for use with cellular based therapies in a wide variety of disease, disorders, or conditions in which the replacement, regeneration, expansion, reprogramming, programming, modulation and/or maintenance of a given cellular state is desirable or beneficial in treating, reducing the risk of, or reducing the symptoms associated with the disease, disorder or condition.

The methods provided herein are contemplated for use with in vivo and ex vivo therapeutic modalities, alone or in combination with each other. For example, in certain embodiments, in vivo therapeutic modalities may involve localized, in vivo administration, such as direct injection of one or more repressors and/or activators, or a composition comprising the same (including cell culture based compositions, as described elsewhere herein), into a subject, or into a biocompatible material (e.g., an implant) or into a target tissue or target organ of a subject. In other embodiments, in vivo therapeutic modalities may involve system administration of one or more repressors and/or activators, or a composition comprising the same (including cell culture based compositions, as described elsewhere herein). Particular modes of in vivo administration are exemplified elsewhere herein and known to a person skilled in the art.

In certain aspects relating to ex vivo therapy, cells from one or more tissues may be isolated, for example, from the subject to be treated, from another subject, from a tissue culture source, or from any other desirable source of cells. The isolated cells may be contacted in tissue culture with one or more repressors and/or activators, or a composition comprising the same, such as an an antibody or an antibody fragment, an ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a ssDNA; a polypeptide or an active fragment thereof, a peptidomimetic, a peptoid, a small organic molecule, in any number or combination.

For example, the cells of the invention may be dedifferentiated or reprogrammed to a totipotent, pluripotent or multipotent state ex vivo, before being administered to a subject in need thereof. In other embodiments, the cells invention may be differentiated or programmed from a totipotent, pluripotent or multipotent state to a desired mature cellular state before being administered to the subject. In certain embodiments, before or after contacting the cells with a composition comprising one or more repressors and/or activators, the cells may be further purified and/or expanded to achieve one or more populations of desired reprogrammed or programmed cells, such as a particular population of mature somatic cells, multipotent cells, pluripotent cells, and/or totipotent cells. The cells may be administered to the subject to be treated with or without continued administration of a composition comprising one or more repressors and/or activators as provided herein.

The methods of treatment provided herein relate generally to cell based therapies. Examples of cell-based therapies include, but are not limited to cell, tissue, and/or organ transplant therapies, as well as cell, tissue, and/or organ regeneration therapies. Cell based therapies as provided herein may target one or more particular cell types, tissue types, and or organs.

Illustrative cells, tissues, or organs, to be repaired and or regenerated (i.e., targeted) include, but are not limited to, neural cells in tissues (e.g., to treat ischemic injury, spinal cord injury), cardiac cells or tissues (e.g., to treat myocardial infarction or other ischemic injury, congestive heart failure), pancreatic islet cells or pancreatic tissues (e.g., to treat diabetes, such as Type II diabetes), motor neuron cells (e.g., to treat to Parkinson's Disease and provide motor neuron cell regeneration), hepatocyte cells or tissues, renal cells (e.g., to treat liver or kidney transplant and provide liver or kidney regeneration), lung cells or tissues, skin tissues (e.g., to improve wound healing, and provide skin transplants for burn therapy), skeletal muscle tissue, hematopoietic cell transplant, expansion, and/or regeneration (e.g., B-cell regeneration and replacement, immature progenitor cell expansion, reprogramming red blood cell fate to white blood cell fate, modulate homing and engraftment), hair follicles (e.g., improve hair growth), among others known to a person skilled in the art.

Methods of the present invention are suitable for providing therapy to the hematopoietic cell system including, but not limited to, altering the types of hematopoietic cells generated following a transplant by programming a cell toward a desired lineage, such as red blood cells, platelets, B-cells, T-cells, or other specialized immune or hematopoietic cells. For example, individuals with myelodysplastic syndrome suffer from ineffective production of red blood cells, such that altering hematopoietic stem/progenitor cell potency by programming a hematopoietic stem/progenitor cells to red blood cells would provide a beneficial treatment for this condition.

Additional examples include: reprogramming mature somatic cells into primitive hematopoietic stem cells to enhance the engraftment capability of a transplant; reprogramming mature cells in a transplant towards white cell lineages to enhance outcome, augmenting the capabilities of hematopoietic stem such, such as their self-renewal capabilities and their ability to home and engraft, in a given niche; and directly expanding stem cell populations within a transplant, such as by dedifferentiating somatic cells into a multipotent stem cells, pluripotent stem cells, and/or totipotent stem cells.

Further embodiments include therapies directed to reducing adipogenesis, as well as therapies directed to promoting the formation of bone, such as after a traumatic bone injury or during/after bone-related surgery. For example, certain methods may involve incorporation of a composition comprising: i) one or more repressors and/or activators that modulate one or more components of a cellular pathway associated with the developmental potency of a cell; ii) programmed or reprogrammed cells; iii) cell culture-based compositions, as described elsewhere herein; or (iv) any combination of (i)-(iii), into bone implant devices to stimulate and/or improve bone formation. Other methods may involve direct in vivo administration of one or more repressors and/or activators of the invention to the site of injury or regeneration. Certain embodiments may be employed in degenerative bone or joint diseases, such as osteoarthritis, osteoporosis, or osteitis deformans.

Other embodiments include, for example, incorporation of a composition comprising: i) one or more repressors and/or activators that modulate one or more components of a cellular pathway associated with the developmental potency of a cell; ii) programmed or reprogrammed cells; iii) cell culture-based compositions, as described elsewhere herein; or (iv) any combination of (i)-(iii), into artificial tissue matrices to stimulate wound healing. Certain embodiments may also encompass transient delivery of one or more repressors and/or activators according to the present invention during surgical interventions to enhance the outcome of the procedure, such as by introducing one or more repressors and/or activators that modulate one or more components of a cellular pathway associated with the developmental potency of a cell, as provided herein, that could expand progenitor cells in the targeted organs or tissues. In certain embodiments, the surgical procedure may encompass a tissue or organ transplant, such as a liver transplant, heart transplant, neural tissue transplant, kidney transplant, bone marrow transplant, stem cell transplant, skin transplant, or lung transplant.

Certain embodiments of the methods provided herein may be employed to treat neurodegenerative or neurological conditions or disease, including, for example, Alzheimer's disease, amyotrophic lateral sclerosis, ataxia telangiectasia, HIV associated dementia, Huntington's disease, multiple sclerosis, multiple system atrophy, Parkinson's disease, paralysis, Pick's disease, schizophrenia, spinal muscular atrophy, stroke, and prion disease.

In certain embodiments, the methods provided herein may be utilized to treat or manage the symptoms of degenerative muscle diseases, such as muscular dystrophy, duchenne muscular dystrophy, facioscapulohumeral muscular dystrophy, myotome muscular dystrophy, congenital myopathy, or mitochondrial myopathy.

In certain embodiments, the methods provided herein may be utilized to treat or manage the symptoms of degenerative cardiovascular diseases or conditions, such as aneurysms, angina, arryhthmias, atherosclerosis, cardiomyopathy, cerebrovascular disease, congenital heart disease, congestive heart failure, myocarditis, valve disease, dilated cardiomyopathy, myocardial infarction (heart attack), hypertrophic cardiomyopathy, restrictive cardiomyopathy, venous thromboembolism, vascular restenosis, or coronary artery disease with resultant ischemic cardiomyopathy.

In other embodiments, the methods provided herein may be utilized to treat or manage the symptoms of degenerative liver diseases, such as nephritic disease, cirrhosis, alcoholic cirrhosis, fatty liver, alcoholic hepatitis, viral hepatitis, liver carcinoma, post necrotic cirrhosis, biliary cirrhosis, hepatocellular injury or a biliary tract disorder.

Certain embodiments encompass the treatment of degenerative pancreatic diseases, diabetes (e.g., Type I and Type II), diabetes related disorder, hyperglycemia, hyperinsulinemia, hyperlipidaemia, insulin resistance, impaired glucose metabolism, obesity, diabetic retinopathy, macular degeneration, cataracts, diabetic nephropathy, glomerulosclerosis, and diabetic neuropathy.

Certain embodiments encompass methods of increasing or improving cell or tissue regeneration in a subject, wherein the cell or tissue regeneration occurs in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal stem/progenitor cells, mesodermal stem/progenitor cells, neural stem/progenitor cells, or nerve cells.

Other embodiments include methods of treating immune-related diseases, such as diabetes, graft vs. host disease, immunodeficiency disease, hematopoietic malignancy, hematopoietic failure, or hematopoietic stem cell transplantation.

Further embodiments include methods of treating degenerative diseases and other medical conditions that might benefit from regeneration therapies such atherosclerosis, coronary artery disease, obstructive vascular disease, myocardial infarction, dilated cardiomyopathy, heart failure, myocardial necrosis, valvular heart disease, mitral valve prolapse, mitral valve regurgitation, mitral valve stenosis, aortic valve stenosis, and aortic valve regurgitation, carotid artery stenosis, femoral artery stenosis, stroke, claudication, and aneurysm; cancer-related conditions, such as structural defects resulting from cancer or cancer treatments; the cancers such as, but not limited to, breast, ovarian, lung, colon, prostate, skin, brain, and genitourinary cancers; skin disorders such as psoriasis; joint diseases such as degenerative joint disease, rheumatoid arthritis, arthritis, osteoarthritis, osteoporosis and ankylosing spondylitis; eye-related degeneration, such as cataracts, retinal and macular degenerations such as maturity onset; macular degeneration, retinitis pigmentosa, and Stargardt's disease; auralrelated degeneration, such as hearing loss; lung-related disorders, such as chronic obstructive pulmonary disease, cystic fibrosis, interstitial lung disease, emphysema; metabolic disorders, such as diabetes; genitourinary problems, such as renal failure and glomerulonephropathy; neurologic disorders, such as dementia, Alzheimer's disease, vascular dementia and stroke; and endocrine disorders, such as hypothyroidism.

Regeneration therapies from the methods and compositions of the invention may be very useful and beneficial for traumas to skin, bone, joints, eyes, neck, spinal column, and brain, for example, which results in injuries that would normally result in scar formation.

In another embodiment, the present invention provides cells, tissues or organs differentiated ex vivo or in vivo from an induced pluripotent stem cell (e.g., a reprogrammed somatic cell) having a normal karyotype. The cells may be epidermal cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, and chondrocytes.

In a particular embodiment, the cells are selected from a pancreatic islet cell, a CNS cell, a PNS cell, a cardiac cell, a skeletal muscle cell, a smooth muscle cell, a hematopoietic cell, a bone cell, a liver cell, an adipose cell, a renal cell, a lung cell, a chondrocyte, a skin cell, a follicular cell, a vascular cell, an epithelial cell, an immune cell, and an endothelial cell.

In certain embodiments the cells may be myocytes, chondrocytes, epithelial cells, or neurons.

In another embodiment, the tissue may be, without limitation, pancreatic tissue, neural tissue, cardiac tissue, bone marrow, muscle tissue, bone tissue, skin tissue, liver tissue, hair follicles, vascular tissue, adipose tissue, lung tissue, and kidney tissue. In a particular embodiment, the organ is selected from the group consisting of brain, spinal cord, heart, liver, kidney, stomach, intestine, eye, and pancreas.

In another embodiment, the cell, tissue or organ of the present invention is used for transplantation. Preferably, the cell is autogenic, syngeneic, or allogenic to that of a transplanted subject. When the cell, tissue or organ of the present invention is used for transplantation, a desired effect can be achieved because of the normal cell karyotype. In addition, there is advantageously a reduced level of or no immune rejection reaction.

In another embodiment, the present invention provides a composition comprising a cell, tissue or organ differentiated from a pluripotent stem cell having a normal karyotype. The composition can be used for patients having a disease, disorder or condition in need of such a cell (preferably, a differentiated cell), tissue or organ. Such a disease, disorder or condition includes defects/injuries in cells, tissues or organs.

In another embodiment, the present invention provides a composition for ex vivo or in vivo treatment or prophylaxis of a disease or disorder due to a defect in a cell, tissue or organ of a subject, comprising a reprogrammed cell (e.g., multipotent, pluripotent, and totipotent). In this case, the reprogrammed cell itself is used as the therapeutic modality and subsequent reprogramming of the cell (if desired) is achieved depending on the transplanted environment.

Diseases, disorders, and conditions which may be treated by the present invention, may be associated with defects in cells, tissues or organs differentiated from a multipotent, pluripotent, or totipotent cell of the present invention.

In one embodiment, the reprogrammed cells of the invention may be subsequently programmed ex vivo or in vivo to differentiated cells, tissues, or organs of the circulatory system (blood cells, etc.). Examples of the diseases, disorders, and conditions of the circulatory system include, but are not limited to, anemia (e.g., aplastic anemia (particularly, severe aplastic anemia), renal anemia, cancerous anemia, secondary anemia, refractory anemia, etc.), cancer or tumors (e.g., leukemia); and after chemotherapy therefor, hematopoietic failure, thrombocytopenia, acute myelocytic leukemia (particularly, a first remission (high-risk group), a second remission and thereafter), acute lymphocytic leukemia (particularly, a first remission, a second remission and thereafter), chronic myelocytic leukemia (particularly, chronic period, transmigration period), malignant lymphoma (particularly, a first remission (high-risk group), a second remission and thereafter), multiple myeloma (particularly, an early period after the onset), and the like.

In another embodiment, the reprogrammed cells of the invention may be subsequently programmed ex vivo or in vivo to differentiated cells, tissues, or organs of the nervous system. Examples of such diseases, disorders, and conditions of the nervous system include, but are not limited to, dementia, cerebral stroke and sequela thereof, cerebral tumor, spinal injury, and the like.

In another embodiment, the reprogrammed cells of the invention may be subsequently programmed ex vivo or in vivo to differentiated cells, tissues, or organs of the immune system. Examples of such diseases, disorders, and conditions of the immune system include, but are not limited to, T-cell deficiency syndrome, leukemia, and the like.

In another embodiment, the reprogrammed cells of the invention may be subsequently programmed ex vivo or in vivo to differentiated cells, tissues, or organs of the motor organ or skeletal system. Examples of such diseases, disorders, and conditions of the motor organ and skeletal system include, but are not limited to, fracture, osteoporosis, luxation of joints, subluxation, sprain, ligament injury, osteoarthritis, osteosarcoma, Ewing's sarcoma, osteogenesis imperfecta, osteochondrodysplasia, and the like.

In another embodiment, the reprogrammed cells of the invention may be subsequently programmed ex vivo or in vivo to differentiated cells, tissues, or organs of the skin system. Examples of such diseases, disorders, and conditions of the skin system include, but are not limited to, atrichia, melanoma, cutis matignant lympoma, hemangiosarcoma, histiocytosis, hydroa, pustulosis, dermatitis, eczema, and the like.

In another embodiment, the reprogrammed cells of the invention may be subsequently programmed ex vivo or in vivo to differentiated cells, tissues, or organs of the endocrine system. Examples of such diseases, disorders, and conditions of the endocrine system include, but are not limited to, hypothalamus/hypophysis diseases, thyroid gland diseases, accessory thyroid gland (parathyroid) diseases, adrenal cortex/medulla diseases, saccharometabolism abnormality, lipid metabolism abnormality, protein metabolism abnormality, nucleic acid metabolism abnormality, inborn error of metabolism (phenylketonuria, galactosemia, homocystinuria, maple syrup urine disease), analbuminemia, lack of ascorbic acid systhetic ability, hyperbilirubinemia, hyperbilirubinuria, kallikrein deficiency, mast cell deficiency, diabetes insipidus, vasopressin secretion abnormality, dwarfism, Wolman's disease (acid lipase deficiency)), mucopolysaccharidosis VI, and the like.

In another embodiment, the reprogrammed cells of the invention may be subsequently programmed ex vivo or in vivo to differentiated cells, tissues, or organs of the respiratory system. Examples of such diseases, disorders, and conditions of the respiratory system include, but are not limited to, pulmonary diseases (e.g., pneumonia, lung cancer, etc.), bronchial diseases, and the like.

In another embodiment, the reprogrammed cells of the invention may be subsequently programmed ex vivo or in vivo to differentiated cells, tissues, or organs of the digestive system. Examples of such diseases, disorders, and conditions include, but are not limited to, esophagial diseases (e.g., esophagial cancer, etc.), stomach/duodenum diseases (e.g., stomach cancer, duodenum cancer, etc.), small intestine diseases/large intestine diseases (e.g., polyps of the colon, colon cancer, rectal cancer, etc.), bile duct diseases, liver diseases (e.g., liver cirrhosis, hepatitis (A, B, C, D, E, etc.), fulminant hepatitis, chronic hepatitis, primary liver cancer, alcoholic liver disorders, drug induced liver disorders, etc.), pancreatic diseases (acute pancreatitis, chronic pancreatitis, pancreas cancer, cystic pancreas diseases, etc.), peritoneum/abdominal wall/diaphragm diseases (hernia, etc.), Hirschsprung's disease, and the like.

In another embodiment, the reprogrammed cells of the invention may be subsequently programmed ex vivo or in vivo to differentiated cells, tissues, or organs of the urinary system. Examples of such diseases, disorders, and conditions include, but are not limited to, kidney diseases (e.g., renal failure, primary glomerulus diseases, renovascular disorders, tubular function abnormality, interstitial kidney diseases, kidney disorders due to systemic diseases, kidney cancer, etc.), bladder diseases (e.g., cystitis, bladder cancer, etc.), and the like.

In another embodiment, the reprogrammed cells of the invention may be subsequently programmed ex vivo or in vivo to differentiated cells, tissues, or organs of the genital system. Examples of such diseases, disorders, and conditions include, but are not limited to, male genital organ diseases (e.g., male sterility, prostatomegaly, prostate cancer, testicular cancer, etc.), female genital organ diseases (e.g., female sterility, ovary function disorders, hysteromyoma, adenomyosis uteri, uterine cancer, endometriosis, ovarian cancer, villosity diseases, etc.), and the like.

In another embodiment, the reprogrammed cells of the invention may be subsequently programmed ex vivo or in vivo to differentiated cells, tissues, or organs of the circulatory system. Examples of such diseases, disorders, and conditions include, but are not limited to, heart failure, angina pectoris, myocardial infarct, arrhythmia, valvulitis, cardiac muscle/pericardium diseases, congenital heart diseases (e.g., atrial septal defect, arterial canal patency, tetralogy of Fallot, etc.), artery diseases (e.g., arteriosclerosis, aneurysm), vein diseases (e.g., phlebeurysm, etc.), lymphoduct diseases (e.g., lymphedema, etc.), and the like.

In various embodiments, the compositions and methods of the present invention are suitable for treating/preventing cancer. For example, reprogrammed pluripotent stem cells are important in the treatment of cancer based on the finding that cancer cells may have certain stem cell-like properties, specifically, the ability to renew themselves. Thus, the present invention contemplates, in part, to circumvent this property by cell-specifically targeting the cancer cells for differentiation or programming, along with subsequent surgical or chemotherapeutic treatment to ensure removal of the treated cancer cells.

Cancers that are suitable therapeutic targets of the present invention include cancer cells from the bladder, blood, bone, bone marrow, brain, breast, colon, esophagus, eye, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Contemplated useful secondary or adjunctive therapeutic agents in this context include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegal1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

Other compounds that are effective in treating cancer are known in the art and described herein that are suitable for use with the compositions and methods of the present invention are described, for example, in the “Physicians Desk Reference, 62nd edition. Oradell, N.J.: Medical Economics Co., 2008 “, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000.”, and “The Merck Index, Fourteenth Edition. Whitehouse Station, N.J.: Merck Research Laboratories, 2006”, incorporated herein by reference in relevant parts

In one embodiment, a method of treating an individual in need thereof, wherein the individual has a disease, disorder, or condition as described herein that is amenable to the cell-based therapies of the present invention, comprises contacting a cell from the individual, ex vivo or in vivo, with a composition comprising: i) one or more repressors and/or activators that modulate one or more components of a cellular pathway associated with the developmental potency of a cell; ii) programmed or reprogrammed cells; iii) cell culture-based compositions, as described elsewhere herein; or (iv) any combination of (i)-(iii), wherein the composition targets cancer cells in cells in need of therapy in the patient, thereby treating the cells.

In particular embodiments, the treated cells are cancer cells, and the composition contacting the cancer cells results in the differentiation or programming of said cancer cells. Without wishing to be bound by a particular theory, the programmed cancer cells can then be chemically or surgically removed, thereby treating the cancer patient.

The practice of the present invention will employ, unless indicated specifically to the contrary, conventional methods of chemistry, biochemistry, organic chemistry, molecular biology, microbiology, recombinant DNA techniques, genetics, immunology, cell biology, stem cell protocols, cell culture and transgenic biology that are within the skill of the art, many of which are described below for the purpose of illustration. Such techniques are explained fully in the literature. See, e.g., Sambrook, et al., Molecular Cloning: A Laboratory Manual (3^{rd} Edition, 2001); Sambrook, et al., Molecular Cloning: A Laboratory Manual (2^{nd} Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-interscience; Glover, DNA Cloning: A Practical Approach, vol. I & II (IRL Press, Oxford, 1985); Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Fire et al., RNA Interference Technology: From Basic Science to Drug Development (Cambridge University Press, Cambridge, 2005); Schepers, RNA Interference in Practice (Wiley-VCH, 2005); Engelke, RNA Interference (RNAi): The Nuts & Bolts of siRNA Technology (DNA Press, 2003); Gott, RNA Interference, Editing, and Modification: Methods and Protocols (Methods in Molecular Biology; Human Press, Totowa, N.J., 2004); Sohail, Gene Silencing by RNA Interference: Technology and Application (CRC, 2004); Clarke and Sanseau, microRNA: Biology, Function & Expression (Nuts & Bolts series; DNA Press, 2006); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kurstad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008); Hogan et al., Methods of Manipulating the Mouse Embyro (2^{nd} Edition, 1994); Nagy et al., Methods of Manipulating the Mouse Embryo (3^{rd} Edition, 2002), and The zebrafish book. A guide for the laboratory use of zebrafish (Danio rerio), 4th Ed., (Univ. of Oregon Press, Eugene, Oreg., 2000).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase “consisting essentially of indicates that the listed elements are required or mandatory, but that no other elements are optional and may or may not be present depending upon whether or not they affect the activity or action of the listed elements.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1

### Increasing Concentration of Lipofectamine with siRNAs Related to Multi- or Pluri-Potency Increases Toxicity of Somatic Human Cells in Ex Vivo Treatment

In order to produce clinical grade cells (either from donors or from syngenic sources) with less toxicity for the cell source, the siRNA transfection reagent is tested with and without siRNA to determine the toxic effect of the reagent. Somatic human cells are incubated at various concentrations of Lipofectamine (5 to 8 concentrations, up to 500 ug/ml) at 37° C. in media conditions appropriate for the cells of choice and siRNA administration, Incubation is carried out with and without siRNA, and cell viability is measured after 12, 24, 36, 48 and 72 hours of incubation and cell culture.

In parallel experiments, the incubation with Lipofectamine, with and without siRNA, is for a period of 12 hours, after which the culture is changed with fresh media lacking transfection reagent. Cell viability is again measured after 12, 24, 36, 48, and 72 hours of cell culture. Treatment with transfection reagent for a shorter time reduces toxicity that may be present due a transfection reagent, such as Lipofectamine, liposomes or the use of cationic lipids.

Although optimizing experiments can be done in small volumes, clinical grade human cells are produced in volumes of 10 L, 100 L, 1,000 L or more. In such case cells may not be adherent and various separation techniques can be used to isolate the cells during changes of media to reduce siRNA transfection reagent concentrations.

### Example 2

### Increasing Multi- or Pluri-Potency by RNAi to Repressor or Downregulators of Oct4

In order to produce clinical grade cells (either from donors or from syngenic sources) with greater potential for multi or pluripotency, human fibroblasts, keratinocytes or other human somatic cells are incubated with 10 nM siRNA [Total] Lipofectamine targeted to one or more of the following influencers of Oct4 expression (proteins of a NuRD (Nucleosome Remodeling and Histone Deacetylation) complexes, Cdx-2, Coup-tf1, GCNF, proteins of the Sin3A and Pml complexes; Mbd3, a core component of the NuRD co-repressor complex or an essential NuRD protein; or Hdac1/2- and Mta1/2 type proteins, including those present in the NODE complex (e.g., for Nanog and Oct4 associated deacetylase). Accession numbers for the influencers are provided herein above.

In one assay, the incubation with siRNA/Lipofectamine targeted to an influencer of Oct4 is for the length of cell culture, and is at 37° C. with media conditions appropriate for the cell line of choice and siRNA administration. Expression of Oct4 is measured via reporter gene, mRNA, or detection of Oct4 protein at 12, 24, 36, 48 and 72 hours of cell culture. An increase in Oct4 expression indicates the siRNA has modulated an influencer of Oct4 expression, thereby altering the potency of the cell to increase potency.

In another assay, the incubation with siRNA/Lipofectamine targeted to an influencer of Oct4 is for a period of 12 hours, after which the cell culture media is replaced with fresh media lacking siRNA/Lipofectamine. Again, Oct4 expression is measured at 12, 24, 36, 48 and 72 hours of cell culture. Treatment with transfection reagent for a shorter period reduces toxicity that may be present due a transfection reagent, such as Lipofectamine, liposomes or the use of cationic lipids.

### Example 3

### Increasing Multi- or Pluri-Potency by RNAi to Repressor or Downregulators of Nanog

In order to produce clinical grade cells (either from donors or from syngenic sources) with greater potential for multi or pluripotency, human fibroblasts, keratinocytes or other human somatic cells are incubated with 10 nM siRNA [Total] Lipofectamine targeted to one or more of the following influencers of Nanog expression (proteins of a NuRD (Nucleosome Remodeling and Histone Deacetylation) complexes, proteins of the Sin3A and Pml complexes; Mbd3, a core component of the NuRD co-repressor complex or an essential NuRD protein; or Hdac1/2- and Mta1/2 type proteins, including those present in the NODE complex (e.g., for Nanog and Oct4 associated deacetylase)). Accession numbers for the influencers are provided herein above.

In one assay, the incubation with siRNA/Lipofectamine targeted to an influencer of Nanog is for the length of cell culture, and is at 37° C. with media conditions appropriate for the cell line of choice and siRNA administration. Expression of Nanog is measured via reporter gene, mRNA, or detection of Nanog protein at 12, 24, 36, 48 and 72 hours of cell culture. An increase in Nanog expression indicates the siRNA has modulated an influencer of Nanog expression, thereby altering the potency of the cell to increase potency.

In another assay, the incubation with siRNA/Lipofectamine targeted to an influencer of Nanog is for a period of 12 hours, after which the cell culture media is replaced with fresh media lacking siRNA/Lipofectamine. Again, Nanog expression is measured at 12, 24, 36, 48 and 72 hours of cell culture. Treatment with transfection reagent for a shorter period reduces toxicity that may be present due a transfection reagent, such as Lipofectamine, liposomes or the use of cationic lipids.

### Example 4

### Increasing Multi- or Pluri-Potency by RNAi to Repressor or Downregulators of Sox2

In order to produce clinical grade cells (either from donors or from syngenic sources) with greater potential for multi or pluripotency, human fibroblasts, keratinocytes or other human somatic cells are incubated with 10 nM siRNA [Total] Lipofectamine targeted to one or more of the following influencers of Sox2 expression (repressors of the type involving heterochromatin type proteins; repressors HP1α and HP1γ, preferably HP1α; also Cdx (and other homeo box regulators); and preferred Sip1 type proteins). Accession numbers for the influencers are provided herein above.

In one assay, the incubation with siRNA/Lipofectamine targeted to an influencer of Sox2 is for the length of cell culture, and is at 37° C. with media conditions appropriate for the cell line of choice and siRNA administration. Expression of Sox2 is measured via reporter gene, mRNA, or detection of Sox2 protein at 12, 24, 36, 48 and 72 hours of cell culture. An increase in Sox2 expression indicates the siRNA has modulated an influencer of Sox2 expression, thereby altering the potency of the cell to increase potency.

In another assay, the incubation with siRNA/Lipofectamine targeted to an influencer of Sox2 is for a period of 12 hours, after which the cell culture media is replaced with fresh media lacking siRNA/Lipofectamine. Again, Sox2 expression is measured at 12, 24, 36, 48 and 72 hours of cell culture. Treatment with transfection reagent for a shorter period reduces toxicity that may be present due a transfection reagent, such as Lipofectamine, liposomes or the use of cationic lipids.

### Example 5

### Increasing Multi- or Pluri-Potency by RNAi to Repressor or Downregulators of Klf-4

In order to produce clinical grade cells (either from donors or from syngenic sources) with greater potential for multi or pluripotency, human fibroblasts, keratinocytes or other human somatic cells are incubated with 10 nM siRNA [Total] Lipofectamine targeted to one or more of the following influencers of Klf-4 expression (repressors of Klf-4 or HDAC-5). Accession numbers for the influencers are provided herein above.

In one assay, the incubation with siRNA/Lipofectamine targeted to an influencer of Klf-4 is for the length of cell culture, and is at 37° C. with media conditions appropriate for the cell line of choice and siRNA administration. Expression of Klf-4 is measured via reporter gene, mRNA, or detection of Klf-4 protein at 12, 24, 36, 48 and 72 hours of cell culture. An increase in Klf-4 expression indicates the siRNA has modulated an influencer of Klf-4 expression, thereby altering the potency of the cell to increase potency.

In another assay, the incubation with siRNA/Lipofectamine targeted to an influencer of Klf-4 is for a period of 12 hours, after which the cell culture media is replaced with fresh media lacking siRNA/Lipofectamine. Again, Klf-4 expression is measured at 12, 24, 36, 48 and 72 hours of cell culture. Treatment with transfection reagent for a shorter period reduces toxicity that may be present due a transfection reagent, such as Lipofectamine, liposomes or the use of cationic lipids.

### Example 6

### Increasing Multi- or Pluri-Potency by RNAi to Repressor or Downregulators of Oct4, Nanog, Sox2, and Klf-4

In order to produce clinical grade cells (either from donors or from syngenic sources) with greater potential for multi or pluripotency, human fibroblasts, keratinocytes or other human somatic cells are incubated with 10 nM siRNA [Total] Lipofectamine targeted to one or more of the following influencers of Oct4, Nanog, Sox2, and Klf-4 expression (e.g., repressors of Oct4, Nanog, Sox2, and Klf-4).

In one assay, the incubation with siRNA/Lipofectamine targeted to influencers of Oct4, Nanog, Sox2, and Klf-4 is for the length of cell culture, and is at 37° C. with media conditions appropriate for the cell line of choice and siRNA administration. Expression of Oct4, Nanog, Sox2, and Klf-4 is measured via reporter gene, mRNA, or detection of Oct4, Nanog, Sox2, and Klf-4 proteins at 12, 24, 36, 48 and 72 hours of cell culture. An increase in Oct4, Nanog, Sox2, and/or Klf-4 expression indicates the siRNA has modulated an influencer of Oct4, Nanog, Sox2, and/or Klf-4 expression, thereby altering the potency of the cell to increase potency.

In another assay, the incubation with siRNA/Lipofectamine targeted to influencers of Oct4, Nanog, Sox2, and Klf-4 expression is for a period of 12 hours, after which the cell culture media is replaced with fresh media lacking siRNA/Lipofectamine. Again, Oct4, Nanog, Sox2, and Klf-4 expression is measured at 12, 24, 36, 48 and 72 hours of cell culture. Treatment with transfection reagent for a shorter period reduces toxicity that may be present due a transfection reagent, such as Lipofectamine, liposomes or the use of cationic lipids.

### Example 7

### Increasing Multi- or Pluri-Potency by RNAi to Repressor or Downregulators of Oct4, Nanog, Sox2, and Klf-4 Using Electroporation Ex Vivo

In order to produce clinical grade cells (either from donors or from syngenic sources) with greater potential for multi or pluripotency, human fibroblasts, keratinocytes or other human somatic cells are incubated with 10 nM siRNA [Total] targeted to one or more of the following influencers of Oct4, Nanog, Sox2, and Klf-4-expression (e.g., repressors of Oct4, Nanog, Sox2, and Klf-4). siRNA is introduced into the cells via electroporation.

In one assay, the incubation with siRNA targeted to influencers of Oct4, Nanog, Sox2, and Klf-4 is for the length of cell culture, and is at 37° C. with media conditions appropriate for the cell line of choice and siRNA administration. Expression of Oct4, Nanog, Sox2, and Klf-4 is measured via reporter gene, mRNA, or detection of Oct4, Nanog, Sox2, and Klf-4 proteins at 12, 24, 36, 48 and 72 hours of cell culture. An increase in Oct4, Nanog, Sox2, and/or Klf-4-expression indicates the siRNA has modulated an influencer of Oct4, Nanog, Sox2, and/or Klf-4-expression, thereby altering the potency of the cell to increase potency.

In another assay, the incubation with siRNA targeted to influencers of Oct4, Nanog, Sox2, and Klf-4 is for a period of 12 hours, after which the cell culture media is replaced with fresh media lacking siRNA. Again, Oct4, Nanog, Sox2, and/or Klf-4-expression is measured at 12, 24, 36, 48 and 72 hours of cell culture.

### Example 8

### Increasing Multi- or Pluri-Potency by RNAi to Repressor or Downregulators of Oct4, Nanog, Sox2, and Klf-4 Using Hypotonic-Poration Ex Vivo

In order to produce clinical grade cells (either from donors or from syngenic sources) with greater potential for multi or pluripotency, human fibroblasts, keratinocytes or other human somatic cells are incubated with 10 nM siRNA [Total] targeted to one or more of the following influencers of Oct4, Nanog, Sox2, and Klf-4 expression (e.g., repressors of Oct4, Nanog, Sox2, and Klf-4). siRNA is introduced into the cells via hypotonic-poration.

In one assay, the incubation with siRNA targeted to influencers of Oct4, Nanog, Sox2, and Klf-4 is for the length of cell culture, and is at 37° C. with media conditions appropriate for the cell line of choice and siRNA administration. Expression of Oct4, Nanog, Sox2, and Klf-4 is measured via reporter gene, mRNA, or detection of Oct4, Nanog, Sox2, and Klf-4 proteins at 12, 24, 36, 48 and 72 hours of cell culture. An increase in Oct4, Nanog, Sox2, and/or Klf-4-expression indicates the siRNA has modulated an influencer of Oct4, Nanog, Sox2, and/or Klf-4-expression, thereby altering the potency of the cell to increase potency.

In another assay, the incubation with siRNA targeted to influencers of Oct4, Nanog, Sox2, and Klf-4 is for a period of 12 hours, after which the cell culture media is replaced with fresh media lacking siRNA. Again, Oct4, Nanog, Sox2, and/or Klf-4 expression is measured at 12, 24, 36, 48 and 72 hours of cell culture.

### Example 9

### Application of RNAi for Hearing Enhancement

Human adult cochlear supporting cells are infected with siRNA targeted to one or more of the following repressors of Atoh1 expression (HES1, HEY2, Id3, Prox1, NGN1, ZIC1). Accession numbers for the influencers are as follows: HES1: NM_{—}005524.2, NP_{—}005515.1; HEY2: NM_{—}001040708.1, NP_{—}001035798.1; Id3: NM_{—}002167.3, NP_{—}002158.3; Prox1: NM_{—}002763.3, NP_{—}002754.2; NGN1: NM_{—}006161.2, NP_{—}006152.2; and ZIC1: NM_{—}003412.3, NP_{—}003403.2. Cells are cultured at 37° C. with appropriate media conditions (such as hES culture media), and expression of Atoh1 is measured by quantitative RT-PCR.

Atoh1 expression is increased in cells infected with siRNA targeted to a repressor of Atoh1. The increase in Atoh1 expression is correlated with an increased differentiation of cochlear supporting cells of the inner ear, including Deiter cells and/or pillar cells, to hair cells as evidenced by the expression of markers indicative of such differentiation including Myo7a and Brn3.1. Differentiated cells are administered to a patient (for example, by grafting, transplanting, implanting, or injecting) to enhance hearing.

### Example 10

### Application of RNAi for Generating Neural Stem Cells

Human adult IPS cells are infected with siRNA targeted to one or more repressors of the following genes of interest: FST, CHRD, and CER1. Targeted repressors of FST expression include one or more of EGR2, SP6, Sox9, and MyoD1 (Accession numbers: EGR2: NM_{—}000399.3; NP_{—}000390.2; SP6: NM_{—}199262.2, NP_{—}954871.1; Sox9: NM_{—}000346.3, NP_{—}000337.1; MyoD1: NM_{—}002478.4, NP_{—}002469.2). Targeted repressors of CHRD expression include one or more of HHEX, PRDM1, and BARX1 (Accession numbers: HHEX: NM_{—}002729.4, NP_{—}002720.1; PRDM1: NM_{—}001198.3, NP_{—}001189.2; BARX1: NM_{—}021570.3, NP_{—}067545.3). HHEX is also a targeted repressor of CER1 expression.

The infected cells are cultured at 37° C. with appropriate media conditions (such as hES culture media), and expression of the applicable genes of interest (FST, CHRD and/or CER1) is measured as appropriate. Expression of FST, CHRD, and CER1 is measured by quantitative RT-PCR. Because CHRD protein can be inactivated by cleavage mediated by BMP1 or TLL1, human IPS cells are infected with siRNA targeted to BMP1 or TLL1 (Accession numbers: BMP1: NM_{—}001199.2, NP_{—}001190.1; TTL1: NM_{—}012464.3, NP_{—}036596.3). Cells are cultured at 37° C. with appropriate media conditions (such as hES culture media), and protein level of intact CHRD is measured by western blot.

Some of the human adult IPS cells infected as described above with one or more repressors of FST, CHRD, and/or CER1 are also infected with siRNA targeted to VentX, a repressor of Sox3 expression (Accession number Ventx: NM_{—}014468.2, NP_{—}055283.1). Cells are cultured at 37° C. with appropriate media conditions (such as hES culture media), and expression of Sox3 is measured by quantitative RT-PCR.

FST, CHRD, CER1, and/or SOX3 expression are increased in cells infected with siRNA targeted to repressors of these genes. The increase in gene of interest (i.e., FST, CHRD, CER1 and/or Sox3) expression is correlated with an increased differentiation of human IPS cells to neural stem cells, as evidenced by the expression of markers indicative of such differentiation including Sox2, NES, MSI1, or NRP1. Co-infection of cells with a repressor to Sox3 expression results in an increase in the percentage of cells differentiated to neural stem cells. The neural stem cells are administered to a patient (for example, by grafting, transplanting, implanting, or injecting) to enhance neuroregeneration.

1. A method of altering the potency of a cell, comprising contacting the cell with one or more repressors, wherein said one or more repressors modulates at least one component of a cellular pathway associated with the potency of the cell, thereby altering the potency of the cell.

2. The method according to claim 1, wherein the one or more repressors is a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a DNAzyme, a ssDNA, polypeptide or active fragment thereof, an antibody, an intrabody, a transbody, a protein, an enzyme, a peptidomimetic, a peptoid, a transcriptional factor, or a small organic molecule, and the like.

3. A method of altering the potency of a cell, comprising contacting the cell with one or more activators, wherein said one or more activators modulates at least one component of a cellular pathway associated with the potency of the cell, thereby altering the potency of the cell.

4. The method according to claim 3, wherein the one or more activators can be any number and/or combination of the following molecules: an antibody or an antibody fragment, an mRNA, a bifunctional antisense oligonucleotide, a dsDNA, a polypeptide or an active fragment thereof, a transcription factor, a peptidomimetic, a peptoid, or a small organic molecule, and the like.

5. The method according to claim 2 or claim 4, wherein the polypeptide or active fragment thereof is a pluripotency factor or a component of a cellular pathway associate with the potency of a cell.

6. The method according to claim 2 or claim 4, wherein the polypeptide is a transcription factor selected from the group consisting of: transcriptional activators, transcriptional repressors, artificial transcription factors, and hormone binding domain transcription factor fusion polypeptides.

7. The method according to claim 2 or claim 4, wherein the modulation comprises a change in epigenetic state, chromatin structure, transcription, mRNA splicing, post-transcriptional modification, mRNA stability and/or half-life, translation, post-translational modification, protein stability and/or half-life and/or protein activity of the at least one component.

8. The method of claim 1, wherein the at least one component is selected from the group consisting of: a members of the Hedgehog pathway, components of the Wnt pathway, receptor tyrosine kinases, non-receptor tyrosine kinases, TGF family members, BMP family members, Jak/Stat family members, Hox family members, Sox family members, Klf family members, Myc family members, Oct family members, components of a chromatin modulation pathway, components of a histone modulation pathway, miRNAs regulated by pluripotency factors, miRNAs that regulate pluripotency factors and/or components of cellular pathway associated with the developmental potency of a cell, members of the NuRD complex, Polycomb group proteins, SWI/SNF chromatin remodeling enzymes, Ac133, Alp, Atbf1, Axin2, BAF155, bFgf, Bmi1, Boc, C/EBPβ, CD9, Cdon, Cdx-2, c-Kit, c-Myc, Coup-Tf1, Coup-Tf2, Cs1, Ctbp, Dax1, Dnmt3A, Dnmt3B, Dnmt3L, Dppa2, Dppa4, Dppa5, Ecat1, Ecat8, Eomes, Eras, Esg1, Esrrb, Fbx15, Fgf2, Fgf4, F1t3, Foxc1, Foxd3, Fzd9, Gbx2, Gcnf, Gdf10, Gdf3, GdfS, Grb2, Groucho, Gsh1, Hand1, Hdac1, Hdac2, HesX1, Hic-5, HoxA10, HoxA11, HoxB1, HP1α, HP1β, HPV16 E6, HPV16 E7, Irx2, Isl1, Jarid2, Jmjd1a, Jmjd2c, Klf-3, Klf-4, Klf-5, Lef1, Lefty-1, Lefty-2, Lif, Lin-28, Mad1, Mad3, Mad4, Mafa, Mbd3, Meis1, MeI-18, Meox2, Mta1, Mxi1, Myf5, Myst3, Nac1, Nanog, Neurog2, Ngn3, Nkx2.2, Noda1, Oct-4, Olig2, Onecut, Otx1, Oxt2, Pax5, Pax6, Pdx1, Pias1, Pias2, Pias3, Piasy, REST, Rex-1, Rfx4, Rif1, Rnf2, Rybp, Sal1l4, Sal1l1, Scf, Scgf, Set, Sip1, Ski1, Smarcad1, Sox-15, Sox-2, Sox-6, Ssea-1, Ssea-2, Ssea-4, Stat3, Stella, SV40 large T antigen, Tbx3, Tcf1, Tcf2, Tcf3, Tcf4, Tcf-7, Tcf711, Tcl1, Tdgf-1, Tert, hTert, Tif1, Tra-1-60, Tra-1-81, Utf-1, Wnt3a, Wnt8a, YY1, Zeb2, Zfhx1b, Zfp281, Zfp57, Zic3, β-catenin, histone acetylases, histone de-acetylases, histone methyltransferases, histone demethylases or substrates, cofactors, co-activators, co-repressors and/or a downstream effectors thereof.

9. The method of claim 8, wherein the at least one component selected from the group consisting of: Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

10. The method of claim 8, wherein the one or more repressors modulates the at least one component by a) repressing the at least one component; b) de-repressing a repressor of the at least one component; or c) repressing an activator of the at least one component.

11. The method of claim 8, wherein the one or more repressors modulates the at least one component by a) de-repressing the at least one component; b) repressing a repressor of the at least one component; or c) de-repressing an activator of the at least one component.

12. (canceled)

13. The method according to claim 1, wherein the potency of the cell is altered to decrease potency.

14. (canceled)

15. The method according to claim 1, wherein the potency of the cell is altered to increase potency.

16. (canceled)

17. The method of claim 1, wherein the one or more repressors modulates the at least one component by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

18. (canceled)

19. The method of claim 1, wherein the cellular pathway is selected from a Wnt pathway, a Hedgehog pathway, a TGF-b pathway, a receptor tyrosine kinase pathway, a Jak/STAT pathway, and a Notch pathway.

20.-26. (canceled)

27. The method according to claim 1, wherein the cell is a stem cell or a progenitor cell.

28. The method of claim 27, wherein the cell is selected from the group consisting of:
(a) an embryonic stem or progenitor cell:
(b) an adult stem cell or progenitor cell; and
(c) an adult somatic cell.

29.-33. (canceled)

34. The method of claim 1, wherein the cell is associated with an in vivo tissue in a subject.

35. The method of claim 34, wherein the tissue is selected from pancreatic tissue, neural tissue, cardiac tissue, bone marrow, muscle tissue, bone tissue, skin tissue, liver tissue, hair follicles, vascular tissue, adipose tissue, lung tissue, and kidney tissue.

36. The method of claim 1, wherein the cell is contacted with the one or more repressors ex vivo, and wherein the method further comprises the step of administering the cell to a subject.

37.-43. (canceled)

44. A method of increasing the totipotency of a cell, comprising contacting the cell with a composition comprising one or more repressors, wherein the one or more repressors modulates at least one component of a cellular pathway associated with the totipotency of the cell, thereby increasing the totipotency of the cell.

45. A method of increasing the pluripotency of a cell, comprising contacting the cell with one or more repressors, wherein the one or more repressors modulates at least one component of a cellular pathway associated with the pluripotency of the cell, thereby increasing the pluripotency of the cell.

46. A method of increasing the multipotency of a cell, comprising contacting the cell with one or more repressors, wherein the one or more repressors modulates at least one component of a cellular pathway associated with the multipotency of the cell, thereby increasing the multipotency of the cell.

47.-57. (canceled)

58. A method of reprogramming a cell, comprising contacting the cell with one or more repressors, wherein the one or more repressors modulates at least one component of a cellular pathway associated with the reprogramming of a cell, thereby reprogramming the cell.

59. A method of in vivo cell therapy, comprising administering to a subject a composition comprising one or more repressors, wherein the one or more repressors modulates at least one component of a cellular pathway associated with the pluripotency of a cell.

60. A method of ex vivo cell therapy, comprising the steps of isolating a cell; contacting the cell with a composition comprising one or more repressors, wherein the one or more repressors modulates at least one component of a cellular pathway associated with the pluripotency of the cell; and administering the cell to a subject.

61.-65. (canceled)

65. A method of in vivo cell therapy, comprising administering to a subject a composition comprising one or more activators, wherein the one or more activators modulates at least one component of a cellular pathway associated with the pluripotency of a cell.

66. A method of ex vivo cell therapy, comprising the steps of isolating a cell; contacting the cell with a composition comprising one or more activators, wherein the one or more activator modulates at least one component of a cellular pathway associated with the pluripotency of the cell; and administering the cell to a subject.

67.-70. (canceled)

71. A culture comprising:
(a) a cell;
(b) a composition comprising one or more repressors in contact with the cell; and
(c) a pharmaceutically acceptable culture medium wherein the one or more repressors modulates at least one component of a cellular pathway associated with the pluripotency of the cell.

72. The culture of claim 71, wherein the one or more repressors modulates the at least one component by a) de-repressing the at least one component; b) repressing a repressor of the at least one component; or c) derepressing an activator of the at least one component.

73.-74. (canceled)

75. The culture of claim 71, wherein the modulation comprises a change in epigenetic state, chromatin structure, transcription, mRNA splicing, post-transcriptional modification, mRNA stability and/or half-life, translation, post-translational modification, protein stability and/or half-life and/or protein activity of the at least one component, wherein the at least one component is selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

76.-78. (canceled)

79. A culture comprising:
(a) a cell;
(b) a composition comprising one or more activators in contact with the cell; and
(c) a pharmaceutically acceptable culture medium wherein the one or more activators modulates at least component of a cellular pathway associated with the pluripotency of the cell.

80.-86. (canceled)

87. The culture of claim 71, wherein the cell is a human cell.

88. (canceled)

89. The culture of claim 87, wherein the cell is isolated from an in vivo tissue in a subject.

90.-91. (canceled)

92. An implant device, comprising a biocompatible material and a cell, and a composition comprising one or more repressors, wherein the one or more repressors modulates at least one component of a cellular pathway associated with the pluripotency of the cell.

93. An implant device, comprising a biocompatible material and a cell, and a composition comprising one or more activators, wherein the one or more activators modulates at least one component of a cellular pathway associated with the pluripotency of the cell.

94.-100. (canceled)

101. A pharmaceutical composition comprising the culture system of claim 71.

102. A method of ex vivo cell therapy, comprising administering the composition of claim 101 to a subject.

103. A composition comprising one or more repressors and a cell, wherein the one or more repressors modulates at least one component of a cellular pathway associated with the pluripotency of a cell.

104. The composition according to claim 103, wherein the one or more repressors is a PNA, an LNA, a ssRNA, a dsRNA, an mRNA, an antisense RNA, a ribozyme, an antisense oligonucleotide, a bifunctional antisense oligonucleotide, a pri-miRNA, an shRNA, an antagomir, an aptamer, an siRNA, a dsDNA, a DNAzyme, a ssDNA, polypeptide or active fragment thereof, an antibody, an intrabody, a transbody, a protein, an enzyme, a peptidomimetic, a peptoid, a transcriptional factor, or a small organic molecule, and the like.

105. A composition comprising one or more activators and a cell, wherein the one or more activators modulates at least one component of a cellular pathway associated with the pluripotency of a cell.

106.-110. (canceled)

111. The composition of claim 101, wherein the at least one component is selected from the group consisting of: Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

112.-128. (canceled)

129. A composition comprising a repressor and a cell, wherein the repressor modulates epigenetic state, chromatin structure, transcription, mRNA splicing, post-transcriptional modification, mRNA stability and/or half-life, translation, post-translational modification, protein stability and/or half-life and/or protein activity of a pluripotency factor, wherein the pluripotency factor is the selected from Oct-4, Nanog, Sox-2, cMyc, Klf-4, Lin-28, Stat-3, Tcf-3, hTERT, Stella, Rex-1, UTF-1, Dax-1, Nac-1, Sal1l4, TDGD-1, and Zfp-281, a histone methyltransferase, a histone demethylase, a histone methyltransferase, a histone demethylase or substrate, cofactor, co-activator, co-repressor and/or a downstream effector thereof.

130. The composition of claim 129, wherein the pluripotency factor is Oct3/4 and/or Nanog and a target of the repressor is one or more of a member of the NuRD complex, Sin3A, a member of the Pml complex, Hdac1/2, Mta1/2, or Mbd3.

131.-133. (canceled)

134. The composition of claim 129, wherein the pluripotency factor is Oct3/4 and wherein a target of the repressor is one or more of Cdx-2, Coup-Tf1, or Genf.

135. The composition of claim 129, wherein the pluripotency factor is Oct3/4 and wherein a target of the repressor is one or more of Piasy, Pias1, Pias2, or Pias3.

136.-142. (canceled)

143. A method of dedifferentiating a cell to a more pluripotent state, comprising contacting the cell with the composition of claim 103, wherein the one or more repressors modulates a component of a cellular pathway associated with the dedifferentiation of the cell to the pluripotent state, thereby dedifferentiating the cell to the pluripotent state.

144. A method of dedifferentiating a cell to a more pluripotent state, comprising contacting the cell with the composition of claim 105, wherein the one or more activators modulates a component of a cellular pathway associated with the dedifferentiation of the cell to the pluripotent state, thereby dedifferentiating the cell to the pluripotent state.

145.-151. (canceled)
